(19) United States
(12) Patent Application Publication
(10) Pub. No.: US 2022/0169632 A1
(43) Pub. Date: Jun. 2, 2022

(54) METHODS AND MATERIALS FOR INCREASING OR MAINTAINING NICOTINAMIDE MONONUCLEOTIDE ADENYLYL TRANSFERASE-2 (NMNAT2) POLYPEPTIDE LEVELS

(71) Applicant: University of Pittsburgh - Of the Commonwealth System of Higher Education, Pittsburgh, PA (US)

(72) Inventors: Beibei Chen, Sewickley, PA (US); Toren Finkel, Pittsburgh, PA (US); Yuan Liu, Sewickley, PA (US)

(21) Appl. No.: 17/432,639

(22) PCT Filed: Feb. 21, 2020
(86) PCT No.: PCT/US2020/019285

§ 371 Date: Aug. 20, 2021

Related U.S. Application Data
(60) Provisional application No. 62/809,339, filed on Feb. 22, 2019.

(51) Int. Cl. C07D 401/14; C07D 405/12; C07D 209/18; C07D 409/12; C07D 209/08; C07D 401/12; C07D 401/04; C07D 491/048; C07D 471/04; C07D 405/14; C07D 487/06; A61K 31/405; A61K 31/4439; A61K 31/5377; A61K 31/4355; A61K 31/444; A61K 31/404; A61K 31/4709; A61K 31/437; A61K 31/496; A61K 31/55; A61K 45/06

(52) U.S. Cl. CPC C07D 401/14; A61K 45/06; C07D 209/18; C07D 409/12; C07D 209/08; C07D 401/12; C07D 401/04; C07D 491/048; C07D 471/04; C07D 405/14; C07D 487/06; A61K 31/405; A61K 31/4439; A61K 31/5377; A61K 31/4355; A61K 31/444; A61K 31/404; A61K 31/4709; A61K 31/437; A61K 31/496; A61K 31/55; C07D 405/12

(57) ABSTRACT

This document provides methods and materials for increasing or maintaining NMNAT2 polypeptide levels within cells. For example, compounds (e.g., organic compounds) having the ability to increase or maintain NMNAT2 polypeptide levels within cells, formulations containing compounds having the ability to increase or maintain NMNAT2 polypeptide levels within cells, methods for making compounds having the ability to increase or maintain NMNAT2 polypeptide levels within cells, methods for making formulations containing compounds having the ability to increase or maintain NMNAT2 polypeptide levels within cells, methods for increasing or maintain NMNAT2 polypeptide levels within cells, and methods for treating mammals (e.g., humans) having a condition responsive to an increase in NMNAT2 polypeptide levels are provided (or for preventing said condition).

### CLAIM OF PRIORITY

This application claims priority to U.S. Provisional Patent Application Ser. No. 62/809,339, filed on Feb. 22, 2019, the entire contents of which are hereby incorporated by reference.

### FEDERALLY SPONSORED RESEARCH AND DEVELOPMENT

This invention was made with government support under grant number 1R35HL139860 and 1R01HL142777 awarded by National Institutes of Health (NIH). The government has certain rights in the invention.

### TECHNICAL FIELD

This document relates to methods and materials for increasing nicotinamide nucleotide adenylyl transferase-2 (NMNAT2) polypeptide levels. For example, this document provides compounds (e.g., organic compounds) having the ability to increase or maintain NMNAT2 polypeptide levels within cells, formulations containing compounds having the ability to increase or maintain NMNAT2 polypeptide levels within cells, methods for making compounds having the ability to increase or maintain NMNAT2 polypeptide levels within cells, methods for increasing or maintaining NMNAT2 polypeptide levels within cells, and methods for treating mammals (e.g., humans) having a condition responsive to an increase in NMNAT2 polypeptide levels.

### BACKGROUND

When neurons (e.g. axons) are damaged by trauma, injury or disease, a process of neuronal cell death called Wallerian degeneration (WD) is often initiated. This process was first described in the 1850's by Augustus Waller, who did his pioneering work examining anatomical response to the transection of nerves in frogs. WD appears to be important in both the peripheral (PNS) and central (CNS) nervous systems and has been implicated in diseases and conditions associated with neurological trauma. Increased activity of NMNAT2 (nicotinamide mononucleotide adenylyl transferase 2) in the nerve cells affords neuronal protection (Conforti et al., Nat Rev Neurosci, 15:394-409 (2014)). This neuronal protection appears to involve the ability of NMNAT2 to maintain cellular levels of the metabolic cofactor nicotinamide adenine dinucleotide (NAD), which plays an important role in cellular generation of energy from nutrients (Id.). Nerve injury causes a rapid decline in cellular NMNAT2 levels and a subsequent fall in NAD levels, followed by cellular bioenergetic crisis and neuronal cell death (Id.).

### SUMMARY

This document provides methods and materials for increasing or maintaining the level of an NMNAT2 polypeptide within cells (e.g., neurons). For example, this document provides compounds (e.g., organic compounds) having the ability to increase or maintain NMNAT2 polypeptide levels within cells, formulations containing compounds having the ability to increase or maintain NMNAT2 polypeptide levels within cells, methods for making compounds having the ability to increase or maintain NMNAT2 polypeptide levels within cells, methods for making formulations containing compounds having the ability to increase or maintain NMNAT2 polypeptide levels within cells, methods for increasing or maintaining NMNAT2 polypeptide levels within cells, and methods for treating mammals (e.g., humans) having a condition responsive to an increase in NMNAT2 polypeptide levels within cells (or methods of preventing said condition).

As described herein, the compounds provided herein can be used to increase or maintain NMNAT2 polypeptide levels within cells (e.g., neurons) in vitro or in vivo. For example, the compounds provided herein can be used to increase the polypeptide levels of endogenously produced NMNAT2 polypeptides within cells of a mammal (e.g., a human). In some cases, the compounds provided herein can be used to treat mammals (e.g., humans) having a disease, disorder, or condition associated with a low cellular and/or nuclear level of NMNAT2 polypeptides (or to prevent said disease, disorder, or condition). In some cases, the compounds provided herein can be used to treat mammals (e.g., humans) having a disease, disorder, or condition that is responsive to an increase in NMNAT2 polypeptide levels within cells (e.g., neurons) (or to prevent said disease, disorder, or condition).

In some embodiments, this document provides a compound of Formula (I):

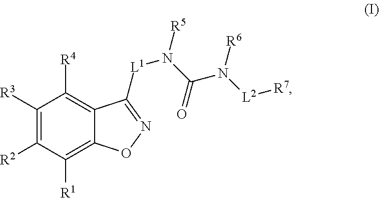

or a pharmaceutically acceptable salt thereof, wherein R¹, R², R³, R⁴, L¹, R⁵, R⁶, L², and R⁷ are as described herein.

In some embodiments, this document provides a compound of Formula (II):

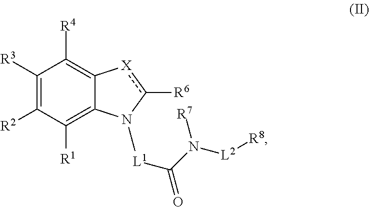

or a pharmaceutically acceptable salt thereof, wherein:

R¹, R², R³, R⁴, X, L¹, L², R⁶, R⁷, and R⁸ are as described herein.

In some embodiments, this document provides a compound of Formula (III):

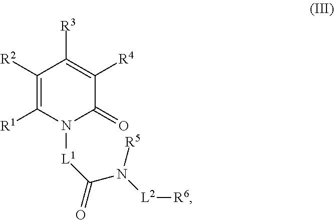

or a pharmaceutically acceptable salt thereof, wherein R¹, R², R³, R⁴, L¹, R⁵, L², and R⁶ are as described herein.

In some embodiments, this document provides a compound of Formula (IV):

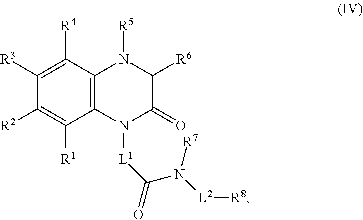

or a pharmaceutically acceptable salt thereof, wherein R¹, R², R³, R⁴, R⁵, R⁶, L¹, R⁷, L², and R⁸ are as described herein.

In some embodiments, this document provides a compound of Formula (V):

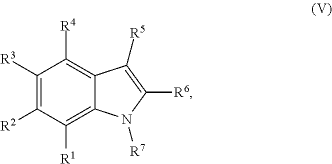

or a pharmaceutically acceptable salt thereof, wherein R¹, R², R³, R⁴, R⁵, R⁶, and R⁷ are as described herein.

In some embodiments, this document provides a compound of Formula (VI):

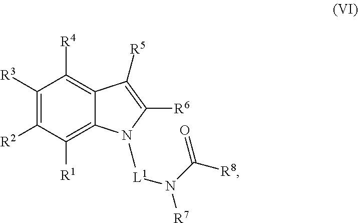

or a pharmaceutically acceptable salt thereof, wherein R¹, R², R³, R⁴, R⁵, R⁶, L¹, R⁷, and R⁸ are as described herein.

In some embodiments, this document provides a compound of Formula (VII):

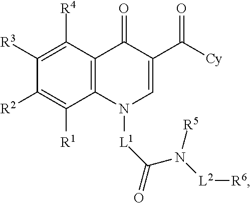

or a pharmaceutically acceptable salt thereof, wherein R¹, R², R³, R⁴, R⁵, R⁶, L¹, L², and Cy are as described herein.

In some embodiments, this document provides a pharmaceutical composition comprising, (or consisting essentially of or consisting of) any of the compounds described herein (or a pharmaceutically acceptable salt thereof) and a pharmaceutically acceptable carrier.

In some embodiments, this document provides a method for increasing or maintaining NMNAT2 polypeptide levels within a cell (e.g., a neuron). The method comprises (or consists essentially of or consists of) administering, to a mammal (e.g., a human) containing the cell, a therapeutically effective amount of any one or more of the compounds described herein (or one or more pharmaceutically acceptable salts thereof), or a pharmaceutical composition containing same.

In some embodiments, this document provides a method for increasing NAD levels within a cell (e.g., a neuron). The method comprises (or consists essentially of or consists of) administering, to a mammal (e.g., a human) containing the cell, a therapeutically effective amount of any one or more of the compounds described herein (or one or more pharmaceutically acceptable salts thereof), or a pharmaceutical composition containing same.

In some embodiments, this document provides a method for treating (or preventing) a disease, disorder, or condition responsive to an increase in a NMNAT2 polypeptide levels within a cell (e.g., a neuron). Examples of such diseases that can be treated (or prevented) as described herein include traumatic nerve injuries (e.g., a neuronal crush injury, a traumatic brain injury, chronic traumatic encephalopathy (CTE), concussion, etc.), neuropathies (e.g., a chemotherapeutic-induced sensory neuropathy, diabetic neuropathy, etc.), neurodegenerative diseases, disorders, or conditions (e.g., amyotrophic lateral sclerosis, multiple sclerosis, Huntington's disease, Alzheimer's disease, Parkinson's disease, Friedreich's ataxia, Lewy body disease, spinal muscular atrophy, frontotemporal dementia, or cerebellar degeneration), central demyelinating disorders (multiple sclerosis, adrenoleukodystrophy, adrenomyeloneuropathy, Leber hereditary optic neuropathy, neuromyelitis optica, acute disseminated encephalomyelitis, etc.), peripheral demyelinating disorders (Charcot-Marie-Tooth disease, Guillain-Barre syndrome, etc.), other primarily inflammatory neuropathies (multifocal motor neuropathy, anti-MAG neuropathies, chronic inflammatory demyelinating polyneuropathy, etc.), glaucoma, ischemic injuries, retinal and optic nerve ischemia, and stroke. The method comprises (or consists essentially of or consists of) administering, to a mammal (e.g., a human) having the disease, disorder, or condition, a therapeutically effective amount of any one or more of the compounds described herein (or one or more pharmaceutically acceptable salts thereof), or a pharmaceutical composition comprising same. In some embodiments, the neurodegenerative disease, disorders or condition is dementia (e.g., mild cognitive dementia, Alzheimer's dementia, frontotemporal dementia, vascular dementia, dementia with Lewy bodies, dementia pugilistica, or mixed dementia). In some embodiments, the neurodegenerative disease, disorders or condition is mild cognitive dementia.

Unless otherwise defined, all technical and scientific terms used herein have the same meaning as commonly understood by one of ordinary skill in the art to which the present application pertains. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1. Beas2B cells were transfected with NMNAT2-V5 plasmid. 24 hours later, cells were treated with compounds in a dose dependent manner (0, 1, 5, and 25 μM) for additional 24 hours. Cells were then collected and assayed for NMNAT2-V5 protein expression.

FIG. 2 contains a synthetic scheme for the preparation of indole-5-carboxamide compounds.

FIG. 3 contains a synthetic scheme for the preparation of indole-3-butyric acid compounds.

FIG. 4 contains a synthetic scheme for the preparation of indole-3-cyclopropyl compounds.

FIG. 5 contains a synthetic scheme for the preparation of indole-5-carboxamide compounds.

FIG. 6 contains a synthetic scheme for the preparation of indole-3-carboxpyrrolidinone compounds.

FIG. 7 contains a synthetic scheme for the preparation of 2,3,4,5-tetrahydro-1H-pyrido[4,3-b]indole compounds.

FIG. 8 contains a synthetic scheme for the preparation of 2,3,4,9-tetrahydro-1H-carbazole compounds.

FIG. 9 contains a synthetic scheme for the preparation of 2,3,4,9-tetrahydro-1H-carbazole compounds.

FIG. 10 contains a synthetic scheme for the preparation of 2,3,4,9-tetrahydro-1H-carbazole compounds.

FIG. 11 contains a synthetic scheme for the preparation of indole-3-carboxamide compounds.

FIG. 12 contains a synthetic scheme for the preparation of indole-2-propanoic acid compounds.

FIG. 13 contains a synthetic scheme for the preparation of 2,3,4,6-tetrahydro-1H-azepino[5,4,3-cd]indol-1-one compounds.

FIG. 14 contains a synthetic scheme for the preparation of 3-fluorobenzyl 2,3,4,6-tetrahydro-1H-azepino[5,4,3-cd]indol-1-one compounds.

### DETAILED DESCRIPTION

This document provides methods and materials for increasing or maintaining NMNAT2 polypeptide levels. For example, this document provides therapeutic compounds (e.g., therapeutic organic compounds) having the ability to increase or maintain NMNAT2 polypeptide levels within cells (e.g., neurons), formulations containing therapeutic compounds having the ability to increase or maintain NMNAT2 polypeptide levels within cells, methods for making therapeutic compounds having the ability to increase or maintain NMNAT2 polypeptide levels within cells, methods for making formulations containing therapeutic compounds having the ability to increase or maintain NMNAT2 polypeptide levels within cells, methods for increasing or maintaining NMNAT2 polypeptide levels within cells, and methods for treating mammals (e.g., humans) having a condition responsive to an increase in NMNAT2 polypeptide levels (or for preventing said condition).

### Methods of Treatment

Neurons are the basic units of the central and peripheral nervous systems. When a neuron or a part of a neuron (e.g., axon) is damaged by trauma or injury, a process of neuronal cell death called Wallerian degeneration (WD) is initiated. This process was first described in the 1850's by Augustus Waller, who did his pioneering work examining anatomical response to the transection of nerves in frogs. WD appears to be important in both the peripheral (PNS) and central (CNS) nervous systems and has been implicated in diseases and conditions associated with neurological trauma. Significant insight into the molecular basis of WD has come from characterization and analysis of a spontaneous mouse mutant known as the WLD^{s} mouse where the process of WD, which normally transpires over a 48 hour period, appears to be markedly slowed for ten days or more. The WLD^{s} mouse appears to be protected from a wide range of PNS and CNS insults. This protection ranges from reduced damage following acute traumatic neuronal crush injuries, blunt traumatic brain injuries, glaucoma, ischemic injury including retinal ischemia, stroke, Parkinson's disease, and many other related acute and chronic neurological conditions (Conforti et al., Nat. Rev. Neurosci., 15:394-409 (2014)). Investigations of the molecular basis for the protection afforded by the spontaneous mutation that naturally occurs in the WLD^{s} mice revealed that these mice carry an autosomal dominant gene rearrangement caused by the fusion of two gene products: nicotinamide mononucleotide adenylyl transferase 1 (Nmnat-1) and ubiquitination factor e4b (Ube4b). The protective effects of this fusion protein are the result of increased NMNAT1 activity resulting from this spontaneous genetic fusion event. The biochemical activity of NMNAT1 that affords neuronal protection appears to involve the ability of NMNAT1 to maintain levels of metabolic intermediate nicotinamide adenine dinucleotide (NAD), which plays an important role in cellular generation of energy from nutrients. Noteworthy, while in the WLD^{s} mouse NMNAT1 is expressed in the nervous system, this is not usually the case in other mammals. Instead, under normal conditions, a related gene product, NMNAT2, is the family member expressed in neurons of mammals (e.g., humans). Forced expression of NMNAT2 recapitulates the phenotype seen in neurons isolated from the WLD^{s} mice. An injury to nerves causes a rapid decline in NMNAT2 levels and a subsequent fall in NAD levels, followed by neuronal bioenergetic crisis and cell death. Accordingly, a therapeutic agent (e.g., an organic molecule) that could increase or maintain NMNAT2 levels in neurons subject to acute or chronic PNS or CNS injuries would have widespread beneficial effects.

Examples of diseases, disorders, or conditions in which PNS or CNS injuries are implicated include traumatic nerve injuries (e.g., a neuronal crash injury, a traumatic brain injury, CTE, concussion, etc.), neuropathies (e.g., chemotherapeutic-induced sensory neuropathy), neurodegenerative diseases (e.g., amyotrophic lateral sclerosis, multiple sclerosis, Huntington's disease, Alzheimer's disease, Parkinson's disease, Friedreich's ataxia, Lewy body disease, spinal muscular atrophy, frontotemporal dementia, or cerebellar degeneration), demyelinating disorders, glaucoma, ischemic injury, retinal ischemia, and stroke. In some embodiments, the neurodegenerative disease is dementia (e.g., mild cognitive dementia, Alzheimer's dementia, frontotemporal dementia, vascular dementia, dementia with Lewy bodies, dementia pugilistica, or mixed dementia). In some embodiments, the neurodegenerative disease is mild cognitive dementia. A therapeutic agent such as a compound set forth in Formula (I)-(VI), or any one of the compounds listed in Table A, or a pharmaceutically acceptable salt thereof, that enhances cellular levels of NMNAT2 polypeptides can be used to treat or reduce the adverse symptoms of the aforementioned diseases, disorders, or conditions (or to prevent said symptoms).

In some cases, increasing or maintaining NMNAT2 polypeptide levels within cells (e.g., neurons) using a compound provided herein (e.g., a compound set forth in Formula (I), (II), (III), (IV), (V), (VI), or (VII), or any one of the compounds listed in Table A, or a pharmaceutically acceptable salt thereof) can result in (a) an increased level of NAD in a cell, (b) increased cellular generation of energy from nutrients, and/or (c) increased cellular (e.g., neuronal) protection against an acute or chronic injury (e.g., a neuronal crash injury or a traumatic brain injury). In some cases, increasing or maintaining NMNAT2 polypeptide levels within cells using a compound provided herein (e.g., a compound set forth in Formula (I), (II), (III), (IV), (V), (VI), or (VII), or any one of the compounds listed in Table A, or a pharmaceutically acceptable salt thereof) as described herein can result in reducing one or more symptoms associated with traumatic nerve injuries (e.g., a neuronal crush injury or a traumatic brain injury), neuropathies (e.g., chemotherapeutic-induced sensory neuropathy), neurodegenerative diseases (e.g., amyotrophic lateral sclerosis, multiple sclerosis, Huntington's disease, Alzheimer's disease, Parkinson's disease, Friedreich's ataxia, Lewy body disease, spinal muscular atrophy, frontotemporal dementia, or cerebellar degeneration), demyelinating disorders (e.g. multiple sclerosis, Charcot-Marie-Tooth disease, etc.), glaucoma, ischemic injury, retinal ischemia, or stroke. In some embodiments, the neurodegenerative disease is dementia (e.g., mild cognitive dementia, Alzheimer's dementia, frontotemporal dementia, vascular dementia, dementia with Lewy bodies, dementia pugilistica, or mixed dementia). In some embodiments, the neurodegenerative disease is mild cognitive dementia.

In some cases, this document provides methods for increasing or maintaining NMNAT2 polypeptide levels within cells by contacting the cell with one or more compounds provided herein (e.g., a compound set forth in Formula (I), (II), (III), (IV), (V), (VI), or (VII), or any one of the compounds listed in Table A, or a pharmaceutically acceptable salt thereof). The increase in NMNAT2 polypeptide levels can be compared to the NMNAT2 polypeptide levels prior to the contacting step. In some cases, methods for increasing or maintaining NMNAT2 polypeptide levels within cells can be performed in vivo. For example, one or more compounds provided herein (e.g., a compound set forth in Formula (I), (II), (III), (IV), (V), (VI), or (VII), or any one of the compounds listed in Table A, or a pharmaceutically acceptable salt thereof) can be administered to a mammal (e.g., a human) to increase or maintain NMNAT2 polypeptide levels within cells within that mammal. In some cases, methods for increasing or maintaining NMNAT2 polypeptide levels within cells can be performed in vitro. For example, one or more compounds provided herein (e.g., a compound set forth in Formula (I), (II), (III), (IV), (V), (VI), or (VII), or any one of the compounds listed in Table A, or a pharmaceutically acceptable salt thereof) can be added to a cell culture containing cells (e.g., human cells) to increase or maintain NMNAT2 polypeptide levels within those cells. In some cases, such intervention can improve the quality of the cell while in culture or subsequently. In some cases, one or more of the compounds provided herein can be used during an ex vivo expansion of cells such as stem cells, neurons, or neuronal progenitor cells, so that the treated cells can exhibit improved in vivo persistence and/or efficacy. Examples of stem cells that can be treated in vitro or ex vivo using a compound provided herein include, without limitation, induced-pluripotent stem cells, primary neuronal cultures or cultures of neuronal stem cell. A range of other cellular products that are to be ultimately infused into a mammal (e.g., a human) can be treated as described herein during their in vitro or ex vivo expansion.

In some cases, this document provides methods for increasing NAD levels in a cell by contacting the cell with one or more compounds provided herein (e.g., a compound set forth in Formula (I), (II), (III), (IV), (V), (VI), or (VII), or any one of the compounds listed in Table A, or a pharmaceutically acceptable salt thereof). The increase in NAD levels can be as compared to the NAD levels prior to the contacting step. In some cases, increasing NAD levels within cells (e.g., neurons) using a compound provided herein (e.g., a compound set forth in Formula (I), (II), (III), (IV), (V), (VI), or (VII), or any one of the compounds listed in Table A, or a pharmaceutically acceptable salt thereof) can result in (a) increased cellular generation of energy from nutrients, and/or (b) increased cellular (e.g., neuronal) protection against an acute or chronic injury (e.g., a neuronal crash injury or a traumatic brain injury). In some cases, methods for increasing NAD levels in a cell can be performed in vivo. For example, one or more compounds provided herein (e.g., a compound set forth in Formula (I), (II), (III), (IV), (V), (VI), or (VII), or any one of the compounds listed in Table A, or a pharmaceutically acceptable salt thereof) can be administered to a mammal (e.g., a human) to increase NAD levels in a cell within that mammal. In some cases, increasing NAD levels within cells (e.g., neurons) using a compound provided herein (e.g., a compound set forth in Formula (I), (II), (III), (IV), (V), (VI), or (VII), or any one of the compounds listed in Table A, or a pharmaceutically acceptable salt thereof) as described herein can result in reducing one or more symptoms associated with traumatic nerve injuries (e.g., a neuronal crush injury or a traumatic brain injury), neuropathies (e.g., chemotherapeutic-induced sensory neuropathy), neurodegenerative diseases (e.g., amyotrophic lateral sclerosis, multiple sclerosis, Huntington's disease, Alzheimer's disease, Parkinson's disease, Friedreich's ataxia, Lewy body disease, spinal muscular atrophy, frontotemporal dementia, or cerebellar degeneration), demyelinating disorders (e.g. multiple sclerosis, Charcot-Marie-Tooth disease, etc.), glaucoma, ischemic injury, retinal ischemia, or stroke. In some embodiments, the neurodegenerative disease is dementia (e.g., mild cognitive dementia, Alzheimer's dementia, frontotemporal dementia, vascular dementia, dementia with Lewy bodies, dementia pugilistica, or mixed dementia). In some embodiments, the neurodegenerative disease is mild cognitive dementia. In some cases, methods for increasing NAD levels in a cell can be performed in vitro. For example, one or more compounds provided herein (e.g., a compound set forth in Formula (I), (II), (III), (IV), (V), (VI), or (VII), or any one of the compounds listed in Table A, or a pharmaceutically acceptable salt thereof) can be added to a cell culture containing cells (e.g., human cells) to increase NAD levels in those cells.

This document also provides methods for treating (or preventing) diseases, disorders, and conditions in a mammal by administering one or more compounds provided herein (e.g., a compound set forth in Formula (I), (II), (III), (IV), (V), (VI), or (VII), or any one of the compounds listed in Table A, or a pharmaceutically acceptable salt thereof) to a mammal in need thereof. In some cases, the disease, disorder, or condition being treated (or prevented) as described herein can be a disease, disorder, or condition that is responsive to an increase in NMNAT2 polypeptide levels (or maintaining the same NMNAT2 polypeptide levels) within cells within the mammal. In some cases, the disease, disorder, or condition being treated (or prevented) can be a disease, disorder, or condition that is associated with low NMNAT2 polypeptide levels within cells.

Examples of diseases, disorders, and conditions that can be treated (or prevented) with one or more compounds provided herein (e.g., a compound set forth in Formula (I), (II), (III), (IV), (V), (VI), or (VII), or any one of the compounds listed in Table A, or a pharmaceutically acceptable salt thereof) include, without limitation, a nerve injury, neuropathy, neurodegenerative conditions, demyelinating disorders, optic nerve damage conditions, ischemic injury, and stroke (e.g., brain damage due to stroke). In some cases, the disease, disorder, or condition can be acute. In some cases, the disease, disorder, or condition can be chronic.

Examples of nerve injury disorders that can be treated (or prevented) with one or more compounds provided herein (e.g., a compound set forth in Formula (I), (II), (III), (IV), (V), (VI), or (VII), or any one of the compounds listed in Table A, or a pharmaceutically acceptable salt thereof) include, without limitation, traumatic nerve injuries, traumatic neuronal crash injuries, traumatic brain injuries (TBIs), acquired brain injuries (ABIs), traumatic peripheral nerve injuries, spinal cord injuries, neurapraxia, axonotmesis (disruption of the neuronal axon), neurotmesis, nerve entrapments, and nerve compressions.

Examples of neurodegenerative conditions that can be treated (or prevented) with one or more compounds provided herein (e.g., a compound set forth in Formula (I), (II), (III), (IV), (V), (VI), or (VII), or any one of the compounds listed in Table A, or a pharmaceutically acceptable salt thereof) include, without limitation, motor neuron diseases, Creutzfeldt-Jakob disease, Machado-Joseph disease, Spino-cerebellar ataxias, multiple sclerosis (MS), amyotrophic lateral sclerosis (ALS), Parkinson's disease, Alzheimer's disease, Huntington's disease, hearing and balance impairments, ataxias, epilepsy, mood disorders such as schizophrenia, bipolar disorders, depressions, dementias, Pick's Disease, CNS hypoxias, cerebral senility, cerebral palsy, Friedreich's ataxia, Lewy body disease, spinal muscular atrophy, frontotemporal dementia, and cerebellar degeneration. In some embodiments, the neurodegenerative conditions is dementia (e.g., mild cognitive dementia, Alzheimer's dementia, frontotemporal dementia, vascular dementia, dementia with Lewy bodies, dementia pugilistica, or mixed dementia). In some embodiments, the neurodegenerative condition is mild cognitive dementia.

Examples of neuropathies that can be treated (or prevented) with one or more compounds provided herein (e.g., a compound set forth in Formula (I), (II), (III), (IV), (V), (VI), or (VII), or any one of the compounds listed in Table A, or a pharmaceutically acceptable salt thereof) include, without limitation, chemotherapeutic-induced sensory neuropathies, diabetic neuropathy, peripheral neuropathies, mononeuropathies, and polyneuropathies. The symptoms of neuropathy depend on whether autonomic, sensory, or motor nerves—or combinations of them—are involved. The compounds provided herein (e.g., a compound set forth in Formula (I), (II), (III), (IV), (V), (VI), or (VII), or any one of the compounds listed in Table A, or a pharmaceutically acceptable salt thereof) can be used to treat (or prevent) neuropathies associated with autonomic, sensory, or motor nerves, or a combination thereof.

Examples of optic nerve damage conditions that can be treated (or prevented) with one or more compounds provided herein (e.g., a compound set forth in Formula (I), (II), (III), (IV), (V), (VI), or (VII), or any one of the compounds listed in Table A, or a pharmaceutically acceptable salt thereof) include, without limitation, optic neuritis, glaucoma, retinal ischemia, ocular herpes, papilledema, giant cell arteritis, toxic amblyopia, optic nerve atrophy, dominant optic atrophy, Leber hereditary optic neuropathy, and blindness.

Examples of ischemic injuries that can be treated (or prevented) with one or more compounds provided herein (e.g., a compound set forth in Formula (I), (II), (III), (IV), (V), (VI), or (VII), or any one of the compounds listed in Table A, or a pharmaceutically acceptable salt thereof) include, without limitation, ischemic nerve injuries, retinal ischemias, brain ischemias, ischemic stroke, transient ischemic attack (TIA), limb ischemia and nerve injuries, lesions of ischemic origin of the peripheral nerves, and ischemic cerebral injuries.

Examples of central or peripheral demyelinating disorders that can be treated (or prevented) with one or more compounds provided herein (e.g., a compound set forth in Formula (I), (II), (III), (IV), (V), (VI), or (VII), or any one of the compounds listed in Table A, or a pharmaceutically acceptable salt thereof) include, without limitation, multiple sclerosis, Charcot-Marie-Tooth disease, adrenoleukodystrophy, adrenomyeloneuropathy, Leber hereditary optic neuropathy, neuromyelitis optica, and acute disseminated encephalomyelitis.

In some cases, one or more compounds provided herein (e.g., a compound set forth in Formula (I), (II), (III), (IV), (V), (VI), or (VII), or any one of the compounds listed in Table A, or a pharmaceutically acceptable salt thereof) can be used as described herein (e.g., to increase or maintain NMNAT2 polypeptide levels within cells and/or to treat (or prevent) a disease, disorder, or condition as described herein) as the sole active ingredient(s). For example, a method for increasing NMNAT2 polypeptide levels within cells can comprise administering, to a mammal (e.g., a human), a composition containing a compound set forth in Formula (I), (II), (III), (IV), (V), (VI), or (VII), or any one of the compounds listed in Table A, or a pharmaceutically acceptable salt thereof, that lacks any other active ingredients that increase NMNAT2 polypeptide levels within the cells. In some cases, a method for treating (or preventing) a disease, disorder, or condition as described herein can comprise administering, to a mammal (e.g., a human), a composition containing a compound set forth in Formula (I), (II), (III), (IV), (V), (VI), or (VII), or any one of the compounds listed in Table A, or a pharmaceutically acceptable salt thereof, that lacks any other active ingredients that are effective to treat (or prevent) that disease, disorder, or condition.

### Therapeutic Compounds

As described herein, any one or more of the compounds provided herein can be used to increase or maintain NMNAT2 polypeptide levels within cells, to increase NAD levels in a cell, and/or to treat (or prevent) a disease, disorder, and condition described herein in a mammal.

In some embodiments, this document provides a compound of Formula (I):

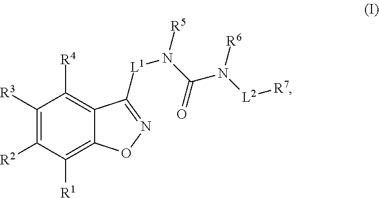

or a pharmaceutically acceptable salt thereof, wherein:

R¹, R², R³, and R⁴ are each independently selected from H, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};

L¹ is selected from C₁₋₆ alkylene, C₂₋₆ alkenylene, and C₂₋₆ alkynylene, each of which is optionally substituted with 1, 2, or 3 substituents selected from R^{g};

R⁵ and R⁶ are each independently selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents selected from R^{g};

or L² is absent;

R⁷ is selected from H, C₁₋₆ alkyl, C₁₋₆ haloalkyl, C₂₋₆ alkenyl, C₂₋₆ alkynyl, Cy¹, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};

or R⁷, L², and R⁶, together with the N atom to which R⁶ and L² are attached, form a 4-membered heterocycloalkyl ring, which is optionally substituted with 1, 2, or 3 substituents independently selected from R^{Cy1};

Cy¹ is selected from C₆₋₁₀ aryl, C₃₋₁₀ cycloalkyl, 5-14 membered heteroaryl, and 4-10 membered heterocycloalkyl, each of which is optionally substituted with 1, 2, 3, 4, or 5 substituents independently selected from R^{Cy1};
each R^{Cy1} is independently selected from halo, CN, NO₂, Cy², C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, NR^{c2}R^{d2} NR^{c2}C(O)R^{b2}, NR^{c2}C(O)OR^{a2}, NR^{c2}S(O)₂R^{b2}, S(O)₂R^{b2}, and S(O)₂NR^{c2}R^{d2}; wherein said C₁₋₆ alkyl, C₂₋₆ alkenyl, and C₂₋₆ alkynyl are each optionally substituted with 1, 2, or 3 substituents independently selected from Cy², halo, CN, NO₂, OR², C(O)R^{b2}, C(O)NR^{c2}R^{d2}, C(O)OR, NR^{c2}R^{d2} NR^{c2}C(O)R^{b2} NR^{c2}C(O)OR^{a2}, NR^{c2}S(O)₂R^{b2}, S(O)₂R^{b2} and S(O)₂NR^{c2}R^{d2};

each Cy² is independently selected from C₆₋₁₀ aryl, C₃₋₁₀ cycloalkyl, 5-10 membered heteroaryl, and 4-10 membered heterocycloalkyl, each of which is optionally substituted with 1, 2, 3, 4, or 5 substituents independently selected from R^{c2};

each R^{Cy2} is independently selected from halo, CN, NO₂, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}S(O)₂R^{b2}, S(O)₂R^{b2}, and S(O)₂NR^{c2}R^{d2}; wherein said C₁₋₆ alkyl, C₂₋₆ alkenyl, and C₂₋₆ alkynyl are each optionally substituted with 1, 2, or 3 substituents independently selected from halo, CN, NO₂, OR^{a2}, C(O)R^{b2}, C(O)NR^{c2}R^{d2}, C(O)OR², NR^{c2}R^{d2}, NR^{c2}C(O)R^{b2}, NR^{c2}C(O)OR^{a2}, NR^{c2}S(O)₂R^{b2}, S(O)₂R^{b2}, and S(O)₂NR^{c2}R^{d2};

each R^{a1}, R^{b1}, R^{c1}, R^{d1}, R^{a1}, R^{b2}, R^{c2}, and R^{d2} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};

or any R^{c1} and R^{d1} together with the N atom to which they are attached form a 4-7 membered heterocycloalkyl, which is optionally substituted with 1, 2, or 3 substituents independently selected from R^{g};

or any R^{c2} and R^{d2} together with the N atom to which they are attached form a 4-7 membered heterocycloalkyl, which is optionally substituted with 1, 2, or 3 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R¹, R², R³, and R⁴ are each independently selected from H, halo, CN, NO₂, C₁₋₆ alkyl, C₁₋₆ haloalkyl, OH, C₁₋₆ alkoxy, C₁₋₆ haloalkoxy, C(O)R^{b1}, C(O)NR^{c1}R^{d1}, C(O)OR^{a1}, NR^{c1}R^{d1}, and NR^{c1}C(O)R^{b1}; wherein said C₁₋₆ alkyl is optionally substituted with 1, 2, or 3 substituents independently selected from CN, NO₂, OH, C₁₋₆ alkoxy, C(O)R^{b1}, C(O)NR^{c1}R^{d1}, C(O)OR^{a1}, NR^{c1}R^{d1}, and NR^{c1}C(O)R^{b1}.

In some embodiments, R¹, R², R³, and R⁴ are each independently selected from H, halo, C₁₋₆ alkyl, C₁₋₆ haloalkyl, C₁₋₆ alkoxy, and C₁₋₆ haloalkoxy.

In some embodiments, R¹, R², and R⁴ are each H; and R³ is selected from H, halo, C₁₋₆ alkyl, C₁₋₆ haloalkyl, C₁₋₆ alkoxy, and C₁₋₆ haloalkoxy.

In some embodiments, R¹, R², and R⁴ are each H; and R³ is selected from H and C₁₋₆ alkyl.

In some embodiments, L¹ is selected from C₁₋₆ alkylene, C₂₋₆ alkenylene, and C₂₋₆ alkynylene, each of which is optionally substituted with 1, 2, or 3 substituents selected from OH, NO₂, CN, halo, C₁₋₆ alkoxy, and C₁₋₆ haloalkoxy.

In some embodiments, L¹ is C₁₋₆ alkylene, which is optionally substituted with 1, 2, or 3 substituents selected from OH, NO₂, CN, halo, C₁₋₆ alkoxy, and C₁₋₆ haloalkoxy.

In some embodiments, L¹ is C₁₋₆ alkylene, which is optionally substituted with 1, 2, or 3 substituents selected from OH, NO₂, CN, halo, C₁₋₆ alkoxy, and C₁₋₆ haloalkoxy.

In some embodiments, L¹ is C₁₋₆ alkylene, which is optionally substituted with 1, 2, or 3 substituents selected from OH, NO₂, CN, and halo.

In some embodiments, L¹ is C₁₋₆ alkylene.

In some embodiments, R⁵ and R⁶ are each H.

In some embodiments, R⁵ is H; and R⁶ is C₁₋₆ alkyl.

In some embodiments, L² is absent.

In some embodiments, L² is selected from C₁₋₆ alkylene, C₂₋₆ alkenylene, and C₂₋₆ alkynylene, each of which is optionally substituted with 1, 2, or 3 substituents selected from OH, NO₂, CN, halo, C₁₋₆ alkoxy, and C₁₋₆ haloalkoxy.

In some embodiments, L² is C₁₋₆ alkylene, which is optionally substituted with 1, 2, or 3 substituents selected from OH, NO₂, CN, halo, C₁₋₆ alkoxy, and C₁₋₆ haloalkoxy.

In some embodiments, L² is C₁₋₆ alkylene, which is optionally substituted with 1, 2, or 3 substituents selected from OH, NO₂, CN, and halo.

In some embodiments, L² is C₁₋₆ alkylene.

In some embodiments, R⁷ is selected from Cy¹, OR^{a1}, and NR^{c1}R^{d1}.

In some embodiments, R⁷ is Cy¹.

In some embodiments, R⁷ is OR^{a1}.

In some embodiments, R⁷ is NR^{c1}R^{d1}.

In some embodiments, Cy¹ is selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from halo, CN, C₆₋₁₀ aryl, C₁₋₆ alkyl, C₁₋₆ haloalkyl, OR^{a2}, SR^{a2}, and C(O)R^{b2}; wherein said C₁₋₆ alkyl is optionally substituted with C₆₋₁₀ aryl.

In some embodiments, Cy¹ is C₆₋₁₀ aryl, optionally substituted with 1, 2, or 3 substituents independently selected from halo, CN, C₁₋₆ alkyl, C₁₋₆ haloalkyl, C₁₋₆ alkoxy, C₁₋₆ alkylthio, and C(O)C₁₋₆ alkyl.

In some embodiments, Cy¹ is C₃₋₁₀ cycloalkyl, optionally substituted with 1 or 2 C₁₋₆ alkyl.

In some embodiments, Cy¹ is 5-14 membered heteroaryl, optionally substituted with C₁₋₆ alkyl.

In some embodiments, Cy¹ is 4-10 membered heterocycloalkyl, optionally substituted with C₆₋₁₀ aryl-C₁₋₆ alkylene.

In some embodiments, each R^{a1}, R^{b1}, R^{c1}, R^{a1}, R^{a2}, R^{b2}, R^{c2}, and R^{d2} is independently selected from H, C₁₋₆ alkyl, C₁₋₄ haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, or 3 substituents independently selected from OH, NO₂, CN, halo, C₁₋₆ alkyl, C₁₋₄ haloalkyl, C₁₋₆ alkoxy, and C₁₋₆ haloalkoxy.

In some embodiments, each R^{a1}, R^{b1}, R^{c1}, R^{a1}, R^{a2}, R^{b2}, R^{c2}, and R^{d2} is independently selected from H, C₁₋₆ alkyl, and C₆₋₁₀ aryl-C₁₋₄ alkylene, wherein said C₁₋₆ alkyl and C₆₋₁₀ aryl-C₁₋₄ alkylene are optionally substituted with 1, 2, or 3 substituents independently selected from OH, NO₂, CN, halo, C₁₋₆ alkyl, C₁₋₄ haloalkyl, C₁₋₆ alkoxy, and C₁₋₆ haloalkoxy.

In some embodiments, each R^{a1}, R^{b1}, R^{c1}, R^{a1}, R^{a2}, R^{b2}, R^{c2}, and R^{d2} is independently selected from H, C₁₋₆ alkyl, and C₆₋₁₀ aryl-C₁₋₄ alkylene.

In some embodiments, each R^{a1}, R^{b1}, R^{c1}, R^{a1}, R^{a2}, R^{b2}, R^{c2}, and R^{d2} is independently selected from H and C₁₋₆ alkyl.

In some embodiments, R^{c1} is H; and R^{d1} is C₆₋₁₀ aryl-C₁₋₄ alkylene.

In some embodiments, R^{c1} is H; and R^{d1} is C₁₋₆ alkyl.

In some embodiments:

R¹, R², R³, and R⁴ are each independently selected from H, halo, CN, NO₂, C₁₋₆ alkyl, C₁₋₆ haloalkyl, OH, C₁₋₆ alkoxy, C₁₋₆ haloalkoxy, C(O)R^{b1}, C(O)NR^{c1}R^{d1}, C(O)OR^{a1}, NR^{c1}R^{d1}, and NR^{c1}C(O)R^{b1}; wherein said C₁₋₆ alkyl is optionally substituted with 1, 2, or 3 substituents independently selected from CN, NO₂, OH, C₁₋₆ alkoxy, C(O)R^{b1}, C(O)NR^{c1}R^{d1}, C(O)OR^{a1}, NR^{c1}R^{d1}, and NR^{c1}C(O)R^{b1};

L¹ is selected from C₁₋₆ alkylene, C₂₋₆ alkenylene, and C₂₋₆ alkynylene, each of which is optionally substituted with 1, 2, or 3 substituents selected from OH, NO₂, CN, halo, C₁₋₆ alkoxy, and C₁₋₆ haloalkoxy;

R⁵ is H;

R⁶ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents selected from OH, NO₂, CN, halo, C₁₋₆ alkoxy, and C₁₋₆ haloalkoxy;

or L² is absent;

R⁷ is selected from Cy¹, OR^{a1}, and NR^{c1}R^{d1}; Cy¹ is selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from halo, CN, C₆₋₁₀ aryl, C₁₋₆ alkyl, C₁₋₆ haloalkyl, OR^{a2} SR^{a2}, and C(O)R^{b2}; wherein said C₁₋₆ alkyl is optionally substituted with C₆₋₁₀ aryl; and

each R^{a1}, R^{b1}, R^{c1}, R^{d1}, R^{a2}, and R^{b2} is independently selected from H, C₁₋₆ alkyl, and C₆₋₁₀ aryl-C₁₋₄ alkylene, wherein said C₁₋₆ alkyl and C₆₋₁₀ aryl-C₁₋₄ alkylene are optionally substituted with 1, 2, or 3 substituents independently selected from OH, NO₂, CN, halo, C₁₋₆ alkyl, C₁₋₄ haloalkyl, C₁₋₆ alkoxy, and C₁₋₆ haloalkoxy.

In some embodiments:

R¹, R², and R⁴ are each H; and R³ is selected from H, halo, C₁₋₆ alkyl, C₁₋₆ haloalkyl, C₁₋₆ alkoxy, and C₁₋₆ haloalkoxy;

L¹ is C₁₋₆ alkylene, which is optionally substituted with 1, 2, or 3 substituents selected from OH, NO₂, CN, halo, C₁₋₆ alkoxy, and C₁₋₆ haloalkoxy;

R⁵ is H;

R⁶ is selected from H and C₁₋₆ alkyl;

L² is C₁₋₆ alkylene, which is optionally substituted with 1, 2, or 3 substituents selected from OH, NO₂, CN, halo, C₁₋₆ alkoxy, and C₁₋₆ haloalkoxy;

or L² is absent;

R⁷ is selected from Cy¹, OR^{a1}, and NR^{c1}R^{d1};

Cy¹ is selected from: (i) C₆₋₁₀ aryl, optionally substituted with 1, 2, or 3 substituents independently selected from halo, CN, C₁₋₆ alkyl, C₁₋₆ haloalkyl, C₁₋₆ alkoxy, C₁₋₆ alkylthio, and C(O)C₁₋₆ alkyl; (ii) C₃₋₁₀ cycloalkyl, optionally substituted with 1 or 2 C₁₋₆ alkyl; and (iii) 4-10 membered heterocycloalkyl, optionally substituted with C₆₋₁₀ aryl-C₁₋₆ alkylene; and

R^{a1}, R^{c1}, and R^{d1} are each independently selected from H, C₁₋₆ alkyl, and C₆₋₁₀ aryl-C₁₋₄ alkylene.

In some embodiments:

R¹, R², and R⁴ are each H;

R³ is selected from H and C₁₋₆ alkyl;

L¹ is C₁₋₆ alkylene; and

L² is absent or C₁₋₆ alkylene.

In some embodiments, the compound of Formula (I) is selected from any one of the compounds listed in Table 1, or a pharmaceutically acceptable salt thereof.

TABLE 1

| | |
|---|---|
| 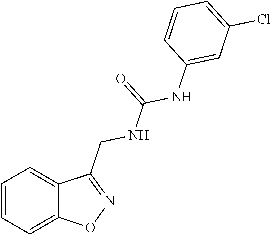 [SMILES: O=C(NCc1noc2ccccc12)Nc1cccc(Cl)c1] | BC181001 |
| 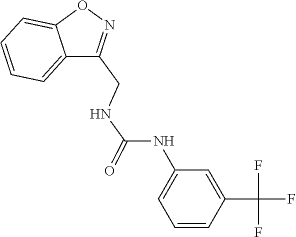 [SMILES: O=C(NCc1noc2ccccc12)Nc1cccc(C(F)(F)F)c1] | BC181002 |
| 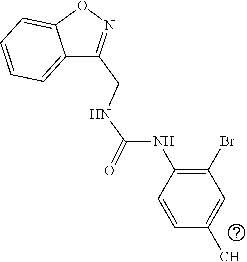 [SMILES: Cc1ccc(NC(=O)NCc2noc3ccccc23)c(Br)c1] | BC181003 |
| 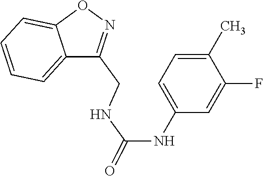 [SMILES: Cc1ccc(NC(=O)NCc2noc3ccccc23)cc1F] | BC181004 |
| 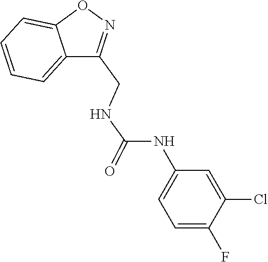 [SMILES: O=C(NCc1noc2ccccc12)Nc1ccc(F)c(Cl)c1] | BC181005 |
| 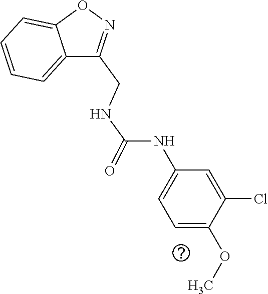 [SMILES: COc1ccc(NC(=O)NCc2noc3ccccc23)cc1Cl] | BC181006 |
| 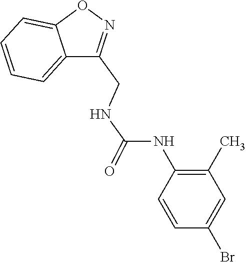 [SMILES: Cc1cc(Br)ccc1NC(=O)NCc1noc2ccccc12] | BC181007 |
| 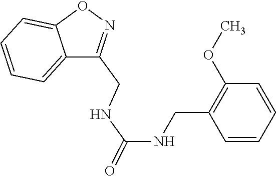 [SMILES: COc1ccccc1CNC(=O)NCc1noc2ccccc12] | BC181008 |
| 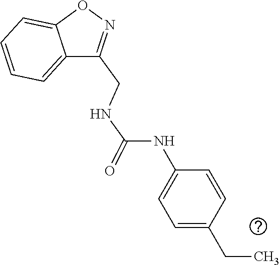 [SMILES: CCc1ccc(NC(=O)NCc2noc3ccccc23)cc1] | BC181009 |
| 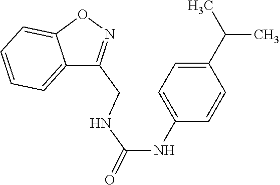 [SMILES: CC(C)c1ccc(NC(=O)NCc2noc3ccccc23)cc1] | BC181010 |
| 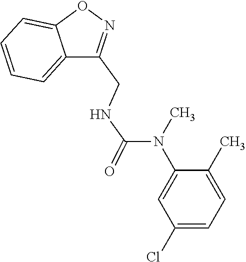 [SMILES: Cc1ccc(Cl)cc1N(C)C(=O)NCc1noc2ccccc12] | BC181011 |
| 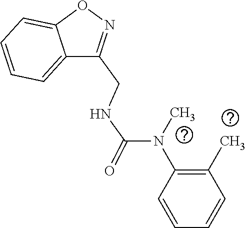 [SMILES: Cc1ccccc1N(C)C(=O)NCc1noc2ccccc12] | BC181012 |
| 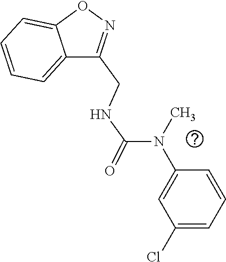 [SMILES: CN(C(=O)NCc1noc2ccccc12)c1cccc(Cl)c1] | BC181013 |
| 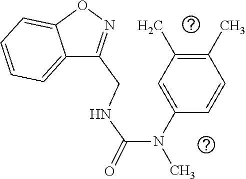 [SMILES: Cc1ccc(N(C)C(=O)NCc2noc3ccccc23)cc1C] | BC181014 |
| 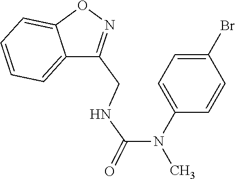 [SMILES: CN(C(=O)NCc1noc2ccccc12)c1ccc(Br)cc1] | BC181015 |
| 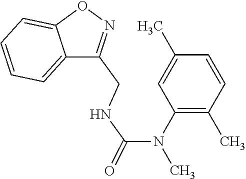 [SMILES: Cc1ccc(C)c(N(C)C(=O)NCc2noc3ccccc23)c1] | BC181016 |
| 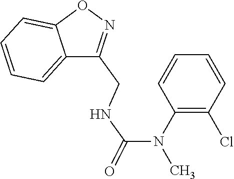 [SMILES: CN(C(=O)NCc1noc2ccccc12)c1ccccc1Cl] | BC181017 |
| 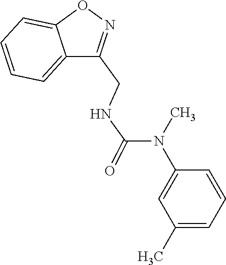 [SMILES: Cc1cccc(N(C)C(=O)NCc2noc3ccccc23)c1] | BC181018 |
| 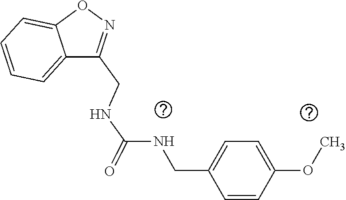 [SMILES: COc1ccc(CNC(=O)NCc2noc3ccccc23)cc1] | BC181019 |
| 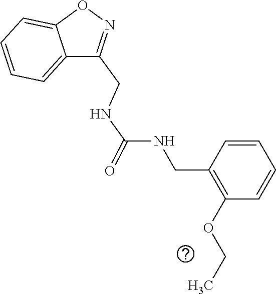 [SMILES: CCOc1ccccc1CNC(=O)NCc1noc2ccccc12] | BC181020 |
| 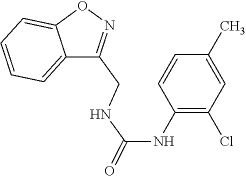 [SMILES: Cc1ccc(NC(=O)NCc2noc3ccccc23)c(Cl)c1] | BC181021 |
| 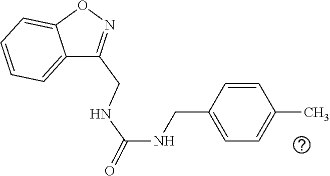 [SMILES: Cc1ccc(CNC(=O)NCc2noc3ccccc23)cc1] | BC181022 |
| 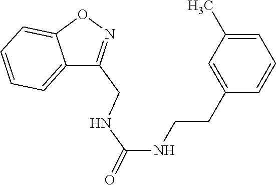 [SMILES: Cc1cccc(CCNC(=O)NCc2noc3ccccc23)c1] | BC181023 |
| 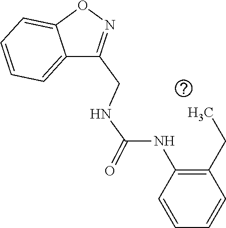 [SMILES: CCc1ccccc1NC(=O)NCc1noc2ccccc12] | BC181024 |
| 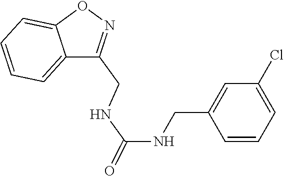 [SMILES: O=C(NCc1cccc(Cl)c1)NCc1noc2ccccc12] | BC181025 |
| 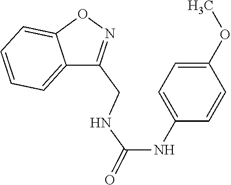 [SMILES: COc1ccc(NC(=O)NCc2noc3ccccc23)cc1] | BC181026 |
| 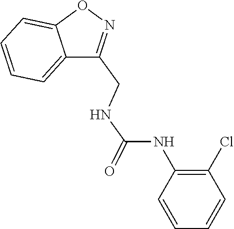 [SMILES: O=C(NCc1noc2ccccc12)Nc1ccccc1Cl] | BC181027 |
| 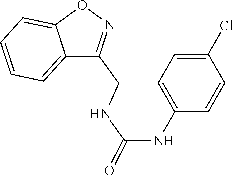 [SMILES: O=C(NCc1noc2ccccc12)Nc1ccc(Cl)cc1] | BC181028 |
| 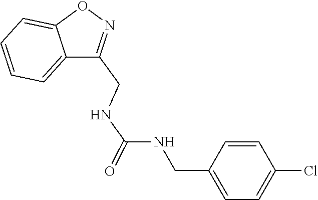 [SMILES: O=C(NCc1ccc(Cl)cc1)NCc1noc2ccccc12] | BC181029 |
| 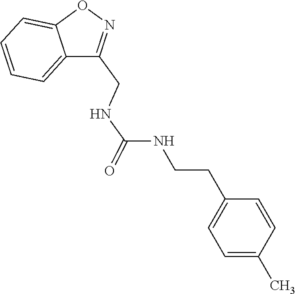 [SMILES: Cc1ccc(CCNC(=O)NCc2noc3ccccc23)cc1] | BC181030 |
| 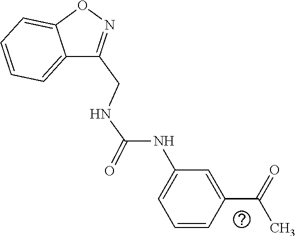 [SMILES: CC(=O)c1cccc(NC(=O)NCc2noc3ccccc23)c1] | BC181031 |
| 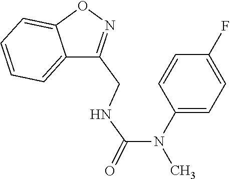 [SMILES: CN(C(=O)NCc1noc2ccccc12)c1ccc(F)cc1] | BC181032 |
| 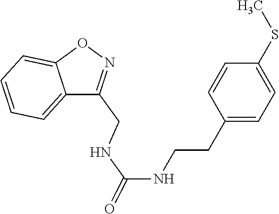 [SMILES: CSc1ccc(CCNC(=O)NCc2noc3ccccc23)cc1] | BC181033 |
| 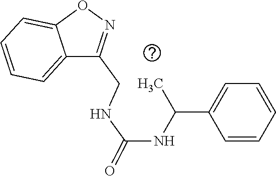 [SMILES: CC(NC(=O)NCc1noc2ccccc12)c1ccccc1] | BC181034 |
| 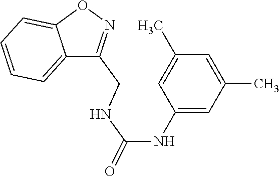 [SMILES: Cc1cc(C)cc(NC(=O)NCc2noc3ccccc23)c1] | BC181035 |
| 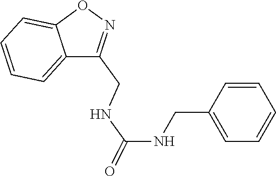 [SMILES: O=C(NCc1ccccc1)NCc1noc2ccccc12] | BC181036 |
| 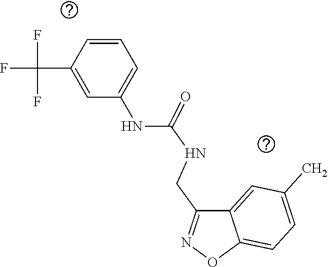 [SMILES: Cc1ccc2onc(CNC(=O)Nc3cccc(C(F)(F)F)c3)c2c1] | BC181037 |
| 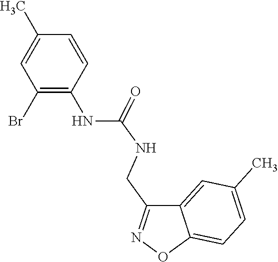 [SMILES: Cc1ccc(NC(=O)NCc2noc3ccc(C)cc23)c(Br)c1] | BC181038 |
| 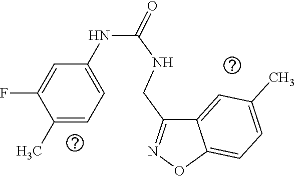 [SMILES: Cc1ccc2onc(CNC(=O)Nc3ccc(C)c(F)c3)c2c1] | BC181039 |
| 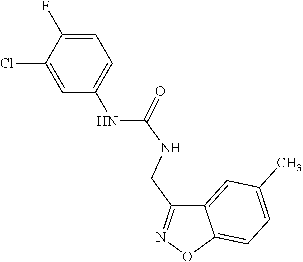 [SMILES: Cc1ccc2onc(CNC(=O)Nc3ccc(F)c(Cl)c3)c2c1] | BC181040 |
| 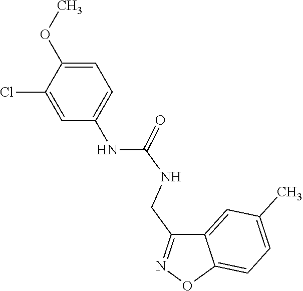 [SMILES: COc1ccc(NC(=O)NCc2noc3ccc(C)cc23)cc1Cl] | BC181041 |
| 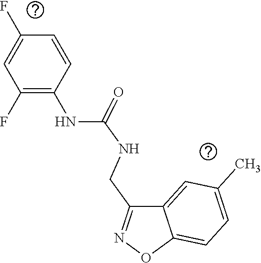 [SMILES: Cc1ccc2onc(CNC(=O)Nc3ccc(F)cc3F)c2c1] | BC181042 |
| 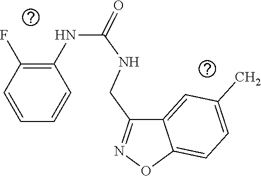 [SMILES: Cc1ccc2onc(CNC(=O)Nc3ccccc3F)c2c1] | BC181043 |
| 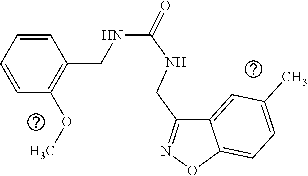 [SMILES: COc1ccccc1CNC(=O)NCc1noc2ccc(C)cc12] | BC181044 |
| 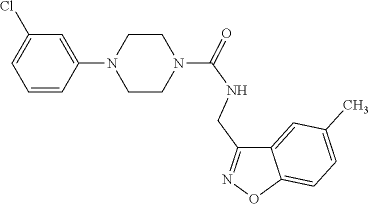 [SMILES: Cc1ccc2onc(CNC(=O)N3CCN(c4cccc(Cl)c4)CC3)c2c1] | BC181045 |
| 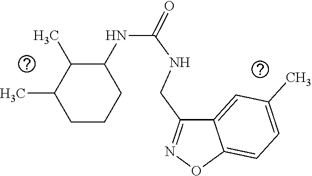 | BC181046 |
| 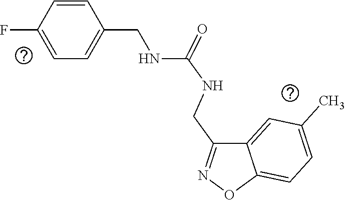 [SMILES: Cc1ccc2onc(CNC(=O)NCc3ccc(F)cc3)c2c1] | BC181047 |
| 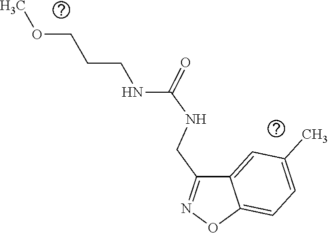 | BC181048 |
| 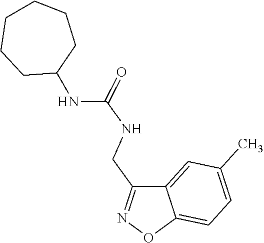 [SMILES: Cc1ccc2onc(CNC(=O)NC3CCCCCC3)c2c1] | BC181049 |
| 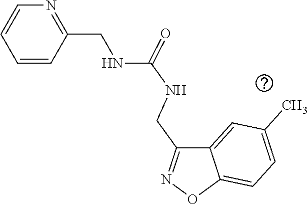 [SMILES: Cc1ccc2onc(CNC(=O)NCc3ccccn3)c2c1] | BC181050 |
| 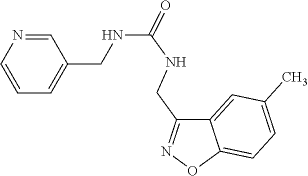 [SMILES: Cc1ccc2onc(CNC(=O)NCc3cccnc3)c2c1] | BC181051 |
| 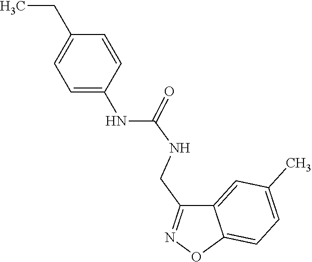 [SMILES: CCc1ccc(NC(=O)NCc2noc3ccc(C)cc23)cc1] | BC181052 |
| 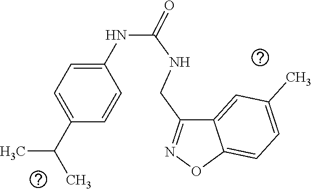 [SMILES: Cc1ccc2onc(CNC(=O)Nc3ccc(C(C)C)cc3)c2c1] | BC181053 |
| 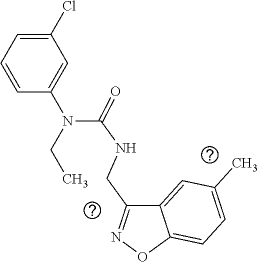 [SMILES: CCN(C(=O)NCc1noc2ccc(C)cc12)c1cccc(Cl)c1] | BC181054 |
| 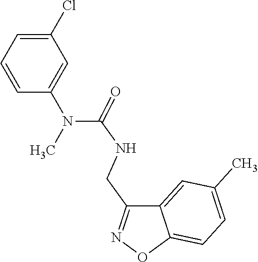 [SMILES: Cc1ccc2onc(CNC(=O)N(C)c3cccc(Cl)c3)c2c1] | BC181055 |
| 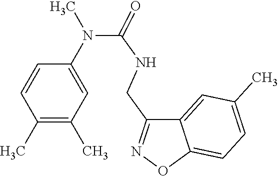 [SMILES: Cc1ccc2onc(CNC(=O)N(C)c3ccc(C)c(C)c3)c2c1] | BC181056 |
| 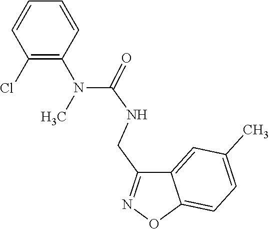 [SMILES: Cc1ccc2onc(CNC(=O)N(C)c3ccccc3Cl)c2c1] | BC181057 |
| 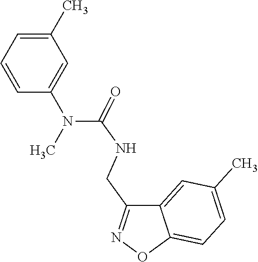 [SMILES: Cc1cccc(N(C)C(=O)NCc2noc3ccc(C)cc23)c1] | BC181058 |
| 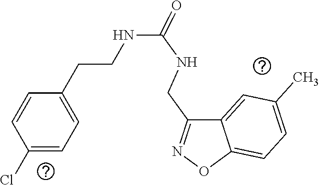 [SMILES: Cc1ccc2onc(CNC(=O)NCCc3ccc(Cl)cc3)c2c1] | BC181059 |
| 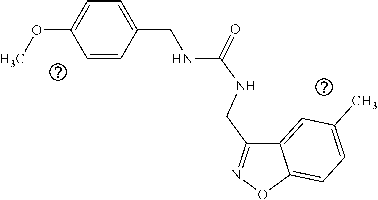 | BC181060 |
| 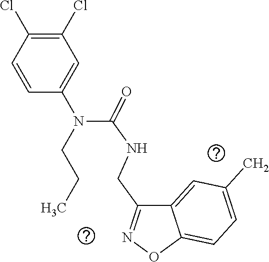 [SMILES: CCCN(C(=O)NCc1noc2ccc(C)cc12)c1ccc(Cl)c(Cl)c1] | BC181061 |
| 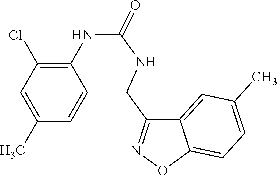 [SMILES: Cc1ccc(NC(=O)NCc2noc3ccc(C)cc23)c(Cl)c1] | BC181062 |
| 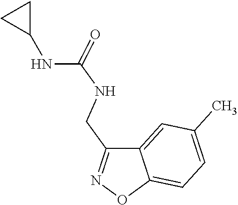 [SMILES: Cc1ccc2onc(CNC(=O)NC3CC3)c2c1] | BC181063 |
| 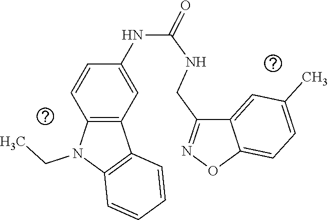 [SMILES: CCn1c2ccccc2c2cc(NC(=O)NCc3noc4ccc(C)cc34)ccc21] | BC181064 |
| 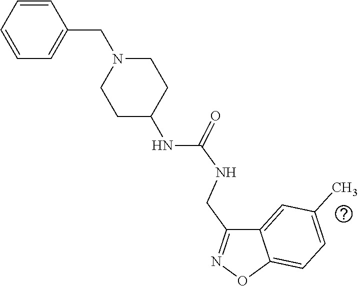 [SMILES: Cc1ccc2onc(CNC(=O)NC3CCN(Cc4ccccc4)CC3)c2c1] | BC181065 |
| 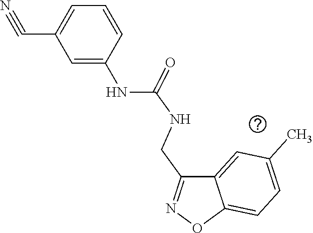 [SMILES: Cc1ccc2onc(CNC(=O)Nc3cccc(C#N)c3)c2c1] | BC181066 |
| 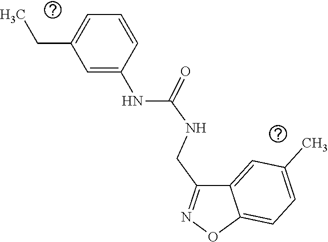 [SMILES: CCc1cccc(NC(=O)NCc2noc3ccc(C)cc23)c1] | BC181067 |
| 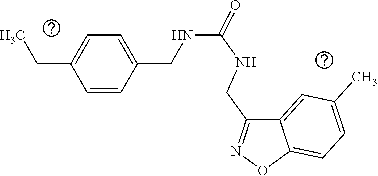 [SMILES: CCc1ccc(CNC(=O)NCc2noc3ccc(C)cc23)cc1] | BC181068 |
| 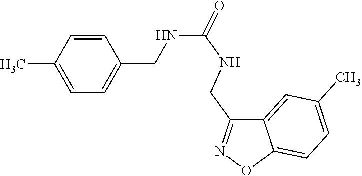 | BC181069 |
| 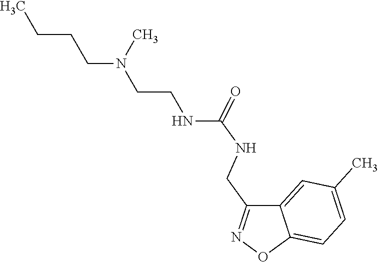 [SMILES: CCCCN(C)CCNC(=O)NCc1noc2ccc(C)cc12] | BC181070 |
| 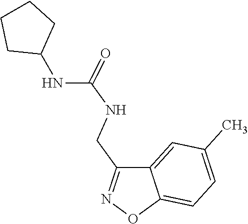 [SMILES: Cc1ccc2onc(CNC(=O)NC3CCCC3)c2c1] | BC181071 |
| 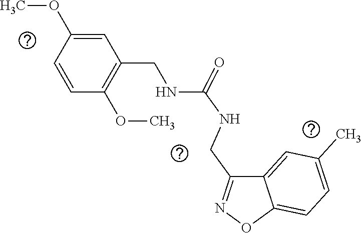 | BC181072 |
| 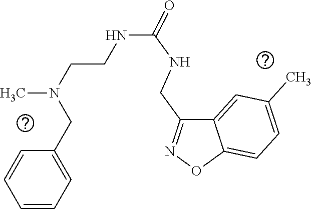 [SMILES: Cc1ccc2onc(CNC(=O)NCCN(C)Cc3ccccc3)c2c1] | BC181073 |
| 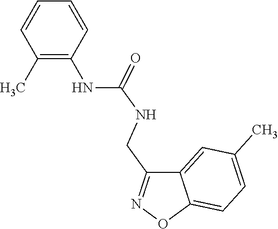 [SMILES: Cc1ccc2onc(CNC(=O)Nc3ccccc3C)c2c1] | BC181074 |
| 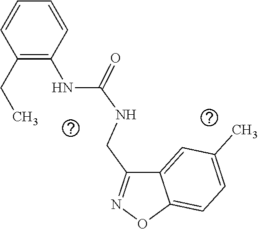 [SMILES: CCc1ccccc1NC(=O)NCc1noc2ccc(C)cc12] | BC181075 |
| 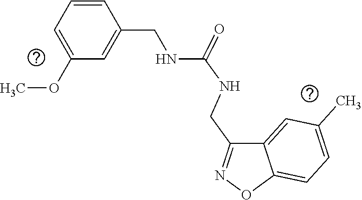 | BC181076 |
| 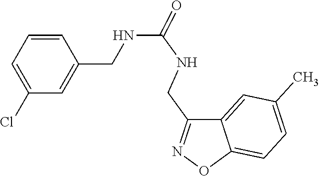 [SMILES: Cc1ccc2onc(CNC(=O)NCc3cccc(Cl)c3)c2c1] | BC181077 |
| 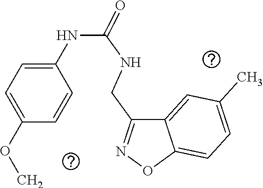 [SMILES: COc1ccc(NC(=O)NCc2noc3ccc(C)cc23)cc1] | BC181078 |
| 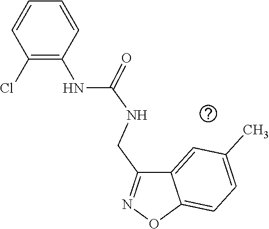 [SMILES: Cc1ccc2onc(CNC(=O)Nc3ccccc3Cl)c2c1] | BC181079 |
| 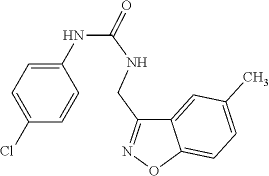 [SMILES: Cc1ccc2onc(CNC(=O)Nc3ccc(Cl)cc3)c2c1] | BC181080 |
| 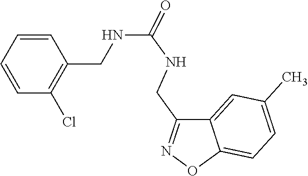 [SMILES: Cc1ccc2onc(CNC(=O)NCc3ccccc3Cl)c2c1] | BC181081 |
| 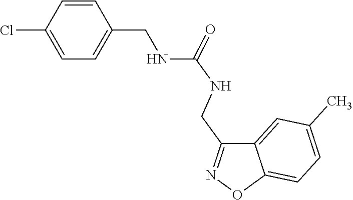 [SMILES: Cc1ccc2onc(CNC(=O)NCc3ccc(Cl)cc3)c2c1] | BC181082 |
| 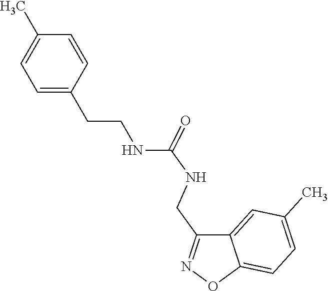 [SMILES: Cc1ccc(CCNC(=O)NCc2noc3ccc(C)cc23)cc1] | BC181083 |
| 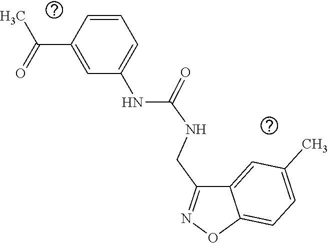 [SMILES: CC(=O)c1cccc(NC(=O)NCc2noc3ccc(C)cc23)c1] | BC181084 |
| 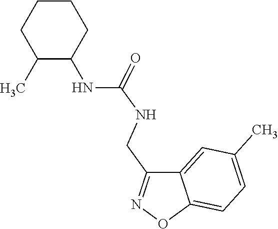 [SMILES: Cc1ccc2onc(CNC(=O)NC3CCCCC3C)c2c1] | BC181085 |
| 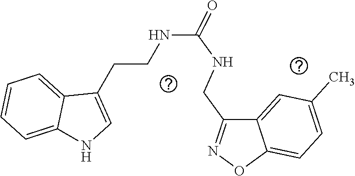 [SMILES: Cc1ccc2onc(CNC(=O)NCCc3c[nH]c4ccccc34)c2c1] | BC181086 |
| 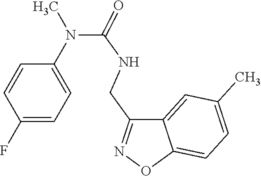 [SMILES: Cc1ccc2onc(CNC(=O)N(C)c3ccc(F)cc3)c2c1] | BC181087 |
| 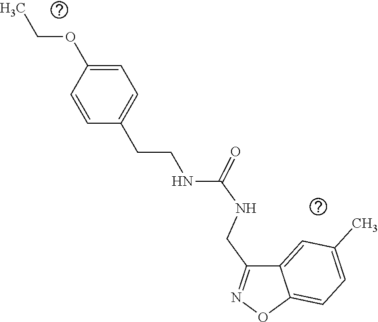 [SMILES: CCOc1ccc(CCNC(=O)NCc2noc3ccc(C)cc23)cc1] | BC181088 |
| 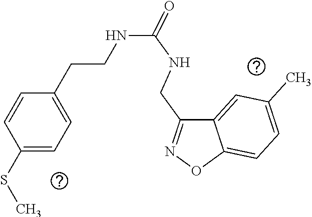 [SMILES: CSc1ccc(CCNC(=O)NCc2noc3ccc(C)cc23)cc1] | BC181089 |
| 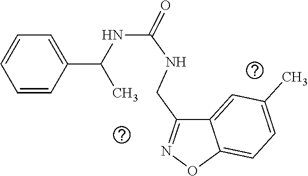 [SMILES: Cc1ccc2onc(CNC(=O)NC(C)c3ccccc3)c2c1] | BC181090 |
| 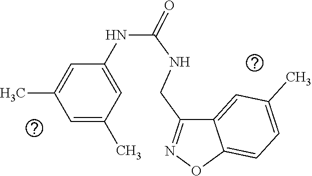 | BC181091 |
| 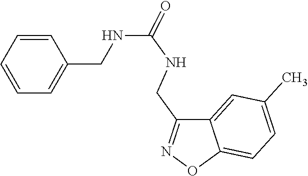 [SMILES: Cc1ccc2onc(CNC(=O)NCc3ccccc3)c2c1] | BC181092 |

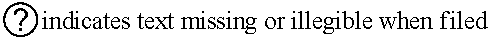

In some embodiments, this document provides a compound of Formula (II):

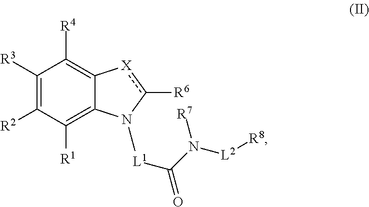

or a pharmaceutically acceptable salt thereof, wherein R¹, R², R³, R⁴, X, R⁶, L¹, R⁷, L², and R⁸ are as described herein.

In some embodiments:

R¹, R², R³, and R⁴ are each independently selected from H, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};

[symbol] is a single bond or a double bond; wherein:

(i) when [symbol] is a double bond, R⁶ is selected from H, C₁₋₆ alkyl, and Cy, and X is selected from N and CR⁵; and

(ii) when [symbol] is a single bond, R⁶ is oxo, X is CR⁵, and R⁵ and R⁴, together with the carbon atoms to which they are attached, form C₆₋₁₀ aryl ring or 5-14 membered heteroaryl ring, each of which is optionally substituted with 1, 2, or 3 substituents independently selected from R^{g};

R⁵ is selected from H, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)C(O)NR^{c1}R^{d1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};

L¹ is selected from C₁₋₆ alkylene, C₂₋₆ alkenylene, and C₂₋₆ alkynylene, each of which is optionally substituted with 1, 2, or 3 substituents selected from R^{g};

R⁷ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents selected from R^{g}; or L² is absent;

R⁸ is selected from H, OR^{a1}, C₆₋₁₀ aryl and 5-10 membered heteroaryl, each of which is optionally substituted with 1, 2, 3, 4, or 5 substituents independently selected from R^{g};

or R⁷, L², and R⁸, together with the N atom to which R⁷ and L² are attached form a 4-7 membered heterocycloalkyl, which is optionally substituted with C(O)Cy and is optionally substituted with 1, 2, or 3 substituents independently selected from R^{g};

Cy is selected from C₆₋₁₀ aryl and 5-10 membered heteroaryl, each of which is optionally substituted with 1, 2, 3, 4, or 5 substituents independently selected from R^{g};

each R^{a1}, R^{b1}, R^{c}, and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};

or any R^{c1} and R^{d1} together with the N atom to which they are attached form a 4-7 membered heterocycloalkyl, which is optionally substituted with 1, 2, or 3 substituents independently selected from R^{g};

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments:

R¹, R², R³, and R⁴ are each independently selected from H, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};

[symbol] is a single bond or a double bond; wherein:

(i) when [symbol] is a double bond, R⁶ is selected from H, C₁₋₆ alkyl, and Cy, and X is selected from N and CR⁵; and

(ii) when [symbol] is a single bond, R⁶ is oxo, X is CR⁵, and R⁵ and R⁴, together with the carbon atoms to which they are attached, form C₆₋₁₀ aryl ring or 5-14 membered heteroaryl ring, each of which is optionally substituted with 1, 2, or 3 substituents independently selected from R^{g};

R⁵ is selected from H, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)C(O)NR^{c1}R^{d1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};

L¹ is selected from C₁₋₆ alkylene, C₂₋₆ alkenylene, and C₂₋₆ alkynylene, each of which is optionally substituted with 1, 2, or 3 substituents selected from R^{g};

R⁷ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents selected from R^{g}; or L² is absent;

R⁸ is selected from H, OR^{a1}, C₆₋₁₀ aryl and 5-10 membered heteroaryl, each of which is optionally substituted with 1, 2, 3, 4, or 5 substituents independently selected from R^{g};

or R⁷, L², and R⁸, together with the N atom to which R⁷ and L² are attached form a 4-7 membered heterocycloalkyl, which is optionally substituted with 1, 2, or 3 substituents independently selected from R^{g};

Cy is selected from C₆₋₁₀ aryl and 5-10 membered heteroaryl, each of which is optionally substituted with 1, 2, 3, 4, or 5 substituents independently selected from R^{g};

each R^{a1}, R^{b1}, R^{c1}, and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};

or any R^{c1} and R^{d1} together with the N atom to which they are attached form a 4-7 membered heterocycloalkyl, which is optionally substituted with 1, 2, or 3 substituents independently selected from R^{g};

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R¹, R², R³, and R⁴ are each independently selected from H, halo, C₁₋₆ alkyl, and OR^{a1} In some embodiments:

R¹ is selected from H, C₁₋₆ alkyl and C₁₋₆ alkoxy;

R² is selected from H, halo and C₁₋₆ alkyl;

R³ is selected from H, halo and C₁₋₆ alkyl; and

R⁴ is H.

In some embodiments, R¹ is H. In some embodiments, R¹ is C₁₋₆ alkyl. In some embodiments, R¹ is C₁₋₆ alkoxy.

In some embodiments, R² is H. In some embodiments, R² is halo. In some embodiments, R² is C₁₋₆ alkyl.

In some embodiments, R³ is H. In some embodiments, R³ is halo. In some embodiments, R³ is C₁₋₆ alkyl.

In some embodiments, R⁴ is H.

In some embodiments, [symbol] is a double bond.

In some embodiments, [symbol] is a single bond.

In some embodiments, X is N.

In some embodiments, X is CR⁵.

In some embodiments, [symbol] is a single bond and R⁶ is oxo.

In some embodiments, [symbol] is a double bond and X is N.

In some embodiments, [symbol] is a double bond and X is CR⁵.

In some embodiments, R⁵ and R⁴, together with the carbon atoms to which they are attached, form C₆₋₁₀ aryl ring or 5-14 membered heteroaryl ring, each of which is optionally substituted with 1, 2, or 3 substituents independently selected from R^{g}.

In some embodiments, R⁵ and R⁴, together with the carbon atoms to which they are attached, form C₆₋₁₀ aryl ring, optionally substituted with 1, 2, or 3 substituents independently selected from R^{g}.

In some embodiments, R⁵ and R⁴, together with the carbon atoms to which they are attached, form 5-14 membered heteroaryl ring, optionally substituted with 1, 2, or 3 substituents independently selected from R^{g}.

In some embodiments, R⁶ is Cy.

In some embodiments, R⁶ is C₁₋₆ alkyl.

In some embodiments, R⁶ is H.

In some embodiments, Cy is 5-10 membered heteroaryl, optionally substituted with 1, 2, or 3 independently selected R^{g}.

In some embodiments, R⁵ is selected from H, halo, C₁₋₆ alkyl, C₁₋₆ haloalkyl, OR^{a1}, C(O)R^{b1}, C(O)NR^{c1}R^{d1}, C(O)C(O)NR^{c1}R^{d1}, C(O)OR^{a1}, and NR^{c1}R^{d1} wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, and NR^{c1}R^{d1}.

In some embodiments, R⁵ is selected from H, C(O)R^{b1}, and C(O)C(O)NR^{c1}R^{d1}.

In some embodiments, R⁵ is C(O)C(O)NR^{c1}R^{d1}.

In some embodiments, R⁵ is C(O)R^{b1}. In some embodiments, R^{b1} is C₆₋₁₀ aryl, optionally substituted with halo. In some embodiments, R⁵ is p-chlorobenzoxy.

In some embodiments, R⁵ is H.

In some embodiments, each R^{a1}, R^{b1}, R^{c1}, and R^{d1} is independently selected from H, C₁₋₆ alkyl, C₁₋₄ haloalkyl, C₆₋₁₀ aryl, 5-10 membered heteroaryl, and C₃₋₁₀ cycloalkyl.

In some embodiments, L¹ is C₁₋₆ alkylene, optionally substituted with 1, 2, or 3 substituents selected from R^{g}. In some embodiments, L¹ is C₁₋₆ alkylene.

In some embodiments, R⁷ is H.

In some embodiments, L² is C₁₋₆ alkylene, optionally substituted with 1, 2, or 3 substituents selected from R^{g}. In some embodiments, L² is C₁₋₆ alkylene.

In some embodiments, L² is absent.

In some embodiments, R⁷, L², and R⁸, together with the N atom to which R⁷ and L² are attached form a 4-7 membered heterocycloalkyl, which is optionally substituted with C(O)Cy.

In some embodiments, R⁷, L², and R⁸, together with the N atom to which R⁷ and L² are attached form a piperidinyl, optionally substituted with 1, 2, or 3 substituents independently selected from R^{g}.

In some embodiments, R⁸ is C₆₋₁₀ aryl or 5-10 membered heteroaryl, each of which is optionally substituted with 1, 2, or 3 substituents independently selected from R^{g}. In some embodiments, R^{g} is halo, C₁₋₆ alkyl, or C₁₋₆ alkoxy.

In some embodiments, R⁸ is C₆₋₁₀ aryl, optionally substituted with 1, 2, or 3 independently selected R^{g}.

In some embodiments, R⁸ is phenyl, optionally substituted with 1, 2, or 3 independently selected R^{g}.

In some embodiments, R⁸ is 5-10 membered heteroaryl (e.g., furanyl or pyridinyl), optionally substituted with 1, 2, or 3 independently selected R^{g}.

In some embodiments, L² is absent and R⁸ is C₆₋₁₀ aryl, optionally substituted with 1, 2, or 3 independently selected R^{g}.

In some embodiments, R⁸ is H.

In some embodiments, R⁸ is C₁₋₆ alkoxy.

In some embodiments, R⁸ is 5-10 membered heteroaryl, optionally substituted with 1, 2, or 3 independently selected R^{g}.

In some embodiments, R⁸ is furanyl (e.g., furan-2-yl or furan-3-yl).

In some embodiments, R⁸ is furan-2-yl, optionally substituted with 1, 2, or 3 independently selected R^{g}.

In some embodiments, R⁸ is furan-2-yl.

In some embodiments, R⁸ is selected from furan-2-yl, indolyl, pyridin-2-yl, pyridin-3-yl, and thiophenyl, each of which is optionally substituted with 1, 2, or 3 independently selected R^{g}.

In some embodiments, R⁹ is selected from halo, C₁₋₆ alkyl, and C₁₋₆ alkoxy.

In some embodiments:

R¹, R², R³, and R⁴ are each independently selected from H, halo, C₁₋₆ alkyl, and OR^{a1};

(i) when [symbol] is a double bond, R⁶ is selected from H, C₁₋₆ alkyl, and 5-10 membered heteroaryl which is optionally substituted with 1, 2, or 3 independently selected R^{g}, and X is selected from N and CR⁵;

(ii) when [symbol] is a single bond, R⁶ is oxo, X is CR⁵, and R⁵ and R⁴, together with the carbon atoms to which they are attached, form C₆₋₁₀ aryl ring, optionally substituted with 1, 2, or 3 substituents independently selected from R^{g};

R⁵ is selected from H, halo, C₁₋₆ alkyl, C₁₋₆ haloalkyl, OR^{a1}, C(O)R^{b1}, C(O)NR^{c1}R^{d1}, C(O)OR^{a1}, and NR^{c1}R^{d1} wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, and NR^{c1}R^{d1};

each R^{a1}, R^{b1}, R^{c1}, and R^{d1} is independently selected from H, C₁₋₆ alkyl, C₁₋₄ haloalkyl, C₆₋₁₀ aryl, and C₃₋₁₀ cycloalkyl;

L¹ is C₁₋₆ alkylene, optionally substituted with 1, 2, or 3 substituents selected from R^{g};

R⁷ is H;

L² is C₁₋₆ alkylene, optionally substituted with 1, 2, or 3 substituents selected from R^{g};

R⁸ is selected from H, C₁₋₆ alkoxy, C₆₋₁₀ aryl and 5-10 membered heteroaryl, each of which is optionally substituted with 1, 2, or 3 substituents independently selected from R^{g}; and

R^{g} is selected from halo, C₁₋₆ alkyl, and C₁₋₆ alkoxy.

In some embodiments:

R¹ is selected from H, C₁₋₆ alkyl and C₁₋₆ alkoxy;

R² is selected from H, halo and C₁₋₆ alkyl;

R³ is selected from H, halo and C₁₋₆ alkyl;

R⁴ is H;

(i) when [symbol] is a double bond, R⁶ is selected from H, C₁₋₆ alkyl, and 5-10 membered heteroaryl which is optionally substituted with 1, 2, or 3 independently selected R^{g}, and X is selected from N and CR⁵; or

(ii) when [symbol] is a single bond, R⁶ is oxo, X is CR⁵, and R⁵ and R⁴, together with the carbon atoms to which they are attached, form C₆₋₁₀ aryl ring, optionally substituted with 1, 2, or 3 substituents independently selected from R^{g};

R⁵ is selected from H and C(O)R^{b1};

R^{b1} is selected from H, C₁₋₆ alkyl, C₁₋₄ haloalkyl, C₆₋₁₀ aryl, and C₃₋₁₀ cycloalkyl;

L¹ is C₁₋₆ alkylene;

R⁷ is H;

L² is C₁₋₆ alkylene;

R⁸ is selected from H, C₁₋₆ alkoxy, C₆₋₁₀ aryl and furan-2-yl, each of which is optionally substituted with 1, 2, or 3 independently selected R^{g}; and

R^{g} is selected from halo, C₁₋₆ alkyl, and C₁₋₆ alkoxy.

In some embodiments, the compound of Formula (II) is selected from any one of the compounds listed in Table 2, or a pharmaceutically acceptable salt thereof.

TABLE 2

| | |
|---|---|
| 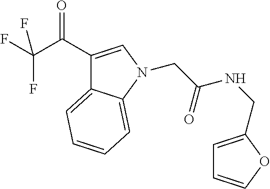 [SMILES: O=C(Cn1cc(C(=O)C(F)(F)F)c2ccccc21)NCc1ccco1] | BC181101 |
| 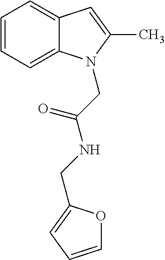 [SMILES: Cc1cc2ccccc2n1CC(=O)NCc1ccco1] | BC181144 |
| 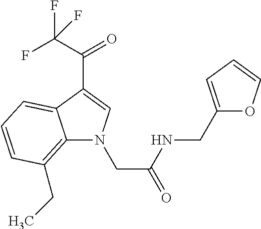 [SMILES: CCc1cccc2c(C(=O)C(F)(F)F)cn(CC(=O)NCc3ccco3)c12] | BC181102 |
| 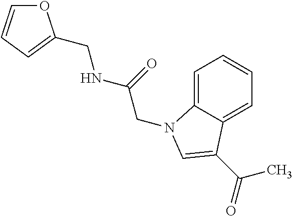 [SMILES: CC(=O)c1cn(CC(=O)NCc2ccco2)c2ccccc12] | BC181145 |
| 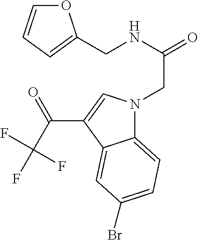 [SMILES: O=C(Cn1cc(C(=O)C(F)(F)F)c2cc(Br)ccc21)NCc1ccco1] | BC181103 |
| 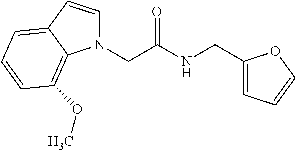 | BC181146 |
| 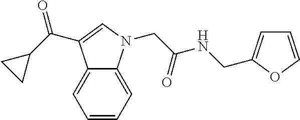 [SMILES: O=C(Cn1cc(C(=O)C2CC2)c2ccccc21)NCc1ccco1] | BC181105 |
| 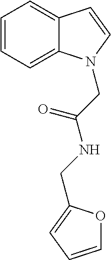 [SMILES: O=C(Cn1ccc2ccccc21)NCc1ccco1] | BC181147 |
| 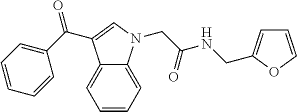 [SMILES: O=C(Cn1cc(C(=O)c2ccccc2)c2ccccc21)NCc1ccco1] | BC181106 |
| 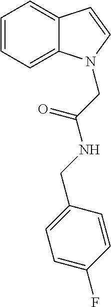 [SMILES: O=C(Cn1ccc2ccccc21)NCc1ccc(F)cc1] | BC181148 |
| 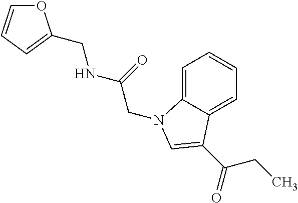 [SMILES: CCC(=O)c1cn(CC(=O)NCc2ccco2)c2ccccc12] | BC181108 |
| 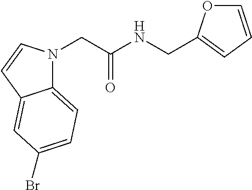 [SMILES: O=C(Cn1ccc2cc(Br)ccc21)NCc1ccco1] | BC181149 |
| 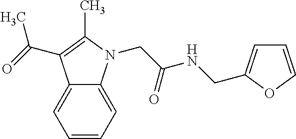 [SMILES: CC(=O)c1c(C)n(CC(=O)NCc2ccco2)c2ccccc12] | BC181109 |
| 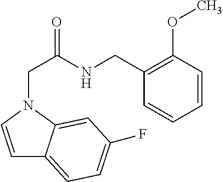 [SMILES: COc1ccccc1CNC(=O)Cn1ccc2ccc(F)cc21] | BC181151 |
| 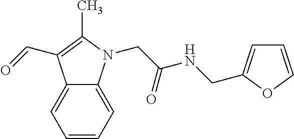 [SMILES: Cc1c(C=O)c2ccccc2n1CC(=O)NCc1ccco1] | BC181110 |
| 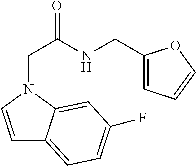 [SMILES: O=C(Cn1ccc2ccc(F)cc21)NCc1ccco1] | BC181152 |
| 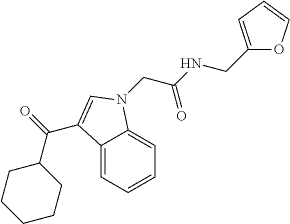 [SMILES: O=C(Cn1cc(C(=O)C2CCCCC2)c2ccccc21)NCc1ccco1] | BC181107 |
| 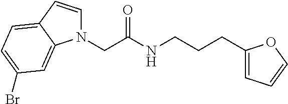 [SMILES: O=C(Cn1ccc2ccc(Br)cc21)NCCCc1ccco1] | BC181153 |
| 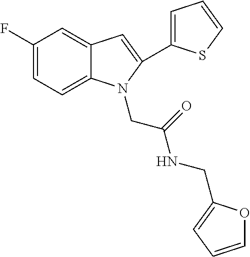 [SMILES: O=C(Cn1c(-c2cccs2)cc2cc(F)ccc21)NCc1ccco1] | BC181118 |
| 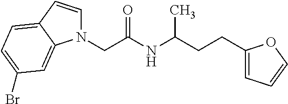 [SMILES: CC(CCc1ccco1)NC(=O)Cn1ccc2ccc(Br)cc21] | BC181154 |
| 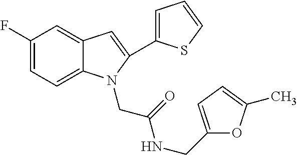 [SMILES: Cc1ccc(CNC(=O)Cn2c(-c3cccs3)cc3cc(F)ccc32)o1] | BC181119 |
| 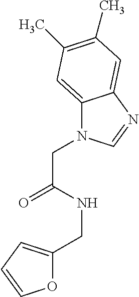 [SMILES: Cc1cc2ncn(CC(=O)NCc3ccco3)c2cc1C] | BC181159 |
| 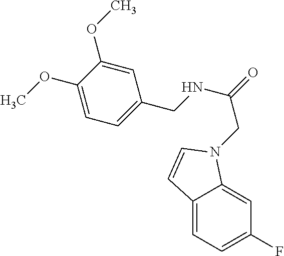 [SMILES: COc1ccc(CNC(=O)Cn2ccc3ccc(F)cc32)cc1OC] | BC181150 |
| 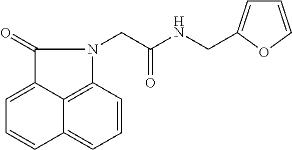 [SMILES: O=C(CN1C(=O)c2cccc3cccc1c23)NCc1ccco1] | BC181104 |
| 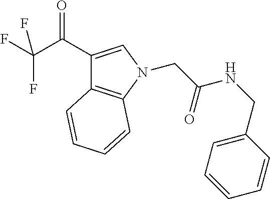 [SMILES: O=C(Cn1cc(C(=O)C(F)(F)F)c2ccccc21)NCc1ccccc1] | BC181201 |
| 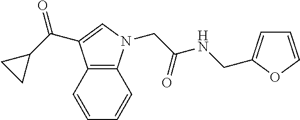 [SMILES: O=C(Cn1cc(C(=O)C2CC2)c2ccccc21)NCc1ccco1] | BC181202 |
| 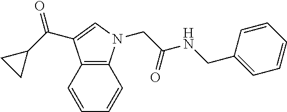 [SMILES: O=C(Cn1cc(C(=O)C2CC2)c2ccccc21)NCc1ccccc1] | BC181203 |
| 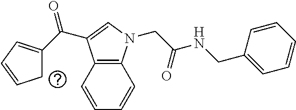 [SMILES: O=C(Cn1cc(C(=O)C2=CC=CC2)c2ccccc21)NCc1ccccc1] | BC181204 |
| 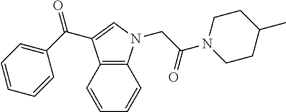 [SMILES: CC1CCN(C(=O)Cn2cc(C(=O)c3ccccc3)c3ccccc32)CC1] | BC181205 |
| 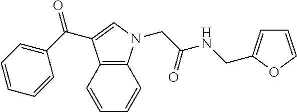 [SMILES: O=C(Cn1cc(C(=O)c2ccccc2)c2ccccc21)NCc1ccco1] | BC181206 |
| 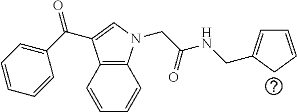 [SMILES: O=C(Cn1cc(C(=O)c2ccccc2)c2ccccc21)NCC1=CC=CC1] | BC181207 |
| 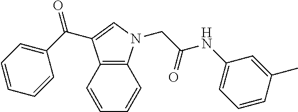 [SMILES: Cc1cccc(NC(=O)Cn2cc(C(=O)c3ccccc3)c3ccccc32)c1] | BC181208 |
| 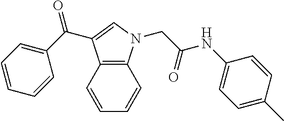 [SMILES: Cc1ccc(NC(=O)Cn2cc(C(=O)c3ccccc3)c3ccccc32)cc1] | BC181209 |
| 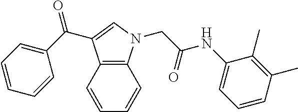 [SMILES: Cc1cccc(NC(=O)Cn2cc(C(=O)c3ccccc3)c3ccccc32)c1C] | BC181210 |
| 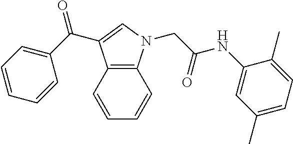 [SMILES: Cc1ccc(C)c(NC(=O)Cn2cc(C(=O)c3ccccc3)c3ccccc32)c1] | BC181211 |
| 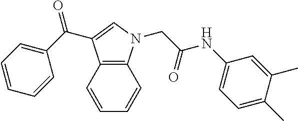 [SMILES: Cc1ccc(NC(=O)Cn2cc(C(=O)c3ccccc3)c3ccccc32)cc1C] | BC181212 |
| 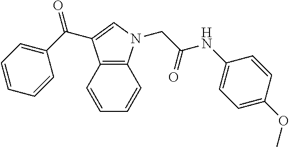 [SMILES: COc1ccc(NC(=O)Cn2cc(C(=O)c3ccccc3)c3ccccc32)cc1] | BC181213 |
| 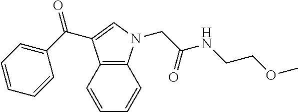 [SMILES: COCCNC(=O)Cn1cc(C(=O)c2ccccc2)c2ccccc21] | BC181214 |
| 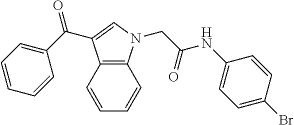 [SMILES: O=C(Cn1cc(C(=O)c2ccccc2)c2ccccc21)Nc1ccc(Br)cc1] | BC181215 |
| 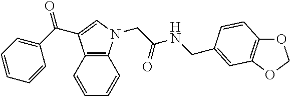 [SMILES: O=C(Cn1cc(C(=O)c2ccccc2)c2ccccc21)NCc1ccc2c(c1)OCO2] | BC181216 |
| 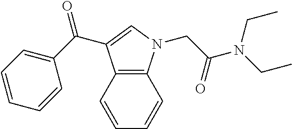 [SMILES: CCN(CC)C(=O)Cn1cc(C(=O)c2ccccc2)c2ccccc21] | BC181217 |
| 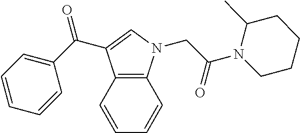 [SMILES: CC1CCCCN1C(=O)Cn1cc(C(=O)c2ccccc2)c2ccccc21] | BC181218 |
| 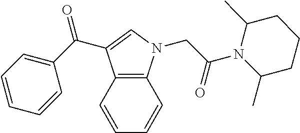 [SMILES: CC1CCCC(C)N1C(=O)Cn1cc(C(=O)c2ccccc2)c2ccccc21] | BC181219 |
| 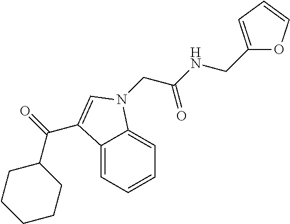 [SMILES: O=C(Cn1cc(C(=O)C2CCCCC2)c2ccccc21)NCc1ccco1] | BC181220 |
| 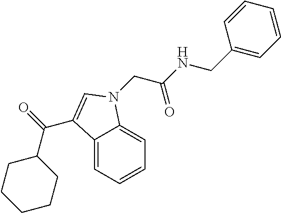 [SMILES: O=C(Cn1cc(C(=O)C2CCCCC2)c2ccccc21)NCc1ccccc1] | BC181221 |
| 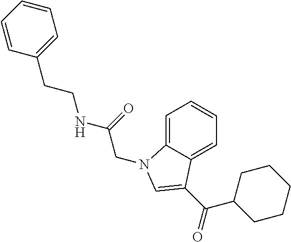 [SMILES: O=C(Cn1cc(C(=O)C2CCCCC2)c2ccccc21)NCCc1ccccc1] | BC181222 |
| 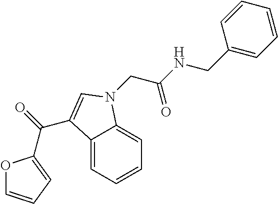 [SMILES: O=C(Cn1cc(C(=O)c2ccco2)c2ccccc21)NCc1ccccc1] | BC181223 |
| 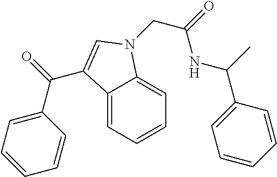 [SMILES: CC(NC(=O)Cn1cc(C(=O)c2ccccc2)c2ccccc21)c1ccccc1] | BC181224 |
| 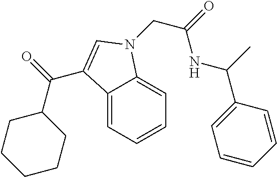 [SMILES: CC(NC(=O)Cn1cc(C(=O)C2CCCCC2)c2ccccc21)c1ccccc1] | BC181225 |
| 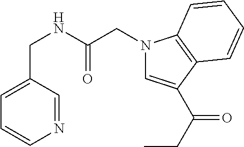 [SMILES: CCC(=O)c1cn(CC(=O)NCc2cccnc2)c2ccccc12] | BC181226 |
| 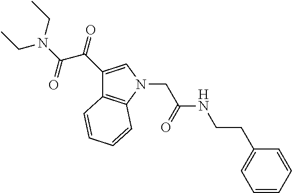 [SMILES: CCN(CC)C(=O)C(=O)c1cn(CC(=O)NCCc2ccccc2)c2ccccc12] | BC181227 |
| 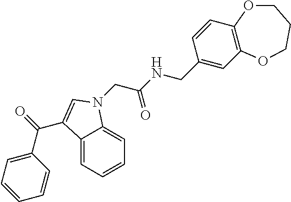 [SMILES: O=C(Cn1cc(C(=O)c2ccccc2)c2ccccc21)NCc1ccc2c(c1)OCCCO2] | BC181257 |
| 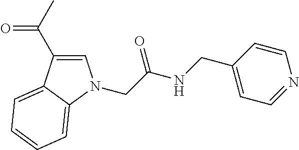 [SMILES: CC(=O)c1cn(CC(=O)NCc2ccncc2)c2ccccc12] | BC181266 |
| 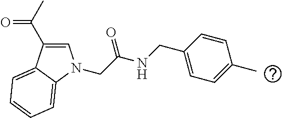 [SMILES: CC(=O)c1cn(CC(=O)NCc2ccc(C)cc2)c2ccccc12] | BC181267 |
| 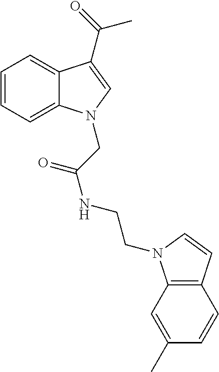 [SMILES: CC(=O)c1cn(CC(=O)NCCn2ccc3ccc(C)cc32)c2ccccc12] | BC181268 |
| 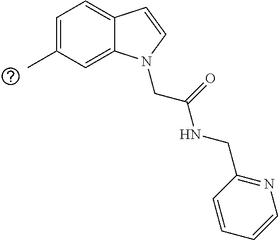 [SMILES: Cc1ccc2ccn(CC(=O)NCc3ccccn3)c2c1] | BC18127 |

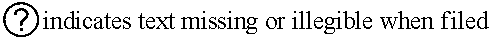

In some embodiments, the compound of Formula (II) is selected from any one of the compounds listed in Table 2a, or a pharmaceutically acceptable salt thereof.

TABLE 2a

| | |
|---|---|
| 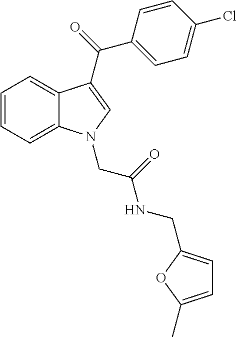 [SMILES: Cc1ccc(CNC(=O)Cn2cc(C(=O)c3ccc(Cl)cc3)c3ccccc32)o1] | BC20176 |
| 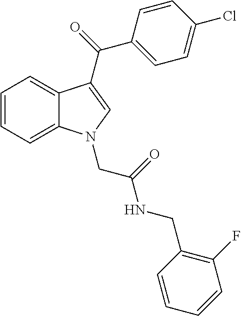 [SMILES: O=C(Cn1cc(C(=O)c2ccc(Cl)cc2)c2ccccc21)NCc1ccccc1F] | BC20177 |
| 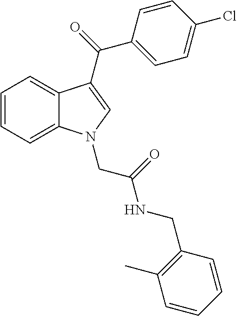 [SMILES: Cc1ccccc1CNC(=O)Cn1cc(C(=O)c2ccc(Cl)cc2)c2ccccc21] | BC20178 |
| 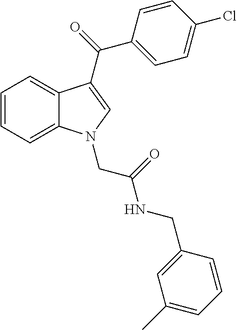 [SMILES: Cc1cccc(CNC(=O)Cn2cc(C(=O)c3ccc(Cl)cc3)c3ccccc32)c1] | BC20179 |
| 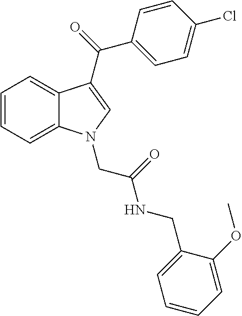 [SMILES: COc1ccccc1CNC(=O)Cn1cc(C(=O)c2ccc(Cl)cc2)c2ccccc21] | BC20180 |
| 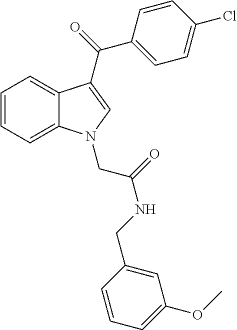 [SMILES: COc1cccc(CNC(=O)Cn2cc(C(=O)c3ccc(Cl)cc3)c3ccccc32)c1] | BC20181 |
| 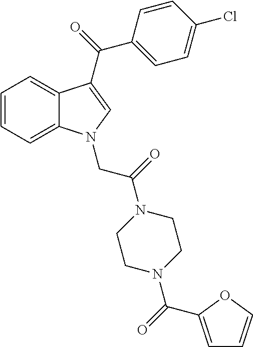 [SMILES: O=C(c1ccc(Cl)cc1)c1cn(CC(=O)N2CCN(C(=O)c3ccco3)CC2)c2ccccc12] | BC20182 |
| 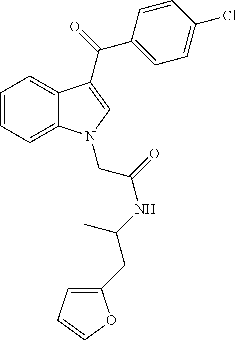 [SMILES: CC(Cc1ccco1)NC(=O)Cn1cc(C(=O)c2ccc(Cl)cc2)c2ccccc21] | BC20214 |
| 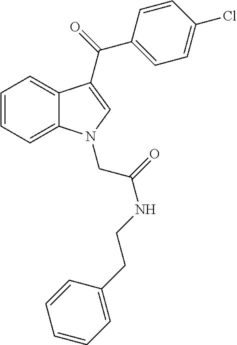 [SMILES: O=C(Cn1cc(C(=O)c2ccc(Cl)cc2)c2ccccc21)NCCc1ccccc1] | BC20215 |
| 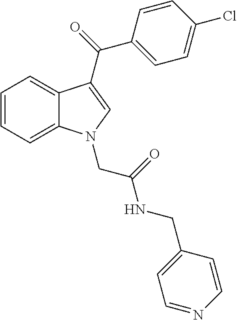 [SMILES: O=C(Cn1cc(C(=O)c2ccc(Cl)cc2)c2ccccc21)NCc1ccncc1] | BC20216 |

In some embodiments, this document provides a compound of Formula (III):

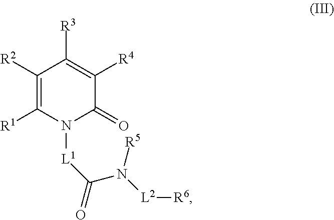

or a pharmaceutically acceptable salt thereof, wherein:

R¹, R², and R³ are each independently selected from H, halo, C₁₋₆ alkyl, C₁₋₆ haloalkyl, C₁₋₆ alkoxy, and C₁₋₆ haloalkoxy;

R⁴ is CN;

or R⁴ and R³, together with the carbon atoms to which they are attached, form a C₆₋₁₀ aryl ring or 5-10 membered heteroaryl ring, each of which is optionally substituted with 1, 2, or 3 substituents independently selected from R⁷;

each R⁷ is independently selected from halo, CN, C₁₋₆ alkyl, C₁₋₆ haloalkyl, C₁₋₆ alkoxy, and C₁₋₆ haloalkoxy;

L¹ is selected from C₁₋₆ alkylene, C₂₋₆ alkenylene, and C₂₋₆ alkynylene, each of which is optionally substituted with 1, 2, or 3 substituents selected from R^{g};

R⁵ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents independently selected from R^{g};

or L² is absent;

R⁶ is selected from H, C₆₋₁₀ aryl and 5-10 membered heteroaryl, each of which is optionally substituted with 1, 2, 3, 4, or 5 substituents independently selected from R⁸; provided that when L² is absent, R⁶ is not H;

each R⁸ is independently selected from halo, CN, NO₂, C₁₋₆ alkyl, C₁₋₆ haloalkyl, C₂₋₆ alkenyl, C₂₋₆ alkynyl, OR^{a1}, SR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};

each R^{a1}, R^{b1}, R^{c1}, and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};

or any R^{c1} and R^{d1} together with the N atom to which they are attached form a 4-7 membered heterocycloalkyl, which is optionally substituted with 1, 2, or 3 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, L¹ is C₁₋₆ alkylene, optionally substituted with 1, 2, or 3 substituents selected from R^{g}.

In some embodiments, L¹ is C₁₋₆ alkylene.

In some embodiments, R⁵ is H.

In some embodiments, R⁵ is C₁₋₆ alkyl.

In some embodiments, L² is absent.

In some embodiments, L² is C₁₋₆ alkylene, optionally substituted with 1, 2, or 3 substituents selected from R^{g}.

In some embodiments, L² is C₁₋₆ alkylene.

In some embodiments, L² is absent or C₁₋₆ alkylene, optionally substituted with 1, 2, or 3 substituents selected from R^{g}.

In some embodiments, R⁶ is selected from C₆₋₁₀ aryl and 5-10 membered heteroaryl, each of which is optionally substituted with 1, 2, or 3 substituents independently selected from R⁸.

In some embodiments, R⁶ is C₆₋₁₀ aryl, optionally substituted with 1, 2, or 3 substituents independently selected from R⁸.

In some embodiments, R⁶ is 5-10 membered heteroaryl, optionally substituted with 1, 2, or 3 substituents independently selected from R⁸.

In some embodiments, R⁶ is selected from phenyl, furan-2-yl, and thiophen-2-yl, each of which is optionally substituted with 1, 2, or 3 substituents independently selected from R⁸.

In some embodiments, R⁶ is selected from phenyl, furanyl, and thiophenyl, each of which is optionally substituted with 1, 2, or 3 substituents independently selected from R⁸.

In some embodiments, R⁶ is phenyl, optionally substituted with 1, 2, or 3 substituents independently selected from R⁸.

In some embodiments, R⁶ is furan-2-yl, optionally substituted with 1, 2, or 3 substituents independently selected from R⁸.

In some embodiments, R⁶ is thiophen-2-yl, optionally substituted with 1, 2, or 3 substituents independently selected from R⁸.

In some embodiments, R⁶ is H.

In some embodiments, R⁶ is a substituent other than H.

In some embodiments, L² is absent or C₁₋₆ alkylene, optionally substituted with 1, 2, or 3 substituents independently selected from R^{g}; and R⁶ is selected from C₆₋₁₀ aryl and 5-10 membered heteroaryl, each of which is optionally substituted with 1, 2, or 3 substituents independently selected from R⁸.

In some embodiments, L² is C₁₋₆ alkylene, optionally substituted with 1, 2, or 3 substituents independently selected from R^{g}; and R⁶ is H.

In some embodiments, L² is absent and R⁶ is a substituent other than H.

In some embodiments, L² is absent and R⁶ is selected from C₆₋₁₀ aryl and 5-10 membered heteroaryl, each of which is optionally substituted with 1, 2, or 3 substituents independently selected from R⁸.

In some embodiments, L² is absent and R⁶ is C₆₋₁₀ aryl, optionally substituted with 1, 2, or 3 substituents independently selected from R⁸.

In some embodiments, L² is absent and R⁶ is 5-10 membered heteroaryl, substituted with 1, 2, or 3 substituents independently selected from R⁸.

In some embodiments, R⁸ is selected from halo, C₁₋₆ alkyl, C₁₋₆ haloalkyl, OR^{a1}, SR^{a1}, C(O)R^{b1}, C(O)NR^{c1}R^{a1}, C(O)OR^{a1}, NR^{c1}R^{d1}, and NR^{c1}C(O)R^{b1} In some embodiments, R⁸ is selected from halo, C₁₋₆ alkyl, OR^{a1}, SR^{a1}, C(O)NR^{c1}R^{d1}.

In some embodiments, each R^{a1}, R^{b1}, R^{c1}, and R^{d1} is independently selected from H, C₁₋₆ alkyl, C₁₋₄ haloalkyl, C₆₋₁₀ aryl-C₁₋₄ alkylene, C₃₋₁₀ cycloalkyl-C₁₋₄ alkylene, and (5-10 membered heteroaryl)-C₁₋₄ alkylene.

In some embodiments, each R^{a1}, R^{b1}, R^{c1}, and R^{d1} is independently selected from H, C₁₋₆ alkyl, and (5-10 membered heteroaryl)-C₁₋₄ alkylene.

In some embodiments, the compound of Formula (III) has formula:

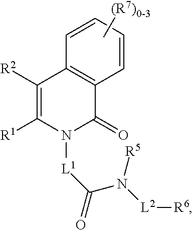

or a pharmaceutically acceptable salt thereof.

In some embodiments, the compound of Formula (III) has formula:

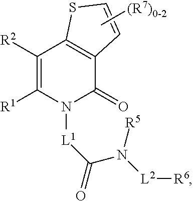

or a pharmaceutically acceptable salt thereof.

In some embodiments, R⁷ is selected from C₁₋₆ alkyl, C₁₋₆ haloalkyl, C₁₋₆ alkoxy, and C₁₋₆ haloalkoxy.

In some embodiments, R⁷ is C₁₋₆ alkyl.

In some embodiments, R¹ and R² are each independently selected from H and C₁₋₆ alkyl.

In some embodiments, R¹ is C₁₋₆ alkyl, and R² is H.

In some embodiments, R¹ is H, and R² is C₁₋₆ alkyl.

In some embodiments, R¹ and R² are each H.

In some embodiments, R¹ and R² are each C₁₋₆ alkyl.

In some embodiments:

R¹ and R² are each independently selected from H and C₁₋₆ alkyl;

R⁷ is selected from C₁₋₆ alkyl, C₁₋₆ haloalkyl, C₁₋₆ alkoxy, and C₁₋₆ haloalkoxy;

L¹ is C₁₋₆ alkylene, optionally substituted with 1, 2, or 3 substituents selected from R^{g};

R⁵ is H;

L² is absent or C₁₋₆ alkylene, optionally substituted with 1, 2, or 3 substituents selected from R^{g}, and R⁶ is selected from C₆₋₁₀ aryl and 5-10 membered heteroaryl, each of which is optionally substituted with 1, 2, or 3 substituents independently selected from R⁸;

or L² is C₁₋₆ alkylene, optionally substituted with 1, 2, or 3 substituents independently selected from R^{g}, and R⁶ is H;

R⁸ is selected from halo, C₁₋₆ alkyl, C₁₋₆ haloalkyl, OR^{a1}, SR^{a1}, C(O)R^{b1}, C(O)NR^{c1}R^{d1}, C(O)OR^{a1}, NR^{c1}R^{d1}, and NR^{c1}C(O)R^{b1}; and

each R^{a1}, R^{b}, R^{c1}, and R^{d1} is independently selected from H, C₁₋₆ alkyl, C₁₋₄ haloalkyl, C₆₋₁₀ aryl-C₁₋₄ alkylene, C₃₋₁₀ cycloalkyl-C₁₋₄ alkylene, and (5-10 membered heteroaryl)-C₁₋₄ alkylene.

In some embodiments:

R¹ is C₁₋₆ alkyl;

R² is H;

R⁷ is C₁₋₆ alkyl;

L¹ is C₁₋₆ alkylene;

R⁵ is H;

L² is absent or C₁₋₆ alkylene, optionally substituted with 1, 2, or 3 substituents selected from R^{g}, and R⁶ is selected from phenyl, furan-2-yl, and thiophen-2-yl, each of which is optionally substituted with 1, 2, or 3 substituents independently selected from R⁸; or L² is C₁₋₆ alkylene, optionally substituted with 1, 2, or 3 substituents independently selected from R^{g}, and R⁶ is H;

R⁸ is selected from halo, C₁₋₆ alkyl, OR^{a1}, SR^{a1}, and C(O)NR^{c1}R^{d1}; and

each R^{a1}, R, and R^{d1} is independently selected from H, C₁₋₆ alkyl, and (5-10 membered heteroaryl)-C₁₋₄ alkylene.

In some embodiments, compound of Formula (III) has formula:

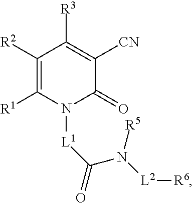

or a pharmaceutically acceptable salt thereof.

In some embodiments, R¹, R², and R³ are each independently selected from H and C₁₋₆ alkyl.

In some embodiments, R¹ and R³ are each C₁₋₆ alkyl, and R² is H.

In some embodiments, R¹ and R³ are each methyl, and R² is H.

In some embodiments:

R¹, R², and R³ are each independently selected from H and C₁₋₆ alkyl;

L¹ is C₁₋₆ alkylene, optionally substituted with 1, 2, or 3 substituents selected from R^{g};

R⁵ is H;

L² is absent or C₁₋₆ alkylene, optionally substituted with 1, 2, or 3 substituents selected from R^{g}, and R⁶ is selected from C₆₋₁₀ aryl and 5-10 membered heteroaryl, each of which is optionally substituted with 1, 2, or 3 substituents independently selected from R⁸;

or L² is C₁₋₆ alkylene, optionally substituted with 1, 2, or 3 substituents independently selected from R^{g}, and R⁶ is H;

R⁸ is selected from halo, C₁₋₆ alkyl, C₁₋₆ haloalkyl, OR^{a1}, SR^{a1}, C(O)R^{b1}, C(O)NR^{c1}R^{d1}, C(O)OR^{a1}, NR^{c1}R^{d1}, and NR^{c1}C(O)R^{b1}; and

each R^{a1}, R^{b1}, R^{c1}, and R^{d1} is independently selected from H, C₁₋₆ alkyl, C₁₋₄ haloalkyl, C₆₋₁₀ aryl-C₁₋₄ alkylene, C₃₋₁₀ cycloalkyl-C₁₋₄ alkylene, and (5-10 membered heteroaryl)-C₁₋₄ alkylene.

In some embodiments:

R¹ and R³ are each C₁₋₆ alkyl;

R² is H;

L¹ is C₁₋₆ alkylene;

R⁵ is H;

L² is absent or C₁₋₆ alkylene, optionally substituted with 1, 2, or 3 substituents independently selected from R^{g} and R⁶ is selected from phenyl, furan-2-yl, and thiophen-2-yl, each of which is optionally substituted with 1, 2, or 3 substituents independently selected from R⁸;

or L² is C₁₋₆ alkylene, optionally substituted with 1, 2, or 3 substituents independently selected from R^{g} and R⁶ is H;

R⁸ is selected from halo, C₁₋₆ alkyl, OR^{a1}, SR^{a1}, and C(O)NR^{c1}R^{d1}; and

each R^{a1}, R^{c1}, and R^{d1} is independently selected from H, C₁₋₆ alkyl, and (5-10 membered heteroaryl)-C₁₋₄ alkylene.

In some embodiments, the compound of Formula (III) is selected from any one of the compounds listed in Table 3, or a pharmaceutically acceptable salt thereof.

TABLE 3

| | |
|---|---|
| 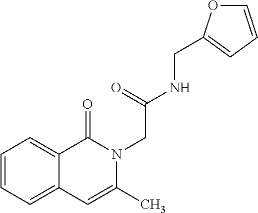 [SMILES: Cc1cc2ccccc2c(=O)n1CC(=O)NCc1ccco1] | BC181112 |
| 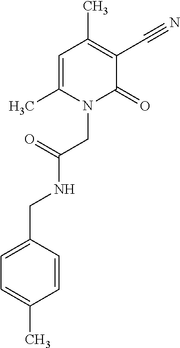 [SMILES: Cc1ccc(CNC(=O)Cn2c(C)cc(C)c(C#N)c2=O)cc1] | BC181121 |
| 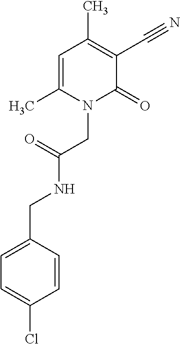 [SMILES: Cc1cc(C)n(CC(=O)NCc2ccc(Cl)cc2)c(=O)c1C#N] | BC181122 |
| 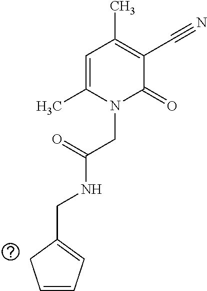 [SMILES: Cc1cc(C)n(CC(=O)NCC2=CC=CC2)c(=O)c1C#N] | BC181129 |
| 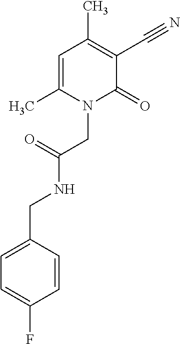 [SMILES: Cc1cc(C)n(CC(=O)NCc2ccc(F)cc2)c(=O)c1C#N] | BC181123 |
| 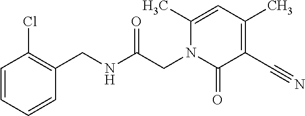 [SMILES: Cc1cc(C)n(CC(=O)NCc2ccccc2Cl)c(=O)c1C#N] | BC181130 |
| 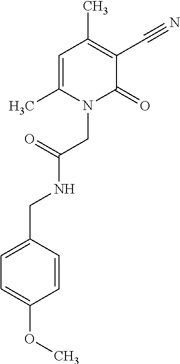 [SMILES: COc1ccc(CNC(=O)Cn2c(C)cc(C)c(C#N)c2=O)cc1] | BC181124 |
| 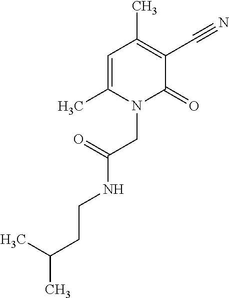 [SMILES: Cc1cc(C)n(CC(=O)NCCC(C)C)c(=O)c1C#N] | BC181131 |
| 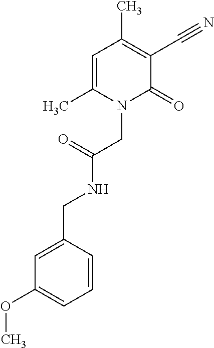 [SMILES: COc1cccc(CNC(=O)Cn2c(C)cc(C)c(C#N)c2=O)c1] | BC181125 |
| 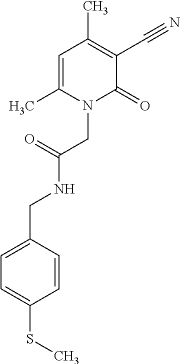 [SMILES: CSc1ccc(CNC(=O)Cn2c(C)cc(C)c(C#N)c2=O)cc1] | BC181132 |
| 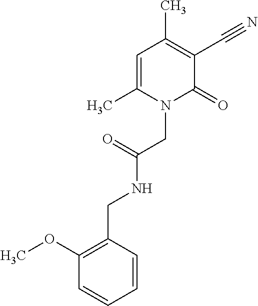 [SMILES: COc1ccccc1CNC(=O)Cn1c(C)cc(C)c(C#N)c1=O] | BC181126 |
| 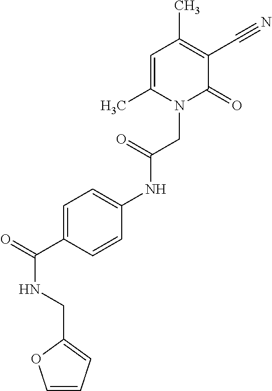 [SMILES: Cc1cc(C)n(CC(=O)Nc2ccc(C(=O)NCc3ccco3)cc2)c(=O)c1C#N] | BC181133 |
| 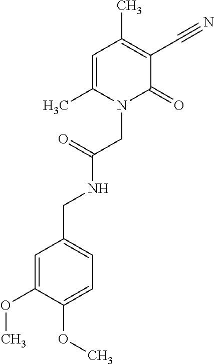 [SMILES: COc1ccc(CNC(=O)Cn2c(C)cc(C)c(C#N)c2=O)cc1OC] | BC181127 |
| 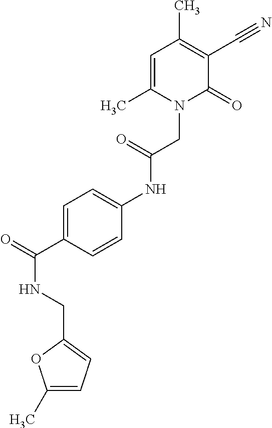 [SMILES: Cc1ccc(CNC(=O)c2ccc(NC(=O)Cn3c(C)cc(C)c(C#N)c3=O)cc2)o1] | BC181134 |
| 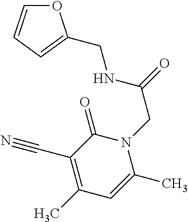 [SMILES: Cc1cc(C)n(CC(=O)NCc2ccco2)c(=O)c1C#N] | BC181128 |
| 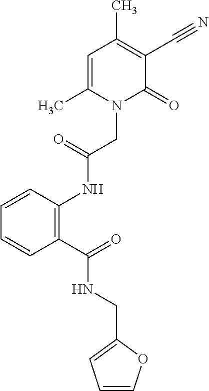 [SMILES: Cc1cc(C)n(CC(=O)Nc2ccccc2C(=O)NCc2ccco2)c(=O)c1C#N] | BC181135 |
| 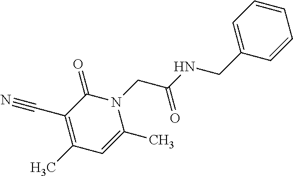 [SMILES: Cc1cc(C)n(CC(=O)NCc2ccccc2)c(=O)c1C#N] | BC181120 |
| 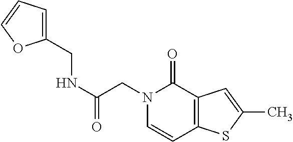 [SMILES: Cc1cc2c(=O)n(CC(=O)NCc3ccco3)ccc2s1] | BC181140 |

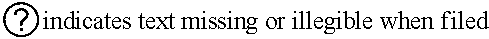

In some embodiments, this document provides a compound of Formula (IV):

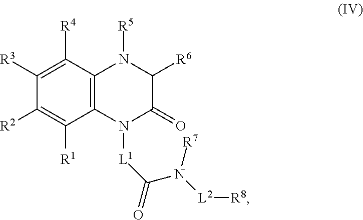

or a pharmaceutically acceptable salt thereof, wherein:

R¹, R², R³, and R⁴ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, and C₁₋₆ haloalkoxy;

R⁵ is selected from C₁₋₆ alkyl, C₁₋₆ haloalkyl, C₂₋₆ alkenyl, and C₂₋₆ alkynyl, each of which is optionally substituted with 1, 2, or 3 substituents independently selected from R^{g};

R⁶ is oxo;

or R⁵ and R⁶, together with N atom to which R⁵ is attached and carbon atom to which R⁶ is attached, form a 4-10 membered heterocycloalkyl ring or a 5-10 membered heteroaryl ring, each of which is optionally substituted with 1, 2, or 3 independently selected R^{g};

L¹ is selected from C₁₋₆ alkylene, C₂₋₆ alkenylene, and C₂₋₆ alkynylene, each of which is optionally substituted with 1, 2, or 3 substituents selected from R^{g};

R⁷ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents selected from R^{g};

R⁸ is 5-10 membered heteroaryl, optionally substituted with 1, 2, 3, 4, or 5 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R¹, R², R³, and R⁴ are each independently selected from H and halo.

In some embodiments, R² is selected from halo, CN, NO₂, OH, C₁₋₆ alkyl, C₁₋₆ haloalkyl, C₂₋₆ alkenyl, C₂₋₆ alkynyl, C₁₋₆ alkoxy, and C₁₋₆ haloalkoxy.

In some embodiments, R² is selected from halo, C₁₋₆ alkyl, C₁₋₆ haloalkyl, C₁₋₆ alkoxy, and C₁₋₆ haloalkoxy.

In some embodiments, R² is selected from halo, C₁₋₆ alkyl, and C₁₋₆ alkoxy.

In some embodiments, R² is selected from H, halo, CN, NO₂, OH, C₁₋₆ alkyl, C₁₋₆ haloalkyl, C₂₋₆ alkenyl, C₂₋₆ alkynyl, C₁₋₆ alkoxy, and C₁₋₆ haloalkoxy; and R¹, R³, and R⁴ are each H.

In some embodiments, R² is selected from H and halo; and R¹, R³, and R⁴ are each H.

In some embodiments, R² is halo; and R¹, R³, and R⁴ are each H.

In some embodiments, R¹, R², R³, and R⁴ are each H.

In some embodiments, the compound of Formula (IV) has formula:

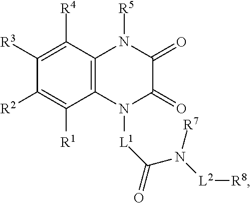

or a pharmaceutically acceptable salt thereof.

In some embodiments, R⁵ is C₁₋₆ alkyl.

In some embodiments, the compound of Formula (IV) has formula:

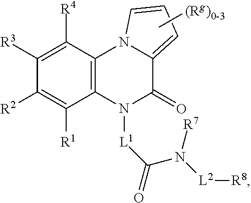

or a pharmaceutically acceptable salt thereof.

In some embodiments, L¹ is C₁₋₆ alkylene, optionally substituted with 1, 2, or 3 substituents selected from R^{g}.

In some embodiments, L¹ is C₁₋₆ alkylene.

In some embodiments, R⁷ is H.

In some embodiments, L² is C₁₋₆ alkylene, optionally substituted with 1, 2, or 3 substituents selected from R^{g}.

In some embodiments, L² is C₁₋₆ alkylene.

In some embodiments, R⁸ is 5-10 membered heteroaryl, optionally substituted with 1, 2, or 3 substituents independently selected from R^{g}. In some aspects of these embodiments, the 5-10 membered heteroaryl is furan-2-yl.

In some embodiments, R⁹ is selected from OH, NO₂, CN, halo, C₁₋₆ alkyl, C₁₋₄ haloalkyl, C₁₋₆ alkoxy, and C₁₋₆ haloalkoxy.

In some embodiments, R⁹ is selected from halo, C₁₋₆ alkyl, and C₁₋₆ alkoxy.

In some embodiments, R⁹ is C₁₋₆ alkyl.

In some embodiments:

R¹, R², R³, and R⁴ are each independently selected from H and halo;

R⁵ is C₁₋₆ alkyl;

L¹ is C₁₋₆ alkylene, optionally substituted with 1, 2, or 3 substituents selected from R^{g};

R⁷ is H;

L² is C₁₋₆ alkylene, optionally substituted with 1, 2, or 3 substituents selected from R^{g};

R⁸ is 5-10 membered heteroaryl, optionally substituted with 1, 2, or 3 substituents independently selected from R^{g}; and

R^{g} is selected from OH, NO₂, CN, halo, C₁₋₆ alkyl, C₁₋₄ haloalkyl, C₁₋₆ alkoxy, and C₁₋₆ haloalkoxy.

In some embodiments:

R² is selected from H and halo;

R¹, R³, and R⁴ are each H;

R⁵ is C₁₋₆ alkyl;

L¹ is C₁₋₆ alkylene;

R⁷ is H;

L² is C₁₋₆ alkylene; and

R⁸ is 5-10 membered heteroaryl, optionally substituted with C₁₋₆ alkyl.

In some embodiments:

R¹, R², R³, and R⁴ are each independently selected from H and halo;

L¹ is C₁₋₆ alkylene, optionally substituted with 1, 2, or 3 substituents selected from R^{g};

R⁷ is H;

L² is C₁₋₆ alkylene, optionally substituted with 1, 2, or 3 substituents selected from R^{g};

R⁸ is 5-10 membered heteroaryl, optionally substituted with 1, 2, or 3 substituents independently selected from R^{g}; and

R^{g} is selected from OH, NO₂, CN, halo, C₁₋₆ alkyl, C₁₋₄ haloalkyl, C₁₋₆ alkoxy, and C₁₋₆ haloalkoxy.

In some embodiments:

R² is selected from H and halo;

R¹, R³, and R⁴ are each H;

R⁵ is C₁₋₆ alkyl;

L¹ is C₁₋₆ alkylene;

R⁷ is H;

L² is C₁₋₆ alkylene; and

R⁸ is 5-10 membered heteroaryl, optionally substituted with C₁₋₆ alkyl.

In some embodiments, the compound of Formula (IV) is selected from any one of the compounds listed in Table 4, or a pharmaceutically acceptable salt thereof.

TABLE 4

| | |
|---|---|
| 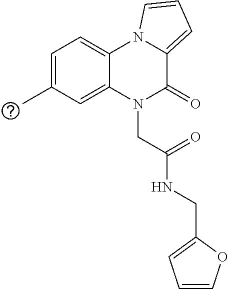 [SMILES: Cc1ccc2c(c1)n(CC(=O)NCc1ccco1)c(=O)c1cccn12] | BC181113 |
| 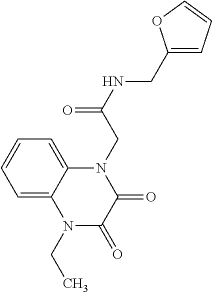 [SMILES: CCn1c(=O)c(=O)n(CC(=O)NCc2ccco2)c2ccccc21] | BC181138 |
| 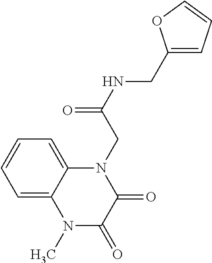 [SMILES: Cn1c(=O)c(=O)n(CC(=O)NCc2ccco2)c2ccccc21] | BC181136 |
| 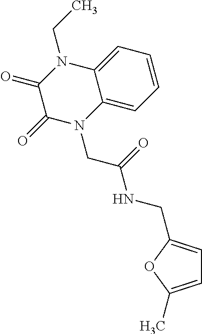 [SMILES: CCn1c(=O)c(=O)n(CC(=O)NCc2ccc(C)o2)c2ccccc21] | BC181139 |
| 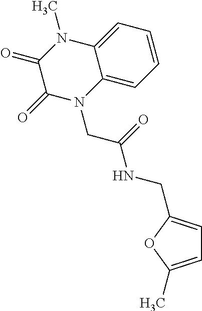 [SMILES: Cc1ccc(CNC(=O)Cn2c(=O)c(=O)n(C)c3ccccc32)o1] | BC181137 |

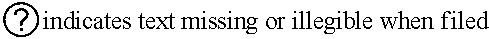

In some embodiments, this document provides a compound of Formula (V):

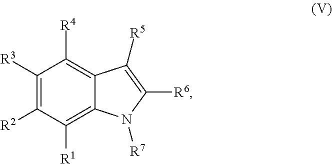

or a pharmaceutically acceptable salt thereof, wherein R¹, R², R³, R⁴, R⁵, R⁶, and R⁷ are as described herein.

In some embodiments:

R¹, R², R³, R⁴, R⁵, and R⁶ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, C₁₋₆ haloalkoxy, wherein said C₁₋₆ alkyl is optionally substituted with 1, 2, or 3 substituents independently selected from OH, NO₂, CN, C₆₋₁₀ alkoxy, C₆₋₁₀ haloalkoxy, and Cy, and a moiety of formula (i):

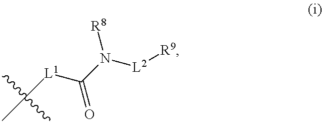

provided that at least one of R¹, R², R³, R⁴, R⁵, and R⁶ is a moiety of formula (i);

or R⁵ and R⁶ together with the carbon atoms to which they are attached from a C₄₋₇ cycloalkyl ring or a 4-7 membered heterocycloalkyl ring, each of which is optionally substituted with 1, 2, or 3 substituents independently selected from OH, NO₂, CN, C₆₋₁₀ alkoxy, C₆₋₁₀ haloalkoxy, Cy, and a moiety of formula (i);

or when R⁴ is a moiety of formula (i), the R⁸ and R⁵, together with the N atom to which R⁸ is attached and the carbon atom to which R⁵ is attached, form a 4-10 membered heterocycloalkyl, which is optionally substituted with 1, 2, 3, 4, or 5 substituents independently selected from R^{g};

R⁷ is selected from C₁₋₆ alkyl, C₁₋₆ haloalkyl, C₂₋₆ alkenyl, and C₂₋₆ alkynyl, wherein said C₁₋₆ alkyl is optionally substituted with 1, 2, or 3 substituents independently selected from OH, NO₂, CN, C₆₋₁₀ alkoxy, C₆₋₁₀ haloalkoxy, Cy, and a moiety of formula (i);

L¹ is selected from C(O), C₁₋₆ alkylene, C(O)—C₁₋₆ alkylene, C₃₋₁₀ cycloalkylene, C₂₋₆ alkenylene, and C₂₋₆ alkynylene, each of which is optionally substituted with 1, 2, or 3 substituents independently selected from R¹⁰; or L¹ is absent;

each R¹⁰ is independently selected from halo, OH, NO₂, CN, C₆₋₁₀ alkoxy, C₆₋₁₀ haloalkoxy, and Cy;

R⁸ is selected from H and C₁₋₆ alkyl, wherein said C₁₋₆ alkyl is optionally substituted with 1, 2, or 3 substituents independently selected from C₁₋₆ alkoxy, amino, C₁₋₆ alkylamino, di(C₁₋₆ alkyl)amino, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, 3, 4, or 5 substituents independently selected from R^{g};

or R⁸ and R¹⁰ together with the N atom to which R⁸ is attached, L¹ to which R¹⁰ is attached, and C(O) between the N and the L¹, form a 4-10 membered heterocycloalkyl, which is optionally substituted with 1, 2, 3, 4, or 5 substituents independently selected from R^{g};

L² is selected from C₁₋₆ alkylene, C₁₋₆ alkylene-NR⁸—C₁₋₆ alkylene, C₂₋₆ alkenylene, and C₂₋₆ alkynylene, each of which is optionally substituted with 1, 2, or 3 substituents independently selected from R^{g}; or L² is absent;

R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, 3, 4, or 5 substituents independently selected from R^{g};

or R⁸ and L²-R⁹, together with the N atom to which R⁸ and L² are attached, form a 4-10 membered heterocycloalkyl, which is optionally substituted with C(O)Cy and is optionally substituted with 1, 2, 3, 4, or 5 substituents independently selected from R^{g};

or R⁹ and R¹⁰, together with the L¹ to which R¹⁰ is attached, L² to which R⁹ is attached, and C(O) and NR⁸ between the L¹ and the L², form a 4-10 membered heterocycloalkyl, which is optionally substituted with 1, 2, 3, 4, or 5 substituents independently selected from R^{g};

Cy is selected from C₆₋₁₀ aryl and 5-10 membered heteroaryl, each of which is optionally substituted with 1, 2, 3, 4, or 5 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, NH₂—C₁₋₃ alkylene, C₁₋₆ alkylamino-C₁₋₃ alkylene, di(C₁₋₆ alkyl)amino-C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments:

R⁷ is selected from C₁₋₆ alkyl, C₁₋₆ haloalkyl, C₂₋₆ alkenyl, and C₂₋₆ alkynyl, wherein said C₁₋₆ alkyl is optionally substituted with 1, 2, or 3 substituents independently selected from OH, NO₂, CN, C₆₋₁₀ alkoxy, C₆₋₁₀ haloalkoxy, and Cy;

L¹ is selected from C(O), C₁₋₆ alkylene, C₂₋₆ alkenylene, and C₂₋₆ alkynylene, each of which is optionally substituted with 1, 2, or 3 substituents independently selected from halo, OH, NO₂, CN, C₆₋₁₀ alkoxy, C₆₋₁₀ haloalkoxy, and Cy; or L¹ is absent;

R⁸ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents independently selected from R^{g};

R⁹ is selected from H, C₁₋₆ alkoxy, C₆₋₁₀ aryl and 5-10 membered heteroaryl, each of which is optionally substituted with 1, 2, 3, 4, or 5 substituents independently selected from R^{g};

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments:

R¹, R², R³, R⁴, R⁵, and R⁶ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, C₁₋₆ haloalkoxy, wherein said C₁₋₆ alkyl is optionally substituted with 1, 2, or 3 substituents independently selected from OH, NO₂, CN, C₆₋₁₀ alkoxy, C₆₋₁₀ haloalkoxy, and Cy, and a moiety of formula

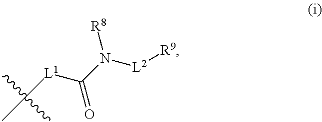

provided that at least one of R¹, R², R³, R⁴, R⁵, and R⁶ is a moiety of formula (i);

R⁷ is selected from C₁₋₆ alkyl, C₁₋₆ haloalkyl, C₂₋₆ alkenyl, and C₂₋₆ alkynyl, wherein said C₁₋₆ alkyl is optionally substituted with 1, 2, or 3 substituents independently selected from OH, NO₂, CN, C₆₋₁₀ alkoxy, C₆₋₁₀ haloalkoxy, and Cy;

L¹ is selected from C(O), C₁₋₆ alkylene, C₂₋₆ alkenylene, and C₂₋₆ alkynylene, each of which is optionally substituted with 1, 2, or 3 substituents independently selected from halo, OH, NO₂, CN, C₆₋₁₀ alkoxy, C₆₋₁₀ haloalkoxy, and Cy; or L¹ is absent;

R⁸ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents independently selected from R^{g}; or L² is absent;

R⁹ is selected from H, C₁₋₆ alkoxy, C₆₋₁₀ aryl and 5-10 membered heteroaryl, each of which is optionally substituted with 1, 2, 3, 4, or 5 substituents independently selected from R^{g};

Cy is selected from C₆₋₁₀ aryl and 5-10 membered heteroaryl, each of which is optionally substituted with 1, 2, 3, 4, or 5 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, the compound of Formula (V) has formula:

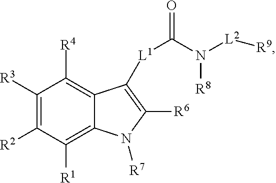

or a pharmaceutically acceptable salt thereof.

In some embodiments, R¹, R², R³, R⁴, and R⁶ are each H.

In some embodiments, R¹, R², R³, R⁴, and R⁶ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, C₁₋₆ haloalkoxy, wherein said C₁₋₆ alkyl is optionally substituted with 1, 2, or 3 substituents independently selected from OH, NO₂, CN, C₆₋₁₀ alkoxy, C₆₋₁₀ haloalkoxy, and Cy.

In some embodiments, R¹, R², R³, R⁴, and R⁶ are each independently selected from H, halo, C₁₋₆ alkyl, C₁₋₆ haloalkyl, C₁₋₆ alkoxy, and C₁₋₆ haloalkoxy.

In some embodiments, the compound of Formula (V) has formula:

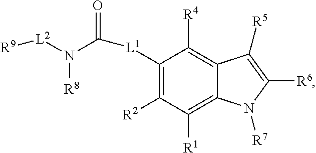

or a pharmaceutically acceptable salt thereof.

In some embodiments, R¹, R², R⁴, R⁵, and R⁶ are each H.

In some embodiments, R¹, R², R⁴, R⁵, and R⁶ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, C₁₋₆ haloalkoxy, wherein said C₁₋₆ alkyl is optionally substituted with 1, 2, or 3 substituents independently selected from OH, NO₂, CN, C₆₋₁₀ alkoxy, C₆₋₁₀ haloalkoxy, and Cy.

In some embodiments, R¹, R², R⁴, R⁵, and R⁶ are each independently selected from H, halo, C₁₋₆ alkyl, C₁₋₆ haloalkyl, C₁₋₆ alkoxy, and C₁₋₆ haloalkoxy. In some embodiments, R¹, R², and R⁴ are each H, and R⁵ and and R⁶ are each C₁₋₆ alkyl. In some embodiments, R¹, R², and R⁴ are each H, and R⁵ and and R⁶ are each independently selected from H and C₁₋₆ alkyl.

In some embodiments, the compound of Formula (V) has formula:

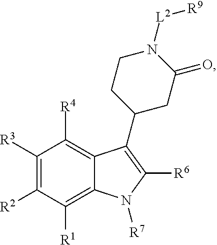

or a pharmaceutically acceptable salt thereof.

In some embodiments, R¹, R², R³, R⁴, and R⁶ are each H. In some embodiments, R¹, R², R⁴, and R⁶ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, C₁₋₆ haloalkoxy, wherein said C₁₋₆ alkyl is optionally substituted with 1, 2, or 3 substituents independently selected from OH, NO₂, CN, C₆₋₁₀ alkoxy, C₆₋₁₀ haloalkoxy, and Cy. In some embodiments, R¹, R², R⁴, and R⁶ are each independently selected from H, halo, C₁₋₆ alkyl, C₁₋₆ haloalkyl, C₁₋₆ alkoxy, and C₁₋₆ haloalkoxy.

In some embodiments, the compound of Formula (V) has formula:

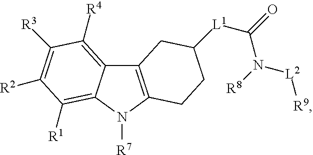

or a pharmaceutically acceptable salt thereof.

In some embodiments, the compound of Formula V has formula:

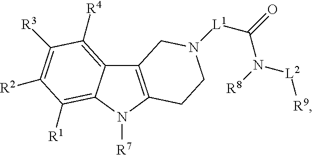

or a pharmaceutically acceptable salt thereof.

In some embodiments, the compound of Formula (V) has formula:

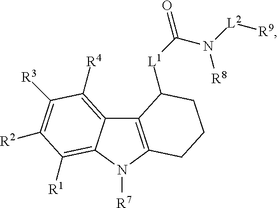

or a pharmaceutically acceptable salt thereof.

In some embodiments, R¹, R², R³, and R⁴ are each H. In some embodiments, R¹, R², R³, and R⁴ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, C₁₋₆ haloalkoxy, wherein said C₁₋₆ alkyl is optionally substituted with 1, 2, or 3 substituents independently selected from OH, NO₂, CN, C₆₋₁₀ alkoxy, C₆₋₁₀ haloalkoxy, and Cy. In some embodiments, R¹, R², R³, and R⁴ are each independently selected from H, halo, C₁₋₆ alkyl, C₁₋₆ haloalkyl, C₁₋₆ alkoxy, and C₁₋₆ haloalkoxy.

In some embodiments, the compound of Formula (V) has formula:

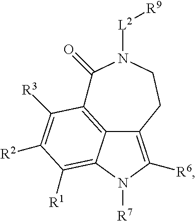

or a pharmaceutically acceptable salt thereof.

In some embodiments, R¹, R², R³, and R⁶ are each H. In some embodiments, R¹, R², R³, and R⁶ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, C₁₋₆ haloalkoxy, wherein said C₁₋₆ alkyl is optionally substituted with 1, 2, or 3 substituents independently selected from OH, NO₂, CN, C₆₋₁₀ alkoxy, C₆₋₁₀ haloalkoxy, and Cy. In some embodiments, R¹, R², R³, and R⁶ are each independently selected from H, halo, C₁₋₆ alkyl, C₁₋₆ haloalkyl, C₁₋₆ alkoxy, and C₁₋₆ haloalkoxy.

In some embodiments, R⁷ is C₁₋₆ alkyl, optionally substituted with Cy.

In some embodiments, R⁷ is C₁₋₆ alkyl.

In some embodiments, R⁷ is a moiety of formula (i). In some embodiments, R⁷ is C₁₋₆ alkyl, optionally substituted a moiety of formula (i).

In some embodiments, R⁷ is C₁₋₆ alkyl, optionally substituted with Cy or a moiety of formula (i).

In some embodiments, L¹ is C(O), C₁₋₆ alkylene, C(O)—C₁₋₆ alkylene, or C₃₋₁₀ cycloalkylene, each of which is optionally substituted with R¹⁰.

In some embodiments, L¹ is C(O). In some embodiments, L¹ is C₁₋₆ alkylene. In some embodiments, L¹ is C(O)—C₁₋₆ alkylene. In some embodiments, L¹ is C₃₋₁₀ cycloalkylene (e.g., cyclopropylene).

In some embodiments, L¹ is absent.

In some embodiments, L¹ is C(O) or C₁₋₆ alkylene, optionally substituted with Cy.

In some embodiments, R⁸ is C₁₋₆ alkyl, which is optionally substituted with C₁₋₆ alkoxy, di(C₁₋₆ alkyl)amino, or 4-10 membered heterocycloalkyl.

In some embodiments, R⁸ is H.

In some embodiments, L² is C₁₋₆ alkylene or C₁₋₆ alkylene-NR⁸—C₁₋₆ alkylene; or L² is absent. In some embodiments, L² is absent.

In some embodiments, L² is C₁₋₆ alkylene.

In some embodiments, L² is C₁₋₆ alkylene; or L² is absent.

In some embodiments, Cy is selected from C₆₋₁₀ aryl and 5-10 membered heteroaryl, each of which is optionally substituted with 1, 2, or 3 independently selected R^{g}.

In some embodiments, Cy is selected from C₆₋₁₀ aryl and 5-10 membered heteroaryl, each of which is optionally substituted with 1, 2, or 3 substituents independently selected from halo, C₁₋₆ alkyl, and C₁₋₆ alkoxy.

In some embodiments, Cy is 5-10 membered heteroaryl.

In some embodiments, Cy is 5-10 membered heteroaryl, optionally substituted with 1, 2, or 3 substituents independently selected from halo, C₁₋₆ alkyl, and C₁₋₆ alkoxy.

In some embodiments, Cy is C₆₋₁₀ aryl, optionally substituted with halo.

In some embodiments, Cy is C₆₋₁₀ aryl, optionally substituted with 1, 2, or 3 substituents independently selected from halo, C₁₋₆ alkyl, and C₁₋₆ alkoxy.

In some embodiments, R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

In some embodiments, R⁹ is selected from H, C₁₋₆ alkoxy, C₆₋₁₀ aryl, and 5-10 membered heteroaryl, optionally substituted with 1, 2, or 3 substituents independently selected from R^{g}.

In some embodiments, R⁹ is H. In some embodiments, R⁹ is C₆₋₁₀ aryl. In some embodiments, R⁹ is C₆₋₁₀ aryloxy. In some embodiments, R⁹ is 5-10 membered heteroaryl. In some embodiments, R⁹ is 4-10 membered heterocycloalkyl. In each of these embodiments, R⁹ is optionally substituted with 1, 2, or 3 substituents independently selected from R⁹.

In some embodiments, R⁵ and R⁶ together with the carbon atoms to which they are attached from a C₄₋₇ cycloalkyl ring or a 4-7 membered heterocycloalkyl ring, each of which is optionally substituted with 1, 2, or 3 substituents independently selected from OH, NO₂, CN, C₆₋₁₀ alkoxy, C₆₋₁₀ haloalkoxy, Cy, and a moiety of formula (i).

In some embodiments, R⁵ and R⁶ together with the carbon atoms to which they are attached from a 4-7 membered heterocycloalkyl ring, which is optionally substituted with a moiety of formula (i).

In some embodiments, R⁵ and R⁶ together with the carbon atoms to which they are attached from a C₃₋₇ cycloalkyl ring, which is optionally substituted with a moiety of formula (i).

In some embodiments, R⁴ is a moiety of formula (i). In some aspects of these embodiments, R⁸ of the formula (i) and R⁵, together with the N atom to which R⁸ is attached and the carbon atom to which R⁵ is attached, form a 4-10 membered heterocycloalkyl, which is optionally substituted with 1, 2, or 3 substituents independently selected from R^{g}.

In some embodiments, R⁸ and R¹⁰ together with the N atom to which R⁸ is attached, L¹ to which R¹⁰ is attached, and C(O) between the N and the L¹, form a 4-10 membered heterocycloalkyl, which is optionally substituted with 1, 2, or 3 substituents independently selected from R^{g}.

In some embodiments, R⁸ and L²-R⁹, together with the N atom to which R⁸ and L² are attached, form a 4-10 membered heterocycloalkyl, which is optionally substituted with C(O)Cy and is optionally substituted with 1, 2, or 3 substituents independently selected from R^{g}.

In some embodiments, R⁸ and L²-R⁹, together with the N atom to which R⁸ and L² are attached, form a 4-10 membered heterocycloalkyl, which is optionally substituted with C(O)Cy. In some aspects of the above embodiments, the heterocycloalkyl is piperazine.

In some embodiments, R⁹ and R¹⁰, together with the L¹ to which R¹⁰ is attached, L² to which R⁹ is attached, and C(O) and NR⁸ between the L¹ and the L², form a 4-10 membered heterocycloalkyl, which is optionally substituted with 1, 2, 3, 4, or 5 substituents independently selected from R^{g}.

In some embodiments, the compound of Formula (V) has formula:

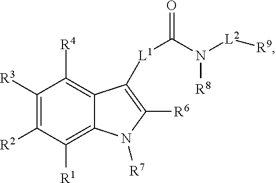

or a pharmaceutically acceptable salt thereof, wherein:

R¹, R², R³, R⁴, and R⁶ are each H;

R⁷ is C₁₋₆ alkyl, optionally substituted with Cy or a moiety of formula (i);

L¹ is selected from C₁₋₆ alkylene, C(O)—C₁₋₆ alkylene, and C₃₋₁₀ cycloalkylene, each of which is optionally substituted with R¹⁰; or L¹ is absent;

each R¹⁰ is Cy;

R⁸ is selected from H and C₁₋₆ alkyl, which is optionally substituted with C₁₋₆ alkoxy, di(C₁₋₆ alkyl)amino, or 4-10 membered heterocycloalkyl;

L² is C₁₋₆ alkylene or C₁₋₆ alkylene-NR⁸—C₁₋₆ alkylene; or L² is absent;

Cy is selected from C₆₋₁₀ aryl and 5-10 membered heteroaryl, each of which is optionally substituted with 1, 2, or 3 independently selected R^{g}; and

R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

In some embodiments:

R¹, R², R³, R⁴, and R⁶ are each H;

R⁷ is C₁₋₆ alkyl, optionally substituted with Cy;

L¹ is C₁₋₆ alkylene, optionally substituted with Cy;

R⁸ is H;

L² is C₁₋₆ alkylene;

Cy is C₆₋₁₀ aryl, optionally substituted with halo; and

R⁹ is selected from H, C₁₋₆ alkoxy, C₆₋₁₀ aryl, and 5-10 membered heteroaryl, each of which is optionally substituted with 1, 2, or 3 substituents independently selected from R^{g}.

In some aspects of these embodiments:

R¹, R², R³, R⁴, and R⁶ are each H;

R⁷ is C₁₋₆ alkyl, optionally substituted with Cy;

L¹ is C₁₋₆ alkylene, optionally substituted with Cy;

R⁸ is H;

L² is C₁₋₆ alkylene;

Cy is phenyl, optionally substituted with halo; and

R⁹ is selected from H, C₁₋₃ alkoxy, phenyl, furan-2-yl, and thiophenyl, each of which is optionally substituted with 1, 2, or 3 substituents independently selected from R^{g}.

In some embodiments, the compound of Formula (V) has formula:

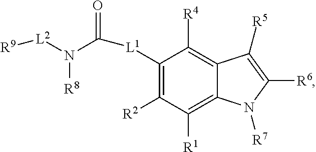

or a pharmaceutically acceptable salt thereof, wherein:

R¹, R², and R⁴ are each H, and R⁵ and R⁶ are each independently selected from H and C₁₋₆ alkyl;

R⁷ is C₁₋₆ alkyl, optionally substituted with Cy or a moiety of formula (i);

L¹ is selected from C(O), C₁₋₆ alkylene, C(O)—C₁₋₆ alkylene, and C₃₋₁₀ cycloalkylene, each of which is optionally substituted with R¹⁰; or L¹ is absent;

each R¹⁰ is Cy;

R⁸ is selected from H and C₁₋₆ alkyl, which is optionally substituted with C₁₋₆ alkoxy, di(C₁₋₆ alkyl)amino, or 4-10 membered heterocycloalkyl;

L² is C₁₋₆ alkylene or C₁₋₆ alkylene-NR⁸—C₁₋₆ alkylene; or L² is absent;

Cy is selected from C₆₋₁₀ aryl and 5-10 membered heteroaryl, each of which is optionally substituted with 1, 2, or 3 independently selected R^{g}; and

R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

In some embodiments:

R¹, R², R⁴, R⁵, and R⁶ are each H;

R⁷ is C₁₋₆ alkyl, optionally substituted with Cy;

L¹ is C₁₋₆ alkylene, optionally substituted with Cy;

R⁸ is H;

L² is C₁₋₆ alkylene; or L² is absent;

Cy is C₆₋₁₀ aryl, optionally substituted with halo; and

R⁹ is 5-10 membered heteroaryl, optionally substituted with 1, 2, or 3 substituents independently selected from R^{g}.

In some aspects of these embodiments:

R¹, R², R⁴, R⁵, and R⁶ are each H;

R⁷ is C₁₋₆ alkyl, optionally substituted with Cy;

L¹ is C₁₋₆ alkylene, optionally substituted with Cy;

R⁸ is H;

L² is C₁₋₆ alkylene;

Cy is phenyl, optionally substituted with halo; and

R⁹ is furan-2-yl, optionally substituted with 1, 2, or 3 substituents independently selected from R^{g}.

In some embodiments, the compound of Formula (V) has formula:

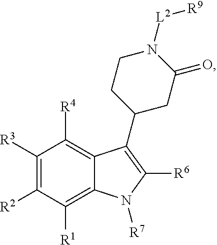

or a pharmaceutically acceptable salt thereof, wherein:

R¹, R², R³, R⁴, and R⁶ are each H;

R⁷ is C₁₋₆ alkyl, optionally substituted with Cy or a moiety of formula (i);

L² is C₁₋₆ alkylene or C₁₋₆ alkylene-NR⁸—C₁₋₆ alkylene; or L² is absent;

R⁸ is selected from H and C₁₋₆ alkyl, which is optionally substituted with C₁₋₆ alkoxy, di(C₁₋₆ alkyl)amino, or 4-10 membered heterocycloalkyl;

Cy is selected from C₆₋₁₀ aryl and 5-10 membered heteroaryl, each of which is optionally substituted with 1, 2, or 3 independently selected R^{g}; and

R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

In some embodiments, the compound of Formula (V) is selected from any one of the following formulae:

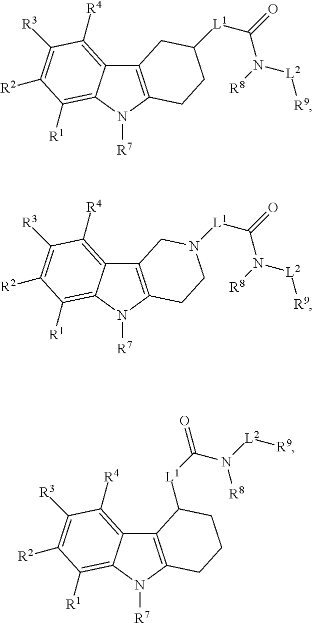

or a pharmaceutically acceptable salt thereof, wherein:

R¹, R², R³, and R⁴ are each H;

R⁷ is C₁₋₆ alkyl, optionally substituted with Cy or a moiety of formula (i);

L¹ is selected from C(O), C₁₋₆ alkylene, C(O)—C₁₋₆ alkylene, and C₃₋₁₀ cycloalkylene, each of which is optionally substituted with R¹⁰; or L¹ is absent;

each R¹⁰ is Cy;

R⁸ is selected from H and C₁₋₆ alkyl, which is optionally substituted with C₁₋₆ alkoxy, di(C₁₋₆ alkyl)amino, or 4-10 membered heterocycloalkyl;

L² is C₁₋₆ alkylene or C₁₋₆ alkylene-NR⁸—C₁₋₆ alkylene; or L² is absent;

Cy is selected from C₆₋₁₀ aryl and 5-10 membered heteroaryl, each of which is optionally substituted with 1, 2, or 3 independently selected R^{g}; and

R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

In some embodiments, the compound of Formula (V) has formula:

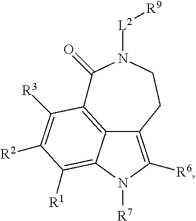

or a pharmaceutically acceptable salt thereof, wherein:

R¹, R², R³, and R⁶ are each H;

R⁷ is C₁₋₆ alkyl, optionally substituted with Cy or a moiety of formula (i);

L² is C₁₋₆ alkylene or C₁₋₆ alkylene-NR⁸—C₁₋₆ alkylene; or L² is absent;

R⁸ is selected from H and C₁₋₆ alkyl, which is optionally substituted with C₁₋₆ alkoxy, di(C₁₋₆ alkyl)amino, or 4-10 membered heterocycloalkyl;

Cy is selected from C₆₋₁₀ aryl and 5-10 membered heteroaryl, each of which is optionally substituted with 1, 2, or 3 independently selected R^{g}; and

R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

In some embodiments, the compound of Formula (V) is selected from any one of the compounds listed in Table 5, or a pharmaceutically acceptable salt thereof.

TABLE 5

| | |
|---|---|
| 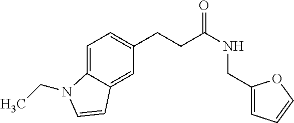 [SMILES: CCn1ccc2cc(CCC(=O)NCc3ccco3)ccc21] | BC181141 |
| 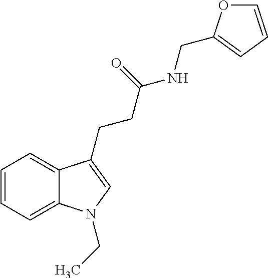 [SMILES: CCn1cc(CCC(=O)NCc2ccco2)c2ccccc21] | BC181115 |
| 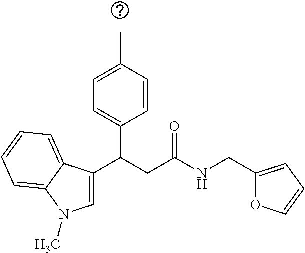 [SMILES: Cc1ccc(C(CC(=O)NCc2ccco2)c2cn(C)c3ccccc23)cc1] | BC181142 |
| 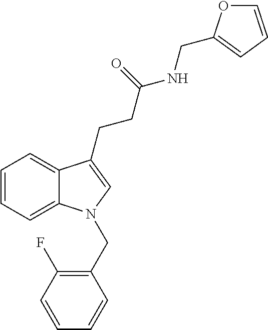 [SMILES: O=C(CCc1cn(Cc2ccccc2F)c2ccccc12)NCc1ccco1] | BC181117 |
| 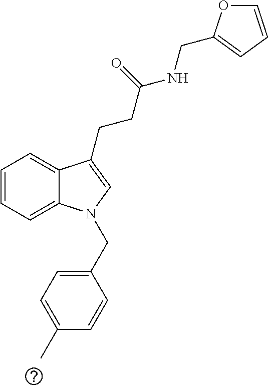 [SMILES: Cc1ccc(Cn2cc(CCC(=O)NCc3ccco3)c3ccccc32)cc1] | BC181116 |
| 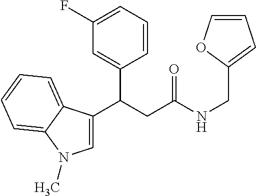 [SMILES: Cn1cc(C(CC(=O)NCc2ccco2)c2cccc(F)c2)c2ccccc21] | BC181143 |
| 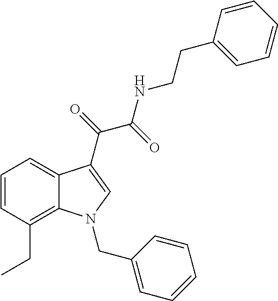 [SMILES: CCc1cccc2c(C(=O)C(=O)NCCc3ccccc3)cn(Cc3ccccc3)c12] | BC181228 |
| 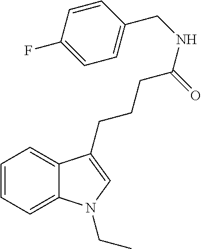 [SMILES: CCn1cc(CCCC(=O)NCc2ccc(F)cc2)c2ccccc21] | BC181229 |
| 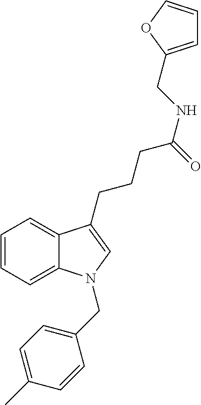 [SMILES: Cc1ccc(Cn2cc(CCCC(=O)NCc3ccco3)c3ccccc32)cc1] | BC181230 |
| 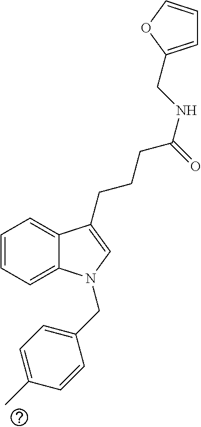 [SMILES: Cc1ccc(Cn2cc(CCCC(=O)NCc3ccco3)c3ccccc32)cc1] | BC181231 |
| 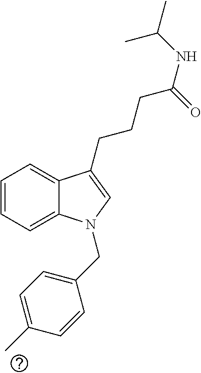 [SMILES: Cc1ccc(Cn2cc(CCCC(=O)NC(C)C)c3ccccc32)cc1] | BC181232 |
| 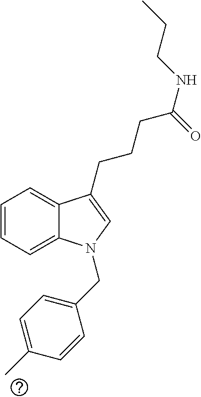 [SMILES: CCCNC(=O)CCCc1cn(Cc2ccc(C)cc2)c2ccccc12] | BC181233 |
| 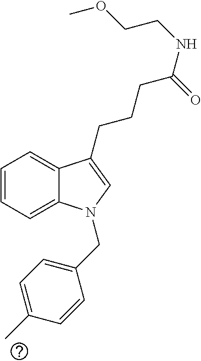 [SMILES: COCCNC(=O)CCCc1cn(Cc2ccc(C)cc2)c2ccccc12] | BC181234 |
| 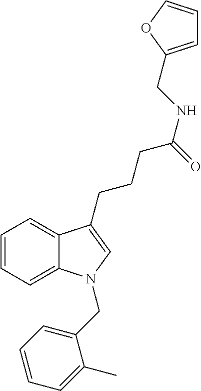 [SMILES: Cc1ccccc1Cn1cc(CCCC(=O)NCc2ccco2)c2ccccc21] | BC181235 |
| 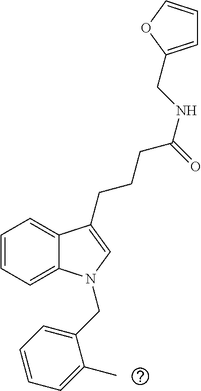 [SMILES: Cc1ccccc1Cn1cc(CCCC(=O)NCc2ccco2)c2ccccc21] | BC181236 |
| 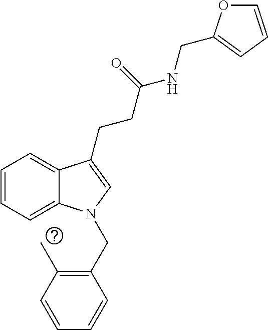 [SMILES: Cc1ccccc1Cn1cc(CCC(=O)NCc2ccco2)c2ccccc21] | BC181237 |
| 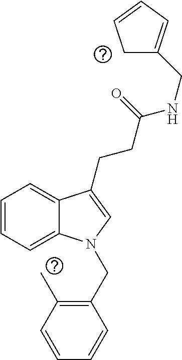 [SMILES: Cc1ccccc1Cn1cc(CCC(=O)NCC2=CC=CC2)c2ccccc21] | BC181238 |
| 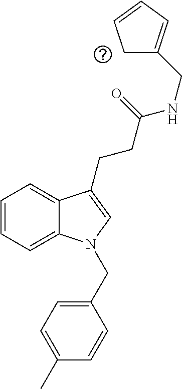 [SMILES: Cc1ccc(Cn2cc(CCC(=O)NCC3=CC=CC3)c3ccccc32)cc1] | BC181239 |
| 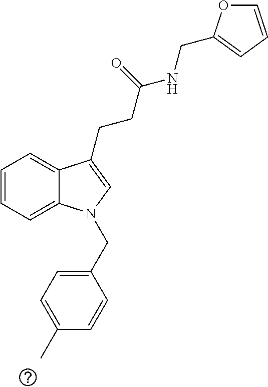 [SMILES: Cc1ccc(Cn2cc(CCC(=O)NCc3ccco3)c3ccccc32)cc1] | BC181240 |
| 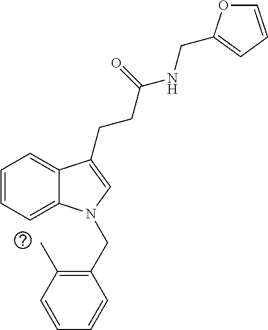 [SMILES: Cc1ccccc1Cn1cc(CCC(=O)NCc2ccco2)c2ccccc21] | BC181241 |
| 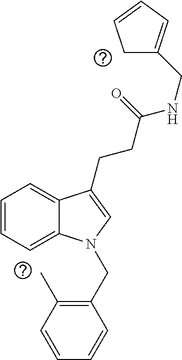 [SMILES: Cc1ccccc1Cn1cc(CCC(=O)NCC2=CC=CC2)c2ccccc21] | BC181242 |
| 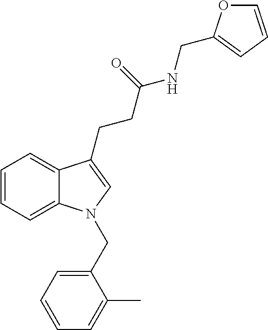 [SMILES: Cc1ccccc1Cn1cc(CCC(=O)NCc2ccco2)c2ccccc21] | BC181243 |
| 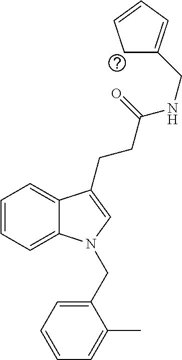 [SMILES: Cc1ccccc1Cn1cc(CCC(=O)NCC2=CC=CC2)c2ccccc21] | BC181244 |
| 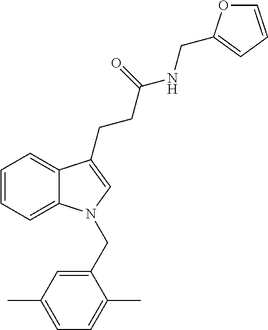 [SMILES: Cc1ccc(C)c(Cn2cc(CCC(=O)NCc3ccco3)c3ccccc32)c1] | BC181245 |
| 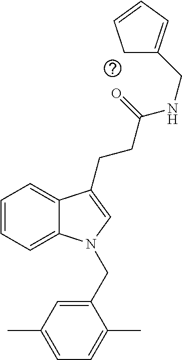 [SMILES: Cc1ccc(C)c(Cn2cc(CCC(=O)NCC3=CC=CC3)c3ccccc32)c1] | BC181246 |
| 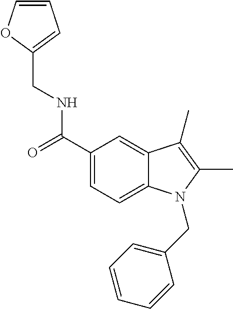 [SMILES: Cc1c(C)n(Cc2ccccc2)c2ccc(C(=O)NCc3ccco3)cc12] | BC181247 |
| 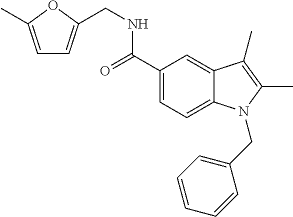 | BC181248 |
| 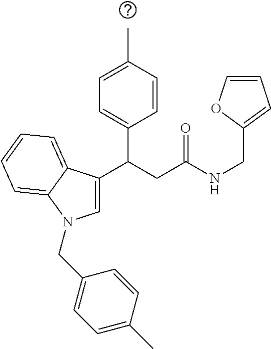 [SMILES: Cc1ccc(Cn2cc(C(CC(=O)NCc3ccco3)c3ccc(C)cc3)c3ccccc32)cc1] | BC181258 |
| 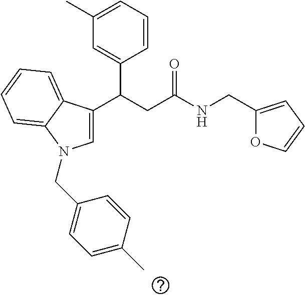 [SMILES: Cc1ccc(Cn2cc(C(CC(=O)NCc3ccco3)c3cccc(C)c3)c3ccccc32)cc1] | BC181259 |
| 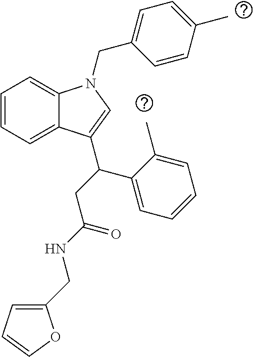 [SMILES: Cc1ccc(Cn2cc(C(CC(=O)NCc3ccco3)c3ccccc3C)c3ccccc32)cc1] | BC181260 |
| 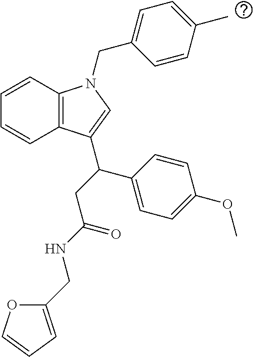 [SMILES: COc1ccc(C(CC(=O)NCc2ccco2)c2cn(Cc3ccc(C)cc3)c3ccccc23)cc1] | BC181261 |
| 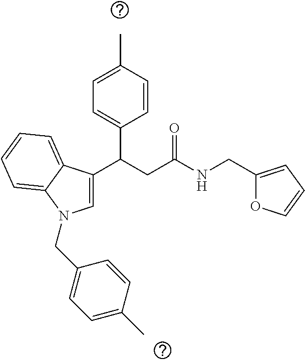 [SMILES: Cc1ccc(Cn2cc(C(CC(=O)NCc3ccco3)c3ccc(C)cc3)c3ccccc32)cc1] | BC181262 |
| 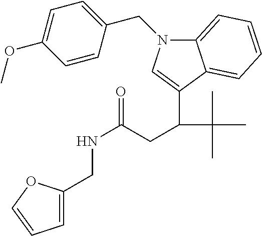 [SMILES: COc1ccc(Cn2cc(C(CC(=O)NCc3ccco3)C(C)(C)C)c3ccccc32)cc1] | BC181263 |
| 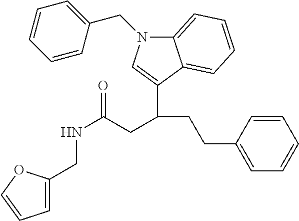 [SMILES: O=C(CC(CCc1ccccc1)c1cn(Cc2ccccc2)c2ccccc12)NCc1ccco1] | BC181264 |
| 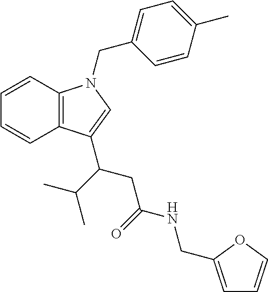 [SMILES: Cc1ccc(Cn2cc(C(CC(=O)NCc3ccco3)C(C)C)c3ccccc32)cc1] | BC181265 |
| 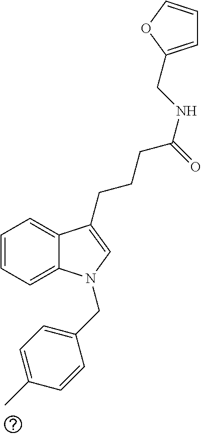 [SMILES: Cc1ccc(Cn2cc(CCCC(=O)NCc3ccco3)c3ccccc32)cc1] | BC181114 |

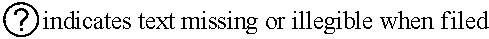

In some embodiments, the compound of Formula (V) is selected from any one of the compounds listed in Table 5a, or a pharmaceutically acceptable salt thereof.

TABLE 5a

| | |
|---|---|
| 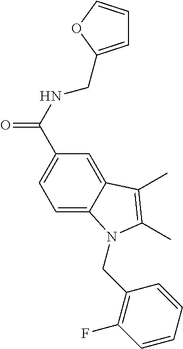 [SMILES: Cc1c(C)n(Cc2ccccc2F)c2ccc(C(=O)NCc3ccco3)cc12] | BC20100 |
| 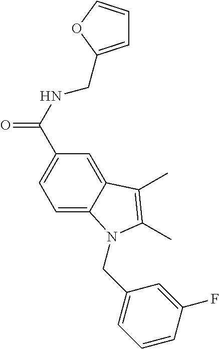 [SMILES: Cc1c(C)n(Cc2cccc(F)c2)c2ccc(C(=O)NCc3ccco3)cc12] | BC20101 |
| 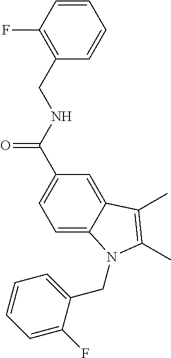 [SMILES: Cc1c(C)n(Cc2ccccc2F)c2ccc(C(=O)NCc3ccccc3F)cc12] | BC20103 |
| 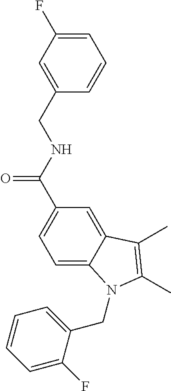 [SMILES: Cc1c(C)n(Cc2ccccc2F)c2ccc(C(=O)NCc3cccc(F)c3)cc12] | BC20104 |
| 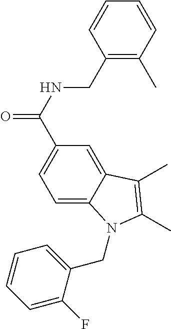 [SMILES: Cc1ccccc1CNC(=O)c1ccc2c(c1)c(C)c(C)n2Cc1ccccc1F] | BC20105 |
| 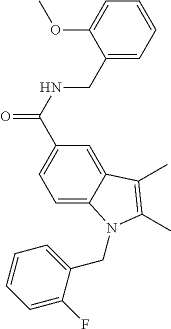 [SMILES: COc1ccccc1CNC(=O)c1ccc2c(c1)c(C)c(C)n2Cc1ccccc1F] | BC20106 |
| 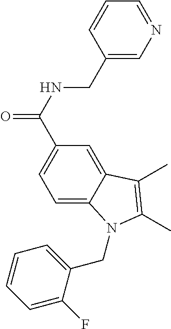 [SMILES: Cc1c(C)n(Cc2ccccc2F)c2ccc(C(=O)NCc3cccnc3)cc12] | BC20108 |
| 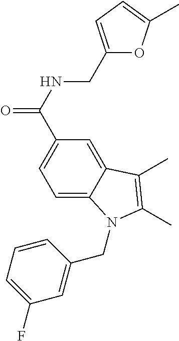 [SMILES: Cc1ccc(CNC(=O)c2ccc3c(c2)c(C)c(C)n3Cc2cccc(F)c2)o1] | BC20109 |
| 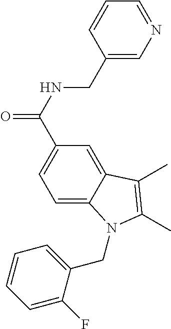 [SMILES: Cc1c(C)n(Cc2ccccc2F)c2ccc(C(=O)NCc3cccnc3)cc12] | BC20111 |
| 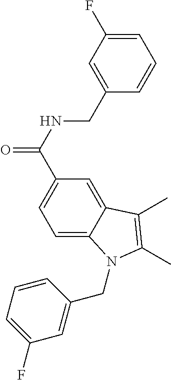 [SMILES: Cc1c(C)n(Cc2cccc(F)c2)c2ccc(C(=O)NCc3cccc(F)c3)cc12] | BC20112 |
| 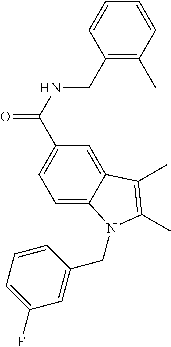 [SMILES: Cc1ccccc1CNC(=O)c1ccc2c(c1)c(C)c(C)n2Cc1cccc(F)c1] | BC20113 |
| 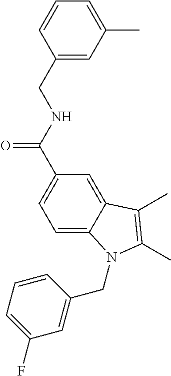 [SMILES: Cc1cccc(CNC(=O)c2ccc3c(c2)c(C)c(C)n3Cc2cccc(F)c2)c1] | BC20114 |
| 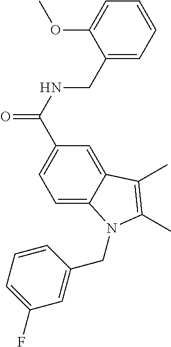 [SMILES: COc1ccccc1CNC(=O)c1ccc2c(c1)c(C)c(C)n2Cc1cccc(F)c1] | BC20115 |
| 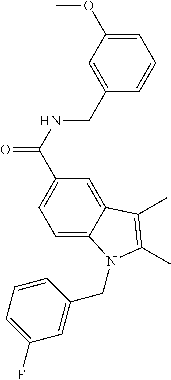 [SMILES: COc1cccc(CNC(=O)c2ccc3c(c2)c(C)c(C)n3Cc2cccc(F)c2)c1] | BC20116 |
| 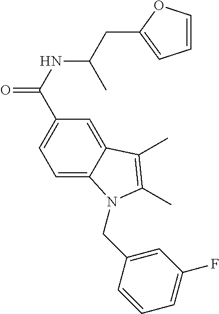 [SMILES: Cc1c(C)n(Cc2cccc(F)c2)c2ccc(C(=O)NC(C)Cc3ccco3)cc12] | BC20118 |
| 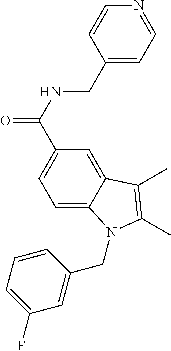 [SMILES: Cc1c(C)n(Cc2cccc(F)c2)c2ccc(C(=O)NCc3ccncc3)cc12] | BC20119 |
| 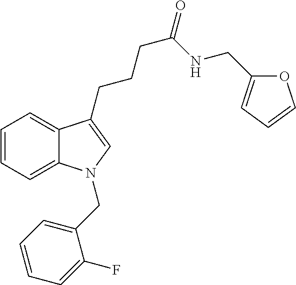 [SMILES: O=C(CCCc1cn(Cc2ccccc2F)c2ccccc12)NCc1ccco1] | BC20121 |
| 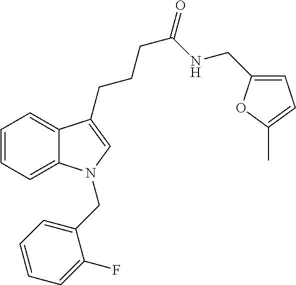 [SMILES: Cc1ccc(CNC(=O)CCCc2cn(Cc3ccccc3F)c3ccccc23)o1] | BC20122 |
| 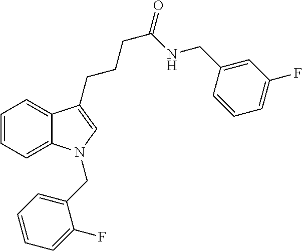 [SMILES: O=C(CCCc1cn(Cc2ccccc2F)c2ccccc12)NCc1cccc(F)c1] | BC20124 |
| 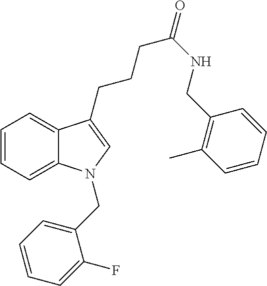 [SMILES: Cc1ccccc1CNC(=O)CCCc1cn(Cc2ccccc2F)c2ccccc12] | BC20125 |
| 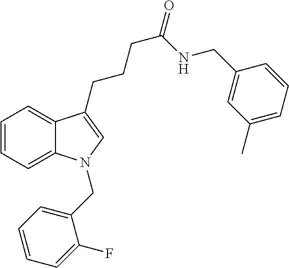 [SMILES: Cc1cccc(CNC(=O)CCCc2cn(Cc3ccccc3F)c3ccccc23)c1] | BC20126 |
| 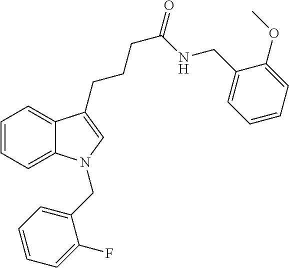 [SMILES: COc1ccccc1CNC(=O)CCCc1cn(Cc2ccccc2F)c2ccccc12] | BC20127 |
| 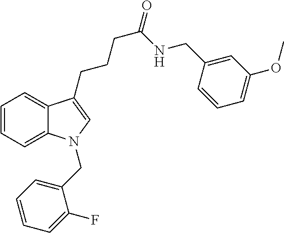 [SMILES: COc1cccc(CNC(=O)CCCc2cn(Cc3ccccc3F)c3ccccc23)c1] | BC20128 |
| 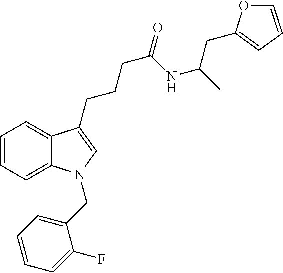 [SMILES: CC(Cc1ccco1)NC(=O)CCCc1cn(Cc2ccccc2F)c2ccccc12] | BC20130 |
| 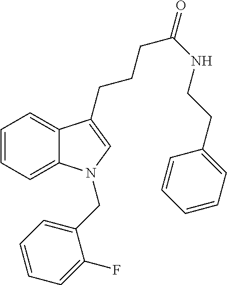 [SMILES: O=C(CCCc1cn(Cc2ccccc2F)c2ccccc12)NCCc1ccccc1] | BC20131 |
| 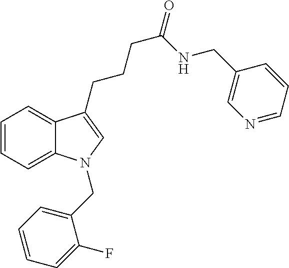 [SMILES: O=C(CCCc1cn(Cc2ccccc2F)c2ccccc12)NCc1cccnc1] | BC20132 |
| 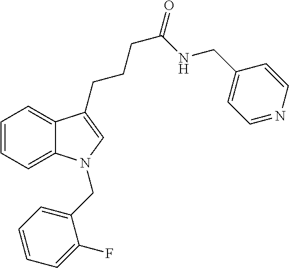 [SMILES: O=C(CCCc1cn(Cc2ccccc2F)c2ccccc12)NCc1ccncc1] | BC20133 |
| 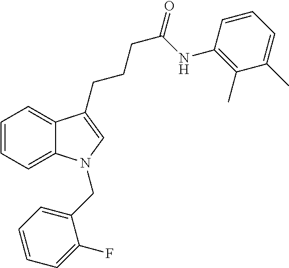 [SMILES: Cc1cccc(NC(=O)CCCc2cn(Cc3ccccc3F)c3ccccc23)c1C] | BC20134 |
| 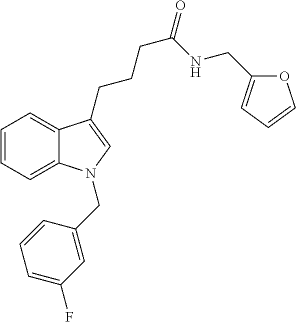 [SMILES: O=C(CCCc1cn(Cc2cccc(F)c2)c2ccccc12)NCc1ccco1] | BC20135 |
| 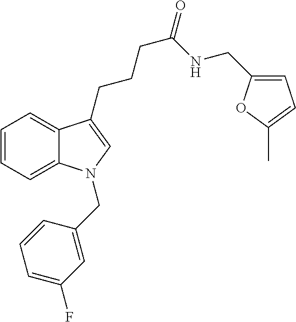 [SMILES: Cc1ccc(CNC(=O)CCCc2cn(Cc3cccc(F)c3)c3ccccc23)o1] | BC20136 |
| 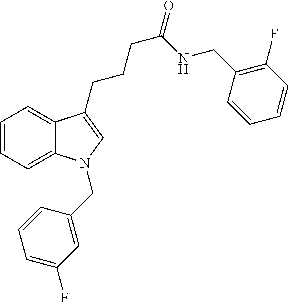 [SMILES: O=C(CCCc1cn(Cc2cccc(F)c2)c2ccccc12)NCc1ccccc1F] | BC20138 |
| 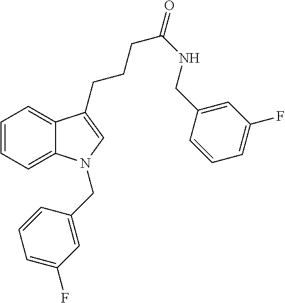 [SMILES: O=C(CCCc1cn(Cc2cccc(F)c2)c2ccccc12)NCc1cccc(F)c1] | BC20139 |
| 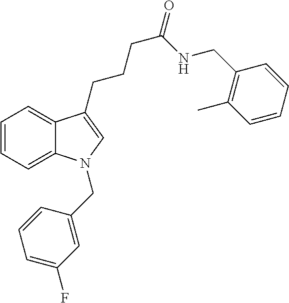 [SMILES: Cc1ccccc1CNC(=O)CCCc1cn(Cc2cccc(F)c2)c2ccccc12] | BC20140 |
| 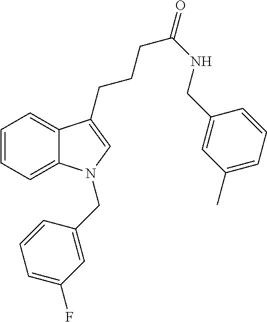 [SMILES: Cc1cccc(CNC(=O)CCCc2cn(Cc3cccc(F)c3)c3ccccc23)c1] | BC20141 |
| 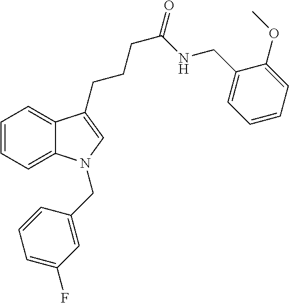 [SMILES: COc1ccccc1CNC(=O)CCCc1cn(Cc2cccc(F)c2)c2ccccc12] | BC20142 |
| 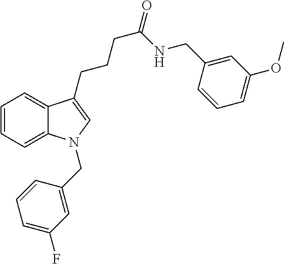 [SMILES: COc1cccc(CNC(=O)CCCc2cn(Cc3cccc(F)c3)c3ccccc23)c1] | BC20143 |
| 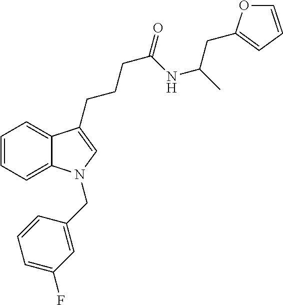 [SMILES: CC(Cc1ccco1)NC(=O)CCCc1cn(Cc2cccc(F)c2)c2ccccc12] | BC20145 |
| 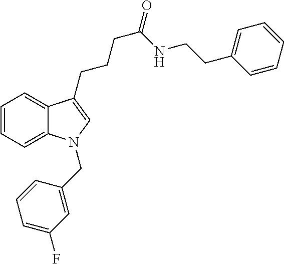 [SMILES: O=C(CCCc1cn(Cc2cccc(F)c2)c2ccccc12)NCCc1ccccc1] | BC20146 |
| 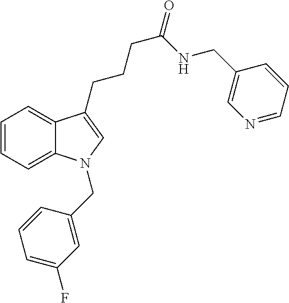 [SMILES: O=C(CCCc1cn(Cc2cccc(F)c2)c2ccccc12)NCc1cccnc1] | BC20147 |
| 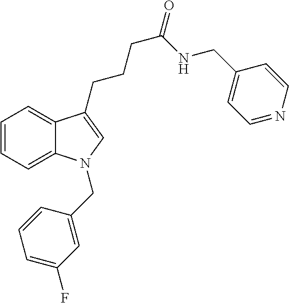 [SMILES: O=C(CCCc1cn(Cc2cccc(F)c2)c2ccccc12)NCc1ccncc1] | BC20148 |
| 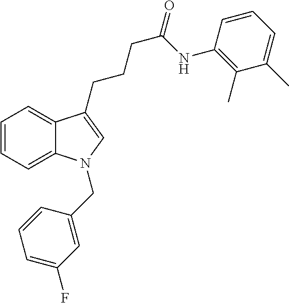 [SMILES: Cc1cccc(NC(=O)CCCc2cn(Cc3cccc(F)c3)c3ccccc23)c1C] | BC20149 |
| 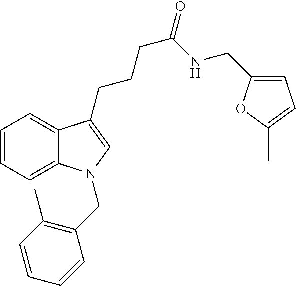 [SMILES: Cc1ccc(CNC(=O)CCCc2cn(Cc3ccccc3C)c3ccccc23)o1] | BC20150 |
| 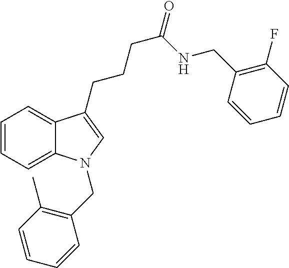 [SMILES: Cc1ccccc1Cn1cc(CCCC(=O)NCc2ccccc2F)c2ccccc21] | BC20151 |
| 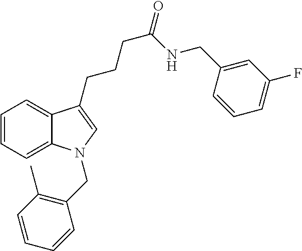 [SMILES: Cc1ccccc1Cn1cc(CCCC(=O)NCc2cccc(F)c2)c2ccccc21] | BC20152 |
| 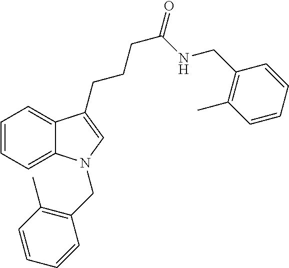 [SMILES: Cc1ccccc1CNC(=O)CCCc1cn(Cc2ccccc2C)c2ccccc12] | BC20153 |
| 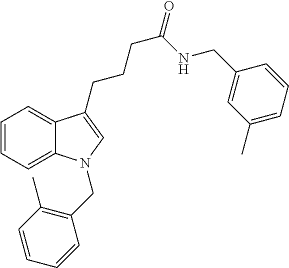 [SMILES: Cc1cccc(CNC(=O)CCCc2cn(Cc3ccccc3C)c3ccccc23)c1] | BC20154 |
| 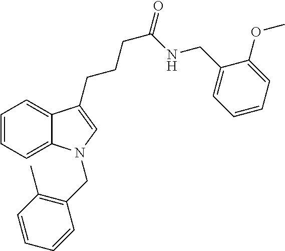 [SMILES: COc1ccccc1CNC(=O)CCCc1cn(Cc2ccccc2C)c2ccccc12] | BC20155 |
| 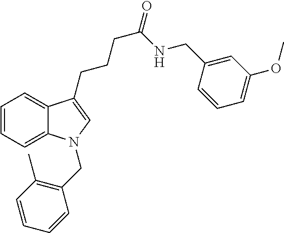 [SMILES: COc1cccc(CNC(=O)CCCc2cn(Cc3ccccc3C)c3ccccc23)c1] | BC20156 |
| 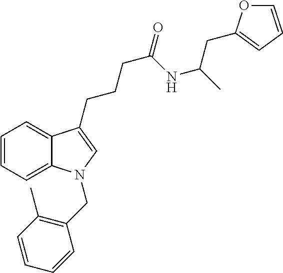 [SMILES: Cc1ccccc1Cn1cc(CCCC(=O)NC(C)Cc2ccco2)c2ccccc21] | BC20159 |
| 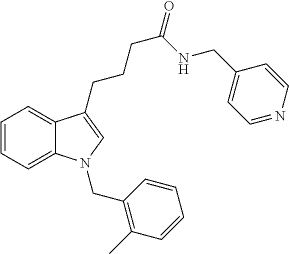 [SMILES: Cc1ccccc1Cn1cc(CCCC(=O)NCc2ccncc2)c2ccccc21] | BC20160 |
| 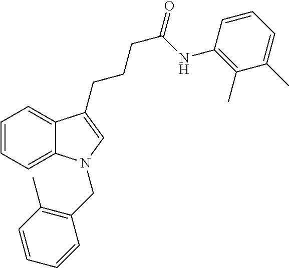 [SMILES: Cc1ccccc1Cn1cc(CCCC(=O)Nc2cccc(C)c2C)c2ccccc21] | BC20161 |
| 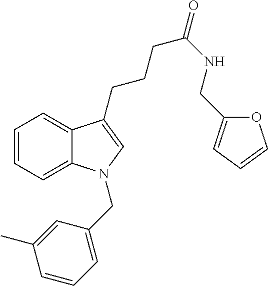 [SMILES: Cc1cccc(Cn2cc(CCCC(=O)NCc3ccco3)c3ccccc32)c1] | BC20163 |
| 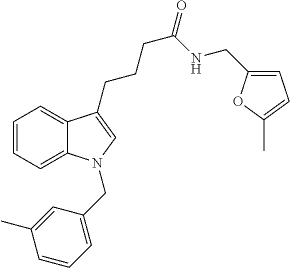 [SMILES: Cc1cccc(Cn2cc(CCCC(=O)NCc3ccc(C)o3)c3ccccc32)c1] | BC20164 |
| 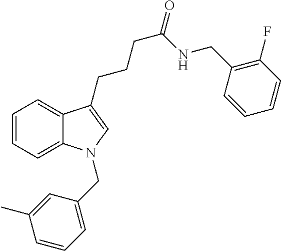 [SMILES: Cc1cccc(Cn2cc(CCCC(=O)NCc3ccccc3F)c3ccccc32)c1] | BC20165 |
| 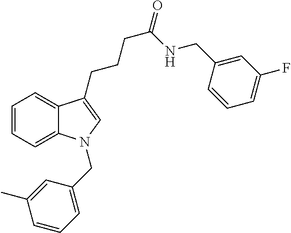 [SMILES: Cc1cccc(Cn2cc(CCCC(=O)NCc3cccc(F)c3)c3ccccc32)c1] | BC20166 |
| 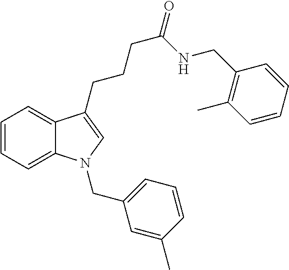 [SMILES: Cc1cccc(Cn2cc(CCCC(=O)NCc3ccccc3C)c3ccccc32)c1] | BC20167 |
| 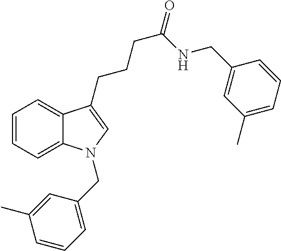 [SMILES: Cc1cccc(CNC(=O)CCCc2cn(Cc3cccc(C)c3)c3ccccc23)c1] | BC20168 |
| 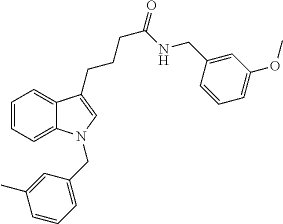 | BC20169 |
| 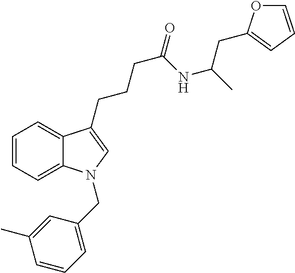 [SMILES: Cc1cccc(Cn2cc(CCCC(=O)NC(C)Cc3ccco3)c3ccccc32)c1] | BC20171 |
| 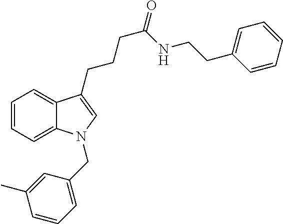 [SMILES: Cc1cccc(Cn2cc(CCCC(=O)NCCc3ccccc3)c3ccccc32)c1] | BC20172 |
| 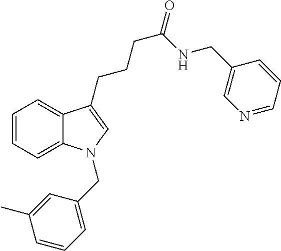 [SMILES: Cc1cccc(Cn2cc(CCCC(=O)NCc3cccnc3)c3ccccc32)c1] | BC20173 |
| 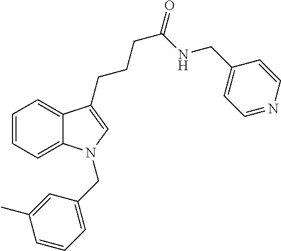 [SMILES: Cc1cccc(Cn2cc(CCCC(=O)NCc3ccncc3)c3ccccc32)c1] | BC20174 |
| 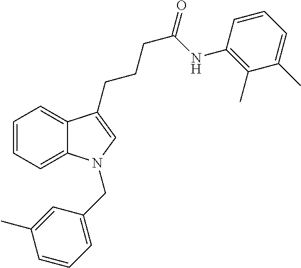 | BC20175 |
| 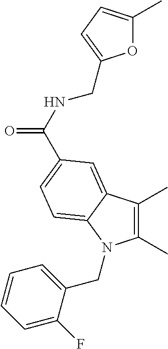 [SMILES: Cc1ccc(CNC(=O)c2ccc3c(c2)c(C)c(C)n3Cc2ccccc2F)o1] | BC20193 |
| 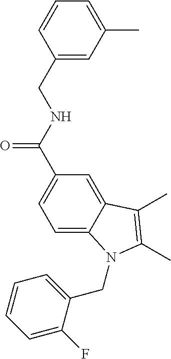 [SMILES: Cc1cccc(CNC(=O)c2ccc3c(c2)c(C)c(C)n3Cc2ccccc2F)c1] | BC20195 |
| 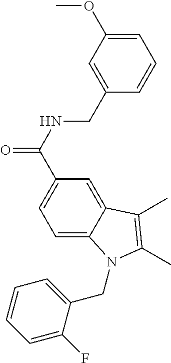 [SMILES: COc1cccc(CNC(=O)c2ccc3c(c2)c(C)c(C)n3Cc2ccccc2F)c1] | BC20196 |
| 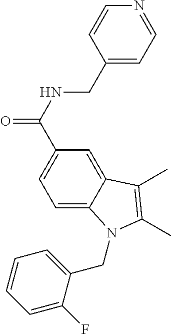 [SMILES: Cc1c(C)n(Cc2ccccc2F)c2ccc(C(=O)NCc3ccncc3)cc12] | BC20197 |
| 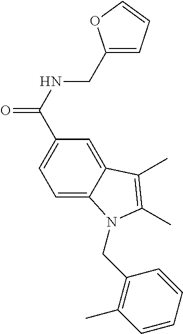 [SMILES: Cc1ccccc1Cn1c(C)c(C)c2cc(C(=O)NCc3ccco3)ccc21] | BC20199 |
| 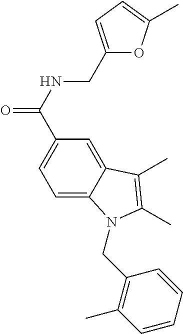 [SMILES: Cc1ccc(CNC(=O)c2ccc3c(c2)c(C)c(C)n3Cc2ccccc2C)o1] | BC20200 |
| 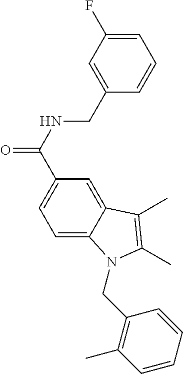 [SMILES: Cc1ccccc1Cn1c(C)c(C)c2cc(C(=O)NCc3cccc(F)c3)ccc21] | BC20202 |
| 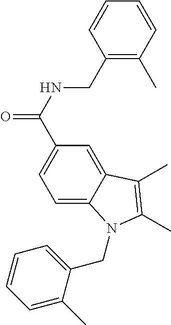 [SMILES: Cc1ccccc1CNC(=O)c1ccc2c(c1)c(C)c(C)n2Cc1ccccc1C] | BC20203 |
| 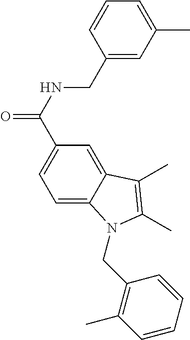 [SMILES: Cc1cccc(CNC(=O)c2ccc3c(c2)c(C)c(C)n3Cc2ccccc2C)c1] | BC20204 |
| 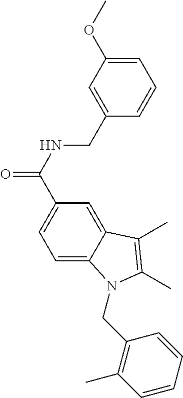 [SMILES: COc1cccc(CNC(=O)c2ccc3c(c2)c(C)c(C)n3Cc2ccccc2C)c1] | BC20205 |
| 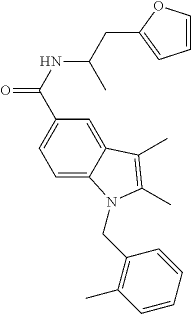 [SMILES: Cc1ccccc1Cn1c(C)c(C)c2cc(C(=O)NC(C)Cc3ccco3)ccc21] | BC20207 |
| 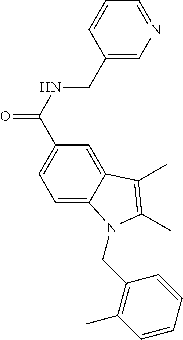 [SMILES: Cc1ccccc1Cn1c(C)c(C)c2cc(C(=O)NCc3cccnc3)ccc21] | BC20208 |
| 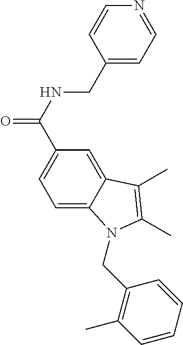 [SMILES: Cc1ccccc1Cn1c(C)c(C)c2cc(C(=O)NCc3ccncc3)ccc21] | BC20209 |
| 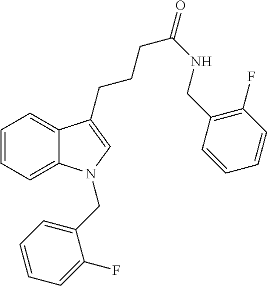 [SMILES: O=C(CCCc1cn(Cc2ccccc2F)c2ccccc12)NCc1ccccc1F] | BC20210 |
| 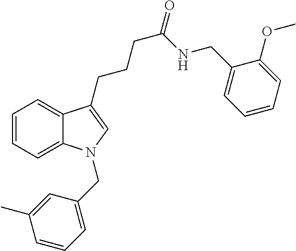 | BC20213 |
| 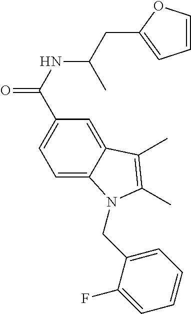 [SMILES: Cc1c(C)n(Cc2ccccc2F)c2ccc(C(=O)NC(C)Cc3ccco3)cc12] | BC20244 |
| 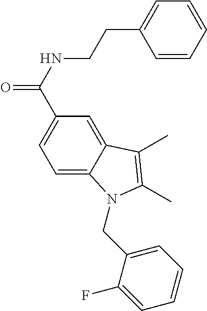 [SMILES: Cc1c(C)n(Cc2ccccc2F)c2ccc(C(=O)NCCc3ccccc3)cc12] | BC20245 |
| 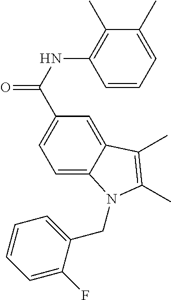 [SMILES: Cc1cccc(NC(=O)c2ccc3c(c2)c(C)c(C)n3Cc2ccccc2F)c1C] | BC20246 |
| 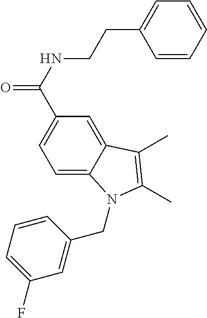 [SMILES: Cc1c(C)n(Cc2cccc(F)c2)c2ccc(C(=O)NCCc3ccccc3)cc12] | BC20248 |
| 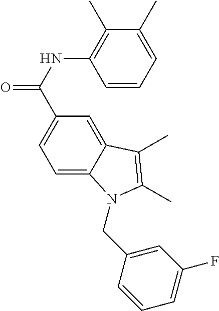 [SMILES: Cc1cccc(NC(=O)c2ccc3c(c2)c(C)c(C)n3Cc2cccc(F)c2)c1C] | BC20249 |
| 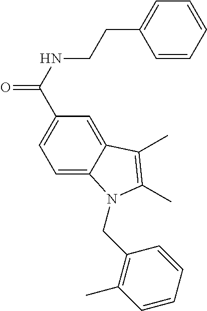 [SMILES: Cc1ccccc1Cn1c(C)c(C)c2cc(C(=O)NCCc3ccccc3)ccc21] | BC20251 |
| 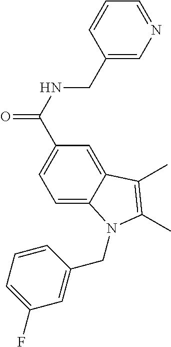 [SMILES: Cc1c(C)n(Cc2cccc(F)c2)c2ccc(C(=O)NCc3cccnc3)cc12] | BC20265 |
| 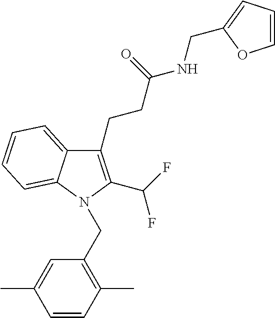 [SMILES: Cc1ccc(C)c(Cn2c(C(F)F)c(CCC(=O)NCc3ccco3)c3ccccc32)c1] | BC20354 |
| 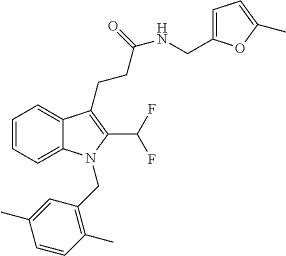 | BC20367 |
| 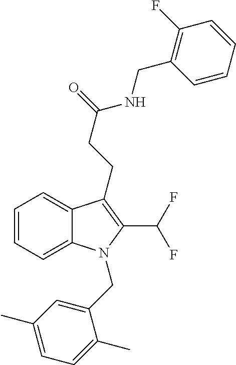 [SMILES: Cc1ccc(C)c(Cn2c(C(F)F)c(CCC(=O)NCc3ccccc3F)c3ccccc32)c1] | BC20393 |
| 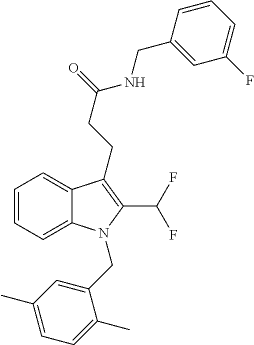 [SMILES: Cc1ccc(C)c(Cn2c(C(F)F)c(CCC(=O)NCc3cccc(F)c3)c3ccccc32)c1] | BC20394 |
| 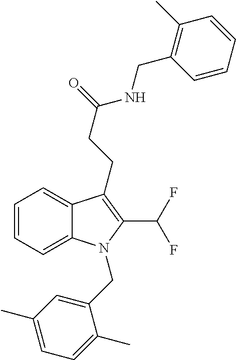 [SMILES: Cc1ccc(C)c(Cn2c(C(F)F)c(CCC(=O)NCc3ccccc3C)c3ccccc32)c1] | BC20395 |
| 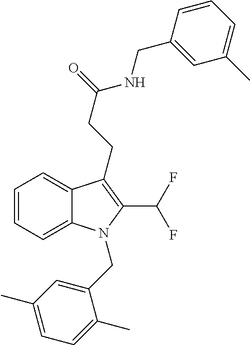 | BC20396 |
| 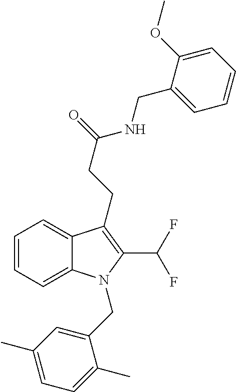 [SMILES: COc1ccccc1CNC(=O)CCc1c(C(F)F)n(Cc2cc(C)ccc2C)c2ccccc12] | BC20397 |
| 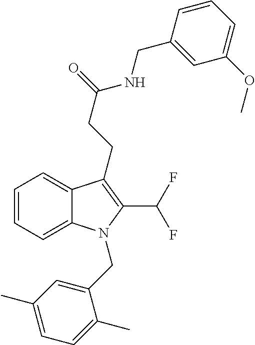 [SMILES: COc1cccc(CNC(=O)CCc2c(C(F)F)n(Cc3cc(C)ccc3C)c3ccccc23)c1] | BC20398 |
| 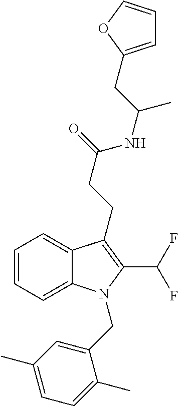 [SMILES: Cc1ccc(C)c(Cn2c(C(F)F)c(CCC(=O)NC(C)Cc3ccco3)c3ccccc32)c1] | BC20401 |
| 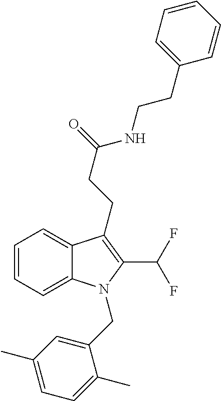 [SMILES: Cc1ccc(C)c(Cn2c(C(F)F)c(CCC(=O)NCCc3ccccc3)c3ccccc32)c1] | BC20402 |
| 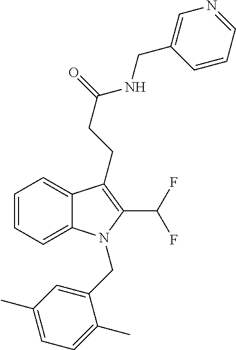 [SMILES: Cc1ccc(C)c(Cn2c(C(F)F)c(CCC(=O)NCc3cccnc3)c3ccccc32)c1] | BC20403 |
| 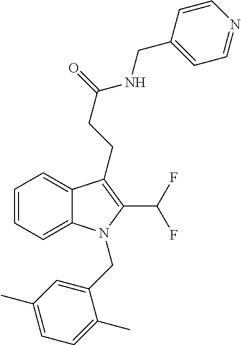 [SMILES: Cc1ccc(C)c(Cn2c(C(F)F)c(CCC(=O)NCc3ccncc3)c3ccccc32)c1] | BC20404 |

In some embodiments, the compound of Formula (V) is selected from any one of the compounds listed in Table 5b, or a pharmaceutically acceptable salt thereof.

TABLE 5b

| | |
|---|---|
| BC20110 | 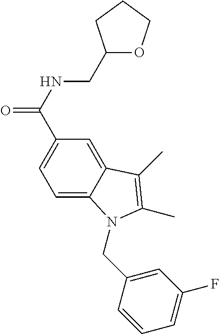 |
| BC20194 | 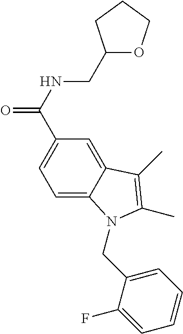 |
| BC20123 | 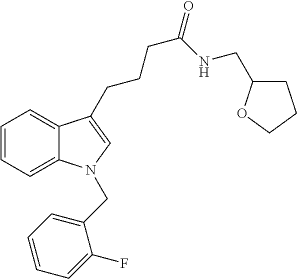 |
| BC20137 | 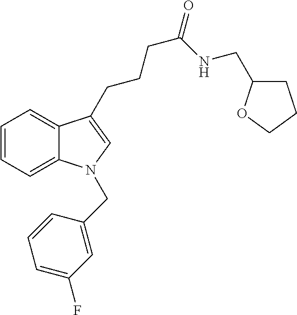 |
| BC20120 | 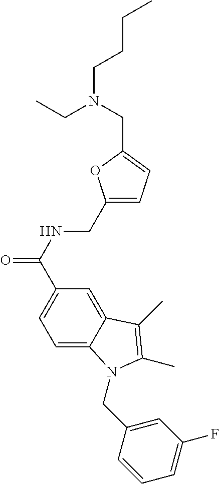 |
| BC20157 | 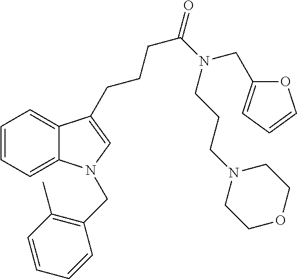 |
| BC20162 | 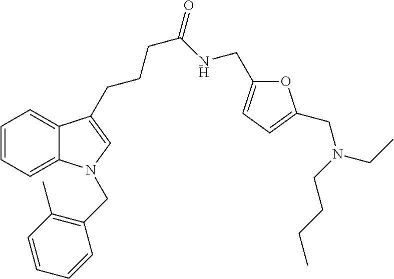 |
| BC20191 | 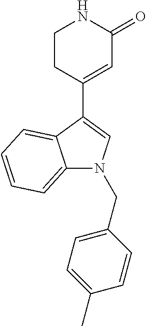 |
| BC20192 | 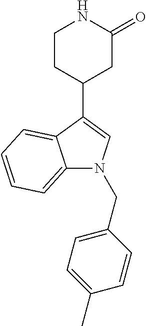 |
| BC20198 | 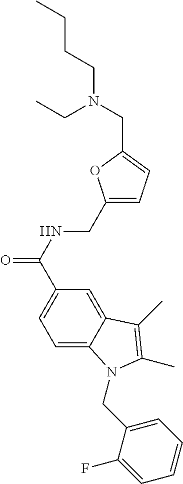 |
| BC20201 | 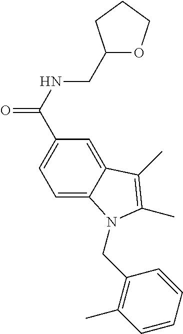 |
| BC20211 | 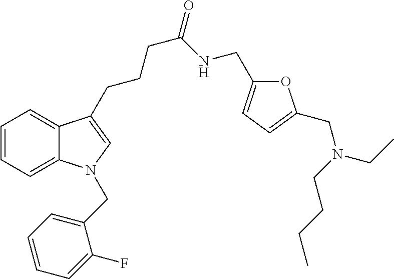 |
| BC20212 | 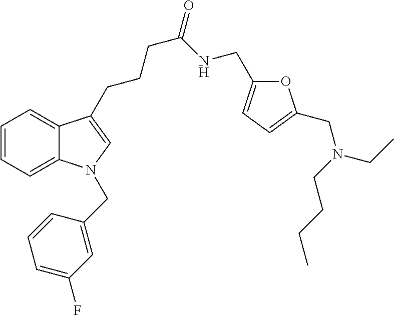 |
| BC20230 | 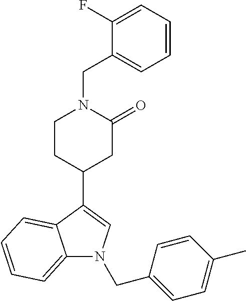 |
| BC20231 | 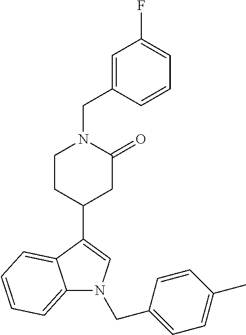 |
| BC20232 | 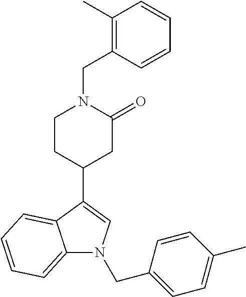 |
| BC20233 | 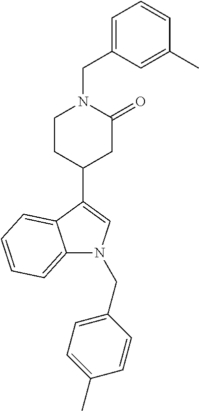 |
| BC20234 | 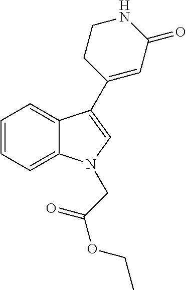 |
| BC20235 | 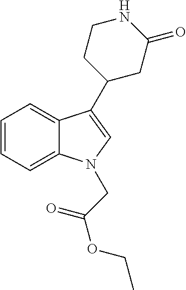 |
| BC20247 | 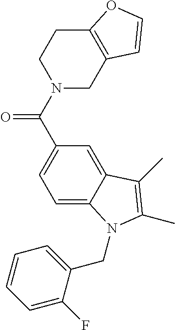 |
| BC20250 | 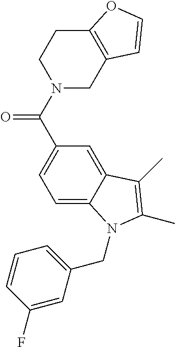 |
| BC20263 | 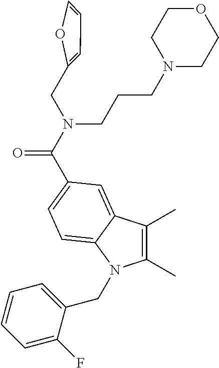 |
| BC20264 | 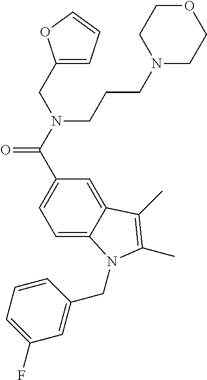 |
| BC20266 | 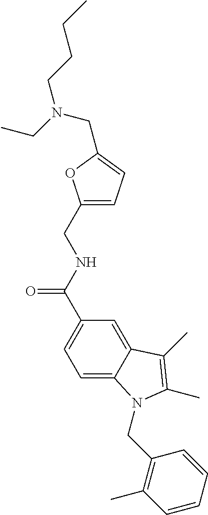 |
| BC20267 | 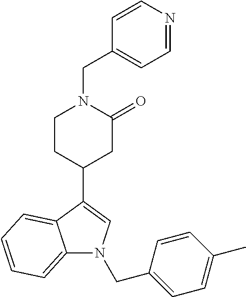 |
| BC20268 | 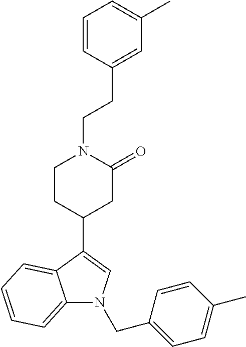 |
| BC20293 | 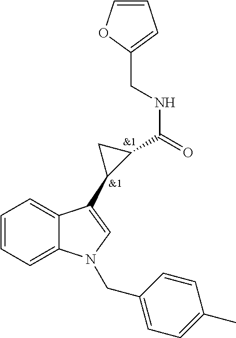 |
| BC20294 | 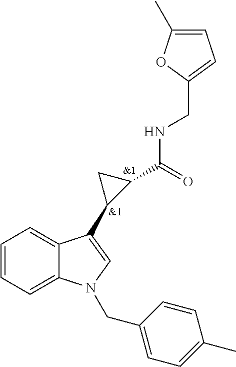 |
| BC20295 | 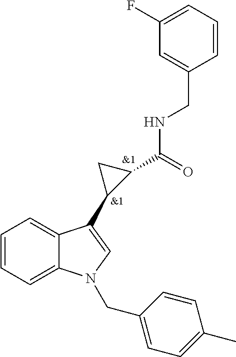 |
| BC20296 | 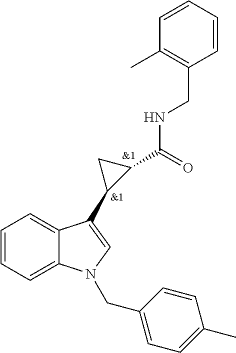 |
| BC20297 | 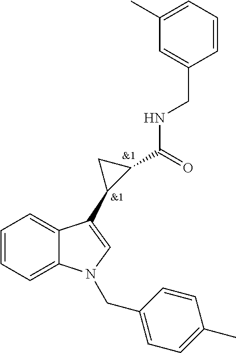 |
| BC20298 | 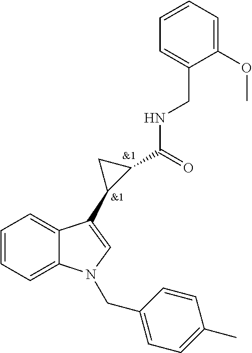 |
| BC20299 | 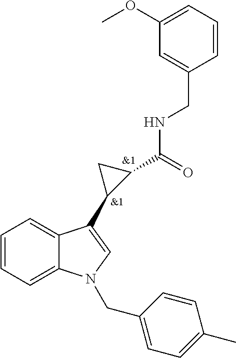 |
| BC20300 | 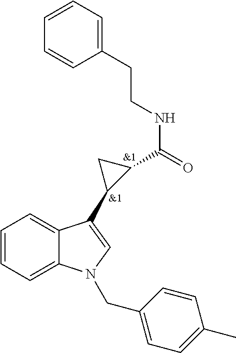 |
| BC20301 | 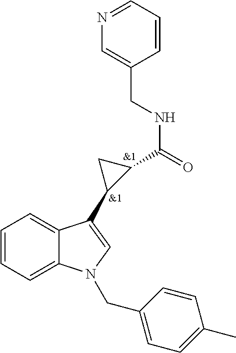 |
| BC20302 | 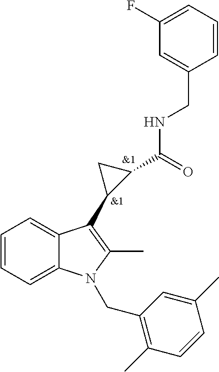 |
| BC20303 | 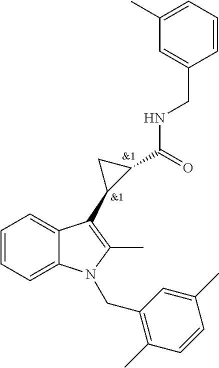 |
| BC20304 | 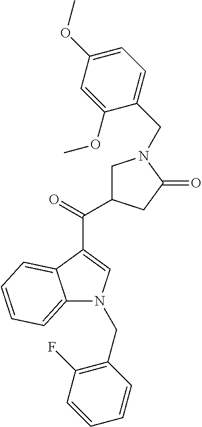 |
| BC20307 | 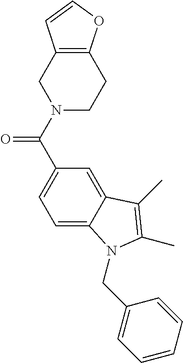 |
| BC20318 | 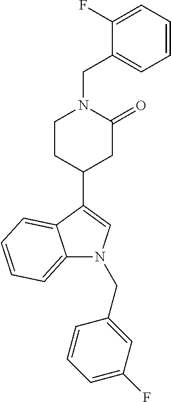 |
| BC20319 | 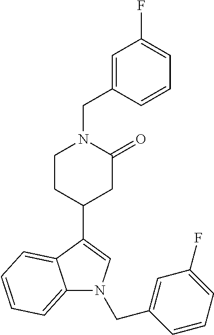 |
| BC20320 | 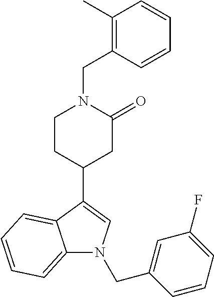 |
| BC20321 | 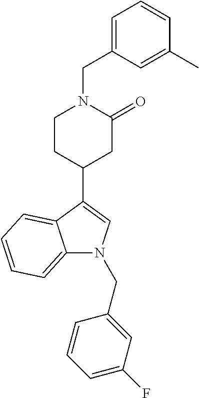 |
| BC20322 | 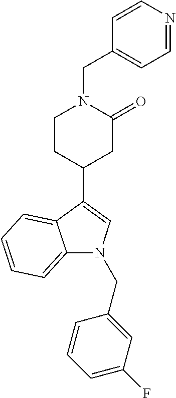 |
| BC20323 | 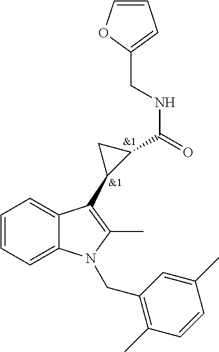 |
| BC20324 | 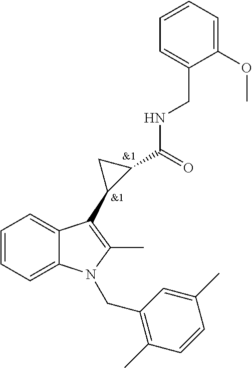 |
| BC20325 | 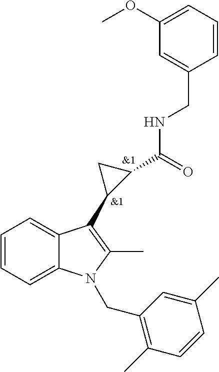 |
| BC20326 | 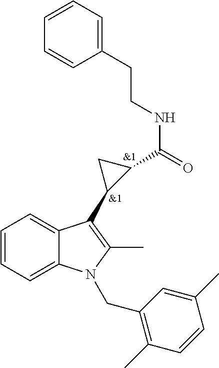 |
| BC20339 | 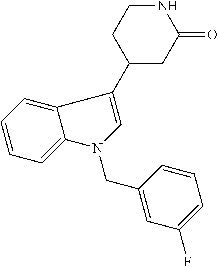 |
| BC20340 | 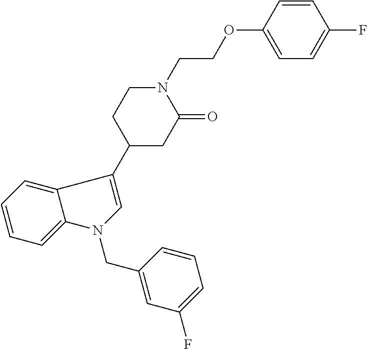 |
| BC20344 | 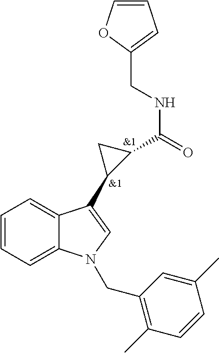 |
| BC20345 | 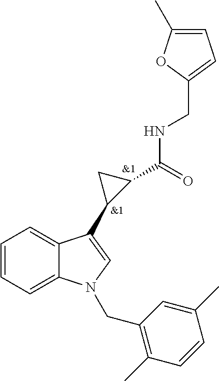 |
| BC20346 | 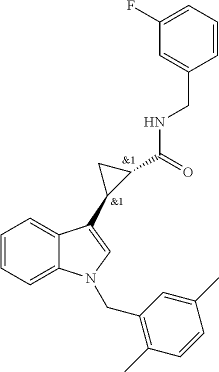 |
| BC20347 | 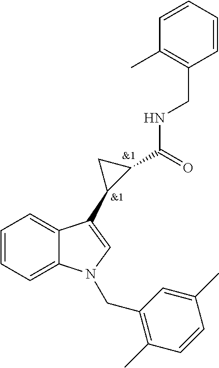 |
| BC20348 | 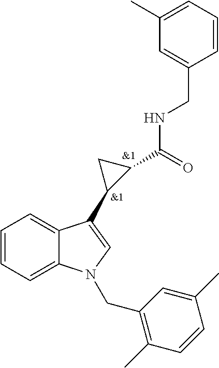 |
| BC20349 | 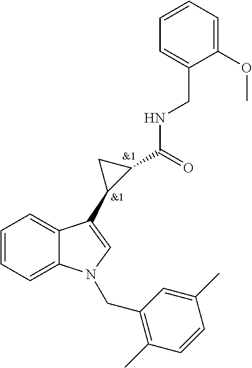 |
| BC20350 | 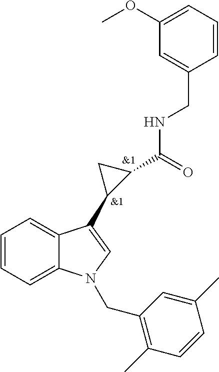 |
| BC20351 | 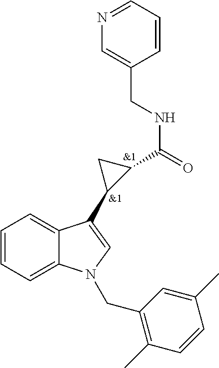 |
| BC20352 | 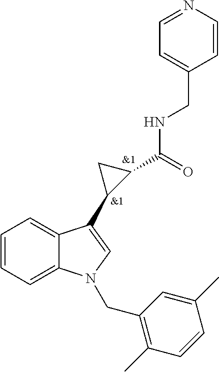 |
| BC20353 | 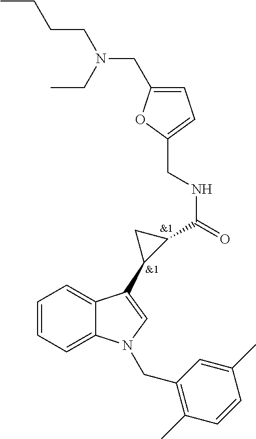 |
| BC20359 | 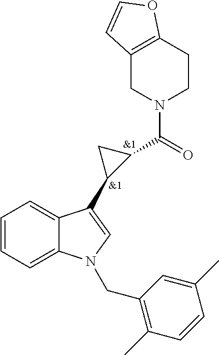 |
| BC20360 | 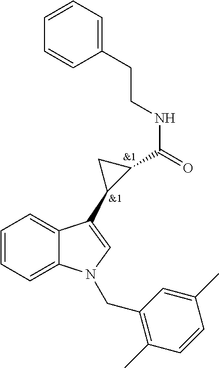 |
| BC20363 | 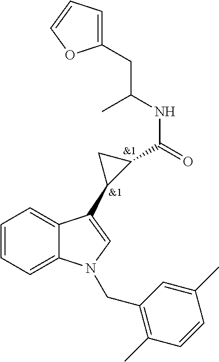 |
| BC20364 | 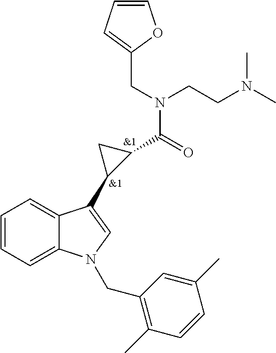 |
| BC20365 | 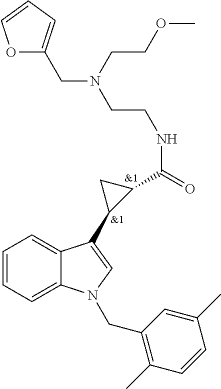 |
| BC20366 | 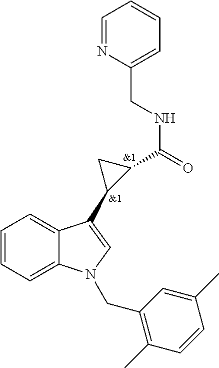 |
| BC20392 | 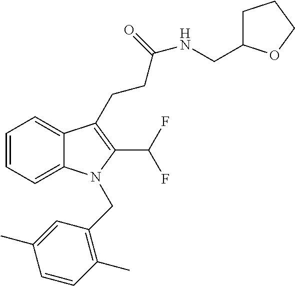 |
| BC20399 | 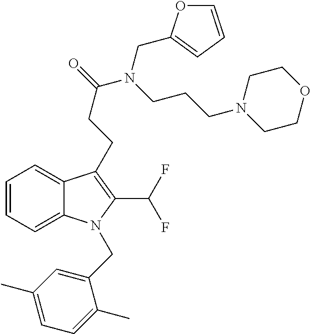 |
| BC20405 | 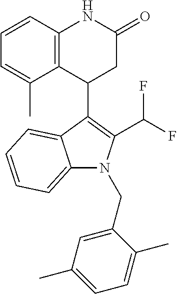 |
| BC20406 | 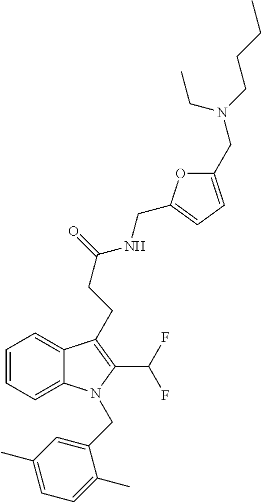 |

In some embodiments, the compound of Formula (V) is selected from any one of the compounds listed in Table 5c, or a pharmaceutically acceptable salt thereof.

TABLE 5c

| | |
|---|---|
| BC20102 | 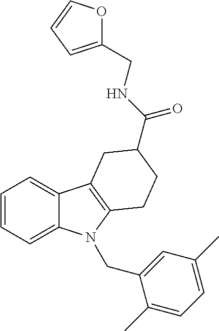 |
| BC20183 | 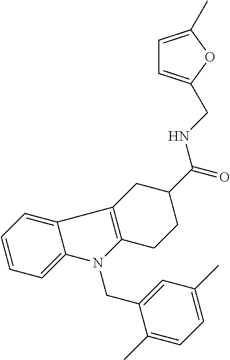 |
| BC20184 | 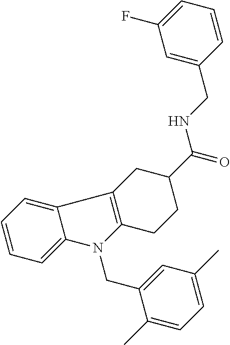 |
| BC20185 | 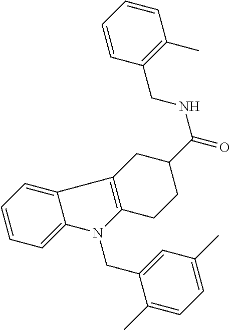 |
| BC20186 | 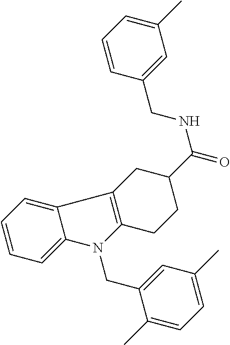 |
| BC20187 | 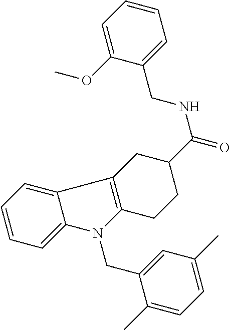 |
| BC20188 | 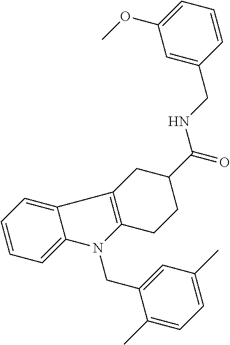 |
| BC20189 | 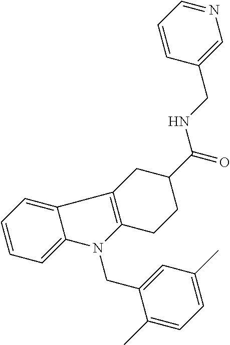 |
| BC20190 | 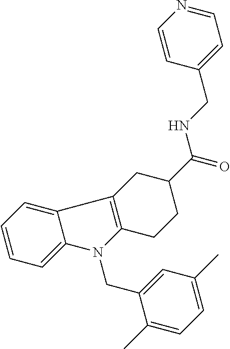 |
| BC20217 | 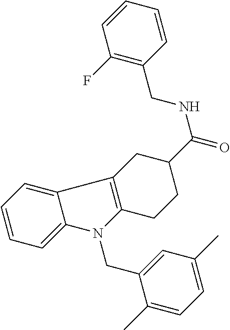 |
| BC20218 | 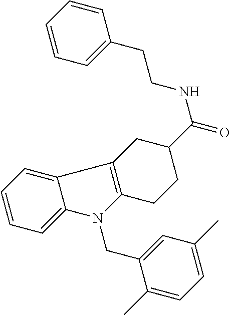 |
| BC20219 | 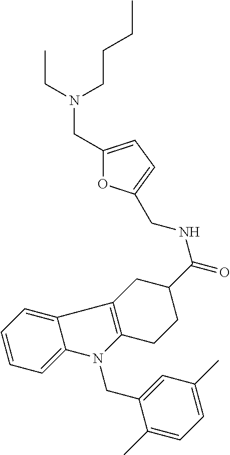 |
| BC20220 | 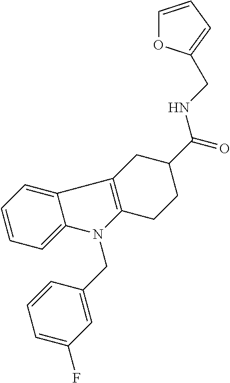 |
| BC20221 | 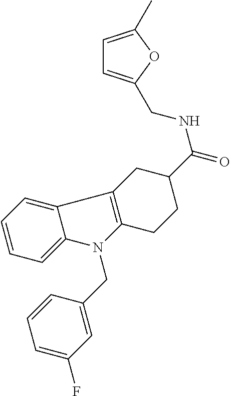 |
| BC20222 | 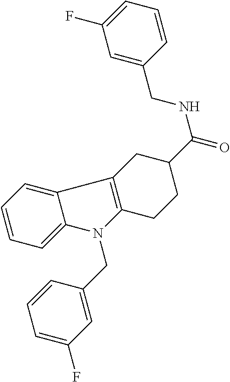 |
| BC20223 | 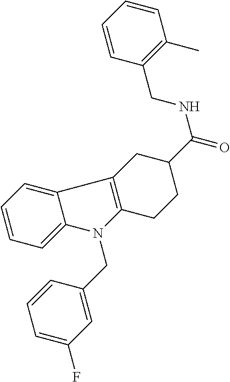 |
| BC20224 | 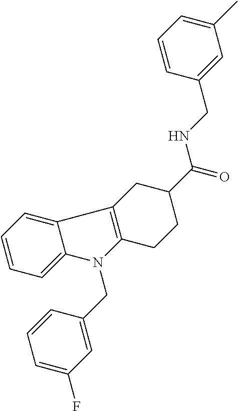 |
| BC20225 | 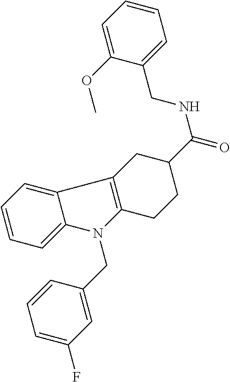 |
| BC20226 | 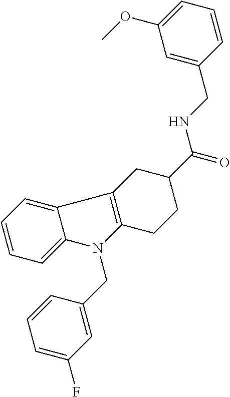 |
| BC20227 | 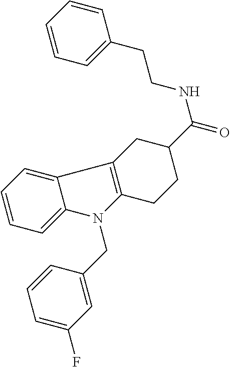 |
| BC20228 | 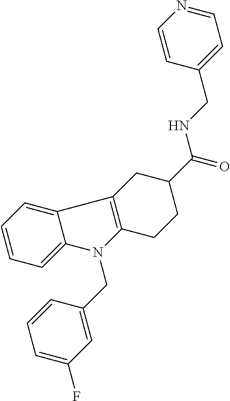 |
| BC20229 | 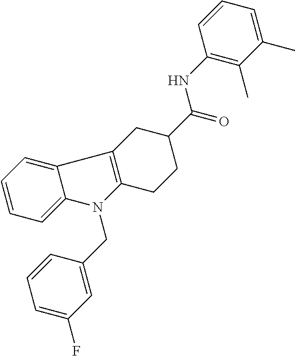 |
| BC20236 | 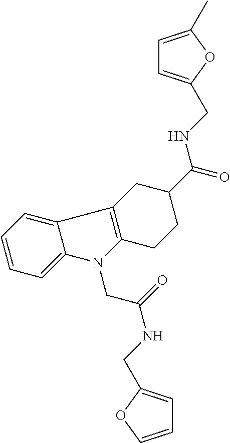 |
| BC20237 | 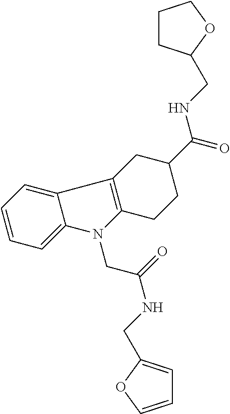 |
| BC20238 | 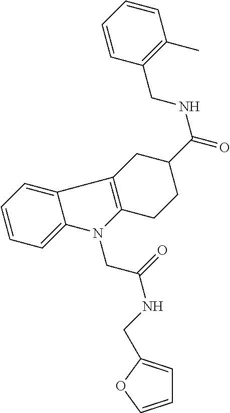 |
| BC20239 | 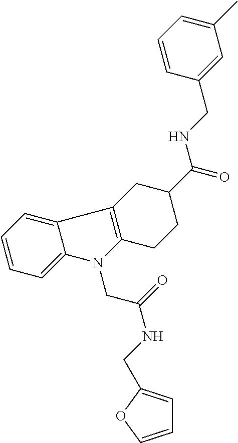 |
| BC20240 | 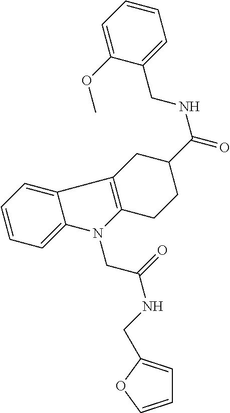 |
| BC20241 | 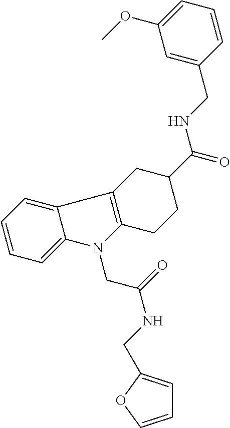 |
| BC20242 | 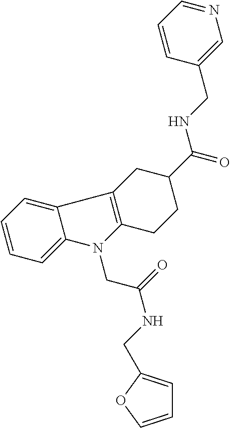 |
| BC20243 | 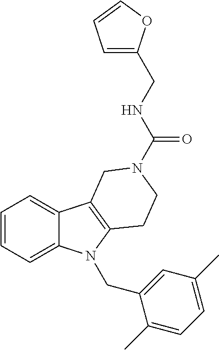 |
| BC20252 | 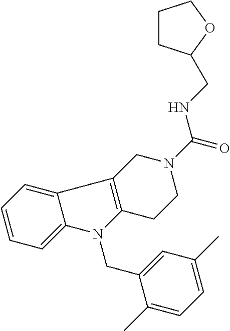 |
| BC20253 | 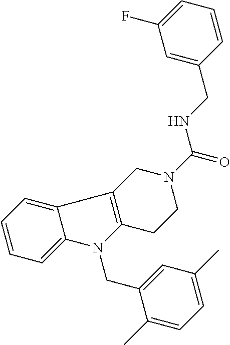 |
| BC20254 | 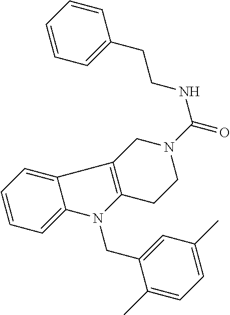 |
| BC20255 | 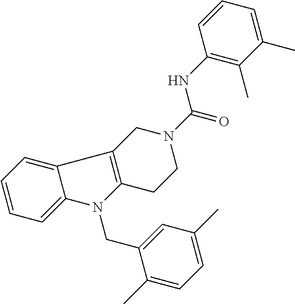 |
| BC20256 | 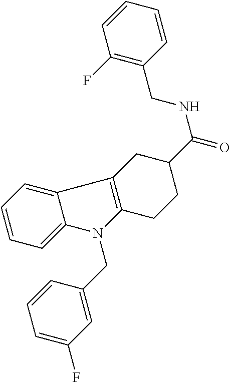 |
| BC20258 | 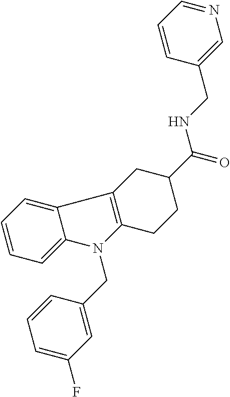 |
| BC20259 | 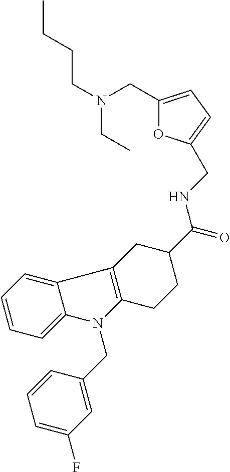 |
| BC20260 | 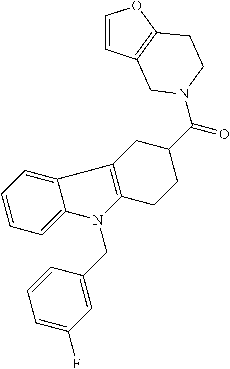 |
| BC20262 | 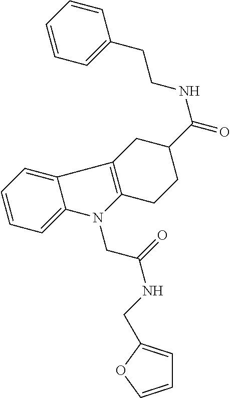 |
| BC20279 | 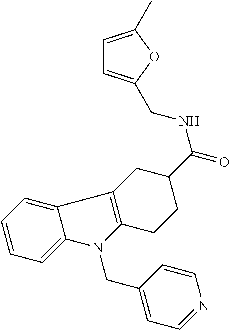 |
| BC20280 | 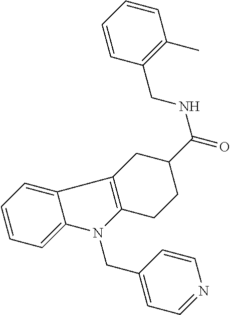 |
| BC20281 | 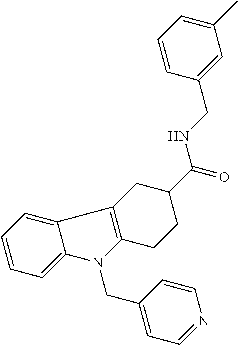 |
| BC20282 | 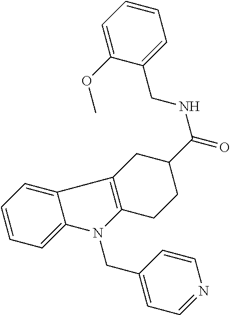 |
| BC20283 | 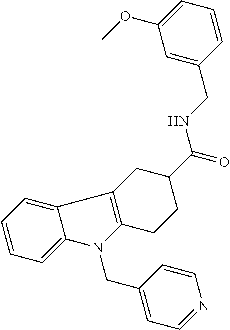 |
| BC20284 | 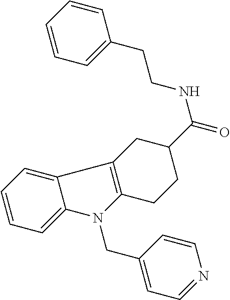 |
| BC20285 | 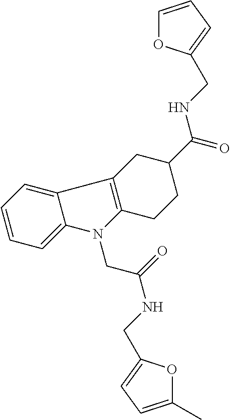 |
| BC20286 | 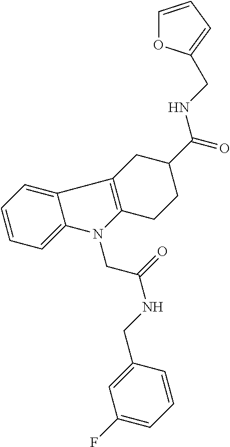 |
| BC20287 | 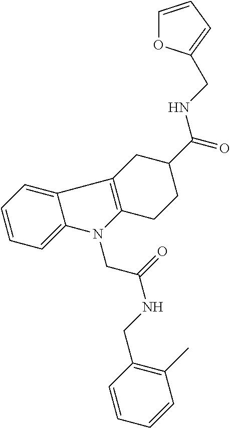 |
| BC20288 | 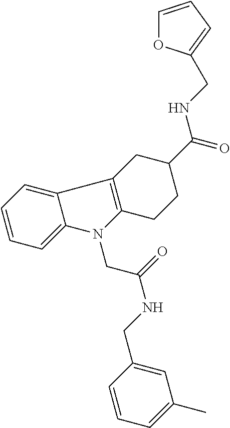 |
| BC20289 | 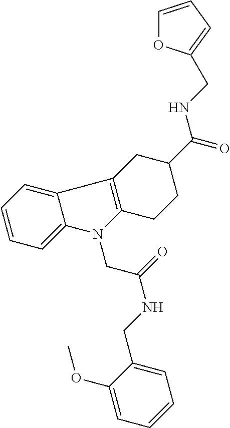 |
| BC20290 | 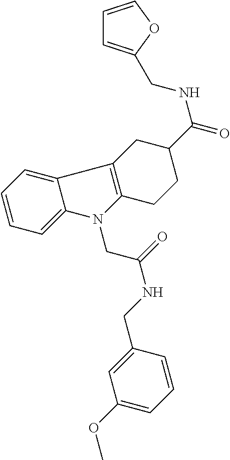 |
| BC20291 | 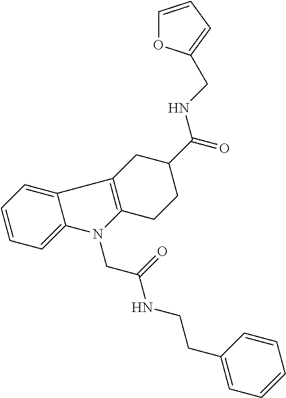 |
| BC20292 | 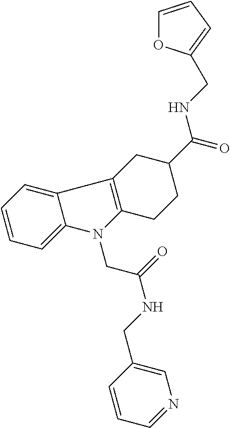 |
| BC20308 | 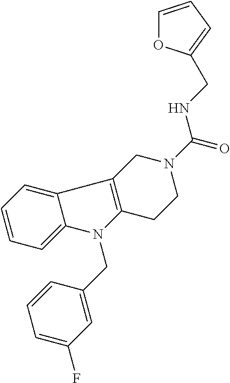 |
| BC20309 | 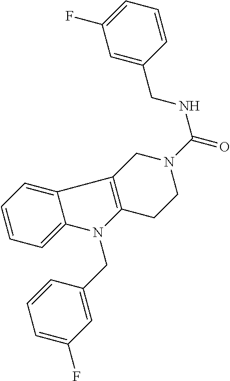 |
| BC20310 | 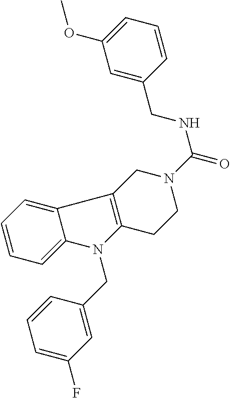 |
| BC20311 | 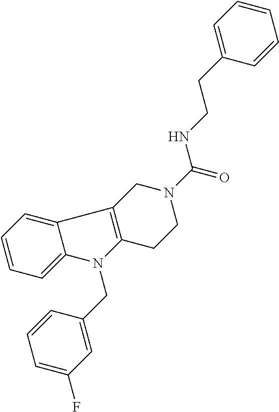 |
| BC20312 | 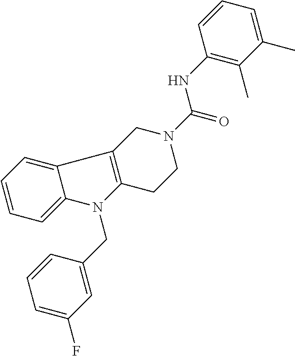 |
| BC20316 | 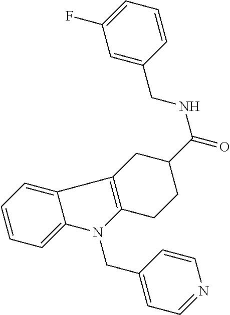 |
| BC20317 | 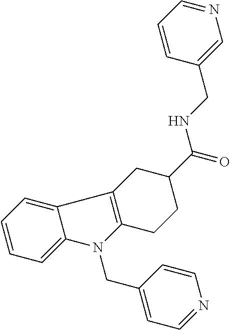 |
| BC20327 | 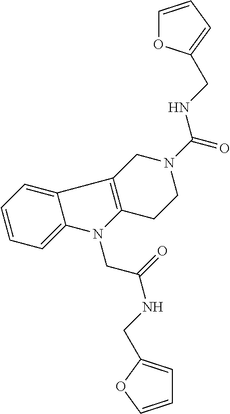 |
| BC20328 | 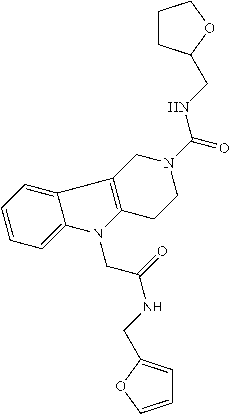 |
| BC20329 | 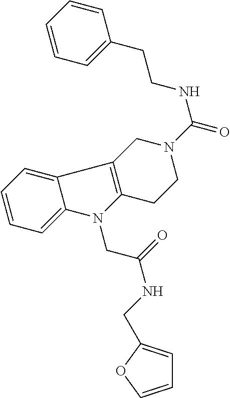 |
| BC20330 | 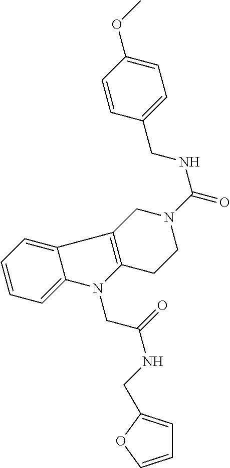 |
| BC20331 | 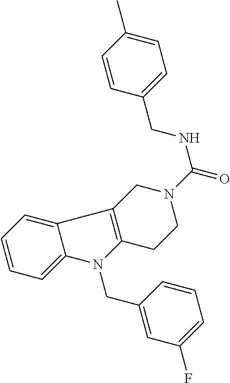 |
| BC20332 | 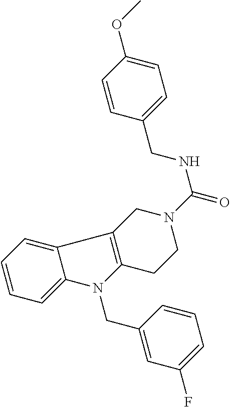 |
| BC20334 | 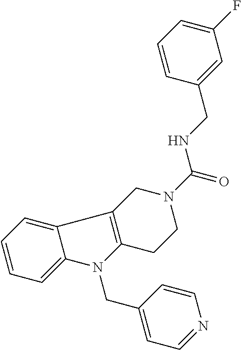 |
| BC20335 | 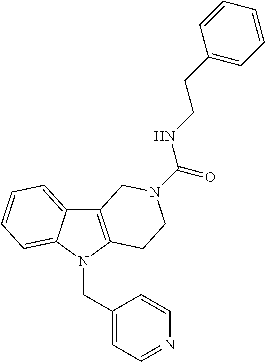 |
| BC20336 | 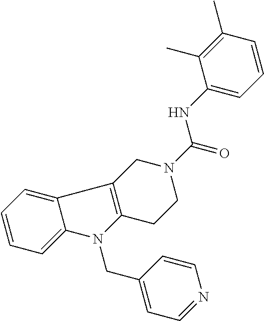 |
| BC20337 | 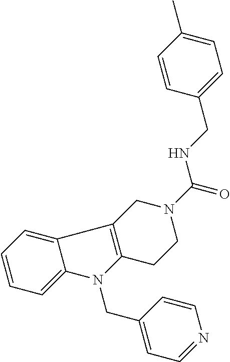 |
| BC20338 | 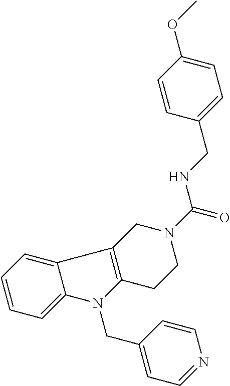 |
| BC20357 | 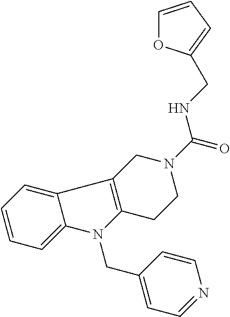 |
| BC20358 | 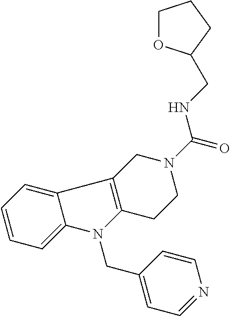 |
| BC20361 | 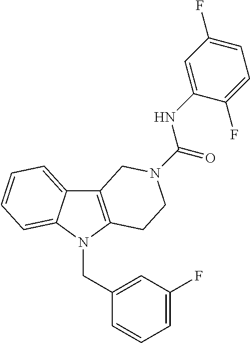 |
| BC20362 | 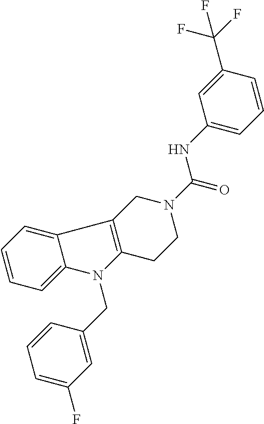 |
| BC20368 | 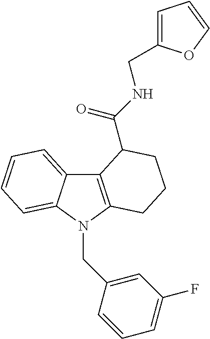 |
| BC20369 | 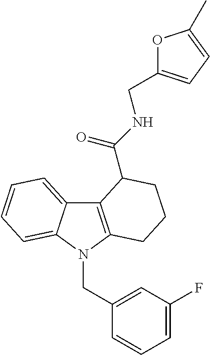 |
| BC20370 | 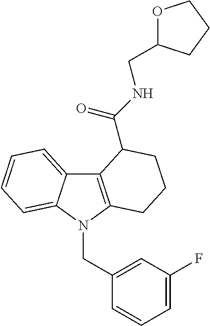 |
| BC20371 | 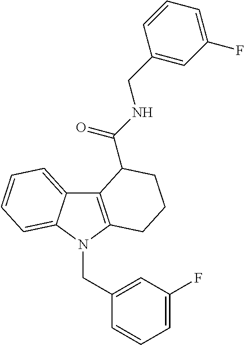 |
| BC20372 | 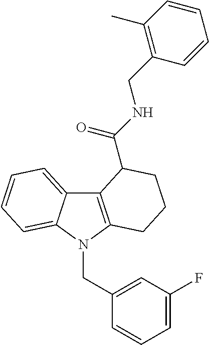 |
| BC20373 | 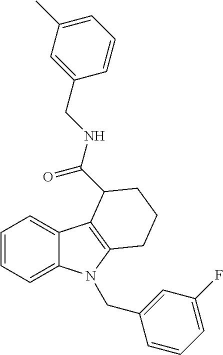 |
| BC20374 | 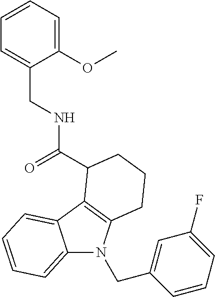 |
| BC20375 | 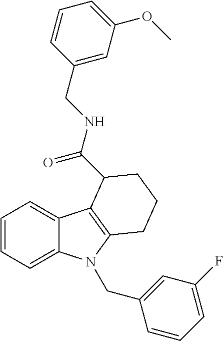 |
| BC20377 | 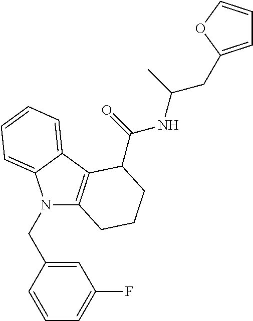 |
| BC20378 | 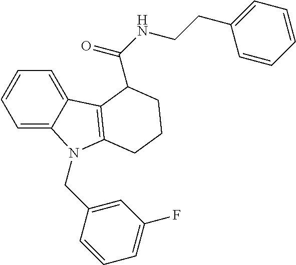 |
| BC20379 | 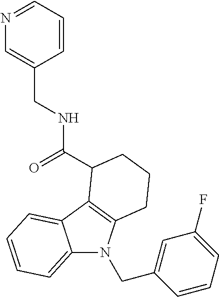 |
| BC20380 | 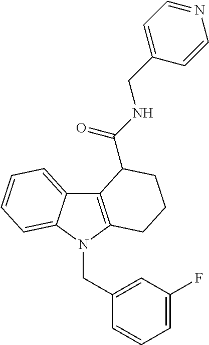 |
| BC20381 | 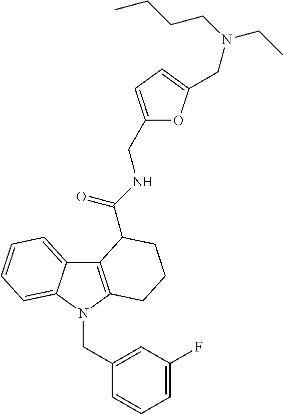 |
| BC20382 | 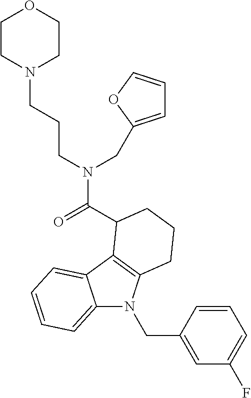 |
| BC20383 | 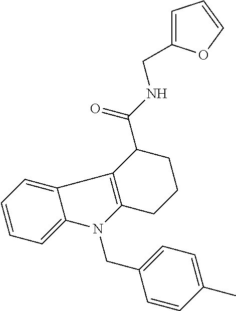 |
| BC20384 | 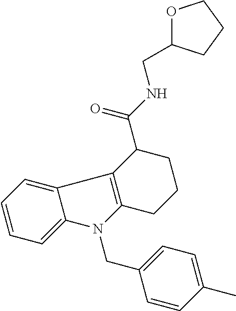 |
| BC20385 | 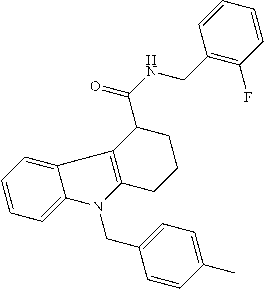 |
| BC20386 | 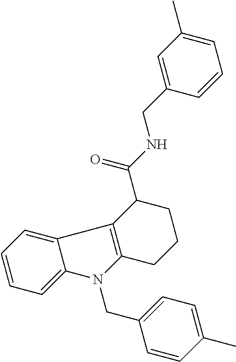 |
| BC20387 | 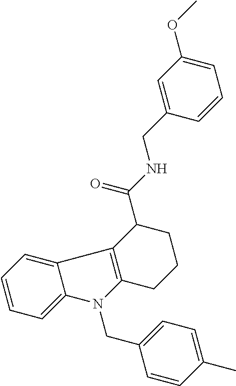 |
| BC20388 | 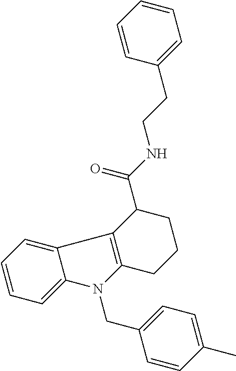 |
| BC20389 | 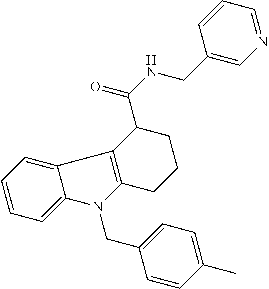 |
| BC20390 | 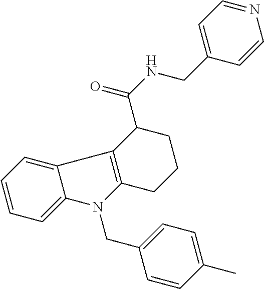 |
| BC20391 | 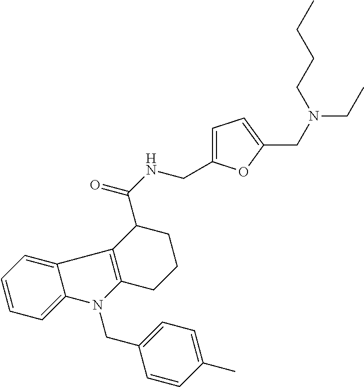 |

In some embodiments, the compound of Formula (V) is selected from any one of the compounds listed in Table 5d, or a pharmaceutically acceptable salt thereof.

TABLE 5d

| | |
|---|---|
| 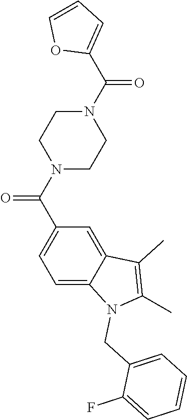 | BC20107 |
| 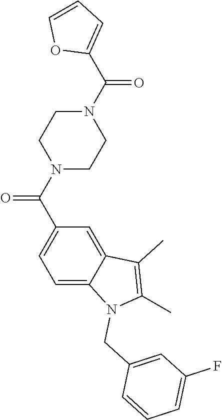 | BC20117 |
| 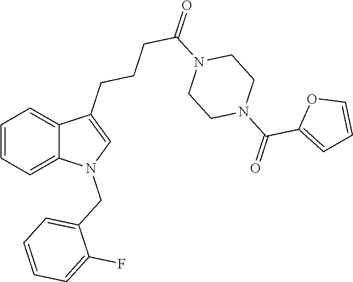 | BC20129 |
| 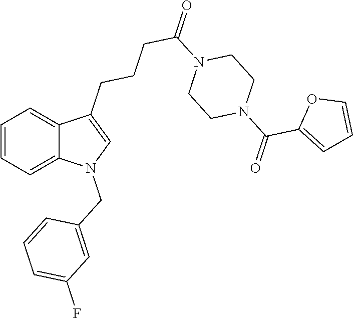 | BC20144 |
| 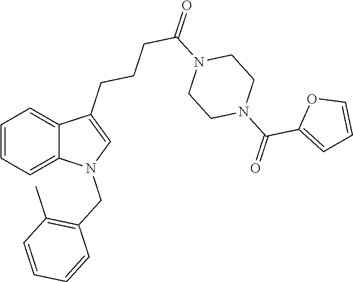 | BC20158 |
| 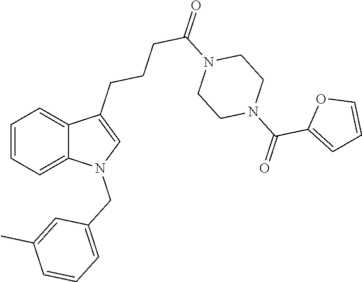 | BC20170 |
| 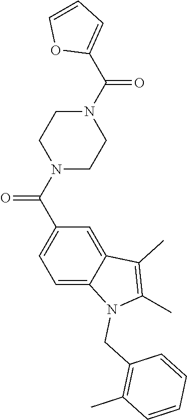 | BC20206 |
| 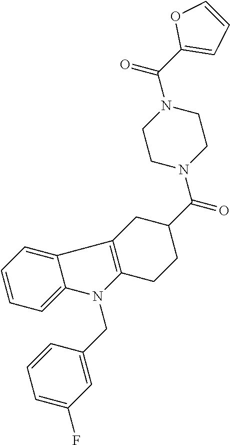 | BC20257 |
| 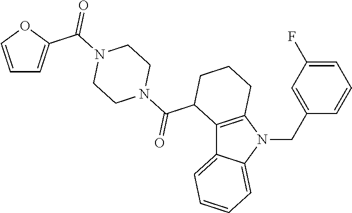 | BC20376 |
| 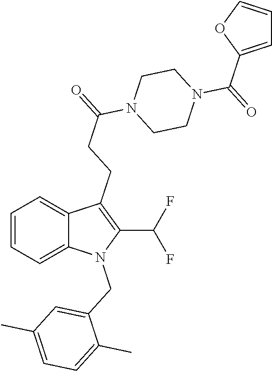 | BC20400 |

In some embodiments, the compound of Formula (V) is selected from any one of the compounds listed in Table 5e, or a pharmaceutically acceptable salt thereof.

TABLE 5e

| | |
|---|---|
| 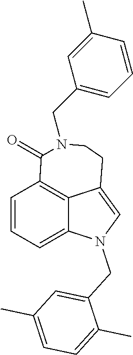 | BC20261 |
| 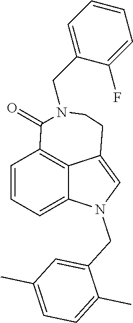 | BC20269 |
| 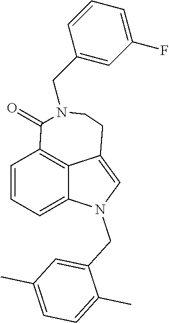 | BC20270 |
| 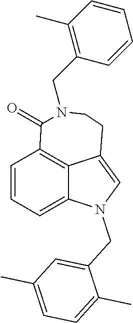 | BC20271 |
| 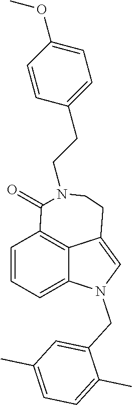 | BC20272 |
| 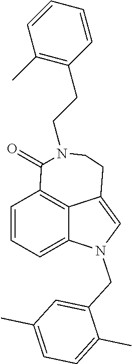 | BC20273 |
| 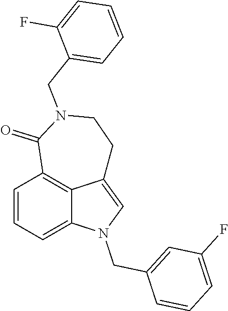 | BC20274 |
| 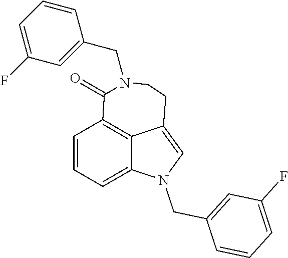 | BC20275 |
| 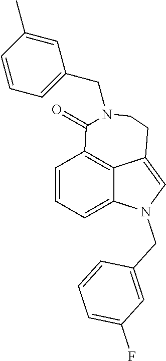 | BC20276 |
| 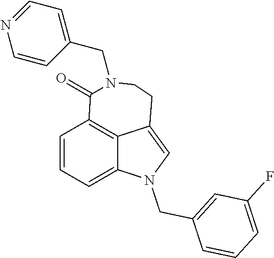 | BC20277 |
| 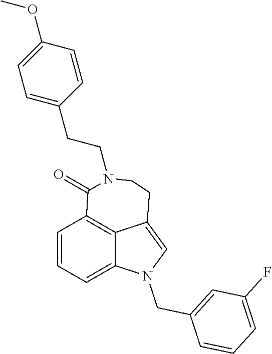 | BC20278 |
| 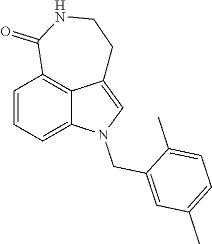 | BC20313 |
| 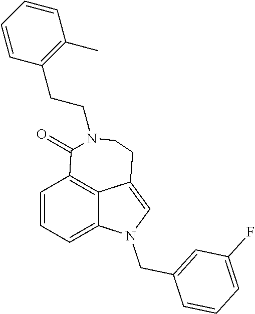 | BC20314 |
| 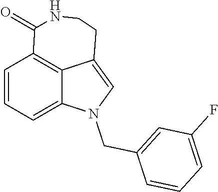 | BC20315 |
| 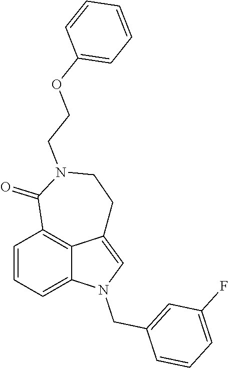 | BC20341 |
| 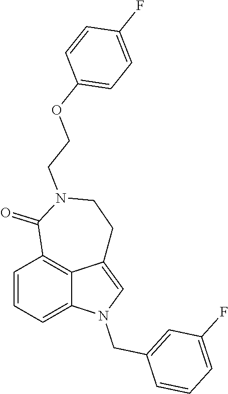 | BC20342 |
| 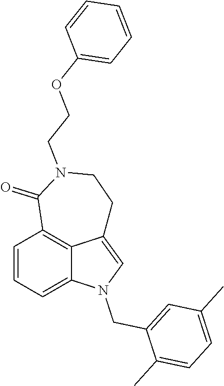 | BC20343 |
| 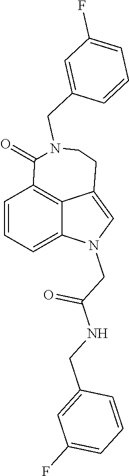 | BC20355 |
| 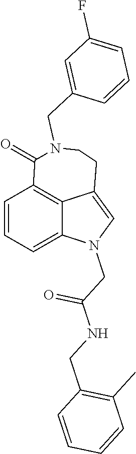 | BC20356 |

In some embodiments, this document provides a compound selected from:

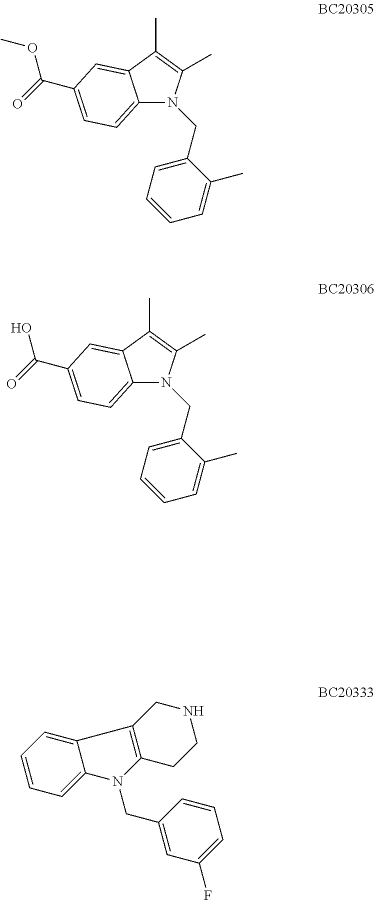

or a pharmaceutically acceptable salt thereof.

In some embodiments, this document provides a compound of Formula (VI):

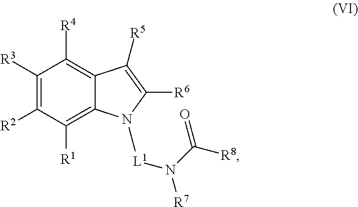

or a pharmaceutically acceptable salt thereof, wherein:

R¹, R², R³, R⁴, R⁵, and R⁶ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, and C₁₋₆ haloalkoxy;

L¹ is selected from C₁₋₆ alkylene, C₂₋₆ alkenylene, and C₂₋₆ alkynylene, each of which is optionally substituted with 1, 2, or 3 substituents independently selected from R^{g};

R⁷ is selected from H and C₁₋₆ alkyl;

R⁹ is selected from C₆₋₁₀ aryl and 5-10 membered heteroaryl, each of which is optionally substituted with 1, 2, 3, 4, or 5 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R¹, R², R³, R⁴, R⁵, and R⁶ are each independently selected from H, halo, and C₁₋₆ alkoxy.

In some embodiments:

R¹ is H;

R² is selected from H and halo;

R³ is selected from H, halo, and C₁₋₆ alkoxy;

R⁴ is selected from H and halo.

In some embodiments, R¹ is H.

In some embodiments, R² is H.

In some embodiments, R² is halo.

In some embodiments, R³ is H.

In some embodiments, R³ is halo.

In some embodiments, R³ is C₁₋₆ alkoxy.

In some embodiments, R⁴ is H.

In some embodiments, R⁴ is halo.

In some embodiments, L¹ is C₁₋₆ alkylene.

In some embodiments, R⁷ is H.

In some embodiments, R⁹ is 5-10 membered heteroaryl, optionally substituted with 1, 2, or 3 substituents independently selected from R^{g}.

In some embodiments, R⁹ is furanyl (e.g., furan-2-yl or furan-3-yl), optionally substituted with 1, 2, or 3 substituents independently selected from R^{g}.

In some embodiments, R⁹ is furan-2-yl.

In some embodiments:

R¹, R², R³, R⁴, R⁵, and R⁶ are each independently selected from H, halo, and C₁₋₆ alkoxy;

L¹ is C₁₋₆ alkylene;

R⁷ is H; and

R⁹ is 5-10 membered heteroaryl, optionally substituted with 1, 2, or 3 substituents independently selected from R^{g}.

In some embodiments:

R¹ is H;

R² is selected from H and halo;

R³ is selected from H, halo, and C₁₋₆ alkoxy;

R⁸ is selected from H and halo;

L¹ is C₁₋₆ alkylene;

R⁷ is H; and

R⁹ is furan-2-yl, optionally substituted with 1, 2, or 3 substituents independently selected from R^{g}.

In some embodiments, the compound of Formula (VI) is selected from any one of the compounds listed in Table 6, or a pharmaceutically acceptable salt thereof.

TABLE 6

| | |
|---|---|
| 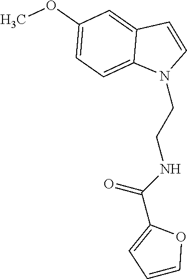 | BC181157 |
| 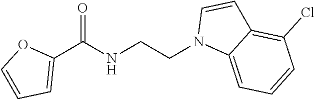 | BC181155 |
| 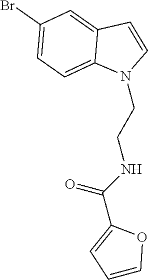 | BC181158 |
| 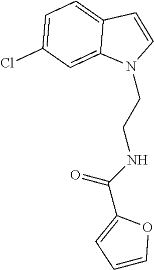 | BC181156 |

In some embodiments, this document provides a compound of Formula (VII):

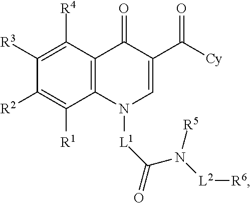

or a pharmaceutically acceptable salt thereof, wherein:

R¹, R², R³, and R⁴ are each independently selected from H, halo, C₁₋₆ alkyl, C₁₋₆ haloalkyl, C₁₋₆ alkoxy, and C₁₋₆ haloalkoxy;

Cy is selected from C₆₋₁₀ aryl and 5-10 membered heteroaryl, each of which is optionally substituted with 1, 2, 3, 4, or 5 substituents independently selected from R^{g};

L¹ is selected from C₁₋₆ alkylene, C₂₋₆ alkenylene, and C₂₋₆ alkynylene, each of which is optionally substituted with 1, 2, or 3 substituents selected from R^{g};

R⁵ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents independently selected from R^{g};

R⁶ is selected from C₆₋₁₀ aryl and 5-10 membered heteroaryl, each of which is optionally substituted with 1, 2, 3, 4, or 5 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R¹, R², R³, and R⁴ are each independently selected from H, halo, and C₁₋₆ alkyl.

In some embodiments, Cy is 5-10 membered heteroaryl, optionally substituted with 1, 2, or 3 substituents independently selected from R^{g}.

In some embodiments, L¹ is C₁₋₆ alkylene, optionally substituted with 1, 2, or 3 substituents selected from R^{g}.

In some embodiments, R⁵ is H.

In some embodiments, L² is C₁₋₆ alkylene, optionally substituted with 1, 2, or 3 substituents selected from R^{g}.

In some embodiments, R⁶ is C₆₋₁₀ aryl, optionally substituted with 1, 2, or 3 substituents independently selected from R^{g}.

In some embodiments, R⁹ is selected from halo and C₁₋₆ alkyl.

In some embodiments:

R¹, R², R³, and R⁴ are each independently selected from H, halo, and C₁₋₆ alkyl;

Cy is 5-10 membered heteroaryl, optionally substituted with 1, 2, or 3 substituents independently selected from R^{g};

L¹ is C₁₋₆ alkylene, optionally substituted with 1, 2, or 3 substituents selected from R^{g};

R⁵ is H;

L² is C₁₋₆ alkylene, optionally substituted with 1, 2, or 3 substituents selected from R^{g};

R⁶ is C₆₋₁₀ aryl, optionally substituted with 1, 2, or 3 substituents independently selected from R^{g}; and

each R^{g} is independently selected from halo and C₁₋₆ alkyl.

In some embodiments, the compound of Formula (VII) is selected from any one of the compounds listed in Table 7, or a pharmaceutically acceptable salt thereof.

TABLE 7

| | |
|---|---|
| 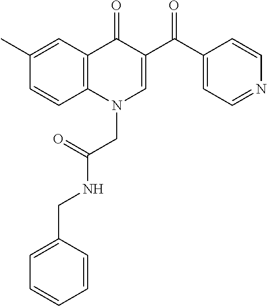 | BC181249 |
| 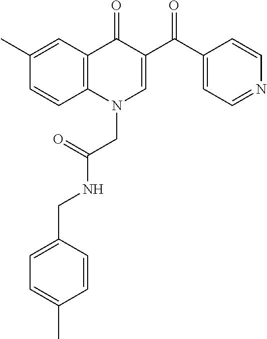 | BC181250 |
| 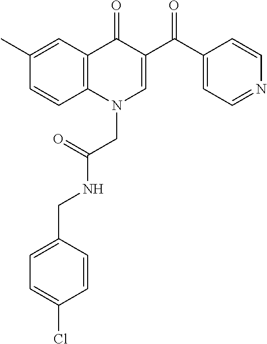 | BC181251 |
| 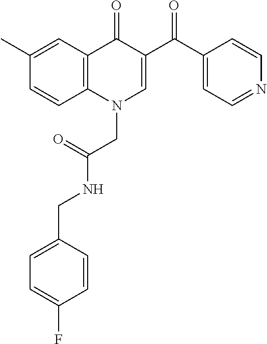 | BC181252 |
| 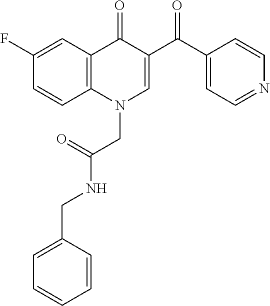 | BC181253 |
| 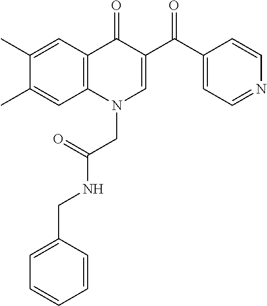 | BC181254 |
| 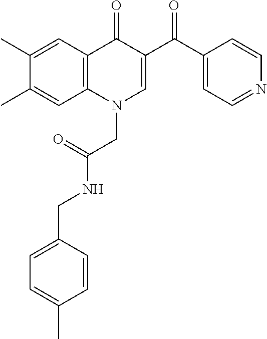 | BC181255 |
| 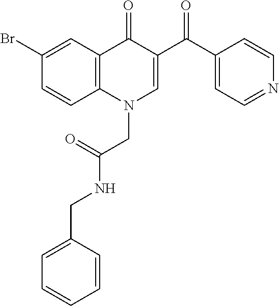 | BC181256 |

In some embodiments, a salt of a compound of any Formulae disclosed herein, or any one of the compounds listed in Table A, is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compound as set forth in Formula (I), (II), (III), (IV), (V), (VI), or (VII), or any one of the compounds listed in Table A, disclosed herein include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and those formed with organic acids such as maleic acid.

In some embodiments, bases commonly employed to form pharmaceutically acceptable salts of the compound as set forth in Formula (I), (II), (III), (IV), (V), (VI), or (VII), or any one of the compounds listed in Table A, include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the compound as set forth in Formula (I), (II), (III), (IV), (V), (VI), or (VII), or any one of the compounds listed in Table A, disclosed herein, or pharmaceutically acceptable salts thereof, are substantially isolated.

In some embodiments, a compound as set forth in Formula (I), (II), (III), (IV), (V), (VI), or (VII), or any one of the compounds listed in Table A, disclosed herein, or a pharmaceutically acceptable salt thereof, can have the ability to increase or maintain NMNAT2 polypeptide levels within a cell. Such cells can be in vitro or in vivo. For example, a compound as set forth in Formula (I), (II), (III), (IV), (V), (VI), or (VII), or any one of the compounds listed in Table A, disclosed herein, or a pharmaceutically acceptable salt thereof, can have the ability to increase or maintain NMNAT2 polypeptide levels within the cells present within a mammal (e.g., a human) following administration to that mammal.

### Methods of Making Therapeutic Compounds

Compounds as set forth in Formula (I), (II), (III), (IV), (V), (VI), or (VII), or any one of the compounds listed in Table A, disclosed herein, including salts thereof, can be prepared using organic synthesis techniques and can be synthesized according to any of numerous possible synthetic routes. A person skilled in the art knows how to select and implement appropriate synthetic protocols, and appreciates that a broad repertoire of synthetic organic reactions is available to be potentially employed in synthesizing compounds provided herein.

Suitable synthetic methods of starting materials, intermediates, and products can be identified by reference to the literature, including reference sources such as: Advances in Heterocyclic Chemistry, Vols. 1-107 (Elsevier, 1963-2012); Journal of Heterocyclic Chemistry Vols. 1-49 (J. Heterocyclic Chemistry, 1964-2012); Carreira et al., (Ed.) Science of Synthesis, Vols. 1-48 (2001-2010) and Knowledge Updates KU2010/1-4; 2011/1-4; 2012/1-2 (Thieme, 2001-2012); Katritzky et al., (Ed.) Comprehensive Organic Functional Group Transformations, (Pergamon Press, 1996); Katritzky et al., (Ed.) Comprehensive Organic Functional Group Transformations II (Elsevier, 2^{nd} Edition, 2004); Katritzky et al., (Ed.) Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.) Comprehensive Organic Synthesis (Pergamon Press, 1991).

The reactions for preparing the compounds provided herein can be carried out in suitable solvents that can be readily selected by one of skill in the art of organic synthesis. Suitable solvents can be substantially non-reactive with the starting materials (reactants), the intermediates, or products at the temperatures at which the reactions are carried out, e.g., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in P. G. M. Wuts and T. W. Greene, Protective Groups in Organic Synthesis, 4^{th} Ed., Wiley & Sons, Inc., New York (2006).

### Pharmaceutical Compositions and Formulations

This document also provides pharmaceutical compositions comprising an effective amount of a compound of any one of Formulae (I)-(VII) disclosed herein, or any one of the compounds listed in Table A, or a pharmaceutically acceptable salt thereof; and a pharmaceutically acceptable carrier. The pharmaceutical composition also can comprise any one of the additional therapeutic agents and/or therapeutic molecules described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants, and vehicles that can be used in the pharmaceutical compositions provided herein include, without limitation, ion exchangers, alumina, aluminum stearate, lecithin, serum proteins (e.g., human serum albumin), buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms can contain any one or more of the compounds or therapeutic agents described herein in the range of 0.005 percent to 100 percent with the balance made up from the suitable pharmaceutically acceptable carriers or excipients. The contemplated compositions can contain from about 0.001 percent to about 100 percent (e.g., from about 0.1 percent to about 95 percent, from about 75 percent to about 85 percent, or from about 20 percent to about 80 percent) of any one or more of the compounds or therapeutic agents provided herein, wherein the balance can be made up of any pharmaceutically acceptable carrier or excipient described herein, or any combination of these carriers or excipients.

### Routes of Administration and Dosage Forms

The therapeutic compounds and/or pharmaceutical compositions provided herein (e.g., a composition containing one or more compounds set forth in Formula (I), (II), (III), (IV), (V), (VI), or (VII), or any one of the compounds listed in Table A, or a pharmaceutically acceptable salt thereof) can include those suitable for any acceptable route of administration. Acceptable routes of administration include, without limitation, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intracranial, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral, vaginal, intravitreal, subretinal or other intraocular routes of administrations.

Compositions and formulations described herein can conveniently be presented in a unit dosage form, e.g., tablets, sustained release capsules, and in liposomes, and can be prepared by any methods well known in the art of pharmacy. See, for example, Remington: The Science and Practice of Pharmacy, Lippincott Williams & Wilkins, Baltimore, Md. (20th ed. 2000). Such preparative methods include, without limitation, the step of bringing into association with the molecule to be administered ingredients such as a carrier that constitutes one or more accessory ingredients. In general, the compositions can be prepared by uniformly and intimately bringing into association the active ingredients with liquid carriers, liposomes, or finely divided solid carriers, or both, and then, if necessary, shaping the product.

In some embodiments, any one or more of the compounds or therapeutic agents described herein can be administered orally. Compositions described herein that are suitable for oral administration can be presented as discrete units such as capsules, sachets, granules, or tablets each containing a predetermined amount (e.g., effective amount) of the active ingredient(s); a powder or granules; a solution or a suspension in an aqueous liquid or a non-aqueous liquid; an oil-in-water liquid emulsion; a water-in-oil liquid emulsion; packed in liposomes; or as a bolus. Soft gelatin capsules can be useful for containing such suspensions, which can beneficially increase the rate of compound absorption. In the case of tablets for oral use, carriers that are commonly used include, without limitation, lactose, sucrose, glucose, mannitol, silicic acid, and starches. Other acceptable excipients can include, without limitation, (a) fillers or extenders such as starches, lactose, sucrose, glucose, mannitol, and silicic acid, (b) binders such as carboxymethylcellulose, alginates, gelatin, polyvinylpyrrolidone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. For oral administration in a capsule form, useful diluents include, without limitation, lactose and dried cornstarch. When aqueous suspensions are administered orally, the active ingredient(s) can be combined with emulsifying and suspending agents. If desired, certain sweetening and/or flavoring and/or coloring agents can be added. Compositions suitable for oral administration include, without limitation, lozenges comprising ingredients in a flavored basis, usually sucrose and acacia or tragacanth; and pastilles comprising the active ingredient(s) in an inert basis such as gelatin and glycerin, or sucrose and acacia.

Compositions suitable for parenteral administration include, without limitation, aqueous and non-aqueous sterile injection solutions or infusion solutions that may contain antioxidants, buffers, bacteriostats, and solutes that render the formulation isotonic with the blood of the intended recipient; and aqueous and non-aqueous sterile suspensions that may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water, for injections, saline (e.g., 0.9% saline solution), or 5% dextrose solution, immediately prior to use. Extemporaneous injection solutions and suspensions can be prepared from sterile powders, granules, and tablets. The injection solutions can be in the form of, for example, a sterile injectable aqueous or oleaginous suspension. This suspension can be formulated according to techniques known in the art using suitable dispersing or wetting agents and suspending agents. A sterile injectable preparation also can be a sterile injectable solution or suspension in a non-toxic parenterally-acceptable diluent or solvent, for example, as a solution in 1,3-butanediol. Among the acceptable vehicles and solvents that can be employed are mannitol, water, Ringer's solution, and isotonic sodium chloride solution. In addition, sterile, fixed oils can be used as a solvent or suspending medium. For this purpose, any bland fixed oil can be used including, without limitation, synthetic mono- or diglycerides. Fatty acids such as oleic acid and its glyceride derivatives can be used to prepare injectables. In some cases, natural pharmaceutically acceptable oils such as olive oil or castor oil, especially in their polyoxyethylated versions, can be used to prepare injectables. These oil solutions or suspensions also can contain a long-chain alcohol diluent or dispersant.

In some cases, a therapeutic compound and/or pharmaceutical composition provided herein can be administered in the form of suppository for rectal administration. These compositions can be prepared by mixing a compound described herein (e.g., a compound set forth in Formula (I), (II), (III), (IV), (V), (VI), or (VII), or any one of the compounds listed in Table A, or a pharmaceutically acceptable salt thereof) with a suitable non-irritating excipient that is solid at room temperature but liquid at the rectal temperature and therefore will melt in the rectum to release the active component(s). Such materials include, without limitation, cocoa butter, beeswax, and polyethylene glycols.

In some cases, a therapeutic compounds and/or pharmaceutical composition provided herein can be administered by nasal aerosol or inhalation. Such compositions can be prepared according to techniques well known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J. Pharm. Pharmacol., 56:3-17 (2004); and Ilium, L., Eur. J. Pharm. Sci., 11:1-18 (2000).

In some cases, a therapeutic compounds and/or pharmaceutical composition provided herein can be prepared as a topical composition and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of a therapeutic compounds and/or pharmaceutical composition provided herein can be useful when the desired treatment involves areas or organs readily accessible by topical application. In some cases, a topical composition can include a combination of any one or more of the compounds or therapeutic agents described herein (e.g., a compound set forth in Formula (I), (II), (III), (IV), (V), (VI), or (VII), or any one of the compounds listed in Table A, or a pharmaceutically acceptable salt thereof), and one or more additional ingredients, carriers, excipients, or diluents including, without limitation, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

In some cases, one or more compounds or therapeutic agent described herein (e.g., a compound set forth in Formula (I), (II), (III), (IV), (V), (VI), or (VII), or any one of the compounds listed in Table A, or a pharmaceutically acceptable salt thereof) can be incorporated into a composition for coating an implantable medical device such as a prosthesis, artificial valve, vascular graft, stent, or catheter. Suitable coatings and the general preparation of coated implantable devices are known in the art and are exemplified in U.S. Pat. Nos. 6,099,562; 5,886,026; and 5,304,121. The coatings can be biocompatible polymeric materials such as a hydrogel polymer, polymethyldisiloxane, polycaprolactone, polyethylene glycol, polylactic acid, ethylene vinyl acetate, or mixture thereof. In some cases, the coating can optionally be further covered by a suitable topcoat of fluorosilicone, polysaccharides, polyethylene glycol, phospholipids or combinations thereof to impart controlled release characteristics in the composition.

In some cases, this document provides an implantable drug release device impregnated with or containing one or more compounds or therapeutic agents described herein (e.g., a compound set forth in Formula (I), (II), (III), (IV), (V), (VI), or (VII), or any one of the compounds listed in Table A, or a pharmaceutically acceptable salt thereof) such that the compound(s) or therapeutic agent(s) are released from the device and are therapeutically active.

### Dosages and Regimens

A composition (e.g., pharmaceutical compositions provided herein) containing a compound provided herein (e.g., a compound set forth in Formula (I), (II), (III), (IV), (V), (VI), or (VII), or any one of the compounds listed in Table A, or a pharmaceutically acceptable salt thereof) can include that compound in an effective amount (e.g., a therapeutically effective amount).

Effective doses can vary, depending on the disease, disorder, or condition being treated (or prevented), the severity of the disease, disorder, or condition, the route of administration, the sex, age and general health condition of the subject, excipient usage, the possibility of co-usage with other therapeutic treatments such as use of other agents, and the judgment of the treating physician.

In some embodiments, an effective amount of a compound of Formula (I), (II), (III), (IV), (V), (VI), or (VII), or any one of the compounds listed in Table A, or a pharmaceutically acceptable salt thereof, can range, for example, from about 0.1 mg to about 1000 mg. In some cases, the effective amount can be from about 0.5 mg to about 500 mg of a compound disclosed herein, or any amount in between these two values, for example, one of about 0.5 mg, about 1 mg, about 2 mg, about 5 mg, about 10 mg, about 20 mg, about 50 mg, about 100 mg, about 200 mg, about 250 mg, about 300 mg, about 400 mg, or about 500 mg. The effective amount can be an amount sufficient to alleviate or reduce one or more of the symptoms associated with a disease, disorder, or condition being treated (or prevented) as described herein.

In some cases, an effective amount of a compound of Formula (I), (II), (III), (IV), (V), (VI), or (VII), or any one of the compounds listed in Table A, or a pharmaceutically acceptable salt thereof, can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; from about 0.1 mg/kg to about 0.5 mg/kg, or from about 0.5 mg/kg to about 500 mg/kg).

In some cases, an effective amount of a compound of Formula (I), (II), (III), (IV), (V), (VI), or (VII), or any one of the compounds listed in Table A, or a pharmaceutically acceptable salt thereof, can be about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or on a non-daily basis (e.g., every other day, every two days, every three days, once weekly, twice weekly, once every two weeks, or once a month). In some cases, the dosages can be administered every 4 hours, 6 hours, 8 hours, 12 hours, or 24 hours.

### Kits

This document also provides pharmaceutical kits useful, for example, to increase or maintain NMNAT2 polypeptide levels within cells within a mammal (e.g., a human). In some cases, this document provides pharmaceutical kits useful, for example, to treat (or prevent) a disease, disorder, or condition referred to herein. Such pharmaceutical kits can include one or more containers containing a pharmaceutical composition that includes a therapeutically effective amount of a compound provided herein (e.g., a compound set forth in Formula (I), (II), (III), (IV), (V), (VI), or (VII), or any one of the compounds listed in Table A, or a pharmaceutically acceptable salt thereof). In some cases, such kits can further include, if desired, one or more of various conventional pharmaceutical kit components such as containers with one or more pharmaceutically acceptable carriers. Instructions, either as inserts or as labels, indicating quantities of the components to be administered, guidelines for administration, and/or guidelines for mixing the components also can be included in a kit provided herein. In some embodiments, the kit comprising at least one additional therapeutic agent as described herein, or a pharmaceutically acceptable salt thereof, or a pharmaceutical composition comprising same.

### Combination Therapy

In some cases, one or more compounds provided herein (e.g., a compound set forth in Formula (I), (II), (III), (IV), (V), (VI), or (VII), or any one of the compounds listed in Table A, or a pharmaceutically acceptable salt thereof) can be combined with one or more additional therapeutic molecules. Examples of therapeutic molecules that can be used in combination with one or more compounds provided herein (e.g., a compound set forth in Formula (I), (II), (III), (IV), (V), (VI), or (VII), or any one of the compounds listed in Table A, or a pharmaceutically acceptable salt thereof) include, without limitation, a diuretic, an anti-seizure drug, a drug to increase NAD levels, an analgesic, a corticosteroid, and a coma-inducing drug. Additional examples of therapeutic molecules that can be used in combination with one or more compounds provided herein (e.g., a compound set forth in Formula (I), (II), (III), (IV), (V), (VI), or (VII), or any one of the compounds listed in Table A, or a pharmaceutically acceptable salt thereof) include, without limitation, anti-inflammatory agents (e.g., steroids and antibodies against IL-6 or TNF-alpha), antimicrobial agents (e.g., antibiotics, anti-mycobacterial drugs, and anti-viral agents), anti-aging agents (e.g., metformin or rapamycin), neurological agents (e.g., L-DOPA, memantine, and riluzole), and therapies for a neurodegenerative disease (e.g., edaravone or tetrabenazine) or agents intended to raise NAD levels (nicotinamide riboside or nicotinamide mononucleotide).

One or more compounds provided herein (e.g., a compound set forth in Formula (I), (II), (III), (IV), (V), (VI), or (VII), or any one of the compounds listed in Table A, or a pharmaceutically acceptable salt thereof) and the one or more therapeutic molecules can be administered in any order or simultaneously. If simultaneously administered, they can be provided in a single, unified, form or in multiple forms (e.g., either as a single pill or as two separate pills). One of the items can be given in multiple doses, or both can be given as multiple doses. If not simultaneous, the timing between the multiple doses can vary from more than zero weeks to less than four weeks.

### Definitions

As used herein, the term “about” means “approximately” (e.g., plus or minus approximately 10% of the indicated value).

At various places in this document, substituents of compounds provided herein are disclosed in groups or in ranges. It is specifically intended that these groups and ranges include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in this document various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features described herein, which are, for clarity, described in the context of separate embodiments, can also be provided in combination in a single embodiment. Conversely, various features described herein which are, for brevity, described in the context of a single embodiment, also can be provided separately or in any suitable subcombination.

The term “aromatic” refers to a carbocycle or heterocycle having one or more polyunsaturated rings having aromatic character (i.e., having (4n+2) delocalized R (pi) electrons where n is an integer).

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution can be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, without limitation, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms that may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, without limitation, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, without limitation, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl-linking group having n to m carbons. Examples of alkylene groups include, without limitation, ethan-1,1-diyl, ethan-1,2-diyl, propan-1,1-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, without limitation, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, without limitation, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cₙ₋ₘ alkoxycarbonyl” refers to a group of formula —C(O)O— alkyl, wherein the alkyl group has n to m carbon atoms. In some embodiments, the alkyl group has 1 to 6, 1 to 4, or 1 to 3 carbon atoms. Examples of alkoxycarbonyl groups include, without limitation, methoxycarbonyl, ethoxycarbonyl, propoxycarbonyl (e.g., n-propoxycarbonyl and isopropoxycarbonyl), butoxycarbonyl (e.g., n-butoxycarbonyl and tert-butoxycarbonyl), and the like.

As used herein, the term “Cₙ₋ₘ alkylcarbonyl” refers to a group of formula —C(O)— alkyl, wherein the alkyl group has n to m carbon atoms. In some embodiments, the alkyl group has 1 to 6, 1 to 4, or 1 to 3 carbon atoms. Examples of alkylcarbonyl groups include, without limitation, methylcarbonyl, ethylcarbonyl, propylcarbonyl (e.g., n-propylcarbonyl and isopropylcarbonyl), butylcarbonyl (e.g., n-butylcarbonyl and tert-butylcarbonyl), and the like.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms.

In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “carbamyl” to a group of formula —C(O)NH₂.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carbonyl”, employed alone or in combination with other terms, refers to a —C(═O)— group, which may also be written as C(O).

As used herein, the term “carboxy” refers to a —C(O)OH group. In some embodiments, the “carboxy” group also refers to a bioisostere replacement group selected from the group consisting of:

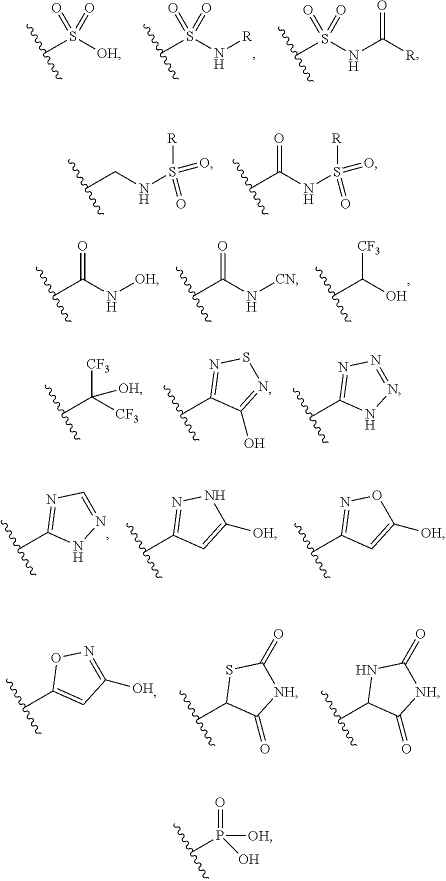

and the like, where R refers to a hydrogen, (C₁-C₈) alkyl, or C₆ aryl.

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which can be monocyclic or polycyclic (e.g., having 2, 3, or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups can have from 6 to 10 carbon atoms. In some embodiments, the aryl group is phenyl or naphthyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3, or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃-10). In some embodiments, the cycloalkyl is a C₃₋₁₀ monocyclic or bicyclic cycloalkyl. In some embodiments, the cycloalkyl is a C₃₋₇ monocyclic cycloalkyl. Example cycloalkyl groups include, without limitation, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur, and oxygen. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from nitrogen, sulfur, and oxygen. In some embodiments, the heteroaryl is a 5-6 monocyclic heteroaryl having 1 or 2 heteroatom ring members independently selected from nitrogen, sulfur, and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls include, without limitation, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls include, without limitation, pyridyl, pyrazinyl, pyrimidinyl, triazinyl, and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9-, or 10-membered heterocycloalkyl groups. Heterocycloalkyl groups can also include spirocycles. Example heterocycloalkyl groups include, without limitation, pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring can be attached at any position of the ring, whereas a pyridin-3-yl ring is attached at the 3-position.

As used herein, the term “oxo” refers to an oxygen atom as a divalent substituent, forming a carbonyl group when attached to a carbon (e.g., C═O), or attached to a heteroatom forming a sulfoxide or sulfone group.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds provided herein that contain asymmetrically substituted carbon atoms can be isolated in optically active or racemic forms. Any appropriate method can be used to prepare optically active forms from, for example, optically inactive starting materials. For example, techniques such as resolution of racemic mixtures or stereoselective synthesis can be used to prepare optically active forms of a compound provided herein. Many geometric isomers of olefins, C═N double bonds, N═N double bonds, and the like also can be present in a compound described herein, and all such stable isomers are contemplated herein. Cis and trans geometric isomers of the compounds provided herein are described and can be isolated as a mixture of isomers or as separated isomeric forms. In some embodiments, a compound provided herein has the (R)-configuration. In some embodiments, a compound provided herein has the (S)-configuration.

Compounds provided herein also include tautomeric forms. Tautomeric forms result from the swapping of a single bond with an adjacent double bond together with the concomitant migration of a proton. Tautomeric forms include prototropic tautomers that are isomeric protonation states having the same empirical formula and total charge. Example prototropic tautomers include, without limitation, ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H-, and 4H-1,2,4-triazole, 1H- and 2H-isoindole, and 1H- and 2H-pyrazole. Tautomeric forms can be in equilibrium or sterically locked into one form by appropriate substitution. For example, in aqueous solution, pyrazoles can exhibit the following isomeric forms, which are referred to as tautomers of each other:

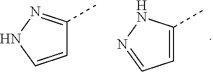

As readily understood by one skilled in the art, a wide variety of functional groups and other structures can exhibit tautomerism, and all tautomers of compounds as described herein are within the scope provided herein.

As used herein, the term “cell” is meant to refer to a cell that is in vitro, ex vivo, or in vivo. In some embodiments, an ex vivo cell can be part of a tissue sample excised from an organism such as a mammal (e.g., a human). In some embodiments, an in vitro cell can be a cell in cell culture. In some embodiments, an in vivo cell is a cell living in an organism such as a mammal (e.g., a human).

As used herein, the term “contacting” refers to the bringing together of indicated moieties or items in an in vitro system, an ex vivo system, or an in vivo system. For example, “contacting” a cell with a compound provided herein includes the act of administering that compound to a mammal (e.g., a human) containing that cell as well as, for example, introducing that compound into a cell culture containing that cell.

As used herein, the term “mammal” includes, without limitation, mice, rats, other rodents, rabbits, dogs, cats, swine, cattle, sheep, horses, elephants, deer, non-human primates (e.g., monkeys and apes), house pets, and humans.

As used herein, the phrase “effective amount” or “therapeutically effective amount” refers to the amount of active compound or pharmaceutical agent that elicits the biological or medicinal response in a tissue, system, mammal, or human that is being sought by a researcher, veterinarian, medical doctor, or other clinician.

As used herein, the term “treating” or “treatment” refers to (a) inhibiting a disease, disorder, or condition, for example, inhibiting a disease, disorder, or condition in a mammal (e.g., human) that is experiencing or displaying the pathology or symptomatology of the disease, disorder, or condition (e.g., arresting further development of the pathology and/or symptomatology), or (b) ameliorating the disease, disorder, or condition, for example, ameliorating a disease, disorder, or condition in a mammal (e.g., a human) that is experiencing or displaying the pathology or symptomatology of the disease, disorder, or condition (e.g., reversing the pathology and/or symptomatology).

As used herein, the term “preventing” or “prevention” of a disease, disorder, or condition refers to decreasing the risk of occurrence of the disease, disorder, or condition in a mammal or group of mammals (e.g., a mammal or group of mammals predisposed to or susceptible to the disease, disorder, or condition). In some embodiments, preventing a disease, disorder, or condition refers to decreasing the possibility of acquiring the disease, disorder, or condition and/or its associated symptoms. In some embodiments, preventing a disease, disorder, or condition refers to completely or almost completely stopping the disease, disorder, or condition from occurring.

### EXAMPLES

### Example 1

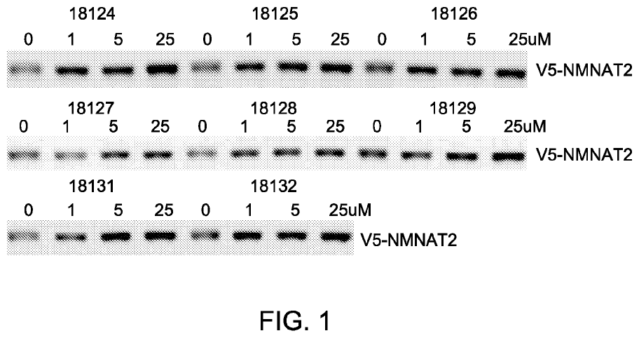

Selected compounds were tested in vitro. Briefly, Beas2B cells were transfected with NMNAT2-V5 plasmid. 24 hours later, cells were treated with a test compound in a dose dependent manner for additional 24 hours. Cells were then collected and assayed for NMNAT2-V5 polypeptide expression. Results of the assay are shown in Table A. Western blots showing levels of NMNAT2-V5 polypeptide for selected compounds are shown in FIG. 1.

TABLE A

| | Compound | A¹ |
|---|---|---|
| BC18115 | 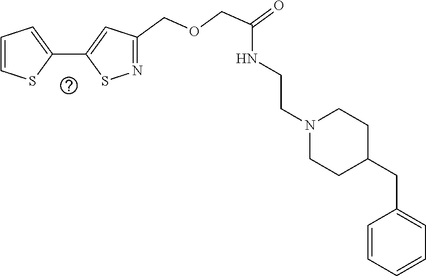 [SMILES: O=C(COCc1cc(-c2cccs2)sn1)NCCN1CCC(Cc2ccccc2)CC1] | ++ |
| BC18116 | 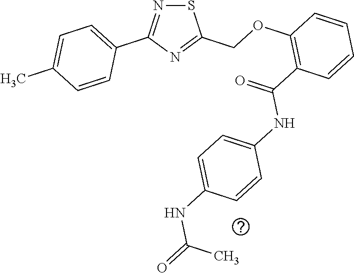 [SMILES: CC(=O)Nc1ccc(NC(=O)c2ccccc2OCc2nc(-c3ccc(C)cc3)ns2)cc1] | + |
| BC18117 | 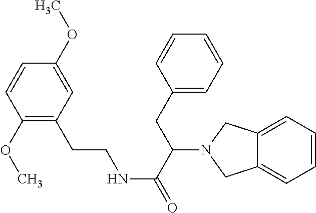 [SMILES: COc1ccc(OC)c(CCNC(=O)C(Cc2ccccc2)N2Cc3ccccc3C2)c1] | ++ |
| BC18118 | 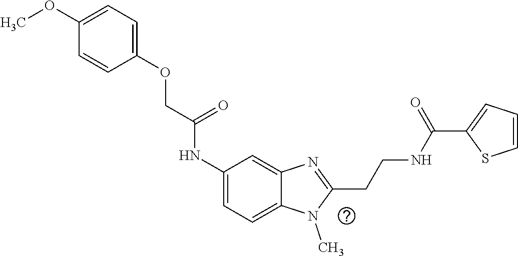 [SMILES: COc1ccc(OCC(=O)Nc2ccc3c(c2)nc(CCNC(=O)c2cccs2)n3C)cc1] | + |
| BC18119 | 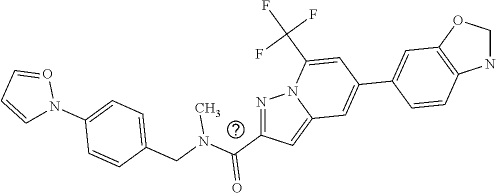 | + |
| BC18120 | 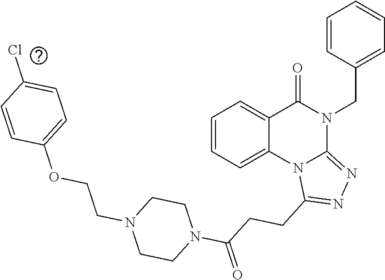 [SMILES: O=C(CCc1nnc2n(Cc3ccccc3)c(=O)c3ccccc3n12)N1CCN(CCOc2ccc(Cl)cc2)CC1] | +++ |
| BC18121 | 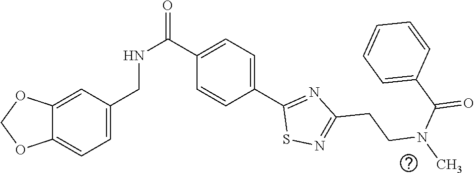 [SMILES: CN(CCc1nsc(-c2ccc(C(=O)NCc3ccc4c(c3)OCO4)cc2)n1)C(=O)c1ccccc1] | + |
| BC18122 | 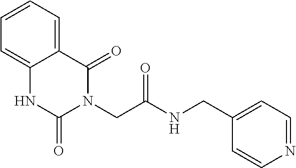 [SMILES: O=C(Cn1c(=O)[nH]c2ccccc2c1=O)NCc1ccncc1] | ++ |
| BC18123 | 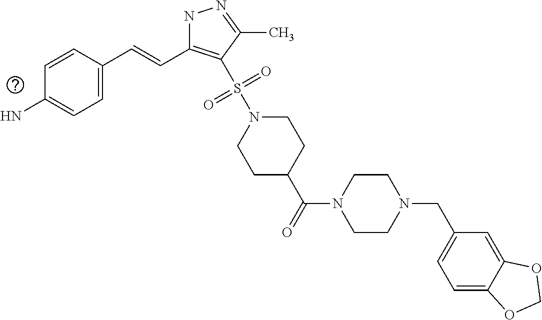 [SMILES: Cc1n[nH]c(/C=C/c2ccc(N)cc2)c1S(=O)(=O)N1CCC(C(=O)N2CCN(Cc3ccc4c(c3)OCO4)CC2)CC1] | +++ |
| BC18124 | 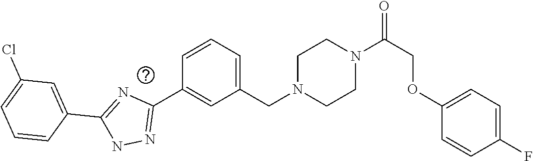 [SMILES: O=C(COc1ccc(F)cc1)N1CCN(Cc2cccc(-c3n[nH]c(-c4cccc(Cl)c4)n3)c2)CC1] | +++ |
| BC18125 | 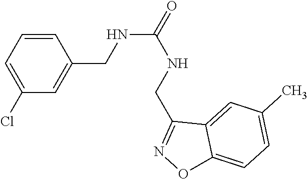 [SMILES: Cc1ccc2onc(CNC(=O)NCc3cccc(Cl)c3)c2c1] | +++ |
| BC18126 (BC181152) | 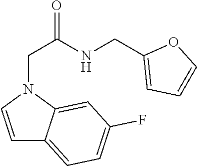 [SMILES: O=C(Cn1ccc2ccc(F)cc21)NCc1ccco1] | +++ |
| BC18127 | 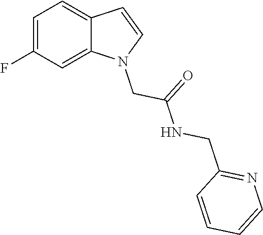 [SMILES: O=C(Cn1ccc2ccc(F)cc21)NCc1ccccn1] | ++ |
| BC18128 (BC181128) | 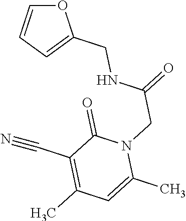 [SMILES: Cc1cc(C)n(CC(=O)NCc2ccco2)c(=O)c1C#N] | +++ |
| BC18129 (BC181050) | 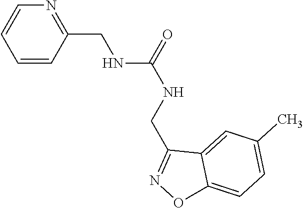 [SMILES: Cc1ccc2onc(CNC(=O)NCc3ccccn3)c2c1] | ++ |
| BC18130 | 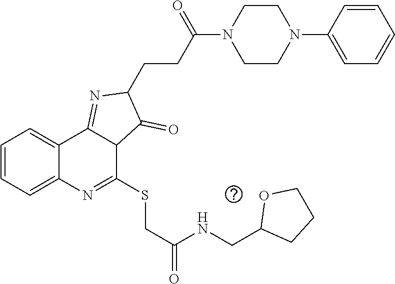 [SMILES: O=C(CSC1=Nc2ccccc2C2=NC(CCC(=O)N3CCN(c4ccccc4)CC3)C(=O)C12)NCC1CCCO1] | ++ |
| BC18131 | 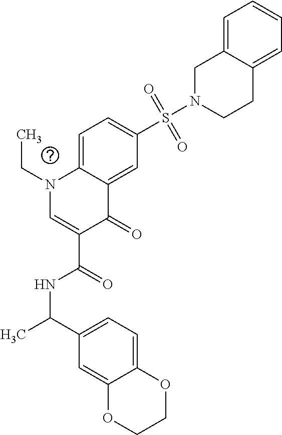 [SMILES: CCn1cc(C(=O)NC(C)c2ccc3c(c2)OCCO3)c(=O)c2cc(S(=O)(=O)N3CCc4ccccc4C3)ccc21] | ++ |
| BC18132 | 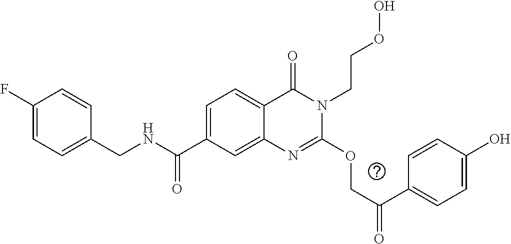 [SMILES: O=C(COc1nc2cc(C(=O)NCc3ccc(F)cc3)ccc2c(=O)n1CCOO)c1ccc(O)cc1] | +++ |
| BC18133 | 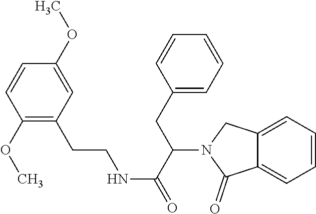 [SMILES: COc1ccc(OC)c(CCNC(=O)C(Cc2ccccc2)N2Cc3ccccc3C2=O)c1] | ++ |
| BC18134 | 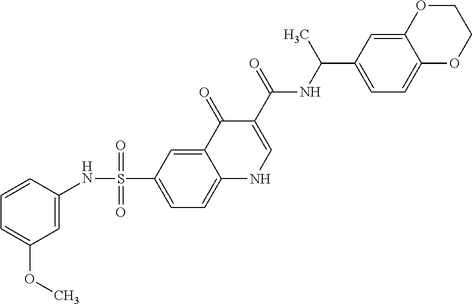 [SMILES: COc1cccc(NS(=O)(=O)c2ccc3[nH]cc(C(=O)NC(C)c4ccc5c(c4)OCCO5)c(=O)c3c2)c1] | + |
| BC18135 | 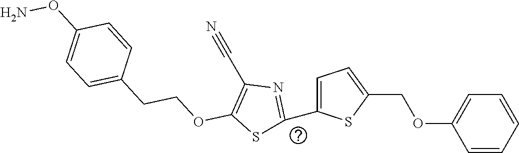 [SMILES: N#Cc1nc(-c2ccc(COc3ccccc3)s2)sc1OCCc1ccc(ON)cc1] | + |
| BC18136 | 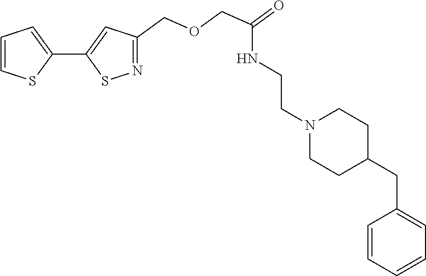 [SMILES: O=C(COCc1cc(-c2cccs2)sn1)NCCN1CCC(Cc2ccccc2)CC1] | + |
| BC18137 | 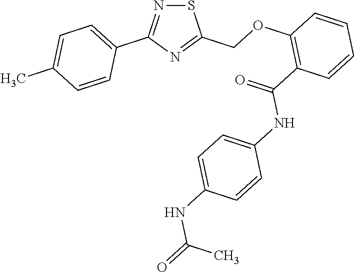 [SMILES: CC(=O)Nc1ccc(NC(=O)c2ccccc2OCc2nc(-c3ccc(C)cc3)ns2)cc1] | ++ |
| BC18138 | 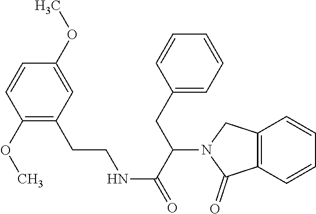 [SMILES: COc1ccc(OC)c(CCNC(=O)C(Cc2ccccc2)N2Cc3ccccc3C2=O)c1] | ++ |
| BC18139 | 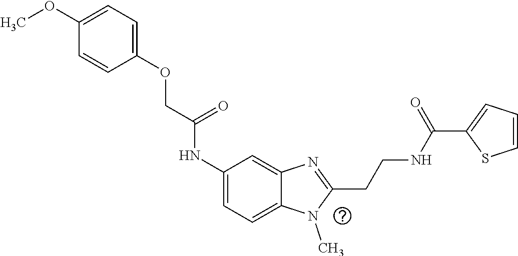 [SMILES: COc1ccc(OCC(=O)Nc2ccc3c(c2)nc(CCNC(=O)c2cccs2)n3C)cc1] | + |

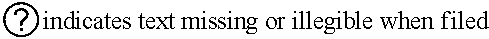

¹A is compound efficacy (IC₅₀) for NMNAT2 activation.
Activity:
“+++” <5 μM;
“++” >5 μM and <25 μM; and
“+” ≥ 25 μM.

### Example 2

Selected compounds were tested in vitro. Briefly, U87MG cells were transfected with NMNAT2-Hibit luc plasmid. Cells were treated with a test compound at 25 μM for 24 hours. Cells were then collected and assayed for nano-luciferase activity. Results of the assay are shown in Table B.

TABLE B

| Compound | B¹ | Compound | B¹ | Compound | B¹ | Compound | B¹ |
|---|---|---|---|---|---|---|---|
| 181001 | + | 181024 | + | 181047 | ++ | 181070 | + |
| 181002 | + | 181025 | + | 181048 | ++ | 181071 | + |
| 181003 | + | 181026 | + | 181049 | + | 181072 | + |
| 181004 | + | 181027 | + | 181050 | + | 181073 | + |
| 181005 | + | 181028 | + | 181051 | + | 181074 | + |
| 181006 | + | 181029 | ++ | 181052 | + | 181075 | + |
| 181007 | + | 181030 | + | 181053 | + | 181076 | + |
| 181008 | + | 181031 | + | 181054 | + | 181077 | ++ |
| 181009 | + | 181032 | + | 181055 | + | 181078 | + |
| 181010 | + | 181033 | ++ | 181056 | + | 181079 | + |
| 181011 | + | 181034 | + | 181057 | + | 181080 | + |
| 181012 | + | 181035 | + | 181058 | + | 181081 | + |
| 181013 | + | 181036 | + | 181059 | + | 181082 | ++ |
| 181014 | + | 181037 | + | 181060 | ++ | 181083 | ++ |
| 181015 | + | 181038 | + | 181061 | + | 181084 | + |
| 181016 | + | 181039 | + | 181062 | + | 181085 | + |
| 181017 | + | 181040 | + | 181063 | + | 181086 | ++ |
| 181018 | + | 181041 | + | 181064 | + | 181087 | + |
| 181019 | + | 181042 | + | 181065 | + | 181088 | + |
| 181020 | + | 181043 | + | 181066 | + | 181089 | ++ |
| 181021 | + | 181044 | + | 181067 | + | 181090 | + |
| 181022 | + | 181045 | + | 181068 | ++ | 181091 | + |
| 181023 | + | 181046 | + | 181069 | ++ | 181092 | + |

¹B is compound efficacy (IC₅₀) for NMNAT2 activation:
“+++” >100% increase in NMNAT2 luciferase activity.
“++” >20% and <100% increase in NMNAT2 luciferase activity.
“+” <20% increase in NMNAT2 luciferase activity.

### Example 3

Selected compounds were tested in vitro. Briefly, U87MG cells were transfected with NMNAT2-Hibit luc plasmid. Cells were treated with a test compound at 25 μM for 24 hours. Cells were then collected and assayed for nano-luciferase activity. Results of the assay are shown in Table C.

TABLE C

| Compound | C¹ | Compound | C¹ | Compound | C¹ | Compound | C¹ |
|---|---|---|---|---|---|---|---|
| 181101 | + | 181115 | + | 181129 | + | 181143 | + |
| 181102 | + | 181116 | ++ | 181130 | + | 181144 | + |
| 181103 | + | 181117 | + | 181131 | + | 181145 | + |
| 181104 | + | 181118 | + | 181132 | + | 181146 | + |
| 181105 | + | 181119 | + | 181133 | + | 181147 | + |
| 181106 | +++ | 181120 | + | 181134 | + | 181148 | ++ |
| 181107 | +++ | 181121 | + | 181135 | + | 181149 | ++ |
| 181108 | + | 181122 | + | 181136 | + | 181150 | + |
| 181109 | + | 181123 | + | 181137 | + | 181151 | + |
| 181110 | + | 181124 | + | 181138 | + | 181152 | + |
| 181111 | + | 181125 | + | 181139 | + | 181153 | ++ |
| 181112 | + | 181126 | + | 181140 | + | 181154 | ++ |
| 181113 | + | 181127 | + | 181141 | ++ | 181155 | + |
| 181114 | +++ | 181128 | + | 181142 | + | 181156 | + |
| | | | | | | 181157 | + |
| | | | | | | 181158 | + |
| | | | | | | 181159 | + |

¹C is Compound efficacy for NMNAT2 activation:
“+++” >100% increase in NMNAT2 luciferase activity.
“++” >20% and <100% increase in NMNAT2 luciferase activity.
“+” <20% increase in NMNAT2 luciferase activity.

### Example 4

Selected compounds were tested in vitro. Briefly, U87MG cells were transfected with NMNAT2-Hibit luciferase plasmid. Cells were treated with test compound at 5 μM for 48 hours. Cells were then collected and assayed for nano-luciferase activity. Results of the assay are shown in Table D.

TABLE D

| Compound | D¹ | Compound | D¹ | Compound | D¹ | Compound | D¹ |
|---|---|---|---|---|---|---|---|
| BC181201 | +++ | BC181218 | + | BC181235 | ++ | BC181252 | ++ |
| BC181202 | + | BC181219 | ++ | BC181236 | ++ | BC181253 | ++ |
| BC181203 | ++ | BC181220 | +++ | BC181237 | ++ | BC181254 | ++ |
| BC181204 | ++ | BC181221 | + | BC181238 | ++ | BC181255 | + |
| BC181205 | + | BC181222 | ++ | BC181239 | + | BC181256 | + |
| BC181206 | ++ | BC181223 | + | BC181240 | + | BC181257 | + |
| BC181207 | ++ | BC181224 | + | BC181241 | + | BC181258 | ++ |
| BC181208 | + | BC181225 | + | BC181242 | + | BC181259 | ++ |
| BC181209 | + | BC181226 | + | BC181243 | ++ | BC181260 | ++ |
| BC181210 | ++ | BC181227 | + | BC181244 | ++ | BC181261 | ++ |
| BC181211 | ++ | BC181228 | + | BC181245 | ++ | BC181262 | ++ |
| BC181212 | + | BC181229 | ++ | BC181246 | ++ | BC181263 | ++ |
| BC181213 | + | BC181230 | +++ | BC181247 | +++ | BC181264 | + |
| BC181214 | + | BC181231 | ++ | BC181248 | ++ | BC181265 | + |
| BC181215 | + | BC181232 | + | BC181249 | + | BC181266 | + |
| BC181216 | + | BC181233 | + | BC181250 | + | BC181267 | + |
| BC181217 | + | BC181234 | ++ | BC181251 | ++ | BC181268 | ++ |

¹D is compound efficacy for NMNAT2 activation:
“+++” >100% increase in NMNAT2 luciferase activity.
“++” >20% and <100% increase in NMNAT2 luciferase activity.
“+” <20% increase in NMNAT2 luciferase activity.

### Example 5

Selected compounds were tested in vitro. U87MG cells were stably transfected with NMNAT2-Hibit luc plasmid. Cells were treated with a test compound at various concentrations for 24 hours. Cells were then collected and assayed for nano-luciferase activity. Results of the assay are shown in Table E.

TABLE E

| comound | E¹ | comound | E¹ | comound | E¹ | comound | E¹ |
|---|---|---|---|---|---|---|---|
| BC20100 | ++ | BC20177 | +++ | BC20254 | ++ | BC20331 | + |
| BC20101 | +++ | BC20178 | ++ | BC20255 | + | BC20332 | ++ |
| BC20102 | ++ | BC20179 | ++ | BC20256 | + | BC20333 | ++ |
| BC20103 | +++ | BC20180 | ++ | BC20257 | + | BC20334 | + |
| BC20104 | ++ | BC20181 | ++ | BC20258 | ++ | BC20335 | + |
| BC20105 | ++ | BC20182 | + | BC20259 | +++ | BC20336 | + |
| BC20106 | ++ | BC20183 | + | BC20260 | ++ | BC20337 | + |
| BC20107 | ++ | BC20184 | + | BC20261 | + | BC20338 | + |
| BC20108 | +++ | BC20185 | ++ | BC20262 | ++ | BC20339 | + |
| BC20109 | ++ | BC20186 | + | BC20263 | ++ | BC20340 | + |
| BC20110 | +++ | BC20187 | + | BC20264 | ++ | BC20341 | + |
| BC20111 | +++ | BC20188 | ++ | BC20265 | ++ | BC20342 | + |
| BC20112 | +++ | BC20189 | ++ | BC20266 | ++ | BC20343 | + |
| BC20113 | ++ | BC20190 | + | BC20267 | ++ | BC20344 | + |
| BC20114 | ++ | BC20191 | ++ | BC20268 | + | BC20345 | + |
| BC20115 | ++ | BC20192 | ++ | BC20269 | + | BC20346 | + |
| BC20116 | ++ | BC20193 | + | BC20270 | + | BC20347 | ++ |
| BC20117 | ++ | BC20194 | + | BC20271 | + | BC20348 | + |
| BC20118 | ++ | BC20195 | + | BC20272 | + | BC20349 | + |
| BC20119 | +++ | BC20196 | + | BC20273 | + | BC20350 | + |
| BC20120 | +++ | BC20197 | ++ | BC20274 | + | BC20351 | + |
| BC20121 | ++ | BC20198 | +++ | BC20275 | + | BC20352 | + |
| BC20122 | ++ | BC20199 | ++ | BC20276 | + | BC20353 | ++ |
| BC20123 | ++ | BC20200 | ++ | BC20277 | + | BC20354 | + |
| BC20124 | ++ | BC20201 | + | BC20278 | + | BC20355 | ++ |
| BC20125 | ++ | BC20202 | ++ | BC20279 | + | BC20356 | + |
| BC20126 | ++ | BC20203 | ++ | BC20280 | + | BC20357 | ++ |
| BC20127 | ++ | BC20204 | ++ | BC20281 | + | BC20358 | + |
| BC20128 | ++ | BC20205 | + | BC20282 | + | BC20359 | + |
| BC20129 | ++ | BC20206 | + | BC20283 | + | BC20360 | ++ |
| BC20130 | ++ | BC20207 | + | BC20284 | + | BC20361 | ++ |
| BC20131 | ++ | BC20208 | ++ | BC20285 | ++ | BC20362 | ++ |
| BC20132 | ++ | BC20209 | ++ | BC20286 | ++ | BC20363 | ++ |
| BC20133 | ++ | BC20210 | + | BC20287 | ++ | BC20364 | +++ |
| BC20134 | ++ | BC20211 | +++ | BC20288 | ++ | BC20365 | + |
| BC20135 | ++ | BC20212 | +++ | BC20289 | + | BC20366 | + |
| BC20136 | ++ | BC20213 | ++ | BC20290 | + | BC20367 | ++ |
| BC20137 | ++ | BC20214 | + | BC20291 | ++ | BC20368 | ++ |
| BC20138 | ++ | BC20215 | ++ | BC20292 | ++ | BC20369 | ++ |
| BC20139 | ++ | BC20216 | ++ | BC20293 | ++ | BC20370 | + |
| BC20140 | + | BC20217 | + | BC20294 | + | BC20371 | ++ |
| BC20141 | + | BC20218 | + | BC20295 | + | BC20372 | + |
| BC20142 | ++ | BC20219 | ++ | BC20296 | ++ | BC20373 | + |
| BC20143 | ++ | BC20220 | ++ | BC20297 | + | BC20374 | + |
| BC20144 | ++ | BC20221 | + | BC20298 | + | BC20375 | + |
| BC20145 | ++ | BC20222 | + | BC20299 | + | BC20376 | + |
| BC20146 | ++ | BC20223 | + | BC20300 | ++ | BC20377 | + |
| BC20147 | ++ | BC20224 | + | BC20301 | + | BC20378 | ++ |
| BC20148 | ++ | BC20225 | + | BC20302 | + | BC20379 | ++ |
| BC20149 | ++ | BC20226 | + | BC20303 | + | BC20380 | + |
| BC20150 | ++ | BC20227 | ++ | BC20304 | + | BC20381 | ++ |
| BC20151 | ++ | BC20228 | ++ | BC20305 | + | BC20382 | ++ |
| BC20152 | ++ | BC20229 | + | BC20306 | + | BC20383 | + |
| BC20153 | ++ | BC20230 | ++ | BC20307 | + | BC20384 | + |
| BC20154 | + | BC20231 | ++ | BC20308 | ++ | BC20385 | ++ |
| BC20155 | ++ | BC20232 | + | BC20309 | + | BC20386 | ++ |
| BC20156 | ++ | BC20233 | ++ | BC20310 | + | BC20387 | + |
| BC20157 | +++ | BC20234 | ++ | BC20311 | + | BC20388 | + |
| BC20158 | ++ | BC20235 | ++ | BC20312 | + | BC20389 | ++ |
| BC20159 | ++ | BC20236 | ++ | BC20313 | + | BC20390 | + |
| BC20160 | ++ | BC20237 | ++ | BC20314 | + | BC20391 | ++ |
| BC20161 | ++ | BC20238 | ++ | BC20315 | + | BC20392 | + |
| BC20162 | +++ | BC20239 | ++ | BC20316 | + | BC20393 | ++ |
| BC20163 | +++ | BC20240 | ++ | BC20317 | + | BC20394 | + |
| BC20164 | +++ | BC20241 | ++ | BC20318 | + | BC20395 | ++ |
| BC20165 | +++ | BC20242 | + | BC20319 | + | BC20396 | ++ |
| BC20166 | ++ | BC20243 | +++ | BC20320 | + | BC20397 | + |
| BC20167 | +++ | BC20244 | + | BC20321 | + | BC20398 | ++ |
| BC20168 | ++ | BC20245 | ++ | BC20322 | ++ | BC20399 | ++ |
| BC20169 | ++ | BC20246 | ++ | BC20323 | + | BC20400 | ++ |
| BC20170 | ++ | BC20247 | + | BC20324 | + | BC20401 | ++ |
| BC20171 | +++ | BC20248 | ++ | BC20325 | + | BC20402 | ++ |
| BC20172 | ++ | BC20249 | +++ | BC20326 | ++ | BC20403 | + |
| BC20173 | ++ | BC20250 | ++ | BC20327 | + | BC20404 | ++ |
| BC20174 | ++ | BC20251 | ++ | BC20328 | + | BC20405 | ++ |
| BC20175 | ++ | BC20252 | + | BC20329 | + | BC20406 | +++ |
| BC20176 | ++ | BC20253 | + | BC20330 | ++ | | |

¹E is comnpound efficacy for NMNAT2 activation.
“+++” >25% increase in NMNAT2 luciferase activity at ≤1 μM.
“++” >25% increase in NMNAT2 luciferase activity at >1 μM ≤25 μM.
“+” >25% increase in NMNAT2 luciferase activity at >25 μM.

### Example 6—Synthetic Preparation of Exemplified Compounds

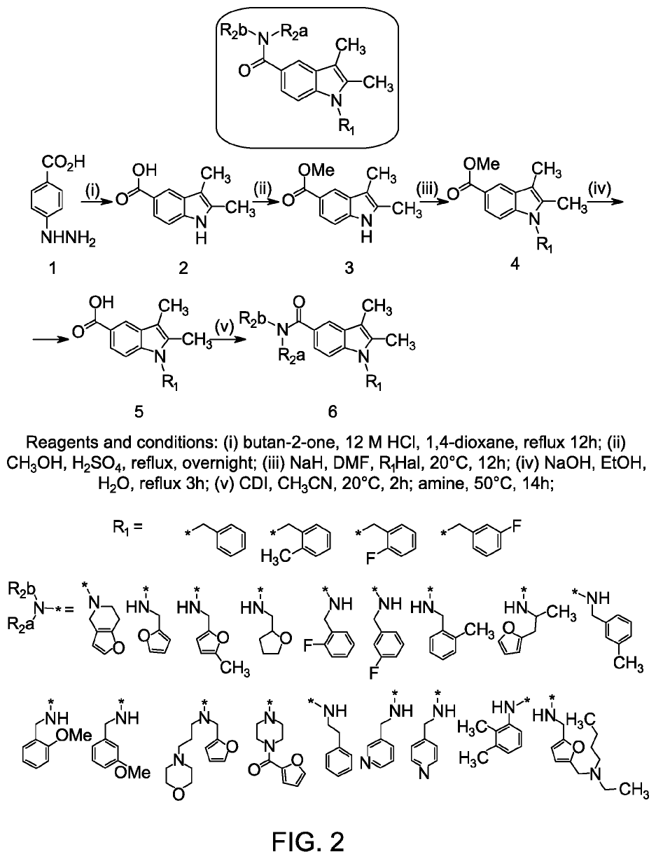

Synthetic scheme for the preparation of indole-5-carboxamide compounds is shown in FIG. 2. Referring to FIG. 2:

Step 1. Synthesis of Compound 2.

A mixture of 4-hydrazinobenzoic acid (10.0 g, 66 mmol), butan-2-one (10.0 g, 72 mmol) and 12M aqueous HCl (10 mL) in 1,4-dioxane (200 mL) was refluxed for 12 h. The reaction mixture was cooled, the resulting precipitate was filtered, washed with water, and the filtrate was concentrated under reduced pressure. Water (200 mL) was added to the residue and stirred for 30 minutes. The precipitate was filtered off, washed with water and dried. The 2,3-dimethyl-1H-indole-5-carboxylic acid (19.2 g, 96%) was obtained white solid and used without further purification.

Step 2. Synthesis of Compounds 3.

To a solution of acid 2 (12.0 g, 63 mmol) in methanol (500 mL) conc. H₂SO₄ (50 mL) was added, and the resulted solution was refluxed overnight. The solvent was evaporated under reduced pressure. The residue was treated with NaHCO₃ solution until the pH of the solution was reached 8-9. The mixture was extracted with EtOAc (3×50 mL), combined organic layers were dried over Na₂SO₄. After filtration the solution was concentrated under reduced pressure. The methyl ester 3 (12.7 g, 98%) was obtained as a colorless oil.

Step 3. Synthesis of Compounds 4.

To a solution of compound 3 (1.8 g, 8.8 mmol) in DMF (20 mL) at the 0° C. was added NaH (60% suspension, 0.4 g, 10.0 mmol) and the mixture was stirred for 30 min. Benzyl bromide (1.67 g, 9.7 mmol) was added and the mixture was stirred overnight at ambient temperature. Water (200 mL) was added, the mixture was extracted with EtOAc (3×50 mL), combined organic layers were washed with water, dried with Na₂SO₄ and evaporated under reduced pressure. A residue was purified by column chromatography (EtOAc/hexane 1:8) to provide compounds 4 (yield 55-78%).

Step 4. Synthesis of Compounds 5.

To a solution of ester 4 (10 mmol) in EtOH (30 mL) a solution of NaOH (0.8 g, 20 mmol) in water (20 mL) was added and the mixture was refluxed for a 3 h. Then mixture was evaporated under reduced pressure, residue was dissolved in water (10 mL) and acidified with acetic acid to pH-6. The acid 5 was filtered off, washed with water, and dried on air. Yield 80-90%.

Step 5. Combinatorial Synthesis of Final Compounds 6 (General Procedure for Amide Coupling).

To a solution of compound 5 (0.3 mmol) in CH₃CN (2 mL), CDI (0.35 mmol) was added. The mixture was stirred at 20° C. for 2 h. Corresponding amine (0.45 mmol) was added and reaction mixture was stirred at 50° C. for 16 h. After cooling, the reaction mixture was poured into water (5 mL) and extracted with CH₂Cl₂. The combined organic extracts were dried over Na₂SO₄, filtered, and concentrated under reduced pressure. The residue was purified by HPLC. Yield 50-70%.

### Example 7—Synthetic Preparation of Exemplified Compounds

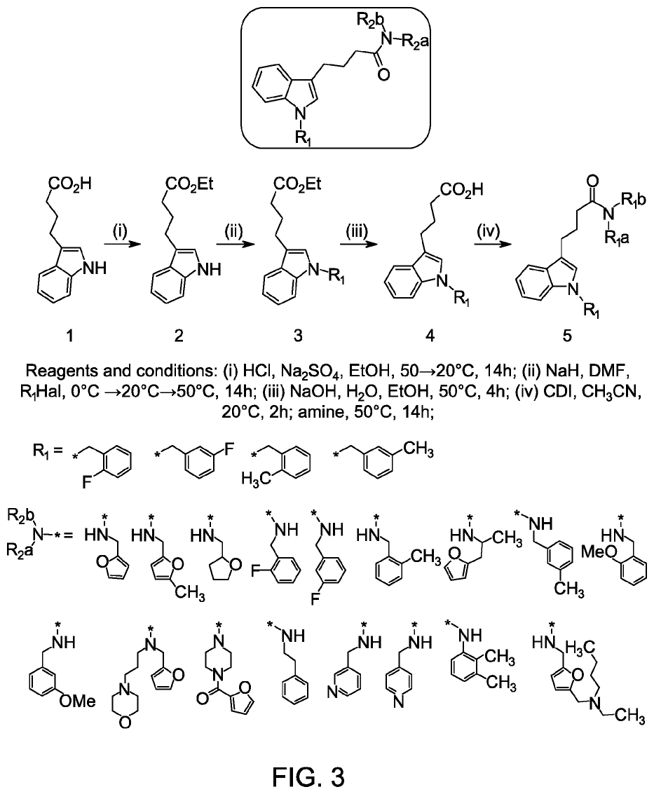

Synthetic scheme for the preparation of indole-3-butyric acid compounds is shown in FIG. 3. Referring to FIG. 3:

Step 1. Synthesis of Compound 2

To a solution of 3-indolebutyric acid 1 (50.0 g, 246.0 mmol) in 100 mL of EtOH, Na₂SO₄ (14 g, 98.0 mmol) and 12M HCl (2.5 mL, 24.6 mmol) were added at 50° C. The resulting solution was stirred for 16 h at ambient temperature. The reaction mixture was evaporated under reduced pressure to dryness. The residue was diluted with saturated solution NaHCO₃ and extracted with Et₂O (4×100 mL). Combined organic layers were dried with Na₂SO₄ and evaporated under reduced pressure. The residue was purified on silica gel with CH₂Cl₂ as an eluent to give 52 g (90%) of the compound 2.

Step 2. Synthesis of Compounds 3 (General Procedure)

A solution of ethyl 4-(1H-indol-3-yl)butanoate 2 (3.0 g, 13.0 mmol) in DMF (20 mL) was cooled to 5-10° C. on an ice bath. Sodium hydride (of 60% suspension, 622 mg, 15.6 mmol) was added portion wise and the reaction mixture was stirred for 1 h. Corresponding benzyl halide (19.5 mmol) was added, the reaction mixture was allowed to warm to ambient temperature and them stirred for 14 h at 50° C. The resulted mixture was poured into cold water (200 mL) and the precipitate was filtered of to afford crude compounds 3 with 80-90% yields that was used for the next step two thought further purification.

Step 3. Synthesis of Compound 4 (General Procedure)

To a solution of compound 3 (13.0 mmol) in 75% aqueous EtOH (80 mL), NaOH (19.5 mmol) was added. The resulting solution was stirred for 4 h at 50° C. The reaction mixture was evaporated under reduced pressure to dryness. The residue was diluted with water, acidified with 10% aqueous H₂SO₄. The formed precipitate was filtered off, washed with water and dried, providing compounds 4 with 85-90% yields.

Step 4. Combinatorial Synthesis of Final Compounds 5 (General Procedure for Amide Coupling).

To a solution of compound 5 (0.3 mmol) in CH₃CN (2 mL), CDI (0.35 mmol) was added. The mixture was stirred at 20° C. for 2 h. Corresponding amine (0.45 mmol) was added and reaction mixture was stirred at 50° C. for 16 h. After cooling, the reaction mixture was poured into water (5 mL) and extracted with CH₂Cl₂. The combined organic layers were dried over Na₂SO₄, filtered, and concentrated under reduced pressure. The residue was purified by HPLC. Yield 50-70%.

### Example 8—Synthetic Preparation of Exemplified Compounds

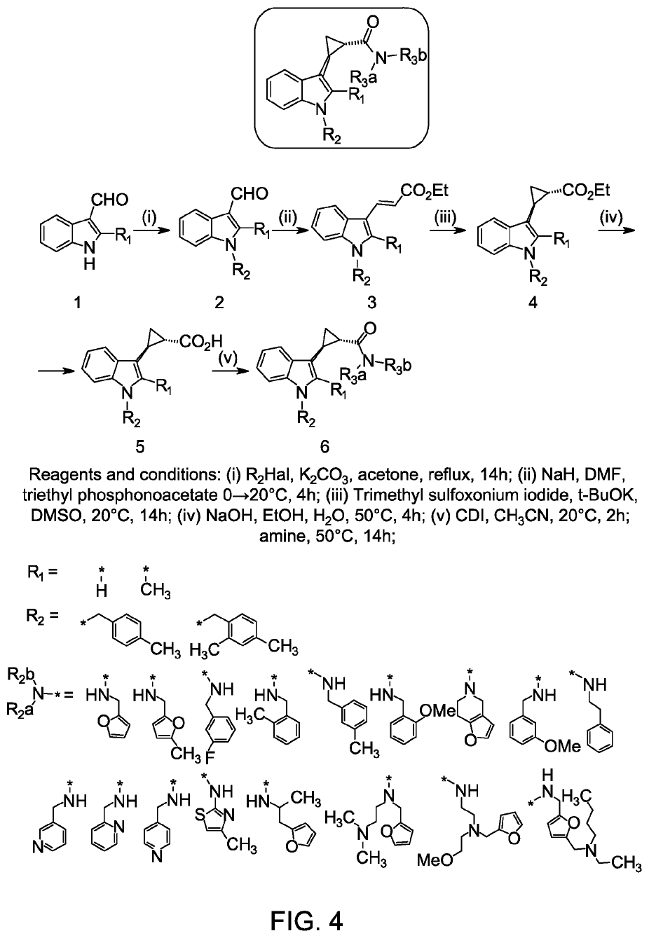

Synthetic scheme for the preparation of indole-3-cyclopropyl compounds is shown in FIG. 4. Referring to FIG. 4:

Step 1. Synthesis of Compounds 2 (General Procedure)

To a solution of compound 1 (103.0 mmol) in 200 mL of acetone, K₂CO₃ (361.0 mmol) and corresponding benzyl halide (124.0 mmol) were added. The resulting mixture was refluxed overnight. The reaction mixture was cooled to ambient temperature and poured into water (600 mL). A formed precipitate was collected by filtration and dried under reduced pressure. Yield 85-95%

Step 2. Synthesis of Compounds 3 (General Procedure)

A suspension of 60% sodium hydride in mineral oil (74.1 mmol) in DMF (500 mL) was cooled on an ice bath to 0-5° C. Triethyl phosphonoacetate (74.1 mmol) was added dropwise and the reaction mixture was stirred for 1 h. Compound 2 (57.0 mmol) was added portionwise to the resulted mixture at 0-5° C. and the reaction mixture was stirred for 1 h. The resulted mixture was poured into cold water, and the formed precipitate was filtered off and dried under reduced pressure providing crude compound 3 that was used for the next step without further purification. Yield 55-65%

Step 3. Synthesis of Compounds 4 (General Procedure)

To a solution of t-BuOK (43 mmol) in DMSO (80 mL), trimethylsulphoxonium iodide (43 mmol) and compound 3 were added. The reaction mixture was stirred at ambient temperature overnight, diluted with water (250 mL), and extracted with Et₂O (5×50 mL). Combined organic layers were washed with water, dried over Na₂SO₄, filtered, and concentrated under reduced pressure. The residue was purified on silica gel with CH₂Cl₂ as an eluent to afford compounds 4 in 20-30% yield.

Step 4. Synthesis of Compounds 5 (General Procedure)

To a solution of compound 4 (8.6 mmol) in 75% aqueous EtOH (40 mL) NaOH (12.9 mmol) was added. The resulting solution was stirred for 4 h at 50° C. The reaction mixture was evaporated under reduced pressure to dryness. The residue was diluted with water, acidified with 10% aqueous H₂SO₄. The formed precipitate was filtered off, washed with water. And dried under reduced pressure to afford compounds 5 in 85-90% yield.

Step 5. Combinatorial Synthesis of Final Compounds 6 (General Procedure for Amide Coupling).

To a solution of compound 5 (0.3 mmol) in CH₃CN (2 mL), CDI (0.35 mmol) was added. The mixture was stirred at 20° C. for 2 h. Corresponding amine (0.45 mmol) was added and the reaction mixture was stirred at 50° C. for 16 h, cooled, poured into water (5 mL), and extracted with CH₂Cl₂. The combined organic layers were dried over Na₂SO₄, filtered, and concentrated under reduced pressure. The residue was purified by HPLC. Yield 50-70%.

### Example 9—Synthetic Preparation of Exemplified Compounds

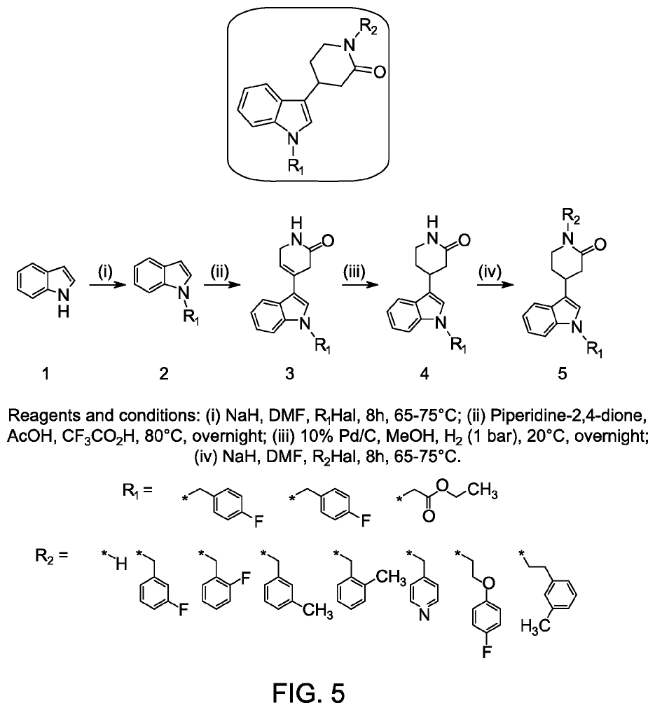

Synthetic scheme for the preparation of indole-5-carboxamide compounds is shown in FIG. 5. Referring to FIG. 5:

Step 1. Synthesis of Compounds 2 (General Procedure)

A suspension of NaH (60% in mineral oil, 2.2 g, 55 mmol) was added to a stirred solution of indole 1 (5.0 g, 42 mmol) in DMF (50 mL), and the reaction mixture was stirred at ambient temperature for 1 h. Corresponding alkyl halide (64 mmol) was added dropwise to the reaction mixture, stirred at 65-70° C. for 10 h, cooled to ambient temperature. Water (300 mL) was added, and the product was extracted with diethyl ether (3×20 mL). Combined organic layers were washed with water, dried with Na₂SO₄, the solvent was concentrated under reduced pressure to dryness. The residue was purified by flash chromatography on silica gel with CHCl₃ as an eluent. The yields of N-substituted indoles 2 were 85-95%.

Step 2. Synthesis of Compounds 3 (General Procedure).

Piperididine-2,4-dione (6.07 g, 53 mmol) was added to a solution of compound 2 (29 mmol) in glacial acetic acid (30 mL) followed by addition dropwise trifluoroacetic acid (3.30 g, 29 mmol). The reaction mixture was heated at 80° C. for 5 h. After cooling to ambient temperature, the volatiles were removed under reduced pressure. The residue was treated with water (30 mL), basified to pH ˜9-10 with 15% aqueous NaOH, and the mixture was extracted with EtOAc (3×50 mL). The combined organic layers were washed with water, brine, dried over MgSO₄, filtered, and concentrated. The resulting residue was purified by silica flash chromatography (CH₂Cl₂/methanol 0→6%). Yield 55-65%.

Step 3. Synthesis of Compounds 4 (General Procedure).

The compound 3 (30 mmol) was dissolved in MeOH (50 mL) and 10% Pd/C was added to the solution. The reaction mixture was stirred at ambient temperature in the hydrogen atmosphere overnight and filtered. The solvent was evaporated under reduced pressure; the residue was purified by flash chromatography on silica gel. Yield 75-85%.

Step 4. Combinatorial Synthesis of Final Compounds 5 (General Procedure for Alkylation).

A suspension of NaH (60% in mineral oil, 15 mg, 0.4 mmol) was added to a solution of compound 4 (0.3 mmol) in DMF (2 mL), and the reaction mixture was stirred at ambient temperature for 1 h. Corresponding alkyl halide (0.42 mmol) was added dropwise to the reaction mixture, and the resulting mixture was stirred at 65-70° C. for 10 h, cooled to ambient temperature, diluted with water, the product was extracted with diethyl ether, the solvent was removed under reduced pressure to dryness. The residue was purified by chromatography on silica gel with EtOAc/hexane as an eluent. Yield 55-75%.

### Example 10—Synthetic Preparation of Exemplified Compounds

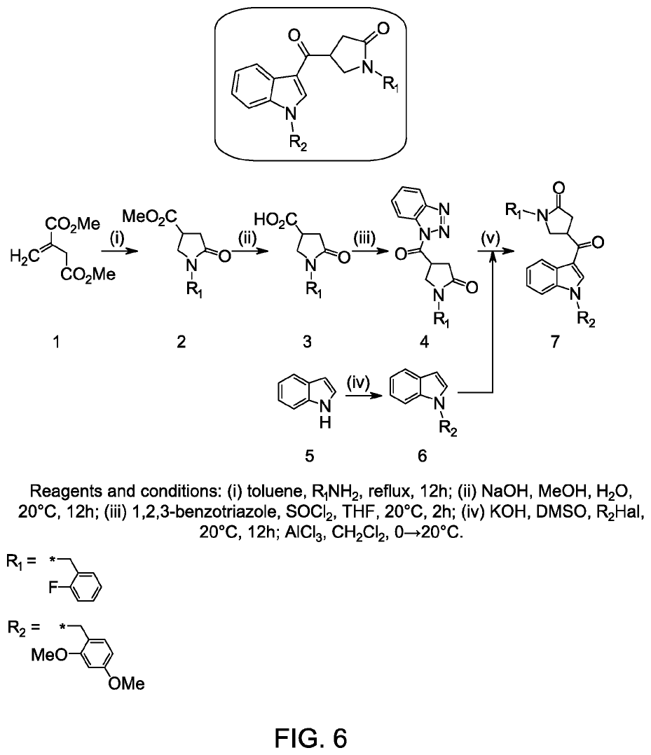

Synthetic scheme for the preparation of indole-3-carboxpyrrolidinone compounds is shown in FIG. 6. Referring to FIG. 6:

Step 1. Synthesis of Compound 2

A solution of dimethyl itaconate 1 (15.8 g, 100 mmol) and 2,4-dimethoxybenzylamine (16.7 g, 100 mmol) in toluene (200 mL) was heated under reflux for 12 h. The mixture was evaporated under reduced pressure, the crude product 2 (28.7 g, 98%) was obtained as a yellow oil and used for the next step without further purification.

Step 2. Synthesis of Compound 3

A solution of NaOH (12.0 g, 300 mmol) in water (70 mL), was added to a solution of compound 2 (28.7 g, 98 mmol) in MeOH (200 mL). the resulted mixture was stirred overnight and concentrated under reduced pressure. The residue was acidified with aq. 1 M HCl to pH 3-4. The formed precipitate was filtered off, washed with water and dried under deduced pressure to afford compound 3 (25 g, 90%) as a white solid that was used for the next step without further purification.

Step 3. Synthesis of Compound 4

Thionyl chloride (730 μL, 10 mmol) was added dropwise to a solution of 1,2,3-benzotriazole (4.76 g, 40 mmol) in THE (20 mL), and the reaction mixture was stirred at 40° C. for 30 min. After cooling to 0° C., the solution of compound 3 (2.75 g, 10 mmol) in THE (10 mL) was added dropwise. Resulted mixture was allowed to warm to ambient temperature and then stirred additionally for 2 h. Formed precipitated solids were filtered off, washed with THF and the filtrate was evaporated under reduced pressure. The residue was purified by column chromatography on silica gel with EtOAc/Hexane (1:2) as an eluent to afford compound 4 (1.75 g, 46%).

Step 4. Synthesis of Compound 6

Indole 5 (5.0 g, 45 mmol) was added to a solution of KOH (4.8 g, 86 mmol) in DMSO (50 mL), and resulting mixture was stirred at ambient temperature for 30 min. Then 2-fluoro-benzylbromide (7.4 g, 51 mmol) was added, and the mixture was stirred at ambient temperature overnight. The mixture was poured into water and extracted with CH₂Cl₂ (3×50 mL). Combined extracts were washed with water, dried with Na₂SO₄, filtered, and evaporated under reduced pressure. The residue was purified by column chromatography on silica gel with EtOAC/Hexane (1:1) as an eluent to afford compound 6 (6.5 g, 62%).

Step 5. Synthesis of Compound 7

AlCl₃ (400 mg, 3.0 mmol) was added portionwise to a stirred solution of compound 4 (860 mg, 2.2 mmol) and compound 6 (0.59 g, 2.6 mmol) in CH₂Cl₂ (20 mL) at 0° C. The resulting mixture was stirred at 20° C. for 3 h, quenched with MeOH (5 mL), stirring for 10 min, and concentrated under reduced pressure. The residue was purified by column chromatography on silica gel using a mixture of EtOAC/hexane (1:1) as an eluent. The product was further purified by HPLC. Yield (36 mg, 2.8%).

### Example 11—Synthetic Preparation of Exemplified Compounds

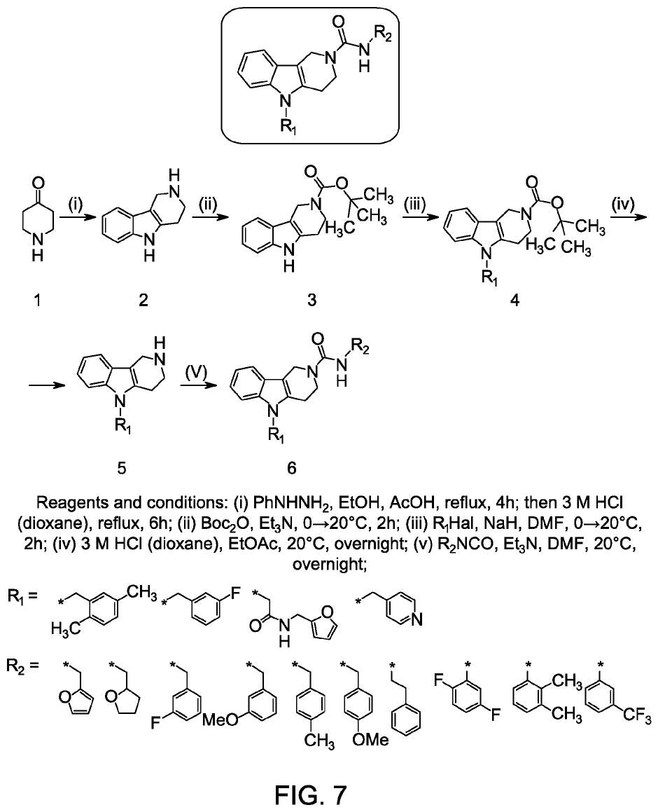

Synthetic scheme for the preparation of 2,3,4,5-tetrahydro-1H-pyrido[4,3-b]indole compounds is shown in FIG. 7. Referring to FIG. 7:

Step 1. Synthesis of Compound 2.

To a stirred mixture of compound 1 hydrochloride (6.0 g, 39 mmol) and phenylhydrazine (4.2 g, 39 mmol) in ethanol (150 mL) acetic acid (5 mL) was added portionwise. The reaction mixture was stirred and heated under reflux for 4 h, cooled to ambient temperature, and 3M HCl solution in dioxane (20 mL) was added. The reaction mixture was heated under reflux for 6 h, cooled to ambient temperature, and concentrated under reduced pressure to ⅓ of initial volume. The resulting solution was cooled to −20° C.; the formed precipitate was filtered off, washed with small portion of cold MeOH and dissolved in water (300 mL). The solution was basified with aq. 6 M NaOH solution to pH 12. The formed precipitate was filtered off, washed with small portion of cold water and dried under reduced pressure to afford the compound 2 (3.3 g, 49%) that was used for the next step without further purification.

Step 2. Synthesis of Compound 3.

A solution of di-tert-butyldicarbonate (7.5 g, 34.4 mmol) in CH₂Cl₂ (50 mL) was added dropwise to a solution of compound 2 (5.4 g, 31.4 mmol) and Et₃N (6.40 g, 63.3 mmol) in CH₂Cl₂ (100 mL) at 0° C. The reaction mixture was allowed to warm up to ambient temperature, stirred for 2 h and quenched with water. The organic layer was separated, the aqueous one was extracted with CH₂Cl₂. The combined organic layers were washed with 2% citric acid, brine, dried over Na₂SO₄, and filtered. The solvent was removed under reduced pressure. The white residue was washed with hexane to provide the compound 3 (7.7 g, 82%) that was used for the next step without further purification.

Step 3. Synthesis of Compounds 4 (General Procedure).

To an ice cold solution of compound 3 (2.1 g, 7.7 mmol) in DMF (40 mL) NaH as 60% suspension in mineral oil (0.40 g, 10 mmol) was added in three portions (double excess of NaH was used in case of 4-chloromethylpyridine hydrochloride), and the reaction mixture was stirred at 0° C. for 30 min. Corresponding alkyl halide (8.0 mmol) was added portionwise, and the resulting mixture was stirred at ambient temperature for 2 h. Then the reaction mixture was poured into cold 2% citric acid solution (150 mL), and the product was extracted with EtOAc (3×75 mL). Combined extracts were washed with NaHCO₃ solution, water, brine, dried over Na₂SO₄, and filtered. The solvent was evaporated under reduced pressure to dryness. The residue was purified by flash chromatography on silica gel (hexane/EtOAc) to afford the compounds 4 in 35-90% yield.

Step 4. Synthesis of Compounds 5 (General Procedure).

To a solution of compound 4 (7.3 mmol) in EtOAc (40 mL) the 3M solution of HCl in dioxane (5 mL) was added, and the reaction mixture was stirred at ambient temperature overnight. The formed solid was filtered off, washed with ether, hexane and dried under reduced pressure to afford the of compound 5, hydrochloride in 75-92% yield.

Step 4. Combinatorial Synthesis of Final Compounds 6 (General Procedure for Carbamoylation).

To a solution of compound 5 hydrochloride (1.0 eq) and Et₃N (1.0 eq) in DMF (1 mL) appropriate isocyanate (1.1 eq) was added. The mixture was stirred at ambient temperature overnight. Water and CH₂Cl₂ were added, the layers were separated, the organic one dried over Na₂SO₄, filtered, and concentrated under reduced pressure. The obtained residue was purified by column chromatography on silica gel to afford target compounds 6 (yield 9-64%)

### Example 12—Synthetic Preparation of Exemplified Compounds

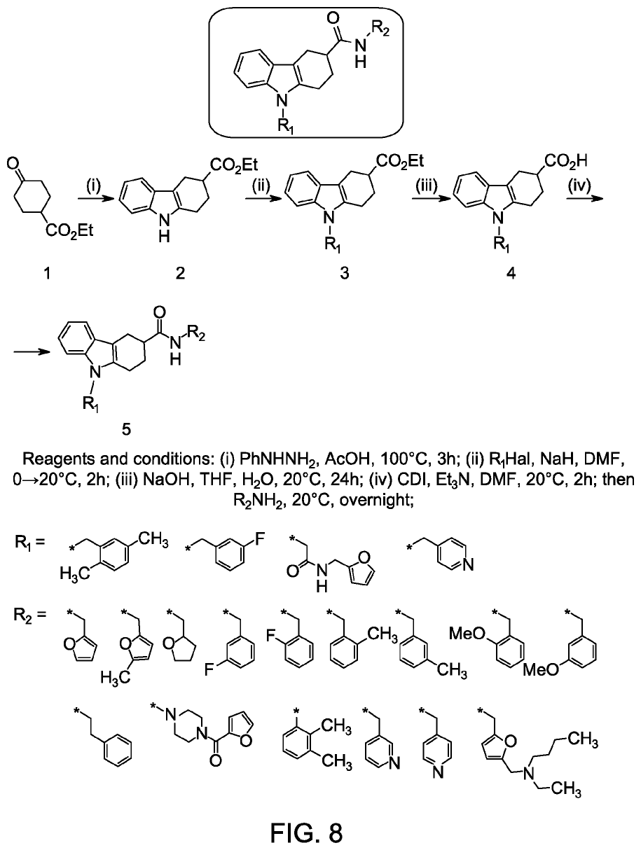

Synthetic scheme for the preparation of 2,3,4,9-tetrahydro-1H-carbazole compounds is shown in FIG. 8. Referring to FIG. 8:

Step 1. Synthesis of Compound 2.

Phenylhydrazine (1.4 g, 13.0 mmol) was added portionwise to a solution of compound 1 (2.2 g, 12.9 mmol) in glacial acetic acid (20 mL), and the resulted mixture was stirred at 100° C. for 3 h. Water (100 mL) was added, and the product was extracted with CH₂Cl₂ (2×100 mL). Combined extracts were washed with aq. NaHCO₃ solution, water, brine, dried over Na₂SO₄, filtered, and concentrated under reduced pressure. The residue was washed with small portion of hexane and dried at atmosphere to afford the compound 2 (2.5 g, 80%) that was used for the next step without further purification.

Step 2. Synthesis of Compounds 3 (General Procedure).

To an ice cooled solution of compound 3 (2.5 g, 10.3 mmol) in DMF (40 mL) 60% NaH suspension in mineral oil (0.52 g, 13.0 mmol) was added in three portions (double excess of NaH was used with of 4-chloromethylpyridine hydrochloride), and the reaction mixture was stirred at 0° C. for 30 min. Corresponding alkyl halide (11.0 mmol) was added portionwise, and the resulting solution was stirred at ambient temperature for 2 h. Then the reaction mixture was poured into ice cooled 2% citric acid aqueous solution (150 mL) and the product was extracted with EtOAc (3×75 mL). Combined extracts were washed with NaHCO₃ solution, water, brine, dried over Na₂SO₄ and filtered. The solvent was evaporated under reduced pressure to dryness. The residue was purified by flash chromatography on silica gel (Hex/EtOAc) to afford the compounds 3 (55-81%) that were used for the next step without further purification.

Step 3. Synthesis of Compounds 4 (General Procedure).

To a solution of compound 3 (4.0 mmol) in THE (35 mL) and H₂O (5 mL) NaOH (0.32 g, 8.0 mmol) was added. The resulted mixture was stirred at ambient temperature for 24 h and then diluted with water (100 mL). The mixture was acidified with 6 M HCl to pH 1, the product was extracted with EtOAc (2×100 mL). The combined organic layers were dried over Na₂SO₄, filtered, and evaporated under reduced pressure to afford the compounds 4, (49-77%).

Step 4. Synthesis of Compounds 5 (General Procedure).

Carbonydiimidazole (1.3 eq) was added to a solution of compound 4 (1.0 eq) in DMF (3 mL), the mixture was stirred for 2 h followed by addition of corresponding amine (1.4 eq). The reaction mixture was stirred at ambient temperature overnight, diluted with water and extracted with EtOAc. Organic layer was washed with water, concentrated under reduced pressure, and the residue was purified by flash-chromatography on silica gel with EtOAc/hexane as an eluent to afford the compounds 5 in 15-99% yields.

### Example 13—Synthetic Preparation of Exemplified Compounds

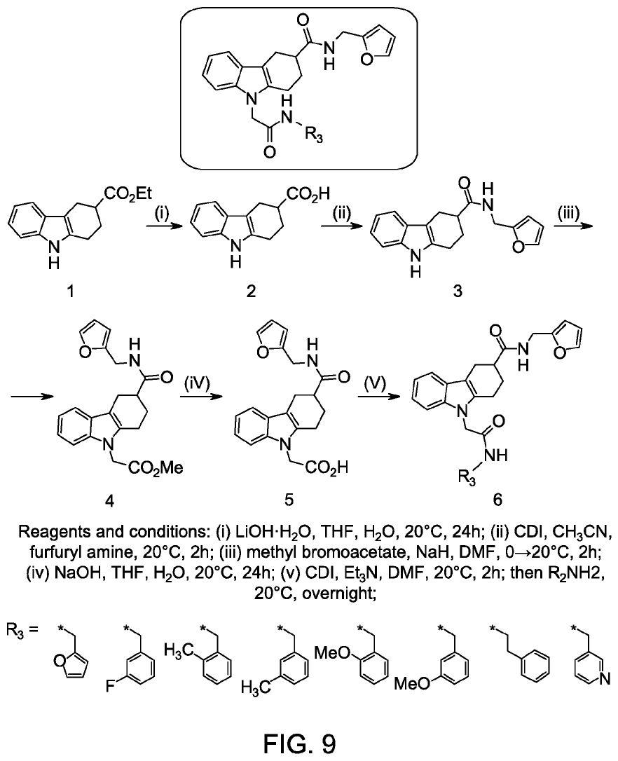

Synthetic scheme for the preparation of 2,3,4,9-tetrahydro-1H-carbazole compounds is shown in FIG. 9. Referring to FIG. 9:

Step 1. Synthesis of Compound 2.

A mixture of compound 2 (2.0 g, 8.22 mmol) and LiOH.H₂O (1.1 g, 24.66 mmol) in THF (20 mL) and water (5 mL) was stirred at ambient temperature for 24 h, then concentrated under reduced pressure to half-a-volume. Water (30 mL) was added and pH of the solution was adjusted to 1 by addition of 6M HCl. The precipitated solid was filtered, washed thoroughly with water and dried under reduced pressure to give the compound 3 (1.4 g, 80%).

Step 2. Synthesis of Compound 3.

Carbonyldiimidazole (3.2 g, 19.8 mmol) was added to a solution of compound 2 (3.10 g, 14.4 mmol) in CH₃CN (50 mL), the mixture was stirred for 2 h followed by addition of furfuryl amine (2.0 g, 20.6 mmol). The reaction mixture was stirred at ambient temperature overnight, then cooled to −20° C. The precipitated solid was filtered, washed with small portion of cold CH₃CN, water and dried under reduced pressure to give the compound 3 (3.6 g, 85%).

Step 3. Synthesis of Compound 4.

To an ice cold solution of compound 3 (2.0 g, 6.79 mmol) in DMF (30 mL) 60% NaH suspension in mineral oil (0.33 g, 8.15 mmol) was added in three portions and the reaction mixture was stirred at 0° C. for 30 min. Methyl bromoacetate (7.8 mmol) was added dropwise, and the resulting solution was stirred at ambient temperature for 2 h. Then the reaction mixture was poured into cold 2% citric acid aqueous solution (150 mL) and the product was extracted with EtOAc (3×75 mL). Combined extracts were washed with NaHCO₃ solution, water, brine, dried over Na₂SO₄ and filtered. The solvent was evaporated under reduced pressure to dryness. The residue was washed with hexane and dried under reduced pressure to afford the crude compound 4 (2.3 g, 93%).

Step 4. Synthesis of Compound 5.

To a stirred solution of compound 3 (2.30 g, 6.28 mmol) in THE (50 mL) and H₂O (10 mL), NaOH (0.35 g, 8.75 mmol) was added. The resulting mixture was stirred at ambient temperature for 24 h and diluted with water (100 mL). The pH of the mixture was adjusted to 1 by addition of 6 M HCl, the product was extracted with EtOAc (2×100 mL). The combined organic layers were dried over Na₂SO₄, filtered and evaporated to dryness under reduced pressure. The residue was washed with hexane and dried to give the compound 5 (1.70 g, 77%).

Step 5. Synthesis of Compounds 6 (General Procedure for Amide Coupling).

Carbonyldiimidazole (55 mg, 0.340 mmol) was added to a solution of compound 4 (90 mg, 0.255 mmol) in DMF (3 mL), the mixture was stirred for 2 h followed by addition of corresponding amine (0.357 mmol). The reaction mixture was stirred at ambient temperature overnight, diluted with water and extracted with EtOAc. Organic layer was washed with water, concentrated under reduced pressure, and the residue was purified by flash chromatography on silica gel (EtOAc/hexane) to afford compounds 6 in 45-64% yields.

### Example 14—Synthetic Preparation of Exemplified Compounds

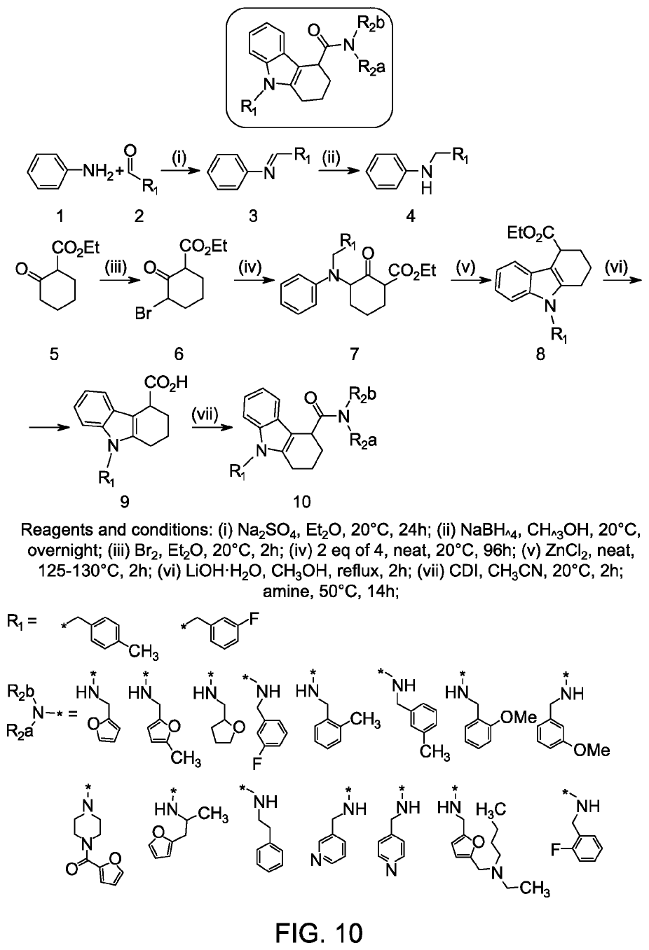

Synthetic scheme for the preparation of 2,3,4,9-tetrahydro-1H-carbazole compounds is shown in FIG. 10. Referring to FIG. 10:

Step 1. Synthesis of Compound 3.

To an equimolar solution of aniline 1 (5 mL, 55 mmol) and aldehyde 2 (55 mmol) in diethyl ether (200 mL), Na₂SO₄ (15 g) was added at ambient temperature and the mixture was stirred at ambient temperature for 24 h. Then the solid was filtered off, washed with ether and discarded. Combined filtrates were concentrated, and the product 3 (quantitative yield) was used without further purification.

Step 2. Synthesis of Compounds 4 (General Procedure).

To an ice cooled solution of compound 3 (55 mmol) in MeOH (200 mL) NaBH₄ (2.27 g, 60 mmol) was added portionwise, and the resulted mixture was stirred at ambient temperature overnight. The mixture was acidified with 1 M HCl, and the volatiles were removed under reduced pressure. The residue was basified with Na₂CO₃ aq. solution to pH 8-9. The product was extracted with diethyl ether (3×50 mL). The combined organic layers were dried over MgSO₄, filtered, and concentrated under reduced pressure to afford compound 4 in 98% yield.

Step 3. Synthesis of Compounds 6 (General Procedure).

To the solution of ketone 5 (12.4 mL, 77 mmol) in ether (100 mL), Br₂ (4.0 mL, 77 mmol) was added dropwise at ambient temperature. The mixture was stirred for 2 h and quenched with an aqueous solution of sodium sulfite. The organic layer was separated, dried over MgSO₄, concentrated under reduced pressure to afford compound 6 in quantitative yield (20 g) that was used further without additional purification.

Step 4. Synthesis of Compounds 7 (General Procedure).

The compound 6 (1 eq) was mixed with amine 4 (2 eq) and the resulted mixture was stirred at ambient temperature for 96 h. Then the reaction mixture was diluted with diethyl ether and the hydrobromide of starting amine 4 was filtered off. The filtrate was concentrated under reduced pressure and crude product 7 (quantitative yield) was used further without additional purification.

Step 5. Synthesis of Compounds 8 (General Procedure).

The crude compound 7 (1 eq) was mixed with anhydrous ZnCl₂ (1 eq), and the resulted mixture was stirred at 125-130° C. for 2 h, then partitioned between water and ethyl acetate. Organic layer was separated, aqueous layer was extracted with EtOAc (3×50 mL). Combined organic layers were dried over MgSO₄, filtered, and concentrated under reduced pressure. The crude product 8 was purified by chromatography on silica gel to afford compound 8 in 58-65% yield.

Step 6. Synthesis of Compounds 9 (General Procedure).

The product 8 (1 eq) was dissolved in methanol (50 mL) and LiOH H₂O (1.5 eq) was added. The mixture was refluxed for 2 h and concentrated under reduced pressure. The residue was dissolved in water and acidified with 1M HCl. The formed acid 9 was filtered and dried on air. Yield 28-66%.

Step 5. Combinatorial Synthesis of Final Compounds 10 (General Procedure for Amide Coupling).

To a solution of compound 9 (0.3 mmol) in CH₃CN (2 mL), CDI (0.35 mmol) was added. The mixture was stirred at 20° C. for 2 h. Corresponding amine (0.45 mmol) was added and reaction mixture was stirred at 50° C. for 16 h. After cooling, the reaction mixture was poured into water (5 mL) and extracted with CH₂Cl₂. The combined organic extracts were dried with Na₂SO₄ filtered and concentrated under reduced pressure. The residue was purified by HPLC. Yield 50-70%.

### Example 15—Synthetic Preparation of Exemplified Compounds

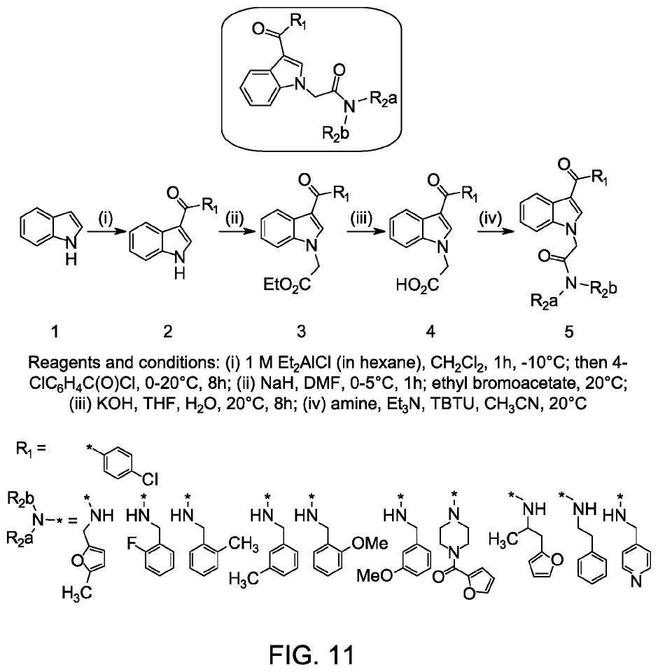

Synthetic scheme for the preparation of indole-3-carboxamide compounds is shown in FIG. 11. Referring to FIG. 11:

Step 1. Synthesis of Compound 2.

To a solution of indole 1 (5.85 g, 50 mmol) in CH₂Cl₂ (75 mL) a 1M hexane solution of Et₂AlCl (55 mL, 55 mmol) was added dropwise at −10° C. within 30 min, and resulted mixture was stirred at −10° C. under argon atmosphere for 1 h. Then a solution of p-chlorobenzoyl chloride (19.63 g, 55 mmol) in 25 mL of CH₂Cl₂ was added at −10° C., and resulted mixture was stirred at ambient temperature for 2 h, poured into cold saturated solution of NH₄Cl. Organic layer was separated, water one was extracted with CH₂Cl₂ (2×50 mL). Combined organic layers was washed with water, dried over Na₂SO₄, filtered, and evaporated under reduced pressure. The residue was purified by column chromatography on silica gel (CCl₄/EtOAc 1:1) to provide compound 2 (6.21 g, 48%).

Step 2. Synthesis of Compound 3.

To a solution of 3-aroyl-indole 2 (5.00 g, 23.65 mmol) in DMF (50 mL) under argon atmosphere at 0° C. NaH (60%, 1.51 g, 37.77 mmol) was added portionwise, and resulted mixture was stirred at ambient temperature for a 30 min, then a solution of ethyl bromoacetate (4.74 g, 28.38 mmol) in THE (10 mL) was added dropwise, and the mixture was stirred at ambient temperature overnight. The reaction mixture was poured into cold water (150 mL) and extracted with EtOAc (3×100 mL). Combined organic extracts were washed with water, dried with Na₂SO₄, filtered, and concentrated under reduced pressure. A residue was purified by column chromatography (EtOAc/hexane 1:4) to provide compound 3 (4.30 g, 82%).

Step 3. Synthesis of Compound 4.

To a mixture of compound 3 (4.30 g, 16.0 mmol), THE (50 mL) and water (10 mL) NaOH (1.28 g, 32.0 mmol) was added, and the resulted mixture was stirred at ambient temperature for a 12 h. The volatiles were removed under reduced pressure, the residue was suspended in 50 mL of water and extracted with CH₂Cl₂ (2×50 mL). Organic layer was discarded, and the water layer was acidified to pH-3 with 1M HCl. The formed precipitate was filtered off, dried under reduced pressure to afford compound 4 (1.96 g, 39%).

Step 4. Combinatorial Synthesis of Final Compounds 5 (General Procedure for Amide Coupling).

A mixture of acid 6 (100 mg, 0.28 mmol), corresponding amine (0.31 mmol), triethylamine (58 μl, 0.42 mmol), and TBTU (134 mg, 0.42 mmol) in dry acetonitrile (2 mL) was stirred at ambient temperature. After reaction completion (monitoring by LC/MS), the mixture was evaporated to dryness, taken up in water and extracted with CH₂Cl₂. Organic layer was dried and filtered, and the filtrate was concentrated under reduced pressure. The residue was subjected to silica flash chromatography with CH₂Cl₂/MeOH as an eluent to afford the compound 7 in moderate to good yields.

### Example 16—Synthetic Preparation of Exemplified Compounds

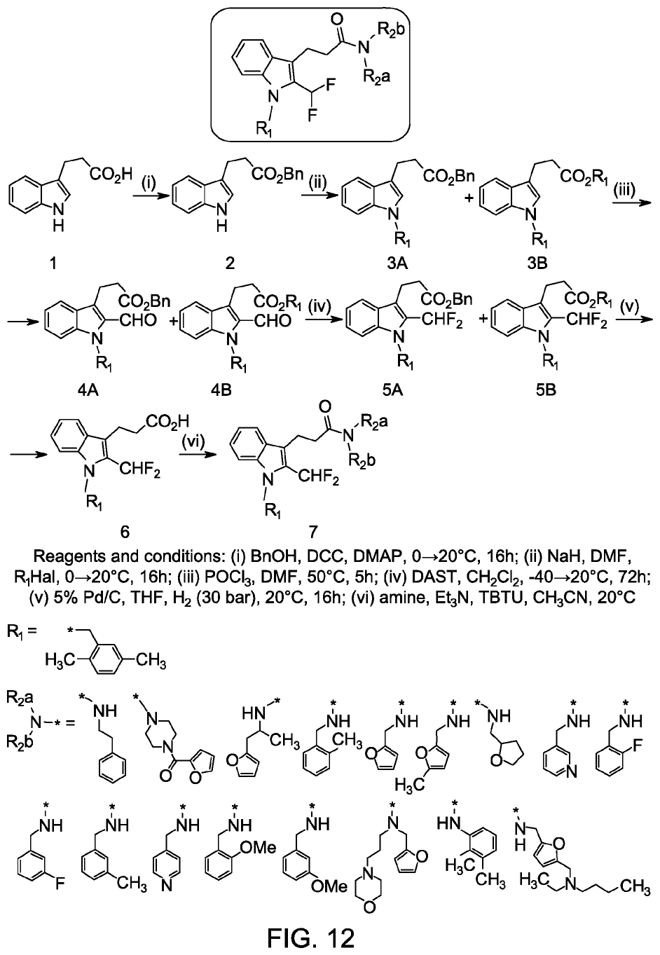

Synthetic scheme for the preparation of indole-2-propanoic acid compounds is shown in FIG. 12. Referring to FIG. 12:

Step 1. Synthesis of Compound 2.

DMAP (388 mg, 3.18 mmol), benzylic alcohol (1.64 mL, 15.9 mmol) in CH₂Cl₂ (30 mL), and DCC (3.43 g, 16.7 mmol) were added to a solution of acid 1 (3.00 g, 5.9 mmol) at 0° C. The mixture was allowed to warm to ambient temperature and stirred for 16 h. Formed precipitate was filtered off and washed with CH₂Cl₂. The combined filtrate was evaporated to dryness and subjected to flash chromatography on silica gel with CH₂Cl₂ as an eluent to afford 3.9 g (88%) of compound 2.

Step 2. Synthesis of Compounds 3A and 3B.

Compound 2 (3.9 g, 14.0 mmol) was dissolved in 20 mL of DMF and cooled on ice bath. Sodium hydride (60% suspension in mineral oil, 643 mg, 16.1 mmol) was added portionwise, and the reaction mixture was allowed to warm to ambient temperature and stirred for 1 h. 2,5-Dimethylbenzyl chloride (2.26 mL, 15.4 mmol) was added, the reaction mixture was stirred for 16 h at ambient temperature. The resulted mixture was poured into 100 mL of ice-cold water and the precipitate was filtered providing crude mixture (5.1 g) of the compounds 3A and 3B.

Step 3. Synthesis of Compound 4A and 4B.

Phosphorus oxychloride (44 mL) was added DMF (67 mL) upon cooling on an ice bath. The mixture was stirred for 1 h at 0-5° C. A solution of compounds 3A and 3B (60.3 g) in DMF (260 mL) was added dropwise and the reaction mixture was further stirred at 50° C. for 5 h. The resulted solution was cooled and poured into 500 mL of water. The mixture was neutralized with solid NaHCO₃ and extracted with EtOAc (3×300 mL). Combined organic layers were washed with water, dried over Na₂SO₄, filtered and concentrated under reduced pressure. The residue was purified on silica gel with hexane/EtOAc as an eluent to give 18.2 g of the mixture of the compounds 4A and 4B.

Step 4. Synthesis of Compound 5A and 5B.

A solution of compounds 4A and 4B (18.2 g) in of CH₂Cl₂ (200 mL) was cooled to −40° C. DAST (16.0 mL, 0.13 mol) was added dropwise. The mixture was allowed to warm to ambient temperature and stirred for 72 h. The resulted mixture was quenched with saturated NaHCO₃ aq. solution (200 mL). Organic layer was separated, dried over Na₂SO₄, filtered, and concentrated under reduced pressure. The residue was purified on silica gel with hexane/EtOAc as an eluent to give 3.65 g of the mixture of the compounds 5A and 5B.

Step 5. Synthesis of Compound 6.

A solution of compounds 5A and 5B (3.65 g) in 50 mL of THF was hydrogenated (30 bar) over 5% Pd(OH)₂/C (365 mg) at 20° C. for 16 h. All insoluble materials were filtered off, and the filtrate was concentrated under reduced pressure providing 2.74 g (94%) of compound 6.

Step 6. Synthesis of Compounds 7 (General Procedure for Amide Coupling).

A mixture of acid 6 (100 mg, 0.28 mmol), corresponding amine (0.31 mmol), triethylamine (58 μl, 0.42 mmol), and TBTU (134 mg, 0.42 mmol) in dry acetonitrile (2 mL) was stirred at ambient temperature. After reaction completion (monitoring by LC/MS), the mixture was evaporated to dryness, poured into water and extracted with CH₂Cl₂. Organic layer was dried and filtered over Na₂SO₄, and the filtrate was concentrated under reduced pressure. The residue was subjected to silica flash chromatography with CH₂Cl₂/MeOH as an eluent to afford the compound 7 in moderate to good yields.

### Example 17—Synthetic Preparation of Exemplified Compounds

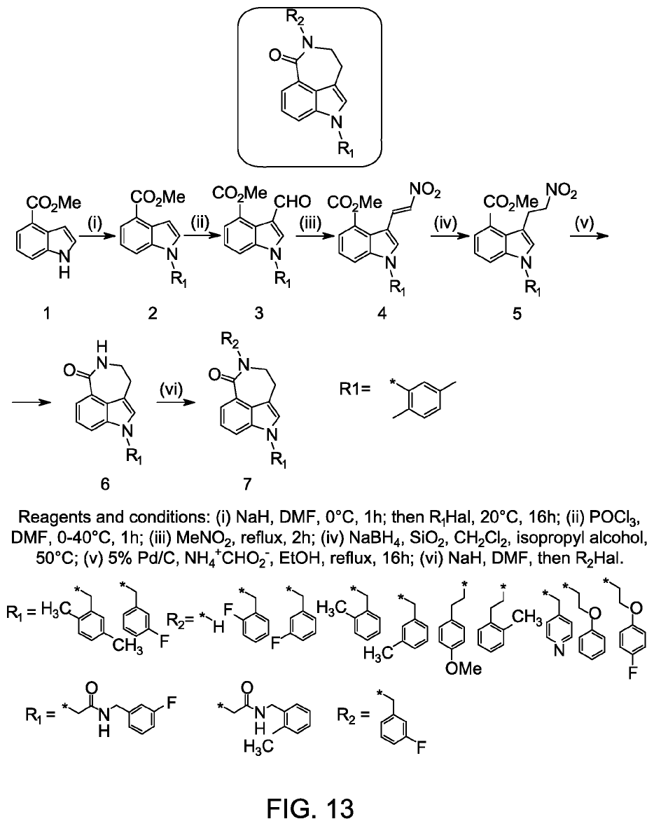

Synthetic scheme for the preparation of 2,3,4,6-tetrahydro-1H-azepino[5,4,3-cd]indol-1-one compounds is shown in FIG. 13. Referring to FIG. 13:

Step 1. Synthesis of Compound 2.

Compound 1 (3.27 g, 18.7 mmol) was dissolved in DMF (20 mL) and cooled on an ice bath. Sodium hydride (60% suspension in mineral oil, 897 mg, 22.4 mmol) was added portionwise, the reaction mixture was stirred for 1 h. Corresponding ylbenzyl chloride (3.18 g, 20.6 mmol) was added, The mixture was allowed to warm to ambient temperature, stirred for 16 h, and into ice-cooled water. The precipitate formed was filtered off providing crude compound 2 (4.74 g, 86%).

Step 2. Synthesis of Compound 3

Phosphorus oxychloride (2.24 mL) was added to cooled at an ice bath DMF (6.73 mL). The mixture was stirred for 1 h. A solution of compound 2 (3.74 g, 12.8 mmol) in DMF (3.74 mL) was added dropwise, and the reaction mixture was further stirred at 40° C. for 1 h. The resulted solution was cooled and poured into water. The mixture was neutralized with solid sodium bicarbonate and the precipitate was filtered off to provide crude compound 3 (2.6 g, 64%).

Step 3. Synthesis of Compound 4

A solution of compound 3 (2.6 g, 8.1 mmol) in nitromethane (46 mL) was refluxed for 2 h. The resulted solution was diluted with CH₂Cl₂ and washed with water. Organic layer was dried and filtered, and the filtrate was concentrated under reduced pressure to afford providing crude compound 4 (2.76 g, 98%).

Step 4. Synthesis of Compound 5

Sodium borohydride (559 mg, 14.7 mmol) was added to a mixture of compound 4 (2.66 g, 7.31 mmol) and silica gel (7.98 g) in a mixture of CH₂Cl₂ (40 mL) and isopropyl alcohol (20 mL). The reaction mixture was stirred for 16 h at 50° C., filtered, and washed with water. Organic layer was separated, dried over Na₂SO₄, filtered and concentrated under reduced pressure to afford crude compound 5 (2 g, 75%).

Step 5. Synthesis of Compound 6

A mixture of compound 5 (2 g, 5.46 mmol), ammonium formate (1.39 g, 22.1 mmol) and 5% Pd/C (200 mg) in EtOH (30 mL) was refluxed for 16 h. The resulted mixture was filtered through a celite plug, and the filtrate was concentrated under reduced pressure. The residue was purified on silica gel with CH₂Cl₂/EtOAc as an eluent to afford 850 mg (45%) of the compound 6.

Step 6. Combinatorial Synthesis of Final Compounds 6 (General Procedure for Alkylation)

Compound 6 (100 mg, 0.33 mmol) was dissolved in DMF (2 mL). Sodium hydride (60% suspension in mineral oil, 16 mg, 0.4 mmol) was added and the reaction mixture was stirred for 1 h. Corresponding alkyl halide (0.4 mmol) was added and the mixture was stirred for 16 h at 20° C. The resulted mixture was concentrated under reduced pressure, poured into water and extracted with CH₂Cl₂. Organic layer was dried and filtered, and the filtrate was concentrated under reduced pressure. The residue was subjected to silica flash chromatography with CH₂Cl₂/MeOH as an eluent to afford compound 7.

These procedures were used for the synthesis of N-(m-fluorobenzyl) substituted indoles (See Example 18).

### Example 18—Synthetic Preparation of Exemplified Compounds

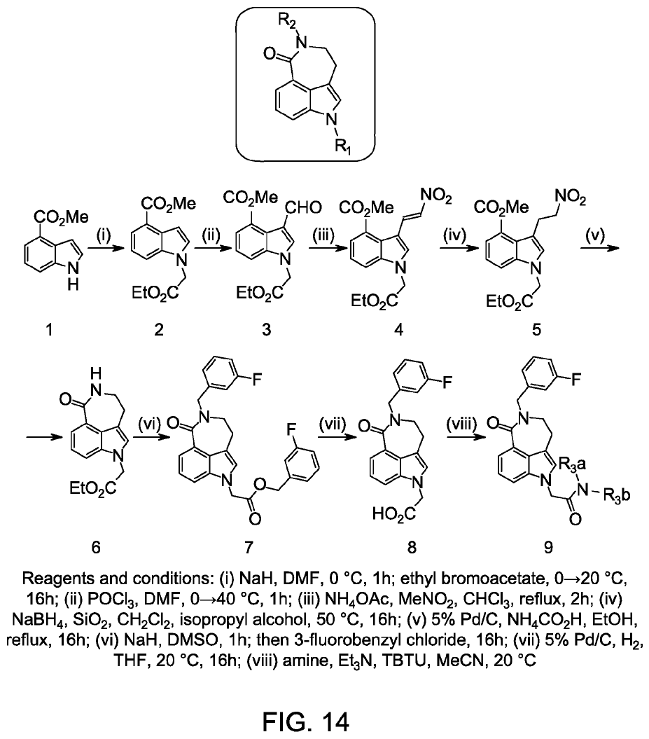

Synthetic scheme for the preparation of 3-fluorobenzyl 2,3,4,6-tetrahydro-1H-azepino[5,4,3-cd]indol-1-one compounds is shown in FIG. 14. Referring to FIG. 14:

Step 1. Synthesis of Compound 2

Compound 1 (15.00 g, 85.8 mmol) was dissolved in DMF (120 mL) and cooled on an ice bath. Sodium hydride (60% suspension in mineral oil, 3.93 g, 98.2 mmol) was added portionwise, and the reaction mixture was stirred for 1 h at 0° C. Ethyl bromoacetate (9.51 g, 85.8 mmol) was added, allowed to worm to ambient temperature, and stirred for 16 h. The resulting mixture was poured into 500 mL of cold water, the precipitate was filtered off and dried to provide crude compound 2 (16.3 g, 73%).

Step 2. Synthesis of Compound 3

Phosphorus oxychloride (10.95 mL) was added to cooled at an ice bath DMF (33 mL). The mixture was stirred for 1 h. A solution of compound 2 (16.3 g, 62.5 mmol) in 33 mL of DMF was added dropwise, and the reaction mixture was further stirred at 40° C. for 1 h, cooled, and poured into 400 mL of water. The mixture was neutralized with solid NaHCO₃ and extracted with EtOAc. Combined organic layers were washed twice with water, dried over Na₂SO₄ and concentrated under reduced pressure to provide crude compound 3 (11.85 g, 66%).

Step 3. Synthesis of Compound 4

To a solution of compound 3 (11.80 g, 40.8 mmol) in chloroform (123 mL) nitromethane (8.72 mL, 163.0 mmol) and ammonium acetate (3.14 g, 40.8 mmol) were added, the reaction mixture was heated under reflux for 16 h, cooled, and washed with water (2×100 mL). Combined organic layers were dried over Na₂SO₄ and filtered, the filtrate was concentrated under reduced pressure and purified on silica gel with CH₂Cl₂ as an eluent to afford 9.10 g (67%) of the compound 4.

Step 4. Synthesis of Compound 5

Sodium borohydride (2.08 g, 54.8 mmol) was added to a mixture of compound 4 (9.10 g, 27.4 mmol) and 27.3 g of silica gel in the mixture of CH₂Cl₂ (100 mL) and isopropyl alcohol (50 mL). The reaction mixture was stirred for 16 h at 50° C., filtered, and washed with water. Organic layer was separated, dried over Na₂SO₄, filtered, and concentrated under reduced pressure to afford crude compound 5 (7.00 g, 76%).

Step 5. Synthesis of Compound 6

A mixture of compound 5 (7.00 g, 21 mmol), ammonium formate (5.28 g, 83.8 mmol) and 5% Pd/C (700 mg) in 100 mL of EtOH was refluxed for 16 h. The resulted mixture was filtered through a celite plug and the filtrate was concentrated in vacuum. The residue was purified on silica gel with CH₂Cl₂/EtOAc as an eluent to give 3.2 g (56%) of the compound 6.

Step 6. Synthesis of Compound 7

Sodium hydride (60% suspension in mineral oil, 403 mg, 10.10 mmol) was added portionwise to a solution of compound 6 (2.37 g, 8.71 mmol) in DMSO (20 mL). The resulted mixture was stirred for 1 h. 3-Fluorobenzyl chloride (1.15 mL, 9.65 mmol) was added, and the reaction mixture was stirred for 16 h at ambient temperature, poured into cold water, and extracted with EtOAc. Combined organic layers were washed twice with water, dried over Na₂SO₄, filtered, and concentrated under reduced pressure. The residue was purified on silica gel with CH₂Cl₂/EtOAc as an eluent to afford 250 mg (8%) of the compound 7.

Step 7. Synthesis of Compound 8

A solution of compound 7 (250 mg, 0.54 mmol) in 10 mL of THF was hydrogenated (1 bar) over 5% Pd/C (25 mg) at ambient temperature for 16 h. All insoluble materials were filtered off, and the filtrate was concentrated under reduced pressure providing 190 mg (quantitative yield) of compound 8.

Step 8. Combinatorial Synthesis of Final Compounds 9 (General Procedure for Amide Coupling).

A mixture of acid 8 (95 mg, 0.21 mmol), corresponding amine (0.23 mmol), Et₃N (43 μl, 0.31 mmol), and TBTU (99 mg, 0.31 mmol) in dry CH₃CN (2 mL) was stirred at ambient temperature. After reaction completion (monitoring by LC/MS), the mixture was evaporated to dryness, poured into water, and extracted with CH₂Cl₂. Organic layer was dried Na₂SO₄ and filtered, the filtrate was concentrated under reduced pressure. The residue was subjected to silica flash chromatography with CH₂Cl₂/MeOH as an eluent to afford compound 9.

### Numbered Paragraphs

Paragraph 1. A method for increasing or maintaining levels of NMNAT2 polypeptide within a cell within a mammal, wherein said method comprises administering, to said mammal, an effective amount of a compound of Formula (I):

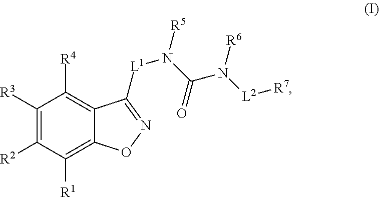

or a pharmaceutically acceptable salt thereof, wherein:

R¹, R², R³, and R⁴ are each independently selected from H, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1} NR^{c1}C(O)OR^{a1}, NR^{c1}S(O)₂R^{b1}, S(O)₂R^{b1}, and S(O)₂NR^{c1}R^{d1};

L¹ is selected from C₁₋₆ alkylene, C₂₋₆ alkenylene, and C₂₋₆ alkynylene, each of which is optionally substituted with 1, 2, or 3 substituents selected from R^{g};

R⁵ and R⁶ are each independently selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents selected from R^{g};

or L² is absent;

R⁷ is selected from H, C₁₋₆ alkyl, C₁₋₆ haloalkyl, C₂₋₆ alkenyl, C₂₋₆ alkynyl, Cy¹, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1} NR^{c1}S(O)₂R^{b1}, S(O)₂R^{b1}, and S(O)₂NR^{c1}R^{d1};

or R⁷, L², and R⁶, together with the N atom to which R⁶ and L² are attached, form a 4-10 membered heterocycloalkyl ring, which is optionally substituted with 1, 2, or 3 substituents independently selected from R^{c1};

Cy¹ is selected from C₆₋₁₀ aryl, C₃₋₁₀ cycloalkyl, 5-14 membered heteroaryl, and 4-10 membered heterocycloalkyl, each of which is optionally substituted with 1, 2, 3, 4, or 5 substituents independently selected from R^{Cy1};

each R^{Cy1} is independently selected from halo, CN, NO₂, Cy², C₁₋₆ alkyl, C₁₋₆ haloalkyl, C₂₋₆ alkenyl, C₂₋₆ alkynyl, OR^{a2}, SR², C(O)R^{b2}, C(O)NR^{c2}R^{d2}, C(O)OR^{a2} NR^{c2}R^{d2}, NR^{c2}C(O)R^{b2}, NR^{c2}C(O)OR^{a2}, NR^{c2}S(O)₂R^{b2}, S(O)₂R^{b2}, and S(O)₂NR^{c2}R^{d2}; wherein said C₁₋₆ alkyl, C₂₋₆ alkenyl, and C₂₋₆ alkynyl are each optionally substituted with 1, 2, or 3 substituents independently selected from Cy², halo, CN, NO₂, OR^{a}, C(O)R^{b2}, C(O)NR^{c2}R^{d2}, C(O)OR^{a2}, NR^{c2}R^{d2} NR^{c2}C(O)R^{b2}, NR^{c2}C(O)OR^{a2} NR^{c2}S(O)₂R^{b2}, S(O)₂R^{b2}, and S(O)₂NR^{c2}R^{d2};

each Cy² is independently selected from C₆₋₁₀ aryl, C₃₋₁₀ cycloalkyl, 5-10 membered heteroaryl, and 4-10 membered heterocycloalkyl, each of which is optionally substituted with 1, 2, 3, 4, or 5 substituents independently selected from R^{Cy2};

each R^{Cy2} is independently selected from halo, CN, NO₂, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, NR^{c2}R^{d2} NR^{c2}C(O)R^{b2} NR^{c2}C(O)OR^{a2}, NR^{c2}S(O)₂R^{b2}, S(O)₂R^{b2}, and S(O)₂NR^{c2}R^{d2}; wherein said C₁₋₆ alkyl, C₂₋₆ alkenyl, and C₂₋₆ alkynyl are each optionally substituted with 1, 2, or 3 substituents independently selected from halo, CN, NO₂, OR^{a2}, C(O)R^{b2}, C(O)NR^{c2}R^{d2}, C(O)OR^{a}, NR^{c2}R^{d2}, NR^{c2}C(O)R^{b2}, NR^{c2}C(O)OR^{a2}, NR²S(O)₂R^{b2}, S(O)₂R^{b2}, and S(O)₂NR^{c2}R^{d2};

each R^{a1}, R^{b1}, R^{c1}, R^{d1}, R^{a2}, R^{b2}, R^{c2}, and R^{d2} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};

or any R^{c1} and R^{d1} together with the N atom to which they are attached form a 4-7 membered heterocycloalkyl, which is optionally substituted with 1, 2, or 3 substituents independently selected from R^{g};

or any R^{c2} and R^{d2} together with the N atom to which they are attached form a 4-7 membered heterocycloalkyl, which is optionally substituted with 1, 2, or 3 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

Paragraph 2. The method of paragraph 1, wherein R¹, R², R³, and R⁴ are each independently selected from H, halo, CN, NO₂, C₁₋₆ alkyl, C₁₋₆ haloalkyl, OH, C₁₋₆ alkoxy, C₁₋₆ haloalkoxy, C(O)R^{b1}, C(O)NR^{c1}R^{d1}, C(O)OR^{a1}, NR^{c1}R^{d1}, and NR^{c1}C(O)R^{b1}; wherein said C₁₋₆ alkyl is optionally substituted with 1, 2, or 3 substituents independently selected from CN, NO₂, OH, C₁₋₆ alkoxy, C(O)R^{b1}, C(O)NR^{c1}R^{d1}, C(O)OR^{a1}, NR^{c1}R^{d1}, and NR^{c1}C(O)R^{b1} Paragraph 3. The method of paragraph 1, wherein R¹, R², and R⁴ are each H; and R³ is selected from H, halo, C₁₋₆ alkyl, C₁₋₆ haloalkyl, C₁₋₆ alkoxy, and C₁₋₆ haloalkoxy.
Paragraph 4. The method of paragraph 1, wherein R¹, R², and R⁴ are each H; and R³ is selected from H and C₁₋₆ alkyl.
Paragraph 5. The method of any one of paragraphs 1-4, wherein L¹ is selected from C₁₋₆ alkylene, C₂₋₆ alkenylene, and C₂₋₆ alkynylene, each of which is optionally substituted with 1, 2, or 3 substituents selected from OH, NO₂, CN, halo, C₁₋₆ alkoxy, and C₁₋₆ haloalkoxy.
Paragraph 6. The method of any one of paragraphs 1-4, wherein L¹ is C₁₋₆ alkylene, which is optionally substituted with 1, 2, or 3 substituents selected from OH, NO₂, CN, halo, C₁₋₆ alkoxy, and C₁₋₆ haloalkoxy.
Paragraph 7. The method of any one of paragraphs 1-4, wherein L¹ is C₁₋₆ alkylene.
Paragraph 8. The method of any one of paragraphs 1-7, wherein R⁵ and R⁶ are each H.
Paragraph 9. The method of any one of paragraphs 1-7, wherein R⁵ is H; and R⁶ is C₁₋₆ alkyl.
Paragraph 10. The method of any one of paragraphs 1-9, wherein L² is absent.
Paragraph 11. The method of any one of paragraphs 1-9, wherein L² is selected from C₁₋₆ alkylene, C₂₋₆ alkenylene, and C₂₋₆ alkynylene, each of which is optionally substituted with 1, 2, or 3 substituents selected from OH, NO₂, CN, halo, C₁₋₆ alkoxy, and C₁₋₆ haloalkoxy.
Paragraph 12. The method of any one of paragraphs 1-9, wherein L² is C₁₋₆ alkylene, which is optionally substituted with 1, 2, or 3 substituents selected from OH, NO₂, CN, halo, C₁₋₆ alkoxy, and C₁₋₆ haloalkoxy.
Paragraph 13. The method of any one of paragraphs 1-9, wherein L² is C₁₋₆ alkylene.
Paragraph 14. The method of any one of paragraphs 1-13, wherein R⁷ is Cy¹.
Paragraph 15. The method of any one of paragraphs 1-13, wherein R⁷ is OR^{a1}. Paragraph 16. The method of any one of paragraphs 1-13, wherein R⁷ is NR^{c1}R^{d1}.
Paragraph 17. The method of any one of paragraphs 1-14, wherein Cy¹ is selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from halo, CN, C₆₋₁₀ aryl, C₁₋₆ alkyl, C₁₋₆ haloalkyl, OR^{a2}, SR^{a2}, and C(O)R^{b2}; wherein said C₁₋₆ alkyl is optionally substituted with C₆₋₁₀ aryl.
Paragraph 18. The method of paragraph 17, wherein Cy¹ is C₆₋₁₀ aryl, optionally substituted with 1, 2, or 3 substituents independently selected from halo, CN, C₁₋₆ alkyl, C₁₋₆ haloalkyl, C₁₋₆ alkoxy, C₁₋₆ alkylthio, and C(O)C₁₋₆ alkyl.
Paragraph 19. The method of paragraph 17, wherein Cy¹ is C₃₋₁₀ cycloalkyl, optionally substituted with 1 or 2 C₁₋₆ alkyl.
Paragraph 20. The method of paragraph 17, wherein Cy¹ is 5-14 membered heteroaryl, optionally substituted with C₁₋₆ alkyl.
Paragraph 21. The method of paragraph 17, wherein Cy¹ is 4-10 membered heterocycloalkyl, optionally substituted with C₆₋₁₀ aryl-C₁₋₆ alkylene.
Paragraph 22. The method of any one of paragraphs 1-17, wherein each R^{a1}, R^{b1}, R^{c1}, R^{d1}, R^{a2}, R^{b2}, R^{c2}, and R^{d2} is independently selected from H, C₁₋₆ alkyl, C₁₋₄ haloalkyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, or 3 substituents independently selected from OH, NO₂, CN, halo, C₁₋₆ alkyl, C₁₋₄ haloalkyl, C₁₋₆ alkoxy, and C₁₋₆ haloalkoxy.
Paragraph 23. The method of paragraph 22, wherein each R^{a1}, R^{b1}, R^{c1}, R^{a1}, R^{a2}, R^{b2}, R^{c2}, and R^{d2} is independently selected from H, C₁₋₆ alkyl, and C₆₋₁₀ aryl-C₁₋₄ alkylene, wherein said C₁₋₆ alkyl and C₆₋₁₀ aryl-C₁₋₄ alkylene are optionally substituted with 1, 2, or 3 substituents independently selected from OH, NO₂, CN, halo, C₁₋₆ alkyl, C₁₋₄ haloalkyl, C₁₋₆ alkoxy, and C₁₋₆ haloalkoxy.
Paragraph 24. The method of paragraph 22, wherein each R^{a1}, R^{b1}, R^{c1}, R^{a1}, R^{a2}, R^{b2}, R^{c2}, and R^{d2} is independently selected from H, C₁₋₆ alkyl, and C₆₋₁₀ aryl-C₁₋₄ alkylene.
Paragraph 25. The method of paragraph 1, wherein:

R¹, R², R³, and R⁴ are each independently selected from H, halo, CN, NO₂, C₁₋₆ alkyl, C₁₋₆ haloalkyl, OH, C₁₋₆ alkoxy, C₁₋₆ haloalkoxy, C(O)R^{b1}, C(O)NR^{c1}R^{d1}, C(O)OR^{a1}, NR^{c1}R^{d1}, and NR^{c1}C(O)R^{b1}; wherein said C₁₋₆ alkyl is optionally substituted with 1, 2, or 3 substituents independently selected from CN, NO₂, OH, C₁₋₆ alkoxy, C(O)R^{b1}, C(O)NR^{c1}R^{d1}, C(O)OR^{a1}, NR^{c1}R^{d1}, and NR^{c1}C(O)R^{b1};

L¹ is selected from C₁₋₆ alkylene, C₂₋₆ alkenylene, and C₂₋₆ alkynylene, each of which is optionally substituted with 1, 2, or 3 substituents selected from OH, NO₂, CN, halo, C₁₋₆ alkoxy, and C₁₋₆ haloalkoxy;

R⁵ is H;

R⁶ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents selected from OH, NO₂, CN, halo, C₁₋₆ alkoxy, and C₁₋₆ haloalkoxy;

or L² is absent;

R⁷ is selected from Cy¹, OR^{a1}, and NR^{c1}R^{d1};

Cy¹ is selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from halo, CN, C₆₋₁₀ aryl, C₁₋₆ alkyl, C₁₋₆ haloalkyl, OR^{a2}, SR^{a2}, and C(O)R^{b2}; wherein said C₁₋₆ alkyl is optionally substituted with C₆₋₁₀ aryl; and

each R^{a1}, R^{b1}, R^{c1}, R^{d1}, R^{a2}, and R^{b2} is independently selected from H, C₁₋₆ alkyl, and C₆₋₁₀ aryl-C₁₋₄ alkylene, wherein said C₁₋₆ alkyl and C₆₋₁₀ aryl-C₁₋₄ alkylene are optionally substituted with 1, 2, or 3 substituents independently selected from OH, NO₂, CN, halo, C₁₋₆ alkyl, C₁₋₄ haloalkyl, C₁₋₆ alkoxy, and C₁₋₆ haloalkoxy.

Paragraph 26. The method of paragraph 1, wherein:

R¹, R², and R⁴ are each H; and R³ is selected from H, halo, C₁₋₆ alkyl, C₁₋₆ haloalkyl, C₁₋₆ alkoxy, and C₁₋₆ haloalkoxy;

L¹ is C₁₋₆ alkylene, which is optionally substituted with 1, 2, or 3 substituents selected from OH, NO₂, CN, halo, C₁₋₆ alkoxy, and C₁₋₆ haloalkoxy;

R⁵ is H;

R⁶ is selected from H and C₁₋₆ alkyl;

L² is C₁₋₆ alkylene, which is optionally substituted with 1, 2, or 3 substituents selected from OH, NO₂, CN, halo, C₁₋₆ alkoxy, and C₁₋₆ haloalkoxy; or L² is absent;

R⁷ is selected from Cy¹, OR^{a1}, and NR^{c1}R^{d1};

Cy¹ is selected from: (i) C₆₋₁₀ aryl, optionally substituted with 1, 2, or 3 substituents independently selected from halo, CN, C₁₋₆ alkyl, C₁₋₆ haloalkyl, C₁₋₆ alkoxy, C₁₋₆ alkylthio, and C(O)C₁₋₆ alkyl; (ii) C₃₋₁₀ cycloalkyl, optionally substituted with 1 or 2 C₁₋₆ alkyl; and (iii) 4-10 membered heterocycloalkyl, optionally substituted with C₆₋₁₀ aryl-C₁₋₆ alkylene; and

R^{a1}, R¹, and R^{d1} are each independently selected from H, C₁₋₆ alkyl, and C₆₋₁₀ aryl-C₁₋₄ alkylene.

Paragraph 27. The method of paragraph 25 or 26, wherein:

R¹, R², and R⁴ are each H;

R³ is selected from H and C₁₋₆ alkyl;

L¹ is C₁₋₆ alkylene; and

L² is absent or C₁₋₆ alkylene.

Paragraph 28. The method of paragraph 1, wherein the compound is selected from any one of the compounds listed in Table 1, or a pharmaceutically acceptable salt thereof.
Paragraph 29. A method for increasing or maintaining levels of NMNAT2 polypeptide within a cell within a mammal, wherein said method comprises administering, to said mammal, an effective amount of a compound of Formula (II):

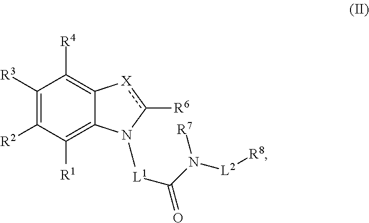

or a pharmaceutically acceptable salt thereof, wherein:

R¹, R², R³, and R⁴ are each independently selected from H, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};

[symbol] is a single bond or a double bond; wherein:

(i) when [symbol] is a double bond, R⁶ is selected from H, C₁₋₆ alkyl, and Cy, and X is selected from N and CR⁵; and

(ii) when [symbol] is a single bond, R⁶ is oxo, X is CR⁵, and R⁵ and R⁴, together with the carbon atoms to which they are attached, form C₆₋₁₀ aryl ring or 5-14 membered heteroaryl ring, each of which is optionally substituted with 1, 2, or 3 substituents independently selected from R^{g};

R⁵ is selected from H, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};

L¹ is selected from C₁₋₆ alkylene, C₂₋₆ alkenylene, and C₂₋₆ alkynylene, each of which is optionally substituted with 1, 2, or 3 substituents selected from R^{g};

R⁷ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents selected from R^{g}; or L² is absent;

R⁸ is selected from H, OR^{a1}, C₆₋₁₀ aryl and 5-10 membered heteroaryl, each of which is optionally substituted with 1, 2, 3, 4, or 5 substituents independently selected from R^{g};

or R⁷, L², and R⁸, together with the N atom to which R⁷ and L² are attached form a 4-7 membered heterocycloalkyl, which is optionally substituted with C(O)Cy and is optionally substituted with 1, 2, or 3 substituents independently selected from R^{g};

Cy is selected from C₆₋₁₀ aryl and 5-10 membered heteroaryl, each of which is optionally substituted with 1, 2, 3, 4, or 5 substituents independently selected from R^{g};

each R^{a1}, R^{b1}, R^{c1}, and R^{d1} is independently selected from H, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};

or any R^{c1} and R^{d1} together with the N atom to which they are attached form a 4-7 membered heterocycloalkyl, which is optionally substituted with 1, 2, or 3 substituents independently selected from R^{g};

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

Paragraph 30. The method of paragraph 29, wherein R⁷, L², and R⁸, together with the N atom to which R⁷ and L² are attached form a 4-7 membered heterocycloalkyl, which is optionally substituted with 1, 2, or 3 substituents independently selected from R^{g};
Paragraph 31. The method of paragraph 29 or 30, wherein R¹, R², R³, and R⁴ are each independently selected from H, halo, C₁₋₆ alkyl, and OR^{a1}.
Paragraph 32. The method of paragraph 31, wherein:

R¹ is selected from H, C₁₋₆ alkyl and C₁₋₆ alkoxy;

R² is selected from H, halo and C₁₋₆ alkyl;

R³ is selected from H, halo and C₁₋₆ alkyl; and

R⁴ is H.

Paragraph 33. The method of any one of paragraphs 29-32, wherein [symbol] is a double bond and X is N.
Paragraph 34. The method of any one of paragraphs 29-32, wherein [symbol] is a double bond and X is CR⁵.
Paragraph 35. The method of any one of paragraphs 29-34, wherein R⁶ is Cy.
Paragraph 36. The method of any one of paragraphs 29-34, wherein R⁶ is C₁₋₆ alkyl.
Paragraph 37. The method of any one of paragraphs 29-34, wherein R⁶ is H.
Paragraph 38. The method of any one of paragraphs 29-35, wherein Cy is 5-10 membered heteroaryl, optionally substituted with 1, 2, or 3 independently selected R^{g}.
Paragraph 39. The method of any one of paragraphs 29-38, wherein R⁵ is selected from H, halo, C₁₋₆ alkyl, C₁₋₆ haloalkyl, OR^{a1}, C(O)R^{b1}, C(O)C(O)NR^{c1}R^{d1}, C(O)NR^{c1}R^{d1}, C(O)OR^{a1}, and NR^{c1}R^{d1} wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, and NR^{c1}R^{d1}, Paragraph 40. The method of paragraph 39, wherein R⁵ is C(O)R^{b1}.
Paragraph 41. The method of paragraph 39, wherein R⁵ is H.
Paragraph 42. The method of any one of paragraphs 29-41, wherein each R^{a1}, R^{b1}, R^{c1}, and R^{d1} is independently selected from H, C₁₋₆ alkyl, C₁₋₄ haloalkyl, C₆₋₁₀ aryl, 5-10 membered heteroaryl, and C₃₋₁₀ cycloalkyl.
Paragraph 43. The method of any one of paragraphs 29-42, wherein L¹ is C₁₋₆ alkylene, optionally substituted with 1, 2, or 3 substituents selected from R^{g}.
Paragraph 44. The method of any one of paragraphs 29-43, wherein R⁷ is H.
Paragraph 45. The method of any one of paragraphs 29-44, wherein L² is C₁₋₆ alkylene, optionally substituted with 1, 2, or 3 substituents selected from R^{g}.
Paragraph 46. The method of any one of paragraphs 29-44, wherein R⁸ is C₆₋₁₀ aryl, optionally substituted with 1, 2, or 3 independently selected R^{g}.
Paragraph 47. The method of any one of paragraphs 29-44, wherein R⁸ is 5-10 membered heteroaryl, optionally substituted with 1, 2, or 3 independently selected R^{g}.
Paragraph 48. The method of paragraph 47, wherein R⁸ is selected from furan-2-yl, indolyl, pyridin-2-yl, pyridin-3-yl, and thiophenyl, each of which is optionally substituted with 1, 2, or 3 independently selected R^{g}.
Paragraph 49. The method of any one of paragraphs 29-48, wherein R^{g} is selected from halo, C₁₋₆ alkyl, and C₁₋₆ alkoxy.
Paragraph 50. The method of paragraph 29, wherein:

R¹, R², R³, and R⁴ are each independently selected from H, halo, C₁₋₆ alkyl, and OR^{a1};

(i) when [symbol] is a double bond, R⁶ is selected from H, C₁₋₆ alkyl, and 5-10 membered heteroaryl which is optionally substituted with 1, 2, or 3 independently selected R^{g}, and X is selected from N and CR⁵;

(ii) when [symbol] is a single bond, R⁶ is oxo, X is CR⁵, and R⁵ and R⁴, together with the carbon atoms to which they are attached, form C₆₋₁₀ aryl ring, optionally substituted with 1, 2, or 3 substituents independently selected from R^{g};

R⁵ is selected from H, halo, C₁₋₆ alkyl, C₁₋₆ haloalkyl, OR^{a1}, C(O)R^{b1}, C(O)NR^{c1}R^{d1}, C(O)OR^{a1}, and NR^{c1}R^{d1} wherein said C₁₋₆ alkyl is optionally substituted with 1, 2, or 3 substituents independently selected from OR^{a1}, C(O)R^{b1}, C(O)NR^{c1}R^{d1}, C(O)OR^{a1}, and NR^{c1}R^{d1}, each R^{a1}, R^{b1}, R^{c1}, and R^{d1} is independently selected from H, C₁₋₆ alkyl, C₁₋₄haloalkyl, C₆₋₁₀ aryl, and C₃₋₁₀ cycloalkyl;

L¹ is C₁₋₆ alkylene, optionally substituted with 1, 2, or 3 substituents selected from R^{g};

R⁷ is H;

L² is C₁₋₆ alkylene, optionally substituted with 1, 2, or 3 substituents selected from R^{g};

R⁸ is selected from H, C₁₋₆ alkoxy, C₆₋₁₀ aryl and 5-10 membered heteroaryl, each of which is optionally substituted with 1, 2, or 3 substituents independently selected from R^{g}; and

R^{g} is selected from halo, C₁₋₆ alkyl, and C₁₋₆ alkoxy.

Paragraph 51. The method of paragraph 29, wherein:

R¹ is selected from H, C₁₋₆ alkyl and C₁₋₆ alkoxy;

R² is selected from H, halo and C₁₋₆ alkyl;

R³ is selected from H, halo and C₁₋₆ alkyl;

R⁴ is H;

(i) when [symbol] is a double bond, R⁶ is selected from H, C₁₋₆ alkyl, and 5-10 membered heteroaryl which is optionally substituted with 1, 2, or 3 independently selected R^{g}, and X is selected from N and CR⁵;

(ii) when [symbol] is a single bond, R⁶ is oxo, X is CR⁵, and R⁵ and R⁴, together with the carbon atoms to which they are attached, form C₆₋₁₀ aryl ring, optionally substituted with 1, 2, or 3 substituents independently selected from R^{g};

R⁵ is selected from H and C(O)R^{b1};

R^{b1} is selected from H, C₁₋₆ alkyl, C₁₋₄haloalkyl, C₆₋₁₀ aryl, and C₃₋₁₀ cycloalkyl;

L¹ is C₁₋₆ alkylene;

R⁷ is H;

L² is C₁₋₆ alkylene;

R⁸ is selected from H, C₁₋₆ alkoxy, C₆₋₁₀ aryl and furan-2-yl, each of which is optionally substituted with 1, 2, or 3 independently selected R^{g}; and

R^{g} is selected from halo, C₁₋₆ alkyl, and C₁₋₆ alkoxy.

Paragraph 52. The method of paragraph 29, wherein the compound is selected from any one of the compounds listed in Table 2 or Table 2a, or a pharmaceutically acceptable salt thereof.
Paragraph 53. The method of paragraph 29, wherein the compound is selected from any one of the compounds listed in Table 2, or a pharmaceutically acceptable salt thereof.
Paragraph 54. A method for increasing or maintaining levels of NMNAT2 polypeptide within a cell within a mammal, wherein said method comprises administering, to said mammal, an effective amount of a compound of Formula (III):

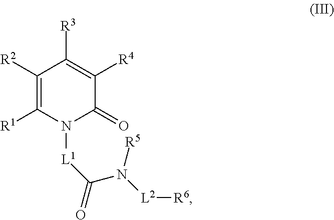

or a pharmaceutically acceptable salt thereof, wherein:

R¹, R², and R³ are each independently selected from H, halo, C₁₋₆ alkyl, C₁₋₆ haloalkyl, C₁₋₆ alkoxy, and C₁₋₆ haloalkoxy;

R⁴ is CN;

or R⁴ and R³, together with the carbon atoms to which they are attached, form a C₆₋₁₀ aryl ring or 5-10 membered heteroaryl ring, each of which is optionally substituted with 1, 2, or 3 substituents independently selected from R⁷;

each R⁷ is independently selected from halo, CN, C₁₋₆ alkyl, C₁₋₆ haloalkyl, C₁₋₆ alkoxy, and C₁₋₆ haloalkoxy;

L¹ is selected from C₁₋₆ alkylene, C₂₋₆ alkenylene, and C₂₋₆ alkynylene, each of which is optionally substituted with 1, 2, or 3 substituents selected from R^{g};

R⁵ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents selected from R^{g}; or L² is absent;

R⁶ is selected from H, C₆₋₁₀ aryl and 5-10 membered heteroaryl, each of which is optionally substituted with 1, 2, 3, 4, or 5 substituents independently selected from R⁸; provided that when L² is absent, then R⁶ is not H;

each R⁸ is independently selected from halo, CN, NO₂, C₁₋₆ alkyl, C₁₋₆ haloalkyl, C₂₋₆ alkenyl, C₂₋₆ alkynyl, OR^{a1}, SR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};

each R^{a1}, R^{b1}, R^{c1}, and R^{d1} is independently selected from H, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};

or any R^{c1} and R^{d1} together with the N atom to which they are attached form a 4-7 membered heterocycloalkyl, which is optionally substituted with 1, 2, or 3 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

Paragraph 55. The method of paragraph 54, wherein L¹ is C₁₋₆ alkylene, optionally substituted with 1, 2, or 3 substituents selected from R^{g}.
Paragraph 56. The method of paragraph 54, wherein L¹ is C₁₋₆ alkylene.
Paragraph 57. The method of any one of paragraphs 54-56, wherein R⁵ is H.
Paragraph 58. The method of any one of paragraphs 54-57, wherein L² is absent or C₁₋₆ alkylene, optionally substituted with 1, 2, or 3 substituents selected from R^{g}; and R⁶ is selected from C₆₋₁₀ aryl and 5-10 membered heteroaryl, each of which is optionally substituted with 1, 2, or 3 substituents independently selected from R⁸.
Paragraph 59. The method of paragraph 58, wherein R⁶ is selected from phenyl, furan-2-yl, and thiophen-2-yl, each of which is optionally substituted with 1, 2, or 3 substituents independently selected from R⁸.
Paragraph 60. The method of any one of paragraphs 54-57, wherein L² is C₁₋₆ alkylene, optionally substituted with 1, 2, or 3 substituents independently selected from R^{g}; and R⁶ is H.
Paragraph 61. The method of any one of paragraphs 54-59, wherein R⁸ is selected from halo, C₁₋₆ alkyl, C₁₋₆ haloalkyl, OR^{a1}, SR^{a1}, C(O)R^{b1}, C(O)NR^{c1}R^{d1}, C(O)OR^{a1}, NR^{c1}R^{d1}, and NR^{c1}C(O)R^{b1}.
Paragraph 62. The method of any one of paragraphs 54-59, wherein R⁸ is selected from halo, C₁₋₆ alkyl, OR^{a1}, SR^{a1}, C(O)NR^{c1}R^{d1}.
Paragraph 63. The method of any one of paragraphs 54-62, wherein each R^{a1}, R^{b1}, R^{c1}, and R^{d1} is independently selected from H, C₁₋₆ alkyl, C₁₋₄haloalkyl, C₆₋₁₀ aryl-C₁₋₄ alkylene, C₃₋₁₀ cycloalkyl-C₁₋₄ alkylene, and (5-10 membered heteroaryl)-C₁₋₄ alkylene.
Paragraph 64. The method of any one of paragraphs 54-62, wherein each R^{a1}, R^{b1}, R^{c}, and R^{d1} is independently selected from H, C₁₋₆ alkyl, and (5-10 membered heteroaryl)-C₁₋₄ alkylene.
Paragraph 65. The method of any one of paragraphs 54-64, wherein the compound of Formula (III) has formula:

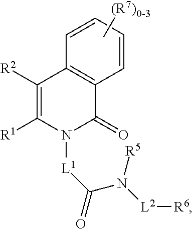

or a pharmaceutically acceptable salt thereof.
Paragraph 66. The method of any one of paragraphs 54-64, wherein the compound of Formula (III) has formula:

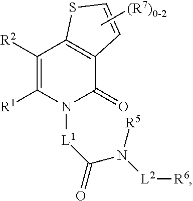

or a pharmaceutically acceptable salt thereof.
Paragraph 67. The method of paragraph 65 or 66, wherein R⁷ is selected from C₁₋₆ alkyl, C₁₋₆ haloalkyl, C₁₋₆ alkoxy, and C₁₋₆ haloalkoxy.
Paragraph 68. The method of paragraph 67, wherein R⁷ is C₁₋₆ alkyl.
Paragraph 69. The method of any one of paragraphs 65-68, wherein R¹ and R² are each independently selected from H and C₁₋₆ alkyl.
Paragraph 70. The method of paragraph 69, wherein R¹ is C₁₋₆ alkyl, and R² is H.
Paragraph 71. The method of any one of paragraphs 54, 65, or 66, wherein:

R¹ and R² are each independently selected from H and C₁₋₆ alkyl;

R⁷ is selected from C₁₋₆ alkyl, C₁₋₆ haloalkyl, C₁₋₆ alkoxy, and C₁₋₆ haloalkoxy;

L¹ is C₁₋₆ alkylene, optionally substituted with 1, 2, or 3 substituents selected from R^{g};

R⁵ is H;

L² is absent or C₁₋₆ alkylene, optionally substituted with 1, 2, or 3 substituents selected from R^{g}, and R⁶ is selected from C₆₋₁₀ aryl and 5-10 membered heteroaryl, each of which is optionally substituted with 1, 2, or 3 substituents independently selected from R⁸;

or L² is C₁₋₆ alkylene, optionally substituted with 1, 2, or 3 substituents independently selected from R^{g}, and R⁶ is H;

R⁸ is selected from halo, C₁₋₆ alkyl, C₁₋₆ haloalkyl, OR^{a1}, SR^{a1}, C(O)R^{b1}, C(O)NR^{c1}R^{d1}, C(O)OR^{a1}, NR^{c1}R^{d1}, and NR^{c1}C(O)R^{b1}; and

each R^{a1}, R^{b1}, R^{c1}, and R^{d1} is independently selected from H, C₁₋₆ alkyl, C₁₋₄haloalkyl, C₆₋₁₀ aryl-C₁₋₄ alkylene, C₃₋₁₀ cycloalkyl-C₁₋₄ alkylene, and (5-10 membered heteroaryl)-C₁₋₄ alkylene.

Paragraph 72. The method of any one of paragraphs 54, 65, or 66, wherein:

R¹ is C₁₋₆ alkyl;

R² is H;

R⁷ is C₁₋₆ alkyl;

L¹ is C₁₋₆ alkylene;

R⁵ is H;

L² is absent or C₁₋₆ alkylene, optionally substituted with 1, 2, or 3 substituents selected from R^{g}, and R⁶ is selected from phenyl, furan-2-yl, and thiophen-2-yl, each of which is optionally substituted with 1, 2, or 3 substituents independently selected from R⁸;

or L² is C₁₋₆ alkylene, optionally substituted with 1, 2, or 3 substituents independently selected from R^{g}, and R⁶ is H;

R⁸ is selected from halo, C₁₋₆ alkyl, OR^{a1}, SR^{a1}, and C(O)NR^{c1}R^{d1}; and

each R^{a1}, R^{c1}, and R^{d1} is independently selected from H, C₁₋₆ alkyl, and (5-10 membered heteroaryl)-C₁₋₄ alkylene.

Paragraph 73. The method of any one of paragraphs 54-64, wherein the compound of Formula (III) has formula:

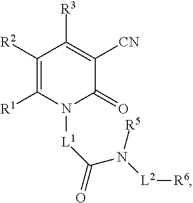

or a pharmaceutically acceptable salt thereof.
Paragraph 74. The method of paragraph 73, wherein R¹, R², and R³ are each independently selected from H, and C₁₋₆ alkyl.
Paragraph 75. The method of paragraph 73, wherein R¹ and R³ are each C₁₋₆ alkyl, and R² is H.
Paragraph 76. The method of paragraph 54 or 73, wherein:

R¹, R², and R³ are each independently selected from H, and C₁₋₆ alkyl;

L¹ is C₁₋₆ alkylene, optionally substituted with 1, 2, or 3 substituents selected from R^{g};

R⁵ is H;

L² is absent or C₁₋₆ alkylene, optionally substituted with 1, 2, or 3 substituents selected from R^{g}, and R⁶ is selected from C₆₋₁₀ aryl and 5-10 membered heteroaryl, each of which is optionally substituted with 1, 2, or 3 substituents independently selected from R⁸;

or L² is C₁₋₆ alkylene, optionally substituted with 1, 2, or 3 substituents independently selected from R^{g}, and R⁶ is H;

R⁸ is selected from halo, C₁₋₆ alkyl, C₁₋₆ haloalkyl, OR^{a1}, SR^{a1}, C(O)R^{b1}, C(O)NR^{c1}R^{d1}, C(O)OR^{a1}, NR^{c1}R^{d1}, and NR^{c1}C(O)R^{b1}; and

each R^{a1}, R^{b1}, R^{c1}, and R^{c1} is independently selected from H, C₁₋₆ alkyl, C₁₋₄haloalkyl, C₆₋₁₀ aryl-C₁₋₄ alkylene, C₃₋₁₀ cycloalkyl-C₁₋₄ alkylene, and (5-10 membered heteroaryl)-C₁₋₄ alkylene.

Paragraph 77. The method of paragraph 54 or 73, wherein:

R¹ and R³ are each C₁₋₆ alkyl;

R² is H;

L¹ is C₁₋₆ alkylene;

R⁵ is H;

L² is absent or C₁₋₆ alkylene, optionally substituted with 1, 2, or 3 substituents selected from R^{g}, and R⁶ is selected from phenyl, furan-2-yl, and thiophen-2-yl, each of which is optionally substituted with 1, 2, or 3 substituents independently selected from R⁸;

or L² is C₁₋₆ alkylene, optionally substituted with 1, 2, or 3 substituents independently selected from R^{g}, and R⁶ is H;

R⁸ is selected from halo, C₁₋₆ alkyl, OR^{a1}, SR^{a1}, and C(O)NR^{c1}R^{d1}; and

each R^{a1}, R^{c1}, and R^{d1} is independently selected from H, C₁₋₆ alkyl, and (5-10 membered heteroaryl)-C₁₋₄ alkylene.

Paragraph 78. The method of paragraph 54, wherein the compound is selected from any one of the compounds listed in Table 3, or a pharmaceutically acceptable salt thereof.
Paragraph 79. A method for increasing or maintaining levels of NMNAT2 polypeptide within a cell within a mammal, wherein said method comprises administering, to said mammal, an effective amount of a compound of Formula (IV):

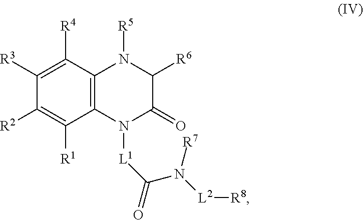

or a pharmaceutically acceptable salt thereof, wherein:

R¹, R², R³, and R⁴ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, and C₁₋₆ haloalkoxy;

R⁵ is selected from C₁₋₆ alkyl, C₁₋₆ haloalkyl, C₂₋₆ alkenyl, and C₂₋₆ alkynyl, each of which is optionally substituted with 1, 2, or 3 substituents independently selected from R^{g};

R⁶ is oxo;

or R⁵ and R⁶, together with N atom to which R⁵ is attached and carbon atom to which R⁶ is attached, form a 4-10 membered heterocycloalkyl ring or a 5-10 membered heteroaryl ring, each of which is optionally substituted with 1, 2, or 3 independently selected R^{g};

L¹ is selected from C₁₋₆ alkylene, C₂₋₆ alkenylene, and C₂₋₆ alkynylene, each of which is optionally substituted with 1, 2, or 3 substituents selected from R^{g};

R⁷ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents selected from R^{g};

R⁸ is 5-10 membered heteroaryl, optionally substituted with 1, 2, 3, 4, or 5 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

Paragraph 80. The method of paragraph 79, wherein R¹, R², R³, and R⁴ are each independently selected from H and halo.
Paragraph 81. The method of paragraph 79, wherein R² is selected from H and halo, and R¹, R³, and R⁴ are each H.
Paragraph 82. The method of any one of paragraphs 79-81, wherein the compound of Formula (IV) has formula:

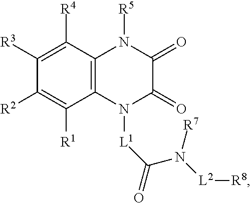

or a pharmaceutically acceptable salt thereof.
Paragraph 83. The method of any one of paragraphs 79-82, wherein R⁵ is C₁₋₆ alkyl.
Paragraph 84. The method of any one of paragraphs 79-81, wherein the compound of Formula (IV) has formula:

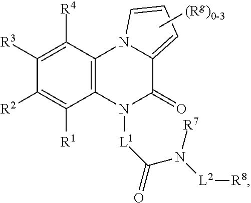

or a pharmaceutically acceptable salt thereof.
Paragraph 85. The method of any one of paragraphs 79-84, wherein L¹ is C₁₋₆ alkylene, optionally substituted with 1, 2, or 3 substituents selected from R^{g}.
Paragraph 86. The method of any one of paragraphs 79-84, wherein L¹ is C₁₋₆ alkylene.
Paragraph 87. The method of any one of paragraphs 79-86, wherein R⁷ is H.
Paragraph 88. The method of any one of paragraphs 79-87, wherein L² is C₁₋₆ alkylene, optionally substituted with 1, 2, or 3 substituents selected from R^{g}.
Paragraph 89. The method of any one of paragraphs 79-87, wherein L² is C₁₋₆ alkylene.
Paragraph 90. The method of any one of paragraphs 79-89, wherein R⁸ is 5-10 membered heteroaryl, optionally substituted with 1, 2, or 3 substituents independently selected from R^{g}.
Paragraph 91. The method of paragraph 90, wherein the 5-10 membered heteroaryl is furan-2-yl.
Paragraph 92. The method of any one of paragraphs 79-91, wherein R^{g} is selected from OH, NO₂, CN, halo, C₁₋₆ alkyl, C₁₋₄ haloalkyl, C₁₋₆ alkoxy, and C₁₋₆ haloalkoxy.
Paragraph 93. The method of paragraph 92, wherein R^{g} is selected from halo, C₁₋₆ alkyl, and C₁₋₆ alkoxy.
Paragraph 94. The method of paragraph 92, wherein R^{g} is C₁₋₆ alkyl.
Paragraph 95. The method of paragraph 82, wherein:

R¹, R², R³, and R⁴ are each independently selected from H and halo;

R⁵ is C₁₋₆ alkyl;

L¹ is C₁₋₆ alkylene, optionally substituted with 1, 2, or 3 substituents selected from R^{g};

R⁷ is H;

L² is C₁₋₆ alkylene, optionally substituted with 1, 2, or 3 substituents selected from R^{g};

R⁸ is 5-10 membered heteroaryl, optionally substituted with 1, 2, or 3 substituents independently selected from R^{g}; and

R^{g} is selected from OH, NO₂, CN, halo, C₁₋₆ alkyl, C₁₋₄ haloalkyl, C₁₋₆ alkoxy, and C₁₋₆ haloalkoxy.

Paragraph 96. The method of paragraph 82, wherein:

R² is selected from H and halo;

R¹, R³, and R⁴ are each H;

R⁵ is C₁₋₆ alkyl;

L¹ is C₁₋₆ alkylene;

R⁷ is H;

L² is C₁₋₆ alkylene; and

R⁸ is 5-10 membered heteroaryl, optionally substituted with C₁₋₆ alkyl.

Paragraph 97. The method of paragraph 84, wherein:

R¹, R², R³, and R⁴ are each independently selected from H and halo;

L¹ is C₁₋₆ alkylene, optionally substituted with 1, 2, or 3 substituents selected from R^{g};

R⁷ is H;

L² is C₁₋₆ alkylene, optionally substituted with 1, 2, or 3 substituents selected from R^{g};

R⁸ is 5-10 membered heteroaryl, optionally substituted with 1, 2, or 3 substituents independently selected from R^{g}; and

R^{g} is selected from OH, NO₂, CN, halo, C₁₋₆ alkyl, C₁₋₄ haloalkyl, C₁₋₆ alkoxy, and C₁₋₆ haloalkoxy.

Paragraph 98. The method of paragraph 84, wherein:

R² is selected from H and halo;

R¹, R³, and R⁴ are each H;

R⁵ is C₁₋₆ alkyl;

L¹ is C₁₋₆ alkylene;

R⁷ is H;

L² is C₁₋₆ alkylene; and

R⁸ is 5-10 membered heteroaryl, optionally substituted with C₁₋₆ alkyl.

Paragraph 99. The method of paragraph 79, wherein the compound of Formula (IV) is selected from any one of the compounds listed in Table 4, or a pharmaceutically acceptable salt thereof.
Paragraph 100. A method for increasing or maintaining levels of NMNAT2 polypeptide within a cell within a mammal, wherein said method comprises administering, to said mammal, an effective amount of a compound of Formula (V):

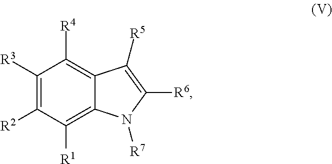

or a pharmaceutically acceptable salt thereof, wherein:

R¹, R², R³, R⁴, R⁵, and R⁶ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, C₁₋₆ haloalkoxy, wherein said C₁₋₆ alkyl is optionally substituted with 1, 2, or 3 substituents independently selected from OH, NO₂, CN, C₆₋₁₀ alkoxy, C₆₋₁₀ haloalkoxy, and Cy, and a moiety of formula (i):

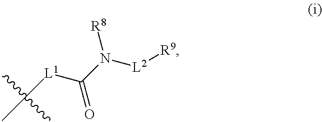

provided that at least one of R¹, R², R³, R⁴, R⁵, and R⁶ is a moiety of formula (i);

or R⁵ and R⁶ together with the carbon atoms to which they are attached from a C₄₋₇ cycloalkyl ring or a 4-7 membered heterocycloalkyl ring, each of which is optionally substituted with 1, 2, or 3 substituents independently selected from OH, NO₂, CN, C₆₋₁₀ alkoxy, C₆₋₁₀ haloalkoxy, Cy, and a moiety of formula (i);

or when R⁴ is a moiety of formula (i), the R⁸ of the moiety of formula (i) and R⁵, together with the N atom to which R⁸ is attached and the carbon atom to which R⁵ is attached, form a 4-10 membered heterocycloalkyl, which is optionally substituted with 1, 2, 3, 4, or 5 substituents independently selected from R^{g};

R⁷ is selected from C₁₋₆ alkyl, C₁₋₆ haloalkyl, C₂₋₆ alkenyl, and C₂₋₆ alkynyl, wherein said C₁₋₆ alkyl is optionally substituted with 1, 2, or 3 substituents independently selected from OH, NO₂, CN, C₆₋₁₀ alkoxy, C₆₋₁₀ haloalkoxy, Cy, and a moiety of formula (i);

L¹ is selected from C(O), C₁₋₆ alkylene, C(O)—C₁₋₆ alkylene, C₃₋₁₀ cycloalkylene, C₂₋₆ alkenylene, and C₂₋₆ alkynylene, each of which is optionally substituted with 1, 2, or 3 substituents independently selected from R¹⁰; or L¹ is absent;

each R¹⁰ is independently selected from halo, OH, NO₂, CN, C₆₋₁₀ alkoxy, C₆₋₁₀ haloalkoxy, and Cy;

R⁸ is selected from H and C₁₋₆ alkyl, wherein said C₁₋₆ alkyl is optionally substituted with 1, 2, or 3 substituents independently selected from C₁₋₆ alkoxy, amino, C₁₋₆ alkylamino, di(C₁₋₆ alkyl)amino, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, 3, 4, or 5 substituents independently selected from R^{g};

or R⁸ and R¹⁰ together with the N atom to which R⁸ is attached, L¹ to which R¹⁰ is attached, and C(O) between the N and the L¹, form a 4-10 membered heterocycloalkyl, which is optionally substituted with 1, 2, 3, 4, or 5 substituents independently selected from R^{g};

L² is selected from C₁₋₆ alkylene, C₁₋₆ alkylene-NR⁸—C₁₋₆ alkylene, C₂₋₆ alkenylene, and C₂₋₆ alkynylene, each of which is optionally substituted with 1, 2, or 3 substituents independently selected from R^{g}; or L² is absent;

R^{g} is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, 3, 4, or 5 substituents independently selected from R^{g};

or R⁸ and L²-R^{g}, together with the N atom to which R⁸ and L² are attached, form a 4-10 membered heterocycloalkyl, which is optionally substituted with C(O)Cy and is optionally substituted with 1, 2, 3, 4, or 5 substituents independently selected from R^{g};

or R⁹ and R¹⁰, together with the L¹ to which R¹⁰ is attached, L² to which R⁹ is attached, and C(O) and NR⁸ between the L¹ and the L², form a 4-10 membered heterocycloalkyl, which is optionally substituted with 1, 2, 3, 4, or 5 substituents independently selected from R^{g};

Cy is selected from C₆₋₁₀ aryl and 5-10 membered heteroaryl, each of which is optionally substituted with 1, 2, 3, 4, or 5 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, NH₂-C₁₋₃ alkylene, C₁₋₆ alkylamino-C₁₋₃ alkylene, di(C₁₋₆ alkyl)amino-C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

Paragraph 101. The method of paragraph 100, wherein:

R⁷ is selected from C₁₋₆ alkyl, C₁₋₆ haloalkyl, C₂₋₆ alkenyl, and C₂₋₆ alkynyl, wherein said C₁₋₆ alkyl is optionally substituted with 1, 2, or 3 substituents independently selected from OH, NO₂, CN, C₆₋₁₀ alkoxy, C₆₋₁₀ haloalkoxy, and Cy;

L¹ is selected from C(O), C₁₋₆ alkylene, C₂₋₆ alkenylene, and C₂₋₆ alkynylene, each of which is optionally substituted with 1, 2, or 3 substituents independently selected from halo, OH, NO₂, CN, C₆₋₁₀ alkoxy, C₆₋₁₀ haloalkoxy, and Cy; or L¹ is absent;

R⁸ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents independently selected from R^{g};

R^{g} is selected from H, C₁₋₆ alkoxy, C₆₋₁₀ aryl and 5-10 membered heteroaryl, each of which is optionally substituted with 1, 2, 3, 4, or 5 substituents independently selected from R^{g};

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

Paragraph 102. The method of paragraph 100, wherein the compound of Formula (V) has formula:

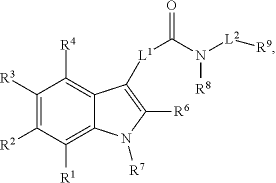

or a pharmaceutically acceptable salt thereof.
Paragraph 103. The method of paragraph 102, wherein R¹, R², R³, R⁴, and R⁶ are each H.
Paragraph 104. The method of paragraph 100, wherein the compound of Formula (V) has formula:

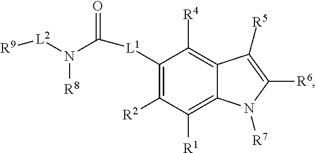

or a pharmaceutically acceptable salt thereof.
Paragraph 105. The method of paragraph 102, wherein R¹, R², R⁴, R⁵, and R⁶ are each H.
Paragraph 106. The method of paragraph 105, wherein R¹, R², and R⁴ are each H, and R⁵ and and R⁶ are each C₁₋₆ alkyl.
Paragraph 107. The method of paragraph 100, wherein the compound of Formula (V) has formula:

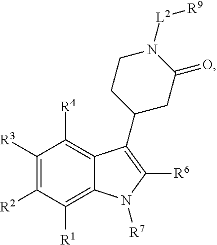

or a pharmaceutically acceptable salt thereof.
Paragraph 108. The method of paragraph 107, wherein R¹, R², R³, R⁴, and R⁶ are each H.
Paragraph 109. The method of paragraph 100, wherein the compound of Formula (V) has formula:

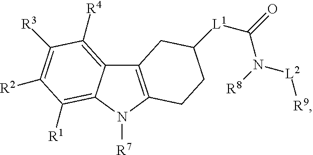

or a pharmaceutically acceptable salt thereof.
Paragraph 110. The method of paragraph 100, wherein the compound of Formula (V) has formula:

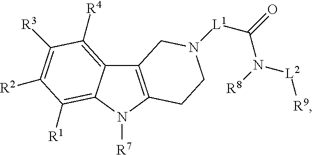

or a pharmaceutically acceptable salt thereof.
Paragraph 111. The method of paragraph 100, wherein the compound of Formula (V) has formula:

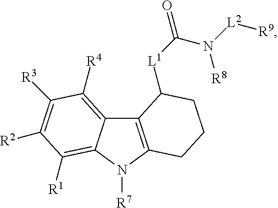

or a pharmaceutically acceptable salt thereof.
Paragraph 112. The method of any one of paragraphs 109-111, wherein R¹, R², R³, and R⁴ are each H.
Paragraph 113. The method of paragraph 100, wherein the compound of Formula (V) has formula:

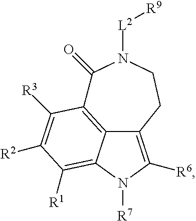

or a pharmaceutically acceptable salt thereof.
Paragraph 114. The method of paragraph 113, wherein R¹, R², R³, and R⁶ are each H.
Paragraph 115. The method of any one of paragraphs 100-114, wherein R⁷ is C₁₋₆ alkyl, optionally substituted with Cy.
Paragraph 116. The method of any one of paragraphs 100-114, wherein R⁷ is a moiety of formula (i).
Paragraph 117. The method of any one of paragraphs 100-116, wherein L¹ is C(O), C₁₋₆ alkylene, C(O)—C₁₋₆ alkylene, or C₃₋₁₀ cycloalkylene, each of which is optionally substituted with R¹⁰.
Paragraph 118. The method of any one of paragraphs 100-116, wherein L¹ is C(O) or C₁₋₆ alkylene, optionally substituted with Cy.
Paragraph 119. The method of any one of paragraphs 100-116, wherein L¹ is absent.
Paragraph 120. The method of any one of paragraphs 100-119, wherein R⁸ is C₁₋₆ alkyl, which is optionally substituted with C₁₋₆ alkoxy, di(C₁₋₆ alkyl)amino, or 4-10 membered heterocycloalkyl.

Paragraph 121. The method of any one of paragraphs 100-119, wherein R⁸ is H.

Paragraph 122. The method of any one of paragraphs 100-121, wherein L² is C₁₋₆ alkylene or C₁₋₆ alkylene-NR⁸—C₁₋₆ alkylene; or L² is absent.
Paragraph 123. The method of any one of paragraphs 100-121, wherein L² is C₁₋₆ alkylene; or L² is absent.
Paragraph 124. The method of any one of paragraphs 100-123, wherein Cy is selected from C₆₋₁₀ aryl and 5-10 membered heteroaryl, each of which is optionally substituted with 1, 2, or 3 independently selected R^{g}.
Paragraph 125. The method of any one of paragraphs 100-123, wherein Cy is selected from C₆₋₁₀ aryl and 5-10 membered heteroaryl, each of which is optionally substituted with 1, 2, or 3 substituents independently selected from halo, C₁₋₆ alkyl, and C₁₋₆ alkoxy.
Paragraph 126. The method of any one of paragraphs 100-123, wherein Cy is C₆₋₁₀ aryl, optionally substituted with halo.
Paragraph 127. The method of any one of paragraphs 100-126, wherein R^{g} is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.
Paragraph 128. The method of any one of paragraphs 100-126, wherein R^{g} is selected from H, C₁₋₆ alkoxy, C₆₋₁₀ aryl and 5-10 membered heteroaryl, each of which is optionally substituted with 1, 2, or 3 substituents independently selected from R^{g}.
Paragraph 129. The method of paragraph 102, wherein:

R¹, R², R³, R⁴, and R⁶ are each H;

R⁷ is C₁₋₆ alkyl, optionally substituted with Cy or a moiety of formula (i);

L¹ is selected from C₁₋₆ alkylene, C(O)—C₁₋₆ alkylene, and C₃₋₁₀ cycloalkylene, each of which is optionally substituted with R¹⁰; or L¹ is absent;

each R¹⁰ is Cy;

R⁸ is selected from H and C₁₋₆ alkyl, which is optionally substituted with C₁₋₆ alkoxy, di(C₁₋₆ alkyl)amino, or 4-10 membered heterocycloalkyl;

L² is C₁₋₆ alkylene or C₁₋₆ alkylene-NR⁸—C₁₋₆ alkylene; or L² is absent;

Cy is selected from C₆₋₁₀ aryl and 5-10 membered heteroaryl, each of which is optionally substituted with 1, 2, or 3 independently selected R^{g}; and

R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

Paragraph 130. The method of paragraph 102, wherein:

R¹, R², R³, R⁴, and R⁶ are each H;

R⁷ is C₁₋₆ alkyl, optionally substituted with Cy;

L¹ is C₁₋₆ alkylene, optionally substituted with Cy;

R⁸ is H;

L² is C₁₋₆ alkylene;

Cy is C₆₋₁₀ aryl, optionally substituted with halo; and

R⁹ is selected from H, C₁₋₆ alkoxy, C₆₋₁₀ aryl and 5-10 membered heteroaryl, each of which optionally substituted with 1, 2, or 3 substituents independently selected from R^{g}.

Paragraph 131. The method of paragraph 102, wherein:

R¹, R², R³, R⁴, and R⁶ are each H;

R⁷ is C₁₋₆ alkyl, optionally substituted with Cy;

L¹ is C(O) or C₁₋₆ alkylene, optionally substituted with Cy;

R⁸ is H;

L² is C₁₋₆ alkylene;

Cy is phenyl, optionally substituted with halo; and

R⁹ is H, C₁₋₃ alkoxy, phenyl, furan-2-yl, or thiophenyl, each of which is optionally substituted with 1, 2, or 3 substituents independently selected from R^{g}.

Paragraph 132. The method of paragraph 104, wherein

R¹, R², and R⁴ are each H, and R⁵ and R⁶ are each independently selected from H and C₁₋₆ alkyl;

R⁷ is C₁₋₆ alkyl, optionally substituted with Cy or a moiety of formula (i);

L¹ is selected from C(O), C₁₋₆ alkylene, C(O)—C₁₋₆ alkylene, and C₃₋₁₀ cycloalkylene, each of which is optionally substituted with R¹⁰; or L¹ is absent; each R¹⁰ is Cy;

R⁸ is selected from H and C₁₋₆ alkyl, which is optionally substituted with C₁₋₆ alkoxy, di(C₁₋₆ alkyl)amino, or 4-10 membered heterocycloalkyl;

L² is C₁₋₆ alkylene or C₁₋₆ alkylene-NR⁸—C₁₋₆ alkylene; or L² is absent;

Cy is selected from C₆₋₁₀ aryl and 5-10 membered heteroaryl, each of which is optionally substituted with 1, 2, or 3 independently selected R^{g}; and

R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

Paragraph 133. The method of paragraph 104, wherein:

R¹, R², R⁴, R⁵, and R⁶ are each H;

R⁷ is C₁₋₆ alkyl, optionally substituted with Cy;

L¹ is C₁₋₆ alkylene, optionally substituted with Cy;

R⁸ is H;

L² is C₁₋₆ alkylene; or L² is absent;

Cy is C₆₋₁₀ aryl, optionally substituted with halo; and

R⁹ is 5-10 membered heteroaryl, optionally substituted with 1, 2, or 3 substituents independently selected from R^{g}.

Paragraph 134. The method of paragraph 104, wherein:

R¹, R², R⁴, R⁵, and R⁶ are each H;

R⁷ is C₁₋₆ alkyl, optionally substituted with Cy;

L¹ is C₁₋₆ alkylene, optionally substituted with Cy;

R⁸ is H;

L² is C₁₋₆ alkylene; or L² is absent;

Cy is phenyl, optionally substituted with halo; and

R⁹ is furan-2-yl, optionally substituted with 1, 2, or 3 substituents independently selected from R^{g}.

Paragraph 135. The method of paragraph 107, wherein:

R¹, R², R³, R⁴, and R⁶ are each H;

R⁷ is C₁₋₆ alkyl, optionally substituted with Cy or a moiety of formula (i);

L² is C₁₋₆ alkylene or C₁₋₆ alkylene-NR⁸—C₁₋₆ alkylene; or L² is absent;

R⁸ is selected from H and C₁₋₆ alkyl, which is optionally substituted with C₁₋₆ alkoxy, di(C₁₋₆ alkyl)amino, or 4-10 membered heterocycloalkyl;

Cy is selected from C₆₋₁₀ aryl and 5-10 membered heteroaryl, each of which is optionally substituted with 1, 2, or 3 independently selected R^{g}; and

R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

Paragraph 136. The method of any one of paragraphs 109-111, wherein:

R¹, R², R³, and R⁴ are each H;

R⁷ is C₁₋₆ alkyl, optionally substituted with Cy or a moiety of formula (i);

L¹ is selected from C(O), C₁₋₆ alkylene, C(O)—C₁₋₆ alkylene, and C₃₋₁₀ cycloalkylene, each of which is optionally substituted with R¹⁰; or L¹ is absent;

each R¹⁰ is Cy;

R⁸ is selected from H and C₁₋₆ alkyl, which is optionally substituted with C₁₋₆ alkoxy, di(C₁₋₆ alkyl)amino, or 4-10 membered heterocycloalkyl;

L² is C₁₋₆ alkylene or C₁₋₆ alkylene-NR⁸—C₁₋₆ alkylene; or L² is absent;

Cy is selected from C₆₋₁₀ aryl and 5-10 membered heteroaryl, each of which is optionally substituted with 1, 2, or 3 independently selected R^{g}; and

R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

Paragraph 137. The method of paragraph 113, wherein:

R¹, R², R³, and R⁶ are each H;

R⁷ is C₁₋₆ alkyl, optionally substituted with Cy or a moiety of formula (i);

L² is C₁₋₆ alkylene or C₁₋₆ alkylene-NR⁸—C₁₋₆ alkylene; or L² is absent;

R⁸ is selected from H and C₁₋₆ alkyl, which is optionally substituted with C₁₋₆ alkoxy, di(C₁₋₆ alkyl)amino, or 4-10 membered heterocycloalkyl;

Cy is selected from C₆₋₁₀ aryl and 5-10 membered heteroaryl, each of which is optionally substituted with 1, 2, or 3 independently selected R^{g}; and

R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

Paragraph 138. The method of paragraph 100, wherein the compound of Formula (V) is selected from any one of the compounds listed in Table 5, Table 5a, Table 5b, Table 5c, Table 5d, and Table 5e, or a pharmaceutically acceptable salt thereof.
Paragraph 139. The method of paragraph 100, wherein the compound of Formula (V) is selected from any one of the compounds listed in Table 5, or a pharmaceutically acceptable salt thereof.
Paragraph 140. A method for increasing or maintaining levels of NMNAT2 polypeptide within a cell within a mammal, wherein said method comprises administering, to said mammal, an effective amount of a compound of Formula (VI):

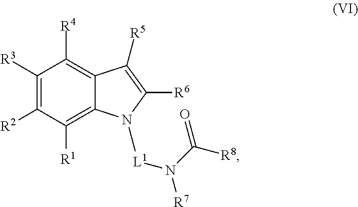

or a pharmaceutically acceptable salt thereof, wherein.

R¹, R², R³, R⁴, R⁵, and R⁶ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, and C₁₋₆ haloalkoxy;

L¹ is selected from C₁₋₆ alkylene, C₂₋₆ alkenylene, and C₂₋₆ alkynylene, each of which is optionally substituted with 1, 2, or 3 substituents independently selected from R^{g};

R⁷ is selected from H and C₁₋₆ alkyl;

R⁹ is selected from C₆₋₁₀ aryl and 5-10 membered heteroaryl, each of which is optionally substituted with 1, 2, 3, 4, or 5 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

Paragraph 141. The method of paragraph 140, wherein R¹, R², R³, R⁴, R⁵, and R⁶ are each independently selected from H, halo, and C₁₋₆ alkoxy.
Paragraph 142. The method of paragraph 140, wherein:

R¹ is H;

R² is selected from H and halo;

R³ is selected from H, halo, and C₁₋₆ alkoxy;

R⁴ is selected from H and halo.

Paragraph 143. The method of any one of paragraphs 140-142, wherein L¹ is C₁₋₆ alkylene.
Paragraph 144. The method of any one of paragraphs 140-143, wherein R⁷ is H.
Paragraph 145. The method of any one of paragraphs 140-144, wherein R⁹ is 5-10 membered heteroaryl, optionally substituted with 1, 2, or 3 substituents independently selected from R^{g}.
Paragraph 146. The method of any one of paragraphs 140-144, wherein R⁹ is furan-2-yl.
Paragraph 147. The method of paragraph 140, wherein:

R¹, R², R³, R⁴, R⁵, and R⁶ are each independently selected from H, halo, and C₁₋₆ alkoxy;

L¹ is C₁₋₆ alkylene;

R⁷ is H; and

R⁹ is 5-10 membered heteroaryl, optionally substituted with 1, 2, or 3 substituents independently selected from R^{g}.

Paragraph 148. The method of paragraph 140, wherein:

R¹ is H;

R² is selected from H and halo;

R³ is selected from H, halo, and C₁₋₆ alkoxy;

R⁴ is selected from H and halo;

L¹ is C₁₋₆ alkylene;

R⁷ is H; and

R⁹ is furan-2-yl, optionally substituted with 1, 2, or 3 substituents independently selected from R^{g}.

Paragraph 149. The method of paragraph 140, wherein the compound of Formula (VI) is selected from any one of the compounds listed in Table 6, or a pharmaceutically acceptable salt thereof.
Paragraph 150. A method for increasing or maintaining levels of NMNAT2 polypeptide within a cell within a mammal, wherein said method comprises administering, to said mammal, an effective amount of a compound of Formula (VII):

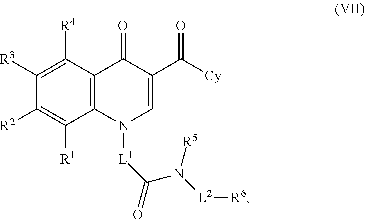

or a pharmaceutically acceptable salt thereof, wherein:

R¹, R², R³, and R⁴ are each independently selected from H, halo, C₁₋₆ alkyl, C₁₋₆ haloalkyl, C₁₋₆ alkoxy, and C₁₋₆ haloalkoxy;

Cy is selected from C₆₋₁₀ aryl and 5-10 membered heteroaryl, each of which is optionally substituted with 1, 2, 3, 4, or 5 substituents independently selected from R^{g};

L¹ is selected from C₁₋₆ alkylene, C₂₋₆ alkenylene, and C₂₋₆ alkynylene, each of which is optionally substituted with 1, 2, or 3 substituents selected from R^{g};

R⁵ is selected from H and C₁₋₆ alkyl;

L² is selected from C₁₋₆ alkylene, C₂₋₆ alkenylene, and C₂₋₆ alkynylene, each of which is optionally substituted with 1, 2, or 3 substituents independently selected from R^{g};

R⁶ is selected from C₆₋₁₀ aryl and 5-10 membered heteroaryl, each of which is optionally substituted with 1, 2, 3, 4, or 5 substituents independently selected from R^{g}; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

Paragraph 151. The method of paragraph 150, wherein R¹, R², R³, and R⁴ are each independently selected from H, halo, and C₁₋₆ alkyl.
Paragraph 152. The method of paragraph 150, wherein, Cy is 5-10 membered heteroaryl, optionally substituted with 1, 2, or 3 substituents independently selected from R^{g}.
Paragraph 153. The method of paragraph 150, wherein L¹ is C₁₋₆ alkylene, optionally substituted with 1, 2, or 3 substituents selected from R^{g}.
Paragraph 154. The method of paragraph 150, wherein R⁵ is H.
Paragraph 155. The method of paragraph 150, wherein L² is C₁₋₆ alkylene, optionally substituted with 1, 2, or 3 substituents selected from R^{g}.
Paragraph 156. The method of paragraph 150, wherein R⁶ is C₆₋₁₀ aryl, optionally substituted with 1, 2, or 3 substituents independently selected from R^{g}.
Paragraph 157. The method of paragraph 150 wherein R^{g} is selected from halo and C₁₋₆ alkyl.
Paragraph 158. The method of paragraph 150, wherein:

R¹, R², R³, and R⁴ are each independently selected from H, halo, and C₁₋₆ alkyl;

Cy is 5-10 membered heteroaryl, optionally substituted with 1, 2, or 3 substituents independently selected from R^{g};

L¹ is C₁₋₆ alkylene, optionally substituted with 1, 2, or 3 substituents selected from R^{g};

R⁵ is H;

L² is C₁₋₆ alkylene, optionally substituted with 1, 2, or 3 substituents selected from R^{g};

R⁶ is C₆₋₁₀ aryl, optionally substituted with 1, 2, or 3 substituents independently selected from R^{g}; and

each R^{g} is independently selected from halo and C₁₋₆ alkyl.

Paragraph 159. The method of paragraph 150, wherein the compound of Formula (VII) is selected from any one of the compounds listed in Table 7, or a pharmaceutically acceptable salt thereof.
Paragraph 160. A pharmaceutical composition comprising a compound selected from:
(i) a compound of Formula (I) as recited in any one of paragraphs 1-28;
(ii) a compound of Formula (II) as recited in any one of paragraphs 29-53;
(iii) a compound of Formula (III) as recited in any one of paragraphs 54-78;
(iv) a compound of Formula (IV) as recited in any one of paragraphs 79-99;
(v) a compound of Formula (V) as recited in any one of paragraphs 100-139;
(vi) a compound of Formula (VI) as recited in any one of paragraphs 140-149;
(vii) a compound of Formula (VII) as recited in any one of paragraphs 150-159; and
(viii) any one of the compounds listed in Table A,
or a pharmaceutically acceptable salt thereof, and a pharmaceutically acceptable carrier.
Paragraph 161. A method selected from:
(a) increasing or maintaining levels of an NMNAT2 polypeptide in a cell; and/or
(b) increasing or maintaining levels of NAD in a cell;
the method comprising contacting the cell with an effective amount of a compound selected from:
(i) a compound of Formula (I) as recited in any one of paragraphs 1-28;
(ii) a compound of Formula (II) as recited in any one of paragraphs 29-53;
(iii) a compound of Formula (III) as recited in any one of paragraphs 54-78;
(iv) a compound of Formula (IV) as recited in any one of paragraphs 79-99;
(v) a compound of Formula (V) as recited in any one of paragraphs 100-139;
(vi) a compound of Formula (VI) as recited in any one of paragraphs 140-149;
(vii) a compound of Formula (VII) as recited in any one of paragraphs 150-159; and
(viii) any one of the compounds listed in Table A,
or a pharmaceutically acceptable salt thereof, or with a pharmaceutical composition of paragraph 160.
Paragraph 162. A method for increasing or maintaining levels of NAD within a cell within a mammal, wherein said method comprises administering, to said mammal, an effective amount of a compound selected from:
(i) a compound of Formula (I) as recited in any one of paragraphs 1-28;
(ii) a compound of Formula (II) as recited in any one of paragraphs 29-53;
(iii) a compound of Formula (III) as recited in any one of paragraphs 54-78;
(iv) a compound of Formula (IV) as recited in any one of paragraphs 79-99;
(v) a compound of Formula (V) as recited in any one of paragraphs 100-139;
(vi) a compound of Formula (VI) as recited in any one of paragraphs 140-149;
(vii) a compound of Formula (VII) as recited in any one of paragraphs 150-159; and
(viii) any one of the compounds listed in Table A,
or a pharmaceutically acceptable salt thereof, or a pharmaceutical composition of paragraph 160.
Paragraph 163. The method of any one of paragraphs 1-159, 161, or 162, wherein the cell is a neuron.
Paragraph 164. A method of treating a mammal having a disease, disorder, or condition responsive to an increase in NMNAT2 polypeptide levels within a cell, said method comprising administering, to said mammal, a compound selected from:
(i) a compound of Formula (I) as recited in any one of paragraphs 1-28;
(ii) a compound of Formula (II) as recited in any one of paragraphs 29-53;
(iii) a compound of Formula (III) as recited in any one of paragraphs 54-78;
(iv) a compound of Formula (IV) as recited in any one of paragraphs 79-99;
(v) a compound of Formula (V) as recited in any one of paragraphs 100-139;
(vi) a compound of Formula (VI) as recited in any one of paragraphs 140-149;
(vii) a compound of Formula (VII) as recited in any one of paragraphs 150-159; and
(viii) any one of the compounds listed in Table A,
or a pharmaceutically acceptable salt thereof, or a pharmaceutical composition of paragraph 160.
Paragraph 165. The method of any one of paragraphs 1-159 or 162-164, wherein said mammal is a human.
Paragraph 166. The method or any one of paragraphs 164-165, wherein said disease, disorder, or condition is a traumatic nerve injury.
Paragraph 167. The method of paragraph 166, wherein the traumatic nerve injury is selected from a traumatic neuronal crush injury, a traumatic brain injury, chronic traumatic encephalopathy (CTE), and concussion.
Paragraph 168. The method of any one of paragraphs 164-165, wherein said disease, disorder, or condition is a neuropathy.
Paragraph 169. The method of paragraph 168, wherein the neuropathy is selected from chemotherapeutic-induced sensory neuropathy, diabetic neuropathy, Leber hereditary optic neuropathy, primarily inflammatory neuropathy, multifocal motor neuropathy, and anti-myelin-associated glycoprotein (MAG) neuropathy.
Paragraph 170. The method of any one of paragraphs 164-165, wherein the disease, disorder, or condition is a neurodegenerative disease, disorder, or condition.
Paragraph 171. The method of paragraph 170, wherein the neurodegenerative disease, disorder, or condition is selected from Huntington's disease, Alzheimer's disease, Parkinson's disease, Friedreich's ataxia, Lewy body disease, spinal muscular atrophy, frontotemporal dementia, and cerebellar degeneration.
Paragraph 172. The method of paragraph 170, wherein the neurodegenerative disease, disorder, or condition is central demyelinating disorder.
Paragraph 173. The method of paragraph 172, wherein the central demyelinating disorder is selected from multiple sclerosis, amyotrophic lateral sclerosis, adrenoleukodystrophy, adrenomyeloneuropathy, neuromyelitis optica, and acute disseminated encephalomyelitis.
Paragraph 174. The method of paragraph 170, wherein the neurodegenerative disease, disorder, or condition is peripheral demyelinating disorder.
Paragraph 175. The method of paragraph 174, wherein the peripheral demyelinating disorder is selected from Charcot-Marie-Tooth disease and Guillain-Barre syndrome.
Paragraph 176. The method of any one of paragraphs 164-165, wherein the disease, disorder, or condition is selected from glaucoma, ischemic injury, retinal ischemia, optic nerve ischemia, chronic inflammatory demyelinating polyneuropathy, and stroke.
Paragraph 177. The method of any one of paragraphs 164-176, wherein the disease, disorder, or condition is chronic.
Paragraph 178. The method of any one of paragraphs 164-176, wherein the disease, disorder, or condition is acute.
Paragraph 179. The method of any one of paragraphs 164-178, wherein the method further comprises administering to the mammal at least one additional therapeutic agent useful in treating or preventing a disease, disorder, or condition responsive to an increase in NMNAT2 polypeptide levels within a cell.
Paragraph 180. The method of paragraph 179, wherein the additional therapeutic agent is selected from a diuretic, an anti-seizure drug, a drug to increase NAD levels, an analgesic, a corticosteroid, and a coma-inducing drug.
Paragraph 181. The method of paragraph 170, wherein the neurodegenerative disease, disorder, or conditions is dementia.
Paragraph 182. The method of paragraph 171, wherein the dementia is mild cognitive dementia, Alzheimer's dementia, frontotemporal dementia, vascular dementia, dementia with Lewy bodies, dementia pugilistica, or mixed dementia
Paragraph 183. The method of paragraph 170, wherein the neurodegenerative disease is mild cognitive dementia.
Paragraph 184. A compound selected from:
(i) a compound of Formula (I) as recited in any one of paragraphs 1-28;
(ii) a compound of Formula (II) as recited in any one of paragraphs 29-53;
(iii) a compound of Formula (III) as recited in any one of paragraphs 54-78;
(iv) a compound of Formula (IV) as recited in any one of paragraphs 79-99;
(v) a compound of Formula (V) as recited in any one of paragraphs 100-139;
(vi) a compound of Formula (VI) as recited in any one of paragraphs 140-149;
(vii) a compound of Formula (VII) as recited in any one of paragraphs 150-159; and
(viii) any one of the compounds listed in Table A,
or a pharmaceutically acceptable salt thereof.
Paragraph 185. A compound selected from any one of the compounds listed in Tables 1, 2, 2a, 3, 4, 5, 5a, 5b, 5c, 5d, 5e, 6, 7, and A, or a pharmaceutically acceptable salt thereof.

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A method for increasing or maintaining levels of NMNAT2 polypeptide within a cell within a mammal, wherein said method comprises administering, to said mammal, an effective amount of a compound of Formula (I): 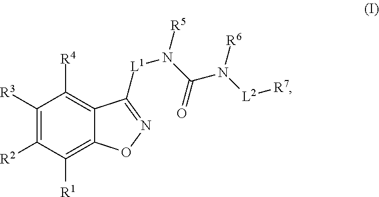
or a pharmaceutically acceptable salt thereof, wherein:
R¹, R², R³, and R⁴ are each independently selected from H, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
L¹ is selected from C₁₋₆ alkylene, C₂₋₆ alkenylene, and C₂₋₆ alkynylene, each of which is optionally substituted with 1, 2, or 3 substituents selected from R^{g};
R⁵ and R⁶ are each independently selected from H and C₁₋₆ alkyl;
L² is selected from C₁₋₆ alkylene, C₂₋₆ alkenylene, and C₂₋₆ alkynylene, each of which is optionally substituted with 1, 2, or 3 substituents selected from R^{g};
or L² is absent;
R⁷ is selected from H, C₁₋₆ alkyl, C₁₋₆ haloalkyl, C₂₋₆ alkenyl, C₂₋₆ alkynyl, Cy¹, halo, CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
or R⁷, L², and R⁶, together with the N atom to which R⁶ and L² are attached, form a 4-10 membered heterocycloalkyl ring, which is optionally substituted with 1, 2, or 3 substituents independently selected from R^{Cy1};
Cy¹ is selected from C₆₋₁₀ aryl, C₃₋₁₀ cycloalkyl, 5-14 membered heteroaryl, and 4-10 membered heterocycloalkyl, each of which is optionally substituted with 1, 2, 3, 4, or 5 substituents independently selected from R^{Cy1};
each R^{Cy1} is independently selected from halo, CN, NO₂, Cy², C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, NR^{c2}R^{d2} NR^{c2}C(O)R^{b2}, NR²C(O)OR^{a2}, NR²S(O)₂R^{b2}, S(O)₂R^{b2}, and S(O)₂NR^{c2}R^{d2}; wherein said C₁₋₆ alkyl, C₂₋₆ alkenyl, and C₂₋₆ alkynyl are each optionally substituted with 1, 2, or 3 substituents independently selected from Cy², halo, CN, NO₂, OR^{a1}, C(O)R^{b2}, C(O)NR^{c2}R^{d2}, C(O)OR^{a2}, NR^{c2}R^{d2}, NR^{c2}C(O)R^{b2} NR^{c2}C(O)OR^{a2}, NR²S(O)₂R^{b2}, S(O)₂R^{b2}, and S(O)₂NR^{c2}R^{d2};
each Cy² is independently selected from C₆₋₁₀ aryl, C₃₋₁₀ cycloalkyl, 5-10 membered heteroaryl, and 4-10 membered heterocycloalkyl, each of which is optionally substituted with 1, 2, 3, 4, or 5 substituents independently selected from R^{Cy2};
each R^{Cy2} is independently selected from halo, CN, NO₂, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}S(O)₂R^{b2}, S(O)₂R^{b2}, and S(O)₂NR^{c2}R^{d2}; wherein said C₁₋₆ alkyl, C₂₋₆ alkenyl, and C₂₋₆ alkynyl are each optionally substituted with 1, 2, or 3 substituents independently selected from halo, CN, NO₂, OR^{a2}, C(O)R^{b2}, C(O)NR^{c2}R^{d2}, C(O)OR^{a2}, NR^{c2}R^{d2}, NR^{c2}C(O)R^{b2}, NR^{c2}C(O)OR^{a2}, NR^{c2}S(O)₂R^{b2}, S(O)₂R^{b2}, and S(O)₂NR^{c2}R^{d2};
each R^{a1}, R^{b1}, R^{c1}, R^{d1}, R^{a2}, R^{b2}, R^{c2}, and R^{d2} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};
or any R^{c1} and R^{d1} together with the N atom to which they are attached form a 4-7 membered heterocycloalkyl, which is optionally substituted with 1, 2, or 3 substituents independently selected from R^{g};
or any R^{c2} and R^{d2} together with the N atom to which they are attached form a 4-7 membered heterocycloalkyl, which is optionally substituted with 1, 2, or 3 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

2. The method of claim 1, wherein:
R¹, R², and R⁴ are each H; and R³ is selected from H, halo, C₁₋₆ alkyl, C₁₋₆ haloalkyl, C₁₋₆ alkoxy, and C₁₋₆ haloalkoxy;
L¹ is C₁₋₆ alkylene, which is optionally substituted with 1, 2, or 3 substituents selected from OH, NO₂, CN, halo, C₁₋₆ alkoxy, and C₁₋₆ haloalkoxy;
R⁵ is H;
R⁶ is selected from H and C₁₋₆ alkyl;
L² is C₁₋₆ alkylene, which is optionally substituted with 1, 2, or 3 substituents selected from OH, NO₂, CN, halo, C₁₋₆ alkoxy, and C₁₋₆ haloalkoxy;
or L² is absent;
R⁷ is selected from Cy¹, OR^{a1}, and NR^{c1}R^{d1};
Cy¹ is selected from: (i) C₆₋₁₀ aryl, optionally substituted with 1, 2, or 3 substituents independently selected from halo, CN, C₁₋₆ alkyl, C₁₋₆ haloalkyl, C₁₋₆ alkoxy, C₁₋₆ alkylthio, and C(O)C₁₋₆ alkyl; (ii) C₃₋₁₀ cycloalkyl, optionally substituted with 1 or 2 C₁₋₆ alkyl; and (iii) 4-10 membered heterocycloalkyl, optionally substituted with C₆₋₁₀ aryl-C₁₋₆ alkylene; and
R^{a1}, R^{c1}, and R^{d1} are each independently selected from H, C₁₋₆ alkyl, and C₆₋₁₀ aryl-C₁₋₄ alkylene.

3. The method of claim 1, wherein the compound is selected from any one of the compounds listed in Table 1, or a pharmaceutically acceptable salt thereof.

4. A method for increasing or maintaining levels of NMNAT2 polypeptide within a cell within a mammal, wherein said method comprises administering, to said mammal, an effective amount of a compound of Formula (II): 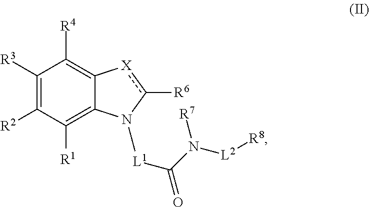
or a pharmaceutically acceptable salt thereof, wherein:
R¹, R², R³, and R⁴ are each independently selected from H, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1} NR^{c1}S(O)₂R^{b1}, S(O)₂R^{b1}, and S(O)₂NR^{c1}R^{d1}; [symbol]
is a single bond or a double bond; wherein:
(i) when [symbol] is a double bond, R⁶ is selected from H, C₁₋₆ alkyl, and Cy, and X is selected from N and CR⁵; and
(ii) when [symbol] is a single bond, R⁶ is oxo, X is CR⁵, and R⁵ and R⁴, together with the carbon atoms to which they are attached, form C₆₋₁₀ aryl ring or 5-14 membered heteroaryl ring, each of which is optionally substituted with 1, 2, or 3 substituents independently selected from R^{g};
R⁵ is selected from H, halo, CN, NO₂, C₁₋₆ alkyl, C₁₋₆ haloalkyl, C₂₋₆ alkenyl, C₂₋₆ alkynyl, OR^{a1}, C(O)R^{b1}, C(O)NR^{c1}R^{d1}, C(O)C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
L¹ is selected from C₁₋₆ alkylene, C₂₋₆ alkenylene, and C₂₋₆ alkynylene, each of which is optionally substituted with 1, 2, or 3 substituents selected from R^{g};
R⁷ is selected from H and C₁₋₆ alkyl;
L² is selected from C₁₋₆ alkylene, C₂₋₆ alkenylene, and C₂₋₆ alkynylene, each of which is optionally substituted with 1, 2, or 3 substituents selected from R^{g};
or L² is absent;
R⁸ is selected from H, OR^{a1}, C₆₋₁₀ aryl and 5-10 membered heteroaryl, each of which is optionally substituted with 1, 2, 3, 4, or 5 substituents independently selected from R^{g};
or R⁷, L², and R⁸, together with the N atom to which R⁷ and L² are attached form a 4-7 membered heterocycloalkyl, which is optionally substituted with C(O)Cy and is optionally substituted with 1, 2, or 3 substituents independently selected from R^{g};
Cy is selected from C₆₋₁₀ aryl and 5-10 membered heteroaryl, each of which is optionally substituted with 1, 2, 3, 4, or 5 substituents independently selected from R^{g};
each R^{a1}, R^{b1}, R^{c1}, and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};
or any R^{c1} and R^{d1} together with the N atom to which they are attached form a 4-7 membered heterocycloalkyl, which is optionally substituted with 1, 2, or 3 substituents independently selected from R^{g};
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

5. The method of claim 4, wherein R⁷, L², and R⁸, together with the N atom to which R⁷ and L² are attached form a 4-7 membered heterocycloalkyl, which is optionally substituted with 1, 2, or 3 substituents independently selected from R^{g}.

6. The method of claim 4, wherein:
R¹, R², R³, and R⁴ are each independently selected from H, halo, C₁₋₆ alkyl, and OR^{a1};
(i) when [symbol] is a double bond, R⁶ is selected from H, C₁₋₆ alkyl, and 5-10 membered heteroaryl which is optionally substituted with 1, 2, or 3 independently selected R^{g}, and X is selected from N and CR⁵;
(ii) when [symbol] is a single bond, R⁶ is oxo, X is CR⁵, and R⁵ and R⁴, together with the carbon atoms to which they are attached, form C₆₋₁₀ aryl ring, optionally substituted with 1, 2, or 3 substituents independently selected from R^{g};
R⁵ is selected from H, halo, C₁₋₆ alkyl, C₁₋₆ haloalkyl, OR^{a1}, C(O)R^{b1}, C(O)NR^{c1}R^{d1}, C(O)OR^{a1}, and NR^{c1}R^{d1} wherein said C₁₋₆ alkyl is optionally substituted with 1, 2, or 3 substituents independently selected from OR^{a1}, C(O)R^{b1}, C(O)NR^{c1}R^{a1}, C(O)OR^{a1}, and NR^{c1}R^{d1};
each R^{a1}, R^{b1}, Rd, and R^{d1} is independently selected from H, C₁₋₆ alkyl, C₁₋₄ haloalkyl, C₆₋₁₀ aryl, and C₃₋₁₀ cycloalkyl;
L¹ is C₁₋₆ alkylene, optionally substituted with 1, 2, or 3 substituents selected from R^{g};
R⁷ is H;
L² is C₁₋₆ alkylene, optionally substituted with 1, 2, or 3 substituents selected from R^{g};
R⁸ is selected from H, C₁₋₆ alkoxy, C₆₋₁₀ aryl and 5-10 membered heteroaryl, each of which is optionally substituted with 1, 2, or 3 substituents independently selected from R^{g}; and
R^{g} is selected from halo, C₁₋₆ alkyl, and C₁₋₆ alkoxy.

7. The method of claim 4, wherein the compound is selected from any one of the compounds listed in Table 2 or Table 2a, or a pharmaceutically acceptable salt thereof.

8. The method of claim 4, wherein the compound is selected from any one of the compounds listed in Table 2, or a pharmaceutically acceptable salt thereof.

9. A method for increasing or maintaining levels of NMNAT2 polypeptide within a cell within a mammal, wherein said method comprises administering, to said mammal, an effective amount of a compound of Formula (III): 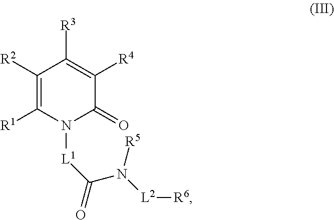
or a pharmaceutically acceptable salt thereof, wherein:
R¹, R², and R³ are each independently selected from H, halo, C₁₋₆ alkyl, C₁₋₆ haloalkyl, C₁₋₆ alkoxy, and C₁₋₆ haloalkoxy;
R⁴ is CN;
or R⁴ and R³, together with the carbon atoms to which they are attached, form a C₆₋₁₀ aryl ring or 5-10 membered heteroaryl ring, each of which is optionally substituted with 1, 2, or 3 substituents independently selected from R⁷;
each R⁷ is independently selected from halo, CN, C₁₋₆ alkyl, C₁₋₆ haloalkyl, C₁₋₆ alkoxy, and C₁₋₆ haloalkoxy;
L¹ is selected from C₁₋₆ alkylene, C₂₋₆ alkenylene, and C₂₋₆ alkynylene, each of which is optionally substituted with 1, 2, or 3 substituents selected from R^{g};
R⁵ is selected from H and C₁₋₆ alkyl;
L² is selected from C₁₋₆ alkylene, C₂₋₆ alkenylene, and C₂₋₆ alkynylene, each of which is optionally substituted with 1, 2, or 3 substituents selected from R^{g};
or L² is absent;
R⁶ is selected from H, C₆₋₁₀ aryl and 5-10 membered heteroaryl, each of which is optionally substituted with 1, 2, 3, 4, or 5 substituents independently selected from R⁸; provided that when L² is absent, then R⁶ is not H;
each R⁸ is independently selected from halo, CN, NO₂, C₁₋₆ alkyl, C₁₋₆ haloalkyl, C₂₋₆ alkenyl, C₂₋₆ alkynyl, OR^{a1}, SR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2, or 3 substituents independently selected from CN, NO₂, OR^{a1}, C(O)R^{b1}, C(O)NR^{c1}R^{d1}, C(O)OR^{a1}, NR^{c1}R^{a1}, NR^{c1}C(O)R^{b1}, NR^{c1}C(O)OR^{a1}, NR^{c1}S(O)₂R^{b1}, S(O)₂R^{b1}, and S(O)₂NR^{c1}R^{d1};
each R^{a1}, R^{b1}, R^{c1}, and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, and (4-10 membered heterocycloalkyl)-C₁₋₄ alkylene are each optionally substituted with 1, 2, 3, 4, or 5 substituents independently selected from R^{g};
or any R^{c1} and R^{d1} together with the N atom to which they are attached form a 4-7 membered heterocycloalkyl, which is optionally substituted with 1, 2, or 3 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

10. The method of claim 9, wherein the compound of Formula (III) has any one of the following formulae: 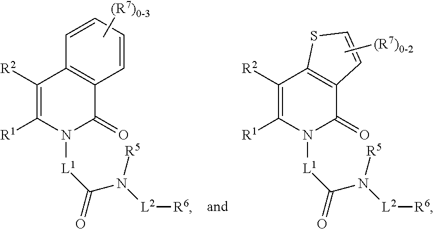
or a pharmaceutically acceptable salt thereof.

11. The method of claim 10, wherein:
R¹ and R² are each independently selected from H and C₁₋₆ alkyl;
R⁷ is selected from C₁₋₆ alkyl, C₁₋₆ haloalkyl, C₁₋₆ alkoxy, and C₁₋₆ haloalkoxy;
L¹ is C₁₋₆ alkylene, optionally substituted with 1, 2, or 3 substituents selected from R^{g};
R⁵ is H;
L² is absent or C₁₋₆ alkylene, optionally substituted with 1, 2, or 3 substituents selected from R^{g}, and R⁶ is selected from C₆₋₁₀ aryl and 5-10 membered heteroaryl, each of which is optionally substituted with 1, 2, or 3 substituents independently selected from R⁸;
or L² is C₁₋₆ alkylene, optionally substituted with 1, 2, or 3 substituents independently selected from R^{g}, and R⁶ is H;
R⁸ is selected from halo, C₁₋₆ alkyl, C₁₋₆ haloalkyl, OR^{a1}, SR^{a1}, C(O)R^{b1}, C(O)NR^{c1}R^{d1}, C(O)OR^{a1}, NR^{c1}R^{d1}, and NR^{c1}C(O)R^{b1}; and
each R^{a1}, R^{b1}, R^{c1}, and R^{c1} is independently selected from H, C₁₋₆ alkyl, C₁₋₄ haloalkyl, C₆₋₁₀ aryl-C₁₋₄ alkylene, C₃₋₁₀ cycloalkyl-C₁₋₄ alkylene, and (5-10 membered heteroaryl)-C₁₋₄ alkylene.

12. The method of claim 9, wherein the compound of Formula (III) has formula: 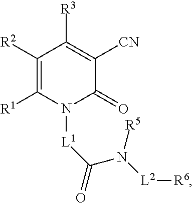
or a pharmaceutically acceptable salt thereof.

13. The method of claim 12, wherein:
R¹, R², and R³ are each independently selected from H, and C₁₋₆ alkyl;
L¹ is C₁₋₆ alkylene, optionally substituted with 1, 2, or 3 substituents selected from R^{g};
R⁵ is H;
L² is absent or C₁₋₆ alkylene, optionally substituted with 1, 2, or 3 substituents selected from R^{g}, and R⁶ is selected from C₆₋₁₀ aryl and 5-10 membered heteroaryl, each of which is optionally substituted with 1, 2, or 3 substituents independently selected from R⁸;
or L² is C₁₋₆ alkylene, optionally substituted with 1, 2, or 3 substituents independently selected from R^{g}, and R⁶ is H;
R⁸ is selected from halo, C₁₋₆ alkyl, C₁₋₆ haloalkyl, OR^{a1}, SR^{a1}, C(O)R^{b1}, C(O)NR^{c1}R^{d1}, C(O)OR^{a1}, NR^{c1}R^{d1}, and NR^{c1}C(O)R^{b1}; and
each R^{a1}, R^{b1}, R^{c1}, and R^{d1} is independently selected from H, C₁₋₆ alkyl, C₁₋₄ haloalkyl, C₆₋₁₀ aryl-C₁₋₄ alkylene, C₃₋₁₀ cycloalkyl-C₁₋₄ alkylene, and (5-10 membered heteroaryl)-C₁₋₄ alkylene.

14. The method of claim 9, wherein the compound is selected from any one of the compounds listed in Table 3, or a pharmaceutically acceptable salt thereof.

15. A method for increasing or maintaining levels of NMNAT2 polypeptide within a cell within a mammal, wherein said method comprises administering, to said mammal, an effective amount of a compound of Formula (IV): 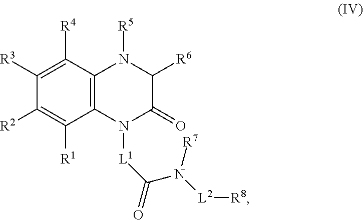
or a pharmaceutically acceptable salt thereof, wherein:
R¹, R², R³, and R⁴ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, and C₁₋₆ haloalkoxy;
R⁵ is selected from C₁₋₆ alkyl, C₁₋₆ haloalkyl, C₂₋₆ alkenyl, and C₂₋₆ alkynyl, each of which is optionally substituted with 1, 2, or 3 substituents independently selected from R^{g};
R⁶ is oxo;
or R⁵ and R⁶, together with N atom to which R⁵ is attached and carbon atom to which R⁶ is attached, form a 4-10 membered heterocycloalkyl ring or a 5-10 membered heteroaryl ring, each of which is optionally substituted with 1, 2, or 3 independently selected R^{g};
L¹ is selected from C₁₋₆ alkylene, C₂₋₆ alkenylene, and C₂₋₆ alkynylene, each of which is optionally substituted with 1, 2, or 3 substituents selected from R^{g};
R⁷ is selected from H and C₁₋₆ alkyl;
L² is selected from C₁₋₆ alkylene, C₂₋₆ alkenylene, and C₂₋₆ alkynylene, each of which is optionally substituted with 1, 2, or 3 substituents selected from R^{g};
R⁸ is 5-10 membered heteroaryl, optionally substituted with 1, 2, 3, 4, or 5 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

16. The method of claim 15, wherein the compound of Formula (IV) has formula: 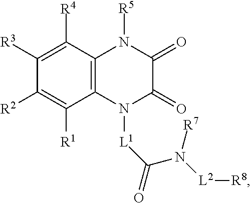
or a pharmaceutically acceptable salt thereof.

17. The method of claim 16, wherein:
R¹, R², R³, and R⁴ are each independently selected from H and halo;
R⁵ is C₁₋₆ alkyl;
L¹ is C₁₋₆ alkylene, optionally substituted with 1, 2, or 3 substituents selected from R^{g};
R⁷ is H;
L² is C₁₋₆ alkylene, optionally substituted with 1, 2, or 3 substituents selected from R^{g};
R⁸ is 5-10 membered heteroaryl, optionally substituted with 1, 2, or 3 substituents independently selected from R^{g}; and
R^{g} is selected from OH, NO₂, CN, halo, C₁₋₆ alkyl, C₁₋₄ haloalkyl, C₁₋₆ alkoxy, and C₁₋₆ haloalkoxy.

18. The method of claim 15, wherein the compound of Formula (IV) has formula: 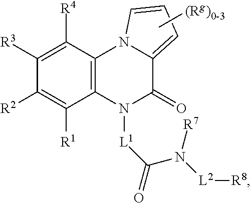
or a pharmaceutically acceptable salt thereof.

19. The method of claim 18, wherein:
R¹, R², R³, and R⁴ are each independently selected from H and halo;
L¹ is C₁₋₆ alkylene, optionally substituted with 1, 2, or 3 substituents selected from R^{g};
R⁷ is H;
L² is C₁₋₆ alkylene, optionally substituted with 1, 2, or 3 substituents selected from R^{g};
R⁸ is 5-10 membered heteroaryl, optionally substituted with 1, 2, or 3 substituents independently selected from R^{g}; and
R^{g} is selected from OH, NO₂, CN, halo, C₁₋₆ alkyl, C₁₋₄ haloalkyl, C₁₋₆ alkoxy, and C₁₋₆ haloalkoxy.

20. The method of claim 15, wherein the compound of Formula (IV) is selected from any one of the compounds listed in Table 4, or a pharmaceutically acceptable salt thereof.

21. A method for increasing or maintaining levels of NMNAT2 polypeptide within a cell within a mammal, wherein said method comprises administering, to said mammal, an effective amount of a compound of Formula (V): 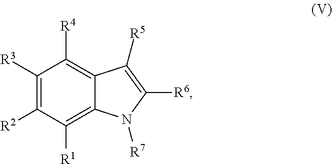
or a pharmaceutically acceptable salt thereof, wherein:
R¹, R², R³, R⁴, R⁵, and R⁶ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, C₁₋₆ haloalkoxy, wherein said C₁₋₆ alkyl is optionally substituted with 1, 2, or 3 substituents independently selected from OH, NO₂, CN, C₆₋₁₀ alkoxy, C₆₋₁₀ haloalkoxy, and Cy, and a moiety of formula (i): 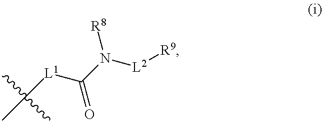
provided that at least one of R¹, R², R³, R⁴, R⁵, and R⁶ is a moiety of formula (i);
or R⁵ and R⁶ together with the carbon atoms to which they are attached from a C₄₋₇ cycloalkyl ring or a 4-7 membered heterocycloalkyl ring, each of which is optionally substituted with 1, 2, or 3 substituents independently selected from OH, NO₂, CN, C₆₋₁₀ alkoxy, C₆₋₁₀ haloalkoxy, Cy, and a moiety of formula (i);
or when R⁴ is a moiety of formula (i), the R⁸ of the moiety of formula (i) and R⁵, together with the N atom to which R⁸ is attached and the carbon atom to which R⁵ is attached, form a 4-10 membered heterocycloalkyl, which is optionally substituted with 1, 2, 3, 4, or 5 substituents independently selected from R^{g};
R⁷ is selected from C₁₋₆ alkyl, C₁₋₆ haloalkyl, C₂₋₆ alkenyl, and C₂₋₆ alkynyl, wherein said C₁₋₆ alkyl is optionally substituted with 1, 2, or 3 substituents independently selected from OH, NO₂, CN, C₆₋₁₀ alkoxy, C₆₋₁₀ haloalkoxy, Cy, and a moiety of formula (i);
L¹ is selected from C(O), C₁₋₆ alkylene, C(O)—C₁₋₆ alkylene, C₃₋₁₀ cycloalkylene, C₂₋₆ alkenylene, and C₂₋₆ alkynylene, each of which is optionally substituted with 1, 2, or 3 substituents independently selected from R¹⁰; or L¹ is absent;
each R¹⁰ is independently selected from halo, OH, NO₂, CN, C₆₋₁₀ alkoxy, C₆₋₁₀ haloalkoxy, and Cy;
R⁸ is selected from H and C₁₋₆ alkyl, wherein said C₁₋₆ alkyl is optionally substituted with 1, 2, or 3 substituents independently selected from C₁₋₆ alkoxy, amino, C₁₋₆ alkylamino, di(C₁₋₆ alkyl)amino, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, 3, 4, or 5 substituents independently selected from R^{g};
or R⁸ and R¹⁰ together with the N atom to which R⁸ is attached, L¹ to which R¹⁰ is attached, and C(O) between the N and the L¹, form a 4-10 membered heterocycloalkyl, which is optionally substituted with 1, 2, 3, 4, or 5 substituents independently selected from R^{g};
L² is selected from C₁₋₆ alkylene, C₁₋₆ alkylene-NR⁸—C₁₋₆ alkylene, C₂₋₆ alkenylene, and C₂₋₆ alkynylene, each of which is optionally substituted with 1, 2, or 3 substituents independently selected from R^{g}; or L² is absent;
R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, 3, 4, or 5 substituents independently selected from R^{g};
or R⁸ and L²-R⁹, together with the N atom to which R⁸ and L² are attached, form a 4-10 membered heterocycloalkyl, which is optionally substituted with C(O)Cy and is optionally substituted with 1, 2, 3, 4, or 5 substituents independently selected from R^{g};
or R⁹ and R¹⁰, together with the L¹ to which R¹⁰ is attached, L² to which R⁹ is attached, and C(O) and NR⁸ between the L¹ and the L², form a 4-10 membered heterocycloalkyl, which is optionally substituted with 1, 2, 3, 4, or 5 substituents independently selected from R^{g};
Cy is selected from C₆₋₁₀ aryl and 5-10 membered heteroaryl, each of which is optionally substituted with 1, 2, 3, 4, or 5 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, NH₂-C₁₋₃ alkylene, C₁₋₆ alkylamino-C₁₋₃ alkylene, di(C₁₋₆ alkyl)amino-C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

22. The method of claim 21, wherein:
R⁷ is selected from C₁₋₆ alkyl, C₁₋₆ haloalkyl, C₂₋₆ alkenyl, and C₂₋₆ alkynyl, wherein said C₁₋₆ alkyl is optionally substituted with 1, 2, or 3 substituents independently selected from OH, NO₂, CN, C₆₋₁₀ alkoxy, C₆₋₁₀ haloalkoxy, and Cy;
L¹ is selected from C(O), C₁₋₆ alkylene, C₂₋₆ alkenylene, and C₂₋₆ alkynylene, each of which is optionally substituted with 1, 2, or 3 substituents independently selected from halo, OH, NO₂, CN, C₆₋₁₀ alkoxy, C₆₋₁₀ haloalkoxy, and Cy; or L¹ is absent;
R⁸ is selected from H and C₁₋₆ alkyl;
L² is selected from C₁₋₆ alkylene, C₂₋₆ alkenylene, and C₂₋₆ alkynylene, each of which is optionally substituted with 1, 2, or 3 substituents independently selected from R^{g};
R⁹ is selected from H, C₁₋₆ alkoxy, C₆₋₁₀ aryl and 5-10 membered heteroaryl, each of which is optionally substituted with 1, 2, 3, 4, or 5 substituents independently selected from R^{g};
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

23. The method of claim 21, wherein the compound of Formula (V) has formula: 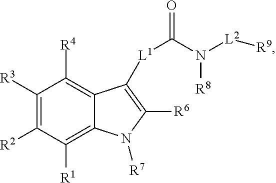
or a pharmaceutically acceptable salt thereof.

24. The method of claim 23, wherein:
R¹, R², R³, R⁴, and R⁶ are each H;
R⁷ is C₁₋₆ alkyl, optionally substituted with Cy or a moiety of formula (i);
L¹ is selected from C₁₋₆ alkylene, C(O)—C₁₋₆ alkylene, and C₃₋₁₀ cycloalkylene, each of which is optionally substituted with R¹⁰; or L¹ is absent;
each R¹⁰ is Cy;
R⁸ is selected from H and C₁₋₆ alkyl, which is optionally substituted with C₁₋₆ alkoxy, di(C₁₋₆ alkyl)amino, or 4-10 membered heterocycloalkyl;
L² is C₁₋₆ alkylene or C₁₋₆ alkylene-NR⁸—C₁₋₆ alkylene; or L² is absent;
Cy is selected from C₆₋₁₀ aryl and 5-10 membered heteroaryl, each of which is optionally substituted with 1, 2, or 3 independently selected R^{g}; and
R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

25. The method of claim 21, wherein the compound of Formula (V) has formula: 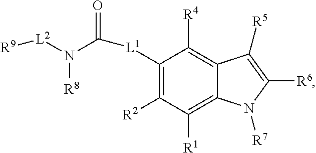
or a pharmaceutically acceptable salt thereof.

26. The method of claim 25, wherein:
R¹, R², and R⁴ are each H, and R⁵ and R⁶ are each independently selected from H and C₁₋₆ alkyl;
R⁷ is C₁₋₆ alkyl, optionally substituted with Cy or a moiety of formula (i);
L¹ is selected from C(O), C₁₋₆ alkylene, C(O)—C₁₋₆ alkylene, and C₃₋₁₀ cycloalkylene, each of which is optionally substituted with R¹⁰; or L¹ is absent;
each R¹⁰ is Cy;
R⁸ is selected from H and C₁₋₆ alkyl, which is optionally substituted with C₁₋₆ alkoxy, di(C₁₋₆ alkyl)amino, or 4-10 membered heterocycloalkyl;
L² is C₁₋₆ alkylene or C₁₋₆ alkylene-NR⁸—C₁₋₆ alkylene; or L² is absent;
Cy is selected from C₆₋₁₀ aryl and 5-10 membered heteroaryl, each of which is optionally substituted with 1, 2, or 3 independently selected R^{g}; and
R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

27. The method of claim 21, wherein the compound of Formula (V) has formula: 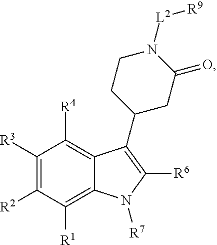
or a pharmaceutically acceptable salt thereof.

28. The method of claim 27, wherein:
R¹, R², R³, R⁴, and R⁶ are each H;
R⁷ is C₁₋₆ alkyl, optionally substituted with Cy or a moiety of formula (i);
L² is C₁₋₆ alkylene or C₁₋₆ alkylene-NR⁸—C₁₋₆ alkylene; or L² is absent;
R⁸ is selected from H and C₁₋₆ alkyl, which is optionally substituted with C₁₋₆ alkoxy, di(C₁₋₆ alkyl)amino, or 4-10 membered heterocycloalkyl;
Cy is selected from C₆₋₁₀ aryl and 5-10 membered heteroaryl, each of which is optionally substituted with 1, 2, or 3 independently selected R^{g}; and
R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

29. The method of claim 21, wherein the compound of Formula (V) has any one of the following formulae:

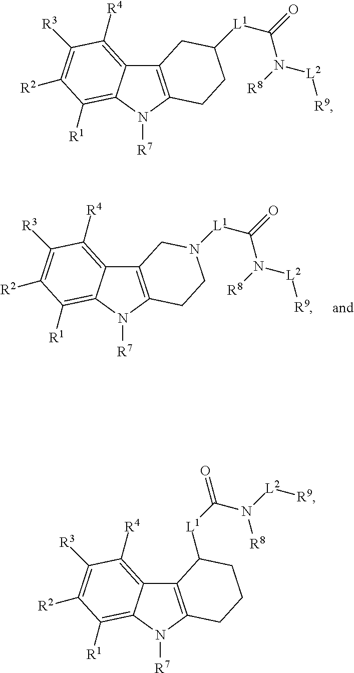

30. The method of claim 29, wherein:
R¹, R², R³, and R⁴ are each H;
R⁷ is C₁₋₆ alkyl, optionally substituted with Cy or a moiety of formula (i);
L¹ is selected from C(O), C₁₋₆ alkylene, C(O)—C₁₋₆ alkylene, and C₃₋₁₀ cycloalkylene, each of which is optionally substituted with R¹⁰; or L¹ is absent;
each R¹⁰ is Cy;
R⁸ is selected from H and C₁₋₆ alkyl, which is optionally substituted with C₁₋₆ alkoxy, di(C₁₋₆ alkyl)amino, or 4-10 membered heterocycloalkyl;
L² is C₁₋₆ alkylene or C₁₋₆ alkylene-NR⁸—C₁₋₆ alkylene; or L² is absent;
Cy is selected from C₆₋₁₀ aryl and 5-10 membered heteroaryl, each of which is optionally substituted with 1, 2, or 3 independently selected R^{g}; and
R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

31. The method of claim 21, wherein the compound of Formula (V) has formula: 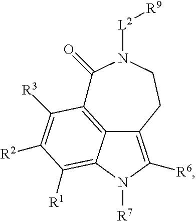
or a pharmaceutically acceptable salt thereof.

32. The method of claim 31, wherein:
R¹, R², R³, and R⁶ are each H;
R⁷ is C₁₋₆ alkyl, optionally substituted with Cy or a moiety of formula (i);
L² is C₁₋₆ alkylene or C₁₋₆ alkylene-NR⁸—C₁₋₆ alkylene; or L² is absent;
R⁸ is selected from H and C₁₋₆ alkyl, which is optionally substituted with C₁₋₆ alkoxy, di(C₁₋₆ alkyl)amino, or 4-10 membered heterocycloalkyl;
Cy is selected from C₆₋₁₀ aryl and 5-10 membered heteroaryl, each of which is optionally substituted with 1, 2, or 3 independently selected R^{g}; and
R⁹ is selected from H, C₁₋₆ alkoxy, C₆₋₁₀ aryl, C₆₋₁₀ aryloxy, 5-10 membered heteroaryl, 5-10 membered heteroaryloxy, and 4-10 membered heterocycloalkyl, each of which is optionally substituted with 1, 2, or 3 substituents independently selected from R^{g}.

33. The method of claim 21, wherein the compound of Formula (V) is selected from any one of the compounds listed in Table 5, Table 5a, Table 5b, Table 5c, Table 5d, and Table 5e, or a pharmaceutically acceptable salt thereof.

34. The method of claim 21, wherein the compound of Formula (V) is selected from any one of the compounds listed in Table 5, or a pharmaceutically acceptable salt thereof.

35. A method for increasing or maintaining levels of NMNAT2 polypeptide within a cell within a mammal, wherein said method comprises administering, to said mammal, an effective amount of a compound of Formula (VI): 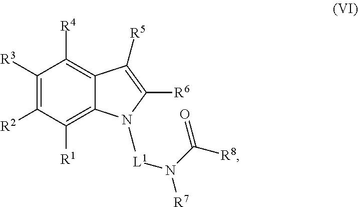
or a pharmaceutically acceptable salt thereof, wherein:
R¹, R², R³, R⁴, R⁵, and R⁶ are each independently selected from H, halo, CN, NO₂, OH, C₁₋₆ alkyl, C₁₋₆ haloalkyl, C₂₋₆ alkenyl, C₂₋₆ alkynyl, C₁₋₆ alkoxy, and C₁₋₆ haloalkoxy;
L¹ is selected from C₁₋₆ alkylene, C₂₋₆ alkenylene, and C₂₋₆ alkynylene, each of which is optionally substituted with 1, 2, or 3 substituents independently selected from R^{g};
R⁷ is selected from H and C₁₋₆ alkyl;
R⁹ is selected from C₆₋₁₀ aryl and 5-10 membered heteroaryl, each of which is optionally substituted with 1, 2, 3, 4, or 5 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

36. The method of claim 35, wherein:
R¹, R², R³, R⁴, R⁵, and R⁶ are each independently selected from H, halo, and C₁₋₆ alkoxy;
L¹ is C₁₋₆ alkylene;
R⁷ is H; and
R⁹ is 5-10 membered heteroaryl, optionally substituted with 1, 2, or 3 substituents independently selected from R^{g}.

37. The method of claim 35, wherein the compound of Formula (VI) is selected from any one of the compounds listed in Table 6, or a pharmaceutically acceptable salt thereof.

38. A method for increasing or maintaining levels of NMNAT2 polypeptide within a cell within a mammal, wherein said method comprises administering, to said mammal, an effective amount of a compound of Formula (VII): 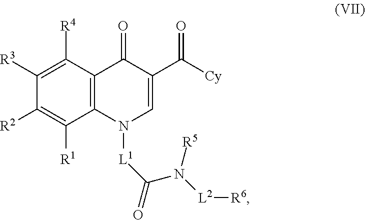
or a pharmaceutically acceptable salt thereof, wherein:
R¹, R², R³, and R⁴ are each independently selected from H, halo, C₁₋₆ alkyl, C₁₋₆ haloalkyl, C₁₋₆ alkoxy, and C₁₋₆ haloalkoxy;
Cy is selected from C₆₋₁₀ aryl and 5-10 membered heteroaryl, each of which is optionally substituted with 1, 2, 3, 4, or 5 substituents independently selected from R^{g};
L¹ is selected from C₁₋₆ alkylene, C₂₋₆ alkenylene, and C₂₋₆ alkynylene, each of which is optionally substituted with 1, 2, or 3 substituents selected from R^{g};
R⁵ is selected from H and C₁₋₆ alkyl;
L² is selected from C₁₋₆ alkylene, C₂₋₆ alkenylene, and C₂₋₆ alkynylene, each of which is optionally substituted with 1, 2, or 3 substituents independently selected from R^{g};
R⁶ is selected from C₆₋₁₀ aryl and 5-10 membered heteroaryl, each of which is optionally substituted with 1, 2, 3, 4, or 5 substituents independently selected from R^{g}; and
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, C₆₋₁₀ aryl, C₆₋₁₀ aryloxy, C₃₋₁₀ cycloalkyl, 5-10 membered heteroaryl, 4-10 membered heterocycloalkyl, C₆₋₁₀ aryl-C₁₋₄ alkylene, C₃₋₁₀ cycloalkyl-C₁₋₄ alkylene, (5-10 membered heteroaryl)-C₁₋₄ alkylene, (4-10 membered heterocycloalkyl)-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

39. The method of claim 38, wherein:
R¹, R², R³, and R⁴ are each independently selected from H, halo, and C₁₋₆ alkyl;
Cy is 5-10 membered heteroaryl, optionally substituted with 1, 2, or 3 substituents independently selected from R^{g};
L¹ is C₁₋₆ alkylene, optionally substituted with 1, 2, or 3 substituents selected from R^{g};
R⁵ is H;
L² is C₁₋₆ alkylene, optionally substituted with 1, 2, or 3 substituents selected from R^{g};
R⁶ is C₆₋₁₀ aryl, optionally substituted with 1, 2, or 3 substituents independently selected from R^{g}; and
each R^{g} is independently selected from halo and C₁₋₆ alkyl.

40. The method of claim 38, wherein the compound of Formula (VII) is selected from any one of the compounds listed in Table 7, or a pharmaceutically acceptable salt thereof.

41. A pharmaceutical composition comprising a compound selected from:
(i) a compound of Formula (I) as recited in any one of claims 1-3;
(ii) a compound of Formula (II) as recited in any one of claims 4-8;
(iii) a compound of Formula (III) as recited in any one of claims 9-14;
(iv) a compound of Formula (IV) as recited in any one of claims 15-20;
(v) a compound of Formula (V) as recited in any one of claims 21-34;
(vi) a compound of Formula (VI) as recited in any one of claims 35-37;
(vii) a compound of Formula (VII) as recited in any one of claims 38-40; and
(viii) any one of the compounds listed in Table A,
or a pharmaceutically acceptable salt thereof, and a pharmaceutically acceptable carrier.

42. A method of treating a mammal having a disease, disorder, or condition responsive to an increase in NMNAT2 polypeptide levels within a cell, said method comprising administering, to said mammal, a compound selected from:
(i) a compound of Formula (I) as recited in any one of claims 1-3;
(ii) a compound of Formula (II) as recited in any one of claims 4-8;
(iii) a compound of Formula (III) as recited in any one of claims 9-14;
(iv) a compound of Formula (IV) as recited in any one of claims 15-20;
(v) a compound of Formula (V) as recited in any one of claims 21-34;
(vi) a compound of Formula (VI) as recited in any one of claims 35-37;
(vii) a compound of Formula (VII) as recited in any one of claims 38-40; and
(viii) any one of the compounds listed in Table A, or a pharmaceutically acceptable salt thereof, or a pharmaceutical composition of claim 41.

43. The method of claim 42, wherein said disease, disorder, or condition is selected from a traumatic nerve injury, a neuropathy, a neurodegenerative disease, disorder, or condition, a glaucoma, an ischemic injury, a retinal ischemia, an optic nerve ischemia, a chronic inflammatory demyelinating polyneuropathy, and a stroke.

44. The method of claim 43, wherein the neurodegenerative disease, disorder, or condition is selected from Huntington's disease, Alzheimer's disease, Parkinson's disease, Friedreich's ataxia, Lewy body disease, spinal muscular atrophy, frontotemporal dementia, cerebellar degeneration, and a demyelinating disorder.

45. The method of claim 42, wherein the method further comprises administering to the mammal at least one additional therapeutic agent selected from a diuretic, an anti-seizure drug, a drug to increase NAD levels, an analgesic, a corticosteroid, and a coma-inducing drug.

46. The method of claim 43, wherein the neurodegenerative disease, disorder, or conditions is dementia.

47. The method of claim 46, wherein the dementia is mild cognitive dementia, Alzheimer's dementia, frontotemporal dementia, vascular dementia, dementia with Lewy bodies, dementia pugilistica, or mixed dementia.

48. The method of claim 43, wherein the neurodegenerative disease is mild cognitive dementia.

49. A compound selected from:
(i) a compound of Formula (I) as recited in any one of claims 1-3;
(ii) a compound of Formula (II) as recited in any one of claims 4-8;
(iii) a compound of Formula (III) as recited in any one of claims 9-14;
(iv) a compound of Formula (IV) as recited in any one of claims 15-20;
(v) a compound of Formula (V) as recited in any one of claims 21-34;
(vi) a compound of Formula (VI) as recited in any one of claims 35-37;
(vii) a compound of Formula (VII) as recited in any one of claims 38-40; and
(viii) any one of the compounds listed in Table A,
or a pharmaceutically acceptable salt thereof.

50. A compound selected from any one of the compounds listed in Tables 1, 2, 2a, 3, 4, 5, 5a, 5b, 5c, 5d, 5e, 6, 7, and A, or a pharmaceutically acceptable salt thereof.
